(19) United States
(12) Patent Application Publication
(10) Pub. No.: US 2011/0098267 A1
(43) Pub. Date: Apr. 28, 2011

(54) TOPICAL FORMULATIONS FOR THE TREATMENT OF PSORIASIS

(75) Inventors: Suresh R. Babu, Superior Township, MI (US); Yumiko Wada, Billerica, MA (US); Jianhua Shen, Andover, MA (US); Nhung Nguyen, Westford, MA (US)

(73) Assignee: Synta Pharmaceuticals Corporation, Lexington, MA (US)

(21) Appl. No.: 12/866,735

(22) PCT Filed: Feb. 6, 2009
(86) PCT No.: PCT/US09/33495

§ 371 Date: Dec. 23, 2010

Related U.S. Application Data
(60) Provisional application No. 61/027,036, filed on Feb. 7, 2008.

(51) Int. Cl. A61K 31/513; A61K 31/5377; A61K 31/541; A61P 17/06

(52) U.S. Cl. 514/210.2; 514/236.2; 514/235.8; 514/235.2; 514/232.2; 514/235.5; 514/227.8

(57) ABSTRACT

The invention relates to a topical composition and a method for treating psoriasis. The topical composition comprises one or more compound that inhibits the production of IL-12 and IL-23.

### CROSS-REFERENCE TO RELATED APPLICATIONS

This application claims the benefit of U.S. Provisional Patent Application No. 61/027,036, filed Feb. 7, 2008, the contents of which are incorporated herein by reference.

### BACKGROUND

Interleukin-12 (IL-12) is a heterodimeric cytokine (p70) which plays key roles in immune responses by bridging innate resistance and antigen-specific adaptive immunity. Trinchieri (1993) Immunol Today 14: 335. For example, it promotes type 1 T helper cell (T_{H}1) responses and, hence, cell-mediated immunity. Chan et al. (1991) J Exp Med 173: 869; Seder et al. (1993) Proc Natl Acad Sci USA 90: 10188; Manetti et al. (1993) J Exp Med 177: 1199; and Hsieh et al. (1993) Science 260: 547. Interleukin-12 (IL-12) is a di-sulfide linked heterodimeric cytokine (p70) composed of two independently regulated subunits, p35 and p40. IL-12 is produced by phagocytic cells and antigen presenting cells, in particular, macrophages and dendritic cells, upon stimulation with bacteria, bacterial products such as lipopolysaccharide (LPS), and intracellular parasites. The well-documented biological functions of IL-12 are induction of interferon-γ expression from T and NK cells and differentiation toward the T_{H}1 T lymphocyte type. IFN-γ, expression of which is induced by IL-12, is a strong and selective enhancer of IL-12 production from monocytes and macrophages. The cytokine IL-23 is a heterodimer composed of a p19 subunit and the same p40 subunit of IL-12. IL-23, similarly to IL-12, is involved in type 1 immune defenses and induces IFN-γ secretion from T cells. IL-27 is formed by the association of EBI3, a polypeptide related to the p40 subunit of IL-12, and p28, a protein related to the p35 subunit of IL-12. IL-27 promotes the growth of T cells and is thought to play a role in the differentiation of T_{H}1 cells. Pflanz et al., Immunity (2002), 16:779-790.

It has been suggested that, particularly in chronic diseases in which there is ongoing production of IFN-γ, IL-12 production is augmented by IFN-γ. It is presumed that after an infective or inflammatory stimulus that provokes IL-12 production, the powerful feedback loop promotes IL-12- and IL-23-induced IFN-γ to further augment IL-12 production, leading to consequent excessive production of pro-inflammatory cytokines. Furthermore, it has been suggested that IL-27 induces the expression of T-bet, a major T_{H}1-specific transcription factor, and its downstream target IL-12R β2, independently of IFN-γ. In addition, IL-27 suppresses the expression of GATA-3. GATA-3 inhibits T_{H}1 development and causes loss of IL-12 signaling through suppression of IL-12R β2 and Stat4 expression. Lucas et al., PNAS (2003), 100:15047-15052.

Psoriasis is a T_{H}1 dominant autoimmune disease that effects about 0.6 to 4.8 percent of the population in the United States. Typical forms of psoriasis include including psoriasis vulgaris, guttate psoriasis, pustular psoriasis, inverse psoriasis, erythrodermic psoriasis, psoriasis arthropathica, psoriasis gravis, and psoriatic arthritis. IL-12 and IL-23 play a critical role in multiple-T_{H}1 dominant autoimmune diseases, and, in particular, have been shown to play a role in psoriasis. See, for example, Gately et al. (1998) Annu Rev Immunol. 16: 495; and Abbas et al. (1996) Nature 383: 787, and Lee et al., J. Exp. Med. (2004), 199(1):125-130.

Inhibiting production of IL-12, or inhibiting the production of cytokines such as IL-23 and IL-27 which promote IL-12 production and/or T_{H}1 development is an approach to treating psoriasis. Trembleau et al. (1995) Immunol. Today 16: 383; and Adorini et al. (1997) Chem. Immunol. 68: 175. For example, U.S. Pat. Nos. 6,384,032, 6,693,097, 7,067,514, 6,660,733, 6,958,332, and 6,858,606 claim compounds that inhibit the excessive or inappropriate production of IL-12 and/or IL-23 and thereby are useful for the treatment of disorders such as psoriasis which are relate to excess T_{H}1 type response. Although these compounds have been shown to be active against psoriasis when administered orally, a need exists for a topical formulation which would decrease systemic exposure to the drug and target the site of psoriatic skin lesions

### SUMMARY

The present invention addresses this need for effective, well tolerated treatements for psoriasis.

In one aspect, the invention provides a pharmaceutical composition for topical administration, comprising:

(a) a compound represented by formula (I):

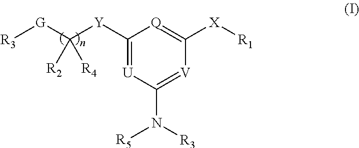

or a pharmaceutically acceptable salt thereof, wherein:
R₁ is optionally substituted aryl, optionally substituted heteroaryl, or a group represented by the following formula:

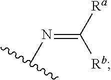

R₂ and R₄, for each occurrence, are independently, H, an optionally substituted alkyl, an optionally substituted alkylcarbonyl, —OR^{k}, —SR^{k}, —NR^{h}R^{j}, hydroxylalkyl, —C(O)R^{c}, —OC(O)R^{c}, —SC(O)R^{c}, —NR^{k}C(O)R^{c}, —C(S)R^{c}, —OC(S)R^{c}, —SC(S)R^{c}, —NR^{k}C(S)R^{c}, —C(NR)R^{c}, —OC(NR)R^{c}, —SC(NR)R^{c}, —NR^{k}C(NR)R^{c}, —SO₂R^{c}, —S(O)R^{c}, —NR^{k}SO₂R^{c}, —OS(O)₂R^{c}, —OP(O)R^{c}R^{c}, —P(O)R^{c}R^{c}, halo, haloalkyl, aminoalkyl, mercaptoalkyl, cyano, nitro, nitroso, azide, an optionally substituted alkylcarbonylalkyl, an optionally substituted cyclyl, an optionally substituted cycloalkyl, an optionally substituted heterocyclyl, an optionally substituted heterocycloalkyl, an optionally substituted aryl, an optionally substituted aralkyl, an optionally substituted heteroaryl, an optionally substituted heteroaralkyl, or isothionitro; or R₂ and R₄ taken together are ═O, ═S, or ═NR;

### R₃ is R^{g};

R₅ and R₆ are each, independently, H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl; or R₅ and R₆ taken together with the N to which they are attached is an optionally substituted heterocyclyl, an optionally substituted heterocycloalkyl, or an optionally substituted heteroaryl;
X is O, S, S(O), S(O)₂, or NR^{k};
Y is (CH(R^{g}))ₘ, C(O), C(NR), O, S, S(O), S(O)₂, N(R^{k}), or absent;
G is a bond, —C(O)NR^{k}NR^{k}—, —NR^{k}NR^{k}C(O)—, —NR^{k}N═CR^{k}—, —CR^{k}═NNR^{k}—, —NR^{k}NR^{k}—, —N(OH)—, —NR^{k}O—, —ONR^{k}—, —C(O)—, —C(NR)—, —NR^{k}C(O)—, —C(O)NR^{k}—, —OC(O)—, —C(O)O—, —OC(O)O—, —NR^{k}C(O)O—, —OC(O)NR^{k}—, —NR^{k}C(S)O—, —OC(S)NR^{k}—, —NR^{k}—C(NR)—NR^{k}—NR^{k}—C(O)—NR^{k}—, —NR^{k}—C(S)—NR^{k}—, —NR^{k}—S(O)₂—NR^{k}—, —P(O)(R^{c})—, —P(O)(R^{c})O—, —OP(O)(R^{c})—, —OP(O)(R^{c})O—, an optionally substituted cycloalkylene, an optionally substituted cyclylene, an optionally substituted heterocycloalkylene, an optionally substituted heterocyclylene, an optionally substituted arylene, an optionally substituted aralkylene, an optionally substituted heteroarylene, an optionally substituted heteroaralkylene, an optionally substituted heteroarylene-NR^{k}—, an optionally substituted heteroarylene-S—, an optionally substituted heteroaralkylene-O—, —Si(OR^{k})₂—, —B(OR^{k})—, —C(NR)—NR^{k}—, —NR^{k}—CR^{g}R^{g}—C(O)—, —C(O)—ONR^{k}—, —C(O)—NR^{k}O—, —C(S)—ONR^{k}—, —C(S)—NR^{k}O—, —C(NR)—ONR^{k}—, —C(NR)—NR^{k}O—, —OS(O)₂—NR^{k}NR^{k}—, —OC(O)—NR^{k}NR^{k}—, —OC(S)—NR^{k}NR^{k}—, —OC(NR)—NR^{k}NR^{k}—, —NR^{k}NR^{k}S(O)₂O—, —NR^{k}NR^{k}C(S)O—, —NR^{k}NR^{k}C(NR)O—, —OP(O)(R^{c})O—, —NR^{k}P(O)(R^{c})O—, —OP(O)(R^{c})NR^{k}—, —NR^{k}P(O)(R^{c})NR^{k}—, —P(O)(R^{c})NR^{k}—, —NR^{k}P(O)(R^{c})—, —O-alkylene-heterocycloalkylene-NR^{k}—, —NR^{k}—CHR^{g}—C(O)—NR^{k}—CHR^{g}—C(O)—, —NR^{k}—CHR^{g}—C(O)—, —NR^{k}—C(O)—CHR^{g}—, or —C(O)—NR^{k}—CHR^{g}—C(O)—; and
each of Q, U, and V are independently N or CR^{g}, wherein at least one of Q, U, or V is N; and each CR^{g} may be the same or different;
R, for each occurrence, is independently H, an optionally substituted alkyl, an optionally substituted cycloalkyl, an optionally substituted cyclyl, an optionally substituted heterocycloalkyl, an optionally substituted heterocyclyl, an optionally substituted heteroaryl, an optionally substituted aralkyl, an optionally substituted heteroaralkyl, —C(O)R^{c}, —OR^{k}, —SR^{k}, —NR^{h}R^{j}, hydroxylalkyl, nitro, cyano, haloalkyl, aminoalkyl, or —S(O)₂R^{c};
each of R^{a} and R^{b}, independently, is H, optionally substituted alkyl, an optionally substituted cycloalkyl, an optionally substituted cyclyl, an optionally substituted heterocycloalkyl, an optionally substituted heterocyclyl, optionally substituted aryl, or optionally substituted heteroaryl;
R^{c}, for each occurrence, is independently, H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl, haloalkyl, —OR^{k}, —SR^{k}, —NR^{h}R^{j}, hydroxylalkyl, alkylcarbonylalkyl, mercaptoalkyl, aminoalkyl, sulfonylalkyl, sulfonylaryl, or thioalkoxy;
R^{g}, for each occurrence, is independently, H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl, haloalkyl, —OR^{k}, —SR^{k}, —NR^{h}R^{j}, hydroxylalkyl, alkylcarbonylalkyl, mercaptoalkyl, aminoalkyl, sulfonylalkyl, sulfonylaryl, thioalkoxy, —C(O)R^{c}, —OC(O)R^{c}, —SC(O)R^{c}, —NR^{k}C(O)R^{c}, —C(S)R^{c}, —OC(S)R^{c}, —SC(S)R^{c}, —NR^{k}C(S)R^{c}, —C(NR)R^{c}, —OC(NR)R^{c}, —SC(NR)R^{c}, —NR^{k}C(NR)R^{c}, —SO₂R^{c}, —S(O)R^{c}, —NR^{k}SO₂R^{c}, —OS(O)₂R^{c}, —OP(O)R^{c}R^{c}, —P(O)R^{c}R^{c}, halo, aminoalkyl, mercaptoalkyl, cyano, nitro, nitroso, or azide;
R^{h} and R^{j}, for each occurrence, are independently H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl; or R^{h} and R^{j} taken together with the N to which they are attached is an optionally substituted heterocyclyl, an optionally substituted heterocycloalkyl, or an optionally substituted heteroaryl;
R^{k}, for each occurrence, is independently H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, or an optionally substituted heteroaryl;
n is 0, 1, 2, 3, 4, 5, 6 or 7; and
m is 0, 1, 2, 3, or 4; and

(b) a pharmaceutically acceptable topical carrier.

In another aspect, the invention provides a pharmaceutical composition for topical administration, comprising:

(a) a compound represented by formula (II):

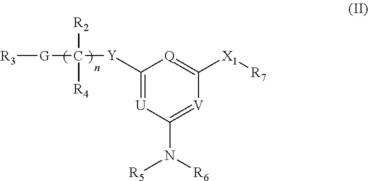

or a pharmaceutically acceptable salt thereof, wherein G, Q, U, V, Y, R₂, R₃, R₄, R₅, R₆, and n are defined as for formula (I);
X₁ is represented by a formula selected from the group consisting of:

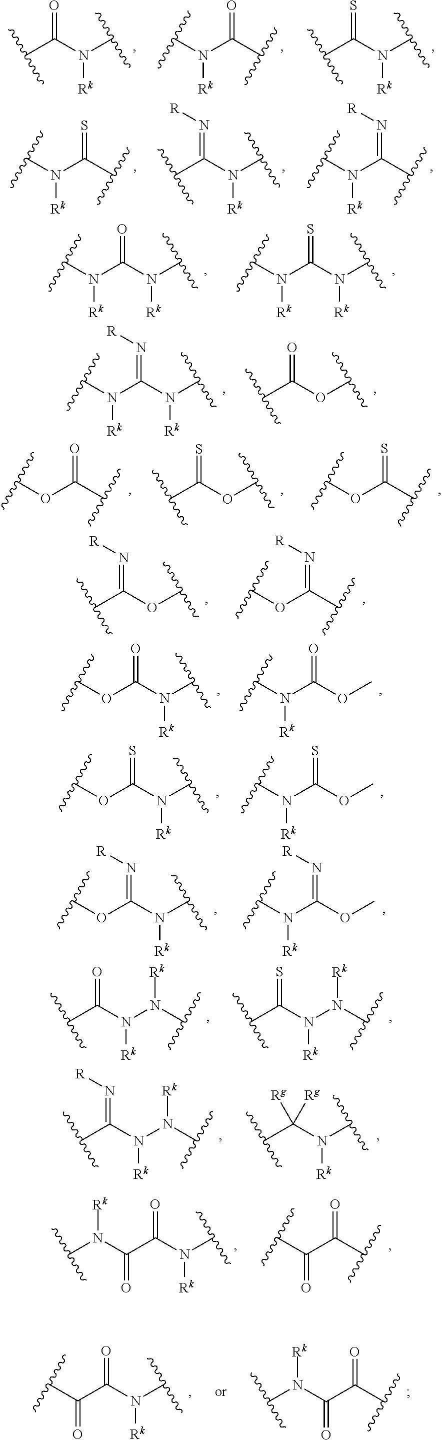

R and R^{k} are defined as for formula (I); and
R₇ is an optionally substituted aryl or an optionally substituted heteroaryl; and

(b) a pharmaceutically acceptable topical carrier.

In another aspect, the invention provides a pharmaceutical composition for topical administration, comprising:

(a) a compound represented by formula (III):

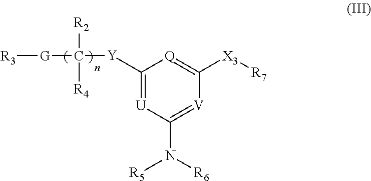

or a pharmaceutically acceptable salt thereof, wherein G, Q, U, V, Y, R₂, R₃, R₄, R₅, R₆, and n are defined as for formula (I);
R₇ is defined as for formula (II);
X₃ is —C(R^{g})═N-A-;
A is O, S, S(O), S(O)₂, C(CR^{g})₂, or NR^{k};
R^{g} and R^{k} are defined as for formula (I); and

(b) a pharmaceutically acceptable topical carrier.

In another aspect, the invention provides a pharmaceutical composition for topical administration, comprising:

(a) a compound represented by formula (IV):

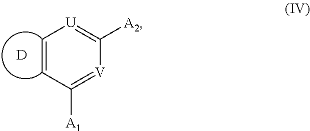

or a pharmaceutically acceptable salt thereof, wherein:
U and V are each, independently, N or CR^{g};
Ring D is a 5 to 9-membered aryl, 3 to 9-membered cycloalkyl, 3 to 9-membered cyclyl, 5 to 9-membered heteroaryl, 3 to 9-membered heterocycloalkyl, or a 3 to 9-membered heterocyclyl, each of which may be further substituted with one or more substituents;
one of A₁ and A₂ is —X₄—R′-L′-R″ and the other is a group represented by the following formula:

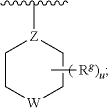

Z is N or CH;
W is O, S, S(O), S(O)₂, NR^{m}, or NC(O)R^{m}, wherein R^{m}, for each occurrence, is independently —H, alkyl, aryl, heteroaryl, cycloalkyl, heterocycloalkyl, or alkylcarbonyl;
u is 0, 1, 2, 3, or 4;
X₄ is O, S, S(O), S(O)₂, N(R^{k}), C(O), C(S), C(S)NR^{k}, C(NR), C(NR)NR^{k}, C(O)NR^{k}, C(O)NR^{k}NR^{k}, C(O)ONR^{k}, C(O)NR^{k}O, C(O)O, OC(O), OC(O)O, (C(R^{g})(R^{g}))_{q}, (C(R^{g})(R_{g}))_{q}NR^{k}, (C(R^{g})(R^{g}))_{q}O, (C(R^{g})(R^{g}))_{q}S(O)ₚ, (C(R^{g})(R^{g}))_{q}N═C(R^{g}), C(R^{g})═N, C(R^{g})═N—O, C(R^{g})═N—S(O)ₚ, C(R^{g})═N—NR^{k}, C(R^{g})═N—C(CR^{g})₂, (C(R^{g})(R^{g}))_{q}C(R^{g})═N, (C(R^{g})(R^{g}))_{q}N═N, (C(R^{g})(R^{g}))_{q}C(R^{g})═C(R^{g}), C(R^{g})═C(R^{g}), N═C(R^{g}), N(R^{k})N═C(R^{g}), N(R^{k})C(R^{g})═N, N(R^{k})C(R^{g})═C(R^{g}), N═N, N(R^{k})N═N, NR^{k}C(O)NR^{k}, NR^{k}C(S)NR^{k}, NR^{k}C(O), NR^{k}C(O)O, NR^{k}C(NR)NR^{k}, NR^{k}C(S)O, NR^{k}S(O)ₚNR^{k}, OC(O)NR^{k}, OC(S)NR^{k}, OC(NR)NR^{k}, OS(O)ₚNR^{k}, C(NR)O, S(O)ₚNR^{k}, or S(O)ₚNR^{k}NR^{k};
R′ is an optionally substituted cycloalkyl, an optionally substituted cyclyl, an optionally substituted heterocycloalkyl, an optionally substituted aryl, an optionally substituted heteroaryl, an optionally substituted aralkyl, an optionally substituted heteroaralkyl, or absent;
L′ is O, S, S(O), S(O)₂, N(R^{k}), C(O), C(S), C(S)NR^{k}, C(NR), C(NR)NR^{k}, C(O)NR^{k}, C(O)NR^{k}NR^{k}, C(O)ONR^{k}, C(O)NR^{k}O, C(O)O, OC(O), OC(O)O, (C(R^{g})(R^{g}))_{q}, (C(R^{g})(R_{g}))_{q}NR^{k}, (C(R^{g})(R^{g}))_{q}O, (C(R^{g})(R^{g}))_{q}S(O)ₚ, (C(R^{g})(R^{g}))_{q}N═C(R^{g}), C(R^{g})═N, C(R^{g})═N—O, C(R^{g})═N—S(O)ₚ, C(R^{g})═N—NR^{k}, C(R^{g})═N—C(CR^{g})₂, (C(R^{g})(R^{g}))_{q}C(R^{g})═N, (C(R^{g})(R^{g}))_{q}N═N, (C(R^{g})(R^{g}))_{q}C(R^{g})═C(R^{g}), C(R^{g})═C(R^{g}), N═C(R^{g}), N(R^{k})N═C(R^{g}), N(R^{k})C(R^{g})═N, N(R^{k})C(R^{g})═C(R^{g}), N═N, N(R^{k})N═N, NR^{k}C(O)NR^{k}, NR^{k}C(S)NR^{k}, NR^{k}C(O), NR^{k}C(O)O, NR^{k}C(NR)NR^{k}, NR^{k}C(S)O, NR^{k}S(O)ₚNR^{k}, OC(O)NR^{k}, OC(S)NR^{k}, OC(NR)NR^{k}, OS(O)ₚNR^{k}, C(NR)O, S(O)ₚNR^{k}, S(O)ₚNR^{k}NR^{k} or absent; and

R″ is H, an optionally substituted alkyl, an optionally substituted cycloalkyl, an optionally substituted cyclyl, an optionally substituted heterocycloalkyl, an optionally substituted heterocyclyl, an optionally substituted aryl, an optionally substituted heteroaryl, an optionally substituted aralkyl, an optionally substituted heteroaralkyl, N(R^{k})(CH₂)_{q}R^{g}, —OR^{k}, —SR^{k}, —NR^{h}R^{j}, hydroxylalkyl, —C(O)R^{c}, —C(S)R^{c}, —C(NR)R^{c}, halo, haloalkyl, aminoalkyl, mercaptoalkyl, cyano, nitro, —S(O)R^{c}, —S(O)₂R^{c}, —P(O)R^{c}R^{c}, —P(S)R^{c}R^{c}, or an optionally substituted alkylcarbonylalkyl;
q, for each occurrence, is independently 1, 2, 3, 4, 5, 6, 7, or 8;
p, for each occurrence, is independently 0, 1, or 2; and
R, R^{c}, R^{g}, R^{h}, R^{j}, and R^{k} are defined as for formula (I); and

(b) a pharmaceutically acceptable topical carrier.

In another aspect, the invention provides a pharmaceutical composition for topical administration, comprising:

(a) a compound represented by formula (X):

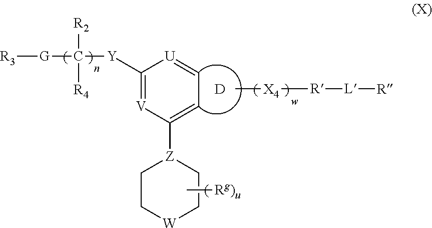

or a pharmaceutically acceptable salt thereof, wherein:
G, Y, R₂, R₃, R₄, and n are defined as for formula (I);
L′, U, V, W, X4, Z, R′, R″, u, and Ring D are defined as for formula (IV); and
w is 0 or 1; and

(b) a pharmaceutically acceptable topical carrier.

In another aspect, the invention provides a pharmaceutical composition for topical administration, comprising:

(a) a compound represented by formula (XIV):

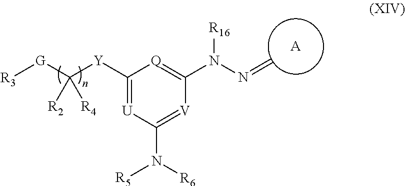

or a pharmaceutically acceptable salt thereof, wherein:
G, Q, U, V, Y, R₂, R₃, R₄, R₅, R₆ and n are defined as for formula (I):
ring A is an optionally substituted cycloalkyl, an optionally substituted cyclyl, an optionally substituted heterocycloalkyl, or an optionally substituted heterocyclyl, wherein the cycloalkyl, cyclyl, heterocycloalkyl, and heterocyclyl are optionally fused to an optionally substituted cycloalkyl, an optionally substituted cyclyl, an optionally substituted heterocycloalkyl, an optionally substituted heterocyclyl, an optionally substituted aryl, or an optionally substituted heteroaryl; and
R₁₆, for each occurrence, is independently, H or a lower alkyl; and

(b) a pharmaceutically acceptable topical carrier.

In another aspect, the invention provides a method of treating psoriasis in a patient, comprising contacting one or more psoriatic skin lesion of the patient with a composition of the invention.

Other features, objects, and advantages of the invention will be apparent from the description and from the claims.

### BRIEF DESCRIPTION OF THE DRAWINGS

FIG. 1A is a graph showing a decrease in IL-12/23 p40 gene expression in the skin biopsies of patients after two weeks of treatment with Compound 50 compared to IL-12/23 p40 gene expression prior to treatment. (Numbers 1034-1038 and 1043-1048 designate patient identification numbers).

FIG. 1B is a graph showing a decrease in IL-23 p19 gene expression in the skin biopsies of patients after two weeks of treatment with Compound 50 compared to IL-23 p19 gene expression prior to treatment.

FIG. 1C is a graph showing an increase in IL-10 mRNA in the skin biopsies of patients after two weeks of treatment with Compound 50 compared to IL-10 mRNA prior to treatment.

FIG. 2 is a graph showing the median ratio of psoriasis area and severity index (PASI), skin thickness and immune cell infiltration compared to baseline (psoriatic lesions prior to treatment) after 12 weeks of treatment with Compound 50.

FIG. 3 is a graph showing clearance of CD11c⁺ cells (dendritic cells) from epidermis in responders. (Numbers 1034-1043 and 1044-1048 designate patient identification numbers. Circles designate responders. Triangles designate nonresponders.)

### DETAILED DESCRIPTION

In one aspect, the invention provides a pharmaceutical composition for topical administration, comprising:

(a) a compound represented by formula (I):

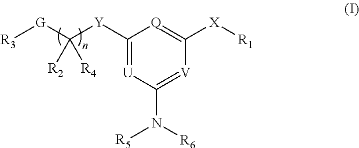

or a pharmaceutically acceptable salt thereof, wherein:
R₁ is optionally substituted aryl, optionally substituted heteroaryl, or a group represented by the following formula:

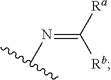

R₂ and R₄, for each occurrence, are independently, H, an optionally substituted alkyl, an optionally substituted alkylcarbonyl, —OR^{k}, —SR^{k}, —NR^{h}R^{j}, hydroxylalkyl, —C(O)R^{c}, —OC(O)R^{c}, —SC(O)R^{c}, —NR^{k}C(O)R^{c}, —C(S)R^{c}, —OC(S)R^{c}, —SC(S)R^{c}, —NR^{k}C(S)R^{c}, —C(NR)R^{c}, —OC(NR)R^{c}, —SC(NR)R^{c}, —NR^{k}C(NR)R^{c}, —SO₂R^{c}, —S(O)R^{c}, —NR^{k}SO₂R^{c}, —OS(O)₂R^{c}, —OP(O)R^{c}R^{c}, —P(O)R^{c}R^{c}, halo, haloalkyl, aminoalkyl, mercaptoalkyl, cyano, nitro, nitroso, azide, an optionally substituted alkylcarbonylalkyl, an optionally substituted cyclyl, an optionally substituted cycloalkyl, an optionally substituted heterocyclyl, an optionally substituted heterocycloalkyl, an optionally substituted aryl, an optionally substituted aralkyl, an optionally substituted heteroaryl, an optionally substituted heteroaralkyl, or isothionitro; or R₂ and R₄ taken together are ═O, ═S, or ═NR;
R₃ is R^{g};
R₅ and R₆ are each, independently, H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl; or R₅ and R₆ taken together with the N to which they are attached is an optionally substituted heterocyclyl, an optionally substituted heterocycloalkyl, or an optionally substituted heteroaryl;
X is O, S, S(O), S(O)₂, or NR^{k};
Y is (CH(R^{g}))ₘ, C(O), C(NR), O, S, S(O), S(O)₂, N(R^{k}), or absent;
G is a bond, —C(O)NR^{k}NR^{k}—, —NR^{k}NR^{k}C(O)—, —NR^{k}N═CR^{k}—, —CR^{k}═NNR^{k}—, —NR^{k}NR^{k}—, —N(OH)—, —NR^{k}O—, —ONR^{k}—, —C(O)—, —C(NR)—, —NR^{k}C(O)—, —C(O)NR^{k}—, —OC(O)—, —C(O)O—, —OC(O)O—, —NR^{k}C(O)O—, —OC(O)NR^{k}—, —NR^{k}C(S)O—, —OC(S)NR^{k}—, —NR^{k}—C(NR)—NR^{k}, —NR^{k}—C(O)—NR^{k}—, —NR^{k}—C(S)—NR^{k}—, —NR^{k}—S(O)₂—NR^{k}—, —P(O)(R^{c})—, —P(O)(R^{c})O—, —OP(O)(R^{c})—, —OP(O)(R^{c})O—, an optionally substituted cycloalkylene, an optionally substituted cyclylene, an optionally substituted heterocycloalkylene, an optionally substituted heterocyclylene, an optionally substituted arylene, an optionally substituted aralkylene, an optionally substituted heteroarylene, an optionally substituted heteroaralkylene, an optionally substituted heteroarylene-NR^{k}—, an optionally substituted heteroarylene-S—, an optionally substituted heteroaralkylene-O—, —Si(OR^{k})₂—, —B(OR^{k})—, —C(NR)—NR^{k}—, —NR^{k}—CR^{g}R^{g}—C(O)—, —C(O)—ONR^{k}—, —C(O)—NR^{k}O—, —C(S)—ONR^{k}—, —C(S)—NR^{k}O—, —C(NR)—ONR^{k}—, —C(NR)—NR^{k}O—, —OS(O)₂—NR^{k}NR^{k}—, —OC(O)—NR^{k}NR^{k}—, —OC(S)—NR^{k}NR^{k}—, —OC(NR)—NR^{k}NR^{k}—, —NR^{k}NR^{k}S(O)₂O—, —NR^{k}NR^{k}C(S)O—, —NR^{k}NR^{k}C(NR)O—, —OP(O)(R^{c})O—, —NR^{k}P(O)(R^{c})O—, —OP(O)(R^{c})NR^{k}—, —NR^{k}P(O)(R^{c})NR^{k}—, —P(O)(R^{c})NR^{k}—, —NR^{k}P(O)(R^{c})—, —O-alkylene-heterocycloalkylene-NR^{k}—, —NR^{k}—CHR^{g}—C(O)—NR^{k}—CHR^{g}—C(O)—, —NR^{k}—CHR^{g}—C(O)—, —NR^{k}—C(O)—CHR^{g}—, or —C(O)—NR^{k}—CHR^{g}—C(O)—; and
each of Q, U, and V are independently N or CR^{g}, wherein at least one of Q, U, or V is N; and each CR^{g} may be the same or different;
R, for each occurrence, is independently H, an optionally substituted alkyl, an optionally substituted cycloalkyl, an optionally substituted cyclyl, an optionally substituted heterocycloalkyl, an optionally substituted heterocyclyl, an optionally substituted heteroaryl, an optionally substituted aralkyl, an optionally substituted heteroaralkyl, —C(O)R^{c}, —OR^{k}, —SR^{k}, —NR^{h}R^{j}, hydroxylalkyl, nitro, cyano, haloalkyl, aminoalkyl, or —S(O)₂R^{c};
each of R^{a} and R^{b}, independently, is H, optionally substituted alkyl, an optionally substituted cycloalkyl, an optionally substituted cyclyl, an optionally substituted heterocycloalkyl, an optionally substituted heterocyclyl, optionally substituted aryl, or optionally substituted heteroaryl;
R^{c}, for each occurrence, is independently, H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl, haloalkyl, —OR^{k}, —SR^{k}, —NR^{h}R^{j}, hydroxylalkyl, alkylcarbonylalkyl, mercaptoalkyl, aminoalkyl, sulfonylalkyl, sulfonylaryl, or thioalkoxy;
R^{g}, for each occurrence, is independently, H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl, haloalkyl, —OR^{k}, —SR^{k}, —NR^{h}R^{j}, hydroxylalkyl, alkylcarbonylalkyl, mercaptoalkyl, aminoalkyl, sulfonylalkyl, sulfonylaryl, thioalkoxy, —C(O)R^{c}, —OC(O)R^{c}, —SC(O)R^{c}, —NR^{k}C(O)R^{c}, —C(S)R^{c}, —OC(S)R^{c}, —SC(S)R^{c}, —NR^{k}C(S)R^{c}, —C(NR)R^{c}, —OC(NR)R^{c}, —SC(NR)R^{c}, —NR^{k}C(NR)R^{c}, —SO₂R^{c}, —S(O)R^{B}, —NR^{k}SO₂R^{c}, —OS(O)₂R^{c}, —OP(O)R^{c}R^{c}, —P(O)R^{c}R^{c}, halo, aminoalkyl, mercaptoalkyl, cyano, nitro, nitroso, or azide;
R^{h} and R^{j}, for each occurrence, are independently H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl; or R^{h} and R^{j} taken together with the N to which they are attached is an optionally substituted heterocyclyl, an optionally substituted heterocycloalkyl, or an optionally substituted heteroaryl;
R^{k}, for each occurrence, is independently H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, or an optionally substituted heteroaryl;
n is 0, 1, 2, 3, 4, 5, 6 or 7; and
m is 0, 1, 2, 3, or 4; and

(b) a pharmaceutically acceptable topical carrier.

In another aspect, the invention provides a pharmaceutical composition for topical administration, comprising:

(a) a compound represented by formula (II):

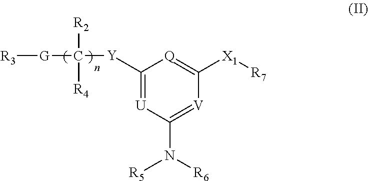

or a pharmaceutically acceptable salt thereof, wherein G, Q, U, V, Y, R₂, R₃, R₄, R₅, R₆, and n are defined as for formula (I);
X₁ is represented by a formula selected from the group consisting of:

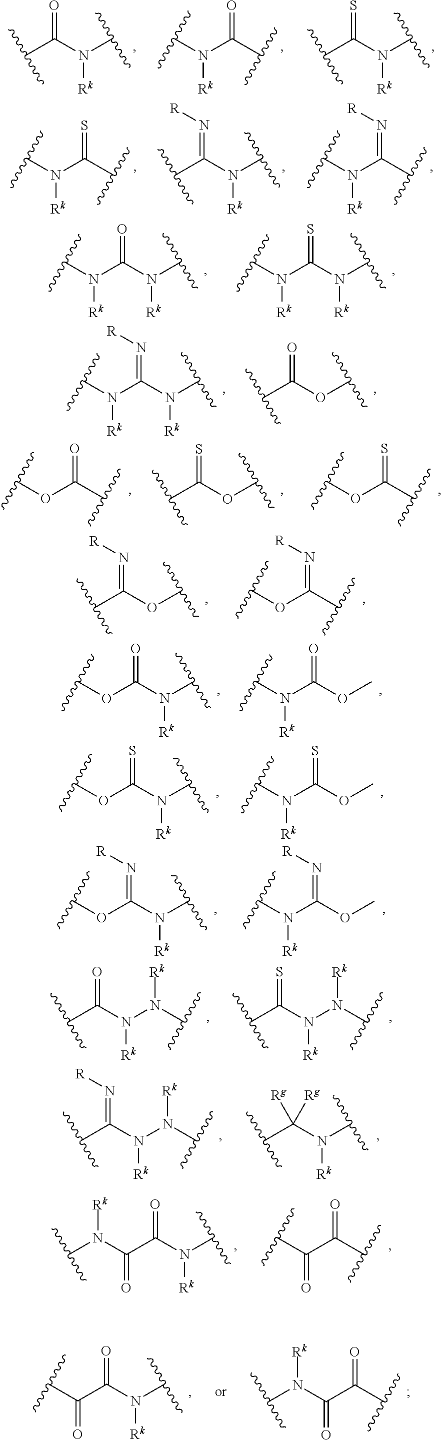

R and R^{k} are defined as for formula (I); and
R₇ is an optionally substituted aryl or an optionally substituted heteroaryl; and

(b) a pharmaceutically acceptable topical carrier.

In another aspect, the invention provides a pharmaceutical composition for topical administration, comprising:

(a) a compound represented by formula (III):

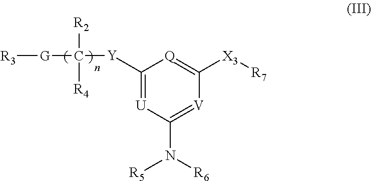

or a pharmaceutically acceptable salt thereof, wherein G, Q, U, V, Y, R₂, R₃, R₄, R₅, R₆, and n are defined as for formula (I);
R₇ is defined as for formula (II);
X₃ is —C(R^{g})=N-A-;
A is O, S, S(O), S(O)₂, C(CR^{g})₂, or NR^{k};
R^{g} and R^{k} are defined as for formula (I); and

(b) a pharmaceutically acceptable topical carrier.

In another aspect, the invention provides a pharmaceutical composition for topical administration, comprising:

(a) a compound represented by formula (IV):

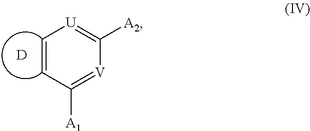

or a pharmaceutically acceptable salt thereof, wherein:
U and V are each, independently, N or CR^{g};
Ring D is a 5 to 9-membered aryl, 3 to 9-membered cycloalkyl, 3 to 9-membered cyclyl, 5 to 9-membered heteroaryl, 3 to 9-membered heterocycloalkyl, or a 3 to 9-membered heterocyclyl, each of which may be further substituted with one or more substituents;
one of A₁ and A₂ is —X₄—R′-L′-R″ and the other is a group represented by the following formula:

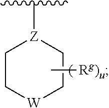

Z is N or CH;
W is O, S, S(O), S(O)₂, NR^{m}, or NC(O)R^{m}, wherein R^{m}, for each occurrence, is independently —H, alkyl, aryl, heteroaryl, cycloalkyl, heterocycloalkyl, or alkylcarbonyl;
u is 0, 1, 2, 3, or 4;
X₄ is O, S, S(O), S(O)₂, N(R^{k}), C(O), C(S), C(S)NR^{k}, C(NR), C(NR)NR^{k}, C(O)NR^{k}, C(O)NR^{k}NR^{k}, C(O)ONR^{k}, C(O)NR^{k}O, C(O)O, OC(O), OC(O)O, (C(R^{g})(R^{g}))_{q}, (C(R^{g})(R_{g}))_{q}NR^{k}, (C(R^{g})(R^{g}))_{q}O, (C(R^{g})(R^{g}))_{q}S(O)ₚ, (C(R^{g})(R^{g}))_{q}N═C(R^{g}), C(R^{g})═N, C(R^{g})═N—O, C(R^{g})═N—S(O)ₚ, C(R^{g})═N—NR^{k}, C(R^{g})═N—C(CR^{g})₂, (C(R^{g})(R^{g}))_{q}C(R^{g})═N, (C(R^{g})(R^{g}))_{q}N═N, (C(R^{g})(R^{g}))_{q}C(R^{g})═C(R^{g}), C(R^{g})═C(R^{g}), N═C(R^{g}), N(R^{k})N═C(R^{g}), N(R^{k})C(R^{g})═N, N(R^{k})C(R^{g})═C(R^{g}), N═N, N(R^{k})N═N, NR^{k}C(O)NR^{k}, NR^{k}C(S)NR^{k}, NR^{k}C(O), NR^{k}C(O)O, NR^{k}C(NR)NR^{k}, NR^{k}C(S)O, NR^{k}S(O)ₚNR^{k}, OC(O)NR^{k}, OC(S)NR^{k}, OC(NR)NR^{k}, OS(O)ₚNR^{k}, C(NR)O, S(O)ₚNR^{k}, or S(O)ₚNR^{k}NR^{k};
R′ is an optionally substituted cycloalkyl, an optionally substituted cyclyl, an optionally substituted heterocycloalkyl, an optionally substituted aryl, an optionally substituted heteroaryl, an optionally substituted aralkyl, an optionally substituted heteroaralkyl, or absent;
L′ is O, S, S(O), S(O)₂, N(R^{k}), C(O), C(S), C(S)NR^{k}, C(NR), C(NR)NR^{k}, C(O)NR^{k}, C(O)NR^{k}NR^{k}, C(O)ONR^{k}, C(O)NR^{k}O, C(O)O, OC(O), OC(O)O, (C(R^{g})(R^{g}))_{q}, (C(R^{g})(R_{g}))_{q}NR^{k}, (C(R^{g})(R^{g}))_{q}O, (C(R^{g})(R^{g}))_{q}S(O)ₚ, (C(R^{g})(R^{g}))_{q}N═C(R^{g}), C(R^{g})═N, C(R^{g})═N—O, C(R^{g})═N—S(O)ₚ, C(R^{g})═N—NR^{k}, C(R^{g})═N—C(CR^{g})₂, (C(R^{g})(R^{g}))_{q}C(R^{g})═N, (C(R^{g})(R^{g}))_{q}N═N, (C(R^{g})(R^{g}))_{q}C(R^{g})═C(R^{g}), C(R^{g})═C(R^{g}), N═C(R^{g}), N(R^{k})N═C(R^{g}), N(R^{k})C(R^{g})═N, N(R^{k})C(R^{g})═C(R^{g}), N═N, N(R^{k})N═N, NR^{k}C(O)NR^{k}, NR^{k}C(S)NR^{k}, NR^{k}C(O), NR^{k}C(O)O, NR^{k}C(NR)NR^{k}, NR^{k}C(S)O, NR^{k}S(O)ₚNR^{k}, OC(O)NR^{k}, OC(S)NR^{k}, OC(NR)NR^{k}, OS(O)ₚNR^{k}, C(NR)O, S(O)ₚNR^{k}, S(O)ₚNR^{k}NR^{k} or absent; and
R″ is H, an optionally substituted alkyl, an optionally substituted cycloalkyl, an optionally substituted cyclyl, an optionally substituted heterocycloalkyl, an optionally substituted heterocyclyl, an optionally substituted aryl, an optionally substituted heteroaryl, an optionally substituted aralkyl, an optionally substituted heteroaralkyl, N(R^{k})(CH₂)_{q}R^{g}, —OR^{k}, —SR^{k}, —NR^{h}R^{j}, hydroxylalkyl, —C(O)R^{c}, —C(S)R^{c}, —C(NR)R^{c}, halo, haloalkyl, aminoalkyl, mercaptoalkyl, cyano, nitro, —S(O)R^{c}, —S(O)₂R^{c}, —P(O)R^{c}R^{c}, —P(S)R^{c}R^{c}, or an optionally substituted alkylcarbonylalkyl;
q, for each occurrence, is independently 1, 2, 3, 4, 5, 6, 7, or 8;
p, for each occurrence, is independently 0, 1, or 2; and
R, R^{c}, R^{g}, R^{h}, R^{j}, and R^{k} are defined as for formula (I); and

(b) a pharmaceutically acceptable topical carrier.

In another aspect, the invention provides a pharmaceutical composition for topical administration, comprising:

(a) a compound represented by formula (X):

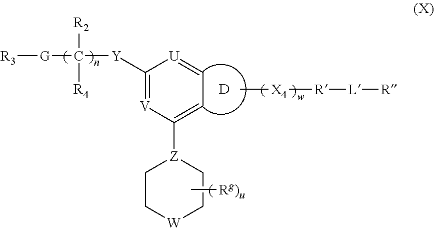

or a pharmaceutically acceptable salt thereof, wherein:
G, Y, R₂, R₃, R₄, and n are defined as for formula (I);
L′, U, V, W, X4, Z, R′, R″, u, and Ring D are defined as for formula (IV); and
w is 0 or 1; and

(b) a pharmaceutically acceptable topical carrier.

In another aspect, the invention provides a pharmaceutical composition for topical administration, comprising:

(a) a compound represented by formula (XIV):

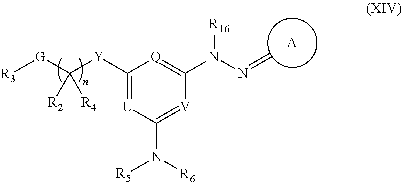

or a pharmaceutically acceptable salt thereof, wherein:
G, Q, U, V, Y, R₂, R₃, R₄, R₅, R₆ and n are defined as for formula (I):
ring A is an optionally substituted cycloalkyl, an optionally substituted cyclyl, an optionally substituted heterocycloalkyl, or an optionally substituted heterocyclyl, wherein the cycloalkyl, cyclyl, heterocycloalkyl, and heterocyclycl are optionally fused to an optionally substituted cycloalkyl, an optionally substituted cyclyl, an optionally substituted heterocycloalkyl, an optionally substituted heterocyclyl, an optionally substituted aryl, or an optionally substituted heteroaryl; and
R₁₆, for each occurrence, is independently, H or a lower alkyl; and

b) a pharmaceutically acceptable topical carrier.

In some embodiments, in the compounds represented by formula (I), (II), (III), or (XIV), Q, U, and V are N.

In some embodiments, in the compounds represented by formula (I), (II), (III), or (XIV), one of Q, U, or V is CR^{g}, and the other two are N.

In some embodiments, in the compounds represented by formula (I), (II), (III), or (XIV), V is CR^{g}, Q and U are N.

In some embodiments, in the compounds represented by formula (I), (II), (III), or (XIV), Q is CR^{g}, V and U are N.

In some embodiments, in the compounds represented by formula (I), (II), (III), or (XIV), U is CR^{g}, V and Q are N.

In some embodiments, in the compounds represented by formula (I), (II), (III), or (XIV), one of Q, U, or V is N, and the other two are CR^{g}.

In some embodiments, in the compounds represented by formula (I), (II), (III), or (XIV), V is N, and Q and U are CR^{g}.

In some embodiments, in the compounds represented by formula (I), (II), (III), or (XIV), Q is N, and V and U are CR^{g}.

In some embodiments, in the compounds represented by formula (I), (II), (III), or (XIV), U is N and Q, and V are CR^{g}.

In some embodiments, in the compounds represented by formula (I), (II), (III), or (XIV), —NR₅R₆ is an optionally substituted morpholino, an optionally substituted thiomorpholino, an optionally substituted 1-oxo-thiomorpholino, an optionally substituted 1,1-dioxo-thiomorpholino, an optionally substituted piperidinyl, or an optionally substituted piperazinyl.

In some embodiments, in the compounds represented by formula (I), X is —NR^{k}—. In a preferred embodiment, the R^{k} of group X is —H or a lower alkyl.

In some embodiments, R₁ in the compounds represented by formula (I) or R₇ in the compounds represented by formula (II) or (III), is an optionally substituted aryl or an optionally substituted heteroaryl.

In some embodiments, R₁ in the compounds represented by formula (I) or R₇ in the compounds represented by formula (II) or (III), is an optionally substituted phenyl, an optionally substituted naphthyl, an optionally substituted anthracenyl, an optionally substituted fluorenyl, an optionally substituted indenyl, an optionally substituted azulenyl, an optionally substituted pyridyl, an optionally substituted 1-oxo-pyridyl, an optionally substituted furanyl, an optionally substituted benzo[1,3]dioxolyl, an optionally substituted benzo[1,4]dioxinyl, an optionally substituted thienyl, an optionally substituted pyrrolyl, an optionally substituted oxazolyl, an optionally substituted imidazolyl, an optionally substituted thiazolyl, an optionally substituted isoxazolyl, an optionally substituted quinolinyl, an optionally substituted pyrazolyl, an optionally substituted isothiazolyl, an optionally substituted pyridazinyl, an optionally substituted pyrimidinyl, an optionally substituted pyrazinyl, an optionally substituted triazinyl, an optionally substituted triazolyl, an optionally substituted thiadiazolyl, an optionally substituted isoquinolinyl, an optionally substituted indazolyl, an optionally substituted benzoxazolyl, an optionally substituted benzofuryl, an optionally substituted indolizinyl, an optionally substituted imidazopyridyl, an optionally substituted tetrazolyl, an optionally substituted benzimidazolyl, an optionally substituted benzothiazolyl, an optionally substituted benzothiadiazolyl, an optionally substituted benzoxadiazolyl, an optionally substituted indolyl, an optionally substituted carbazolyl, an optionally substituted 1,2,3,4-tetrahydro-carbazolyl, an optionally substituted tetrahydroindolyl, an optionally substituted azaindolyl, an optionally substituted indazolyl, an optionally substituted imidazopyridyl, an optionally substituted quinazolinyl, an optionally substituted purinyl, an optionally substituted pyrrolo[2,3]pyrimidinyl, an optionally substituted pyrazolo[3,4]pyrimidinyl, or an optionally substituted benzo(b)thienyl.

In some embodiments, R₁ in the compounds represented by formula (I) or R₇ in the compounds represented by formula (II) or (III), is an optionally substituted phenyl, an optionally substituted indolyl, an optionally substituted indanyl, an optionally substituted carbazolyl, or an optionally substituted 1,2,3,4-tetrahydro-carbazolyl.

In some embodiments, R₁ in the compounds represented by formula (I) or R₇ in the compounds represented by formula (II) or (III) is a group represented by the following formula:

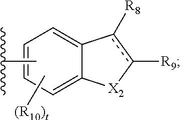

wherein:

the dashed line indicates a double or a single bond;

X₂ is —O—, —S(O)ₚ—, —N(R^{k})—, or —C(R^{g})(R^{g})—;

R₈ and R₉ are each, independently, R^{g}, —C(O)R^{c}, —C(S)R^{c}, —C(NR)R^{c}, —NR^{k}C(O)R^{c}, —OC(O)R^{c}, —SC(O)R^{c}, —NR^{k}C(S)R^{c}, —OC(S)R^{c}, —SC(S)R^{c}, —NR^{k}C(NR)R^{c}, —OC(NR)R^{c}, or —SC(NR)R^{c}; or R₈ and R₉, taken together with the carbons to which they are attached, form a 5- to 7-membered optionally substituted cycloalkyl, a 5- to 7-membered optionally substituted cyclyl, a 5- to 7-membered optionally substituted aryl, a 5- to 7-membered optionally substituted heterocycloalkyl, a 5- to 7-membered optionally substituted heterocyclyl, a 5- to 7-membered optionally substituted heteroaryl;

R₁₀, for each occurrence, is, independently, R^{g}, —C(O)R^{c}, —C(S)R^{c}, —C(NR)R^{c}, —NR^{k}C(O)R^{c}, —OC(O)R^{c}, —SC(O)R^{c}, —NR^{k}C(S)R^{c}, —OC(S)R^{c}, —SC(S)R^{c}, —NR^{k}C(NR)R^{c}, —OC(NR)R^{c}, or —SC(NR)R^{c};

p is 0, 1, or 2; and

t is 0, 1, 2, or, 3.

In some embodiments, R₁ in the compounds represented by formula (I) or R₇ in the compounds represented by formula (II) or (III) is (2,3-dimethyl-1H-indol-5-yl), (1H-indol-5-yl), or (6,7,8,9-tetrahydro-5H-carbazol-3-yl).

In some embodiments, in the compounds represented by formula (II) or (III), R₇ is a group represented by the following formula:

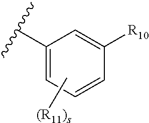

wherein:

R₁₁ and R₁₂, for each occurrence, are, independently, R^{g}, —C(O)R^{c}, —C(S)R^{c}, —C(NR)R^{c}, —NR^{k}C(O)R^{c}, —OC(O)R^{c}, —SC(O)R^{c}, —NR^{k}C(S)R^{c}, —OC(S)R^{c}, —SC(S)R^{c}, —NR^{k}C(NR)R^{c}, —OC(NR)R^{c}, or —SC(NR)R^{c}; and

s is 0, 1, 2, 3, or 4.

In some embodiments, in the compounds represented by formula (I), R₁ is a group represented by the following formula:

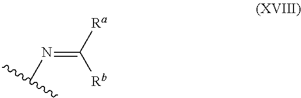

In some embodiments, when R₁ of formula (I) is group (XVIII), one of R^{a} or R^{b} is —H or a lower alkyl, and the other is an optionally substituted aryl or an optionally substituted heteroaryl.

In some embodiments, when R₁ of formula (I) is group (XVIII), one of R^{a} or R^{b} is —H or a lower alkyl, and the other is an optionally substituted phenyl, an optionally substituted naphthyl, an optionally substituted anthracenyl, an optionally substituted fluorenyl, an optionally substituted indenyl, an optionally substituted azulenyl, an optionally substituted pyridyl, an optionally substituted 1-oxo-pyridyl, an optionally substituted furanyl, an optionally substituted benzo[1,3]dioxolyl, an optionally substituted benzo[1,4]dioxinyl, an optionally substituted thienyl, an optionally substituted pyrrolyl, an optionally substituted oxazolyl, an optionally substituted imidazolyl, an optionally substituted thiazolyl, an optionally substituted isoxazolyl, an optionally substituted quinolinyl, an optionally substituted pyrazolyl, an optionally substituted isothiazolyl, an optionally substituted pyridazinyl, an optionally substituted pyrimidinyl, an optionally substituted pyrazinyl, an optionally substituted triazinyl, an optionally substituted triazolyl, an optionally substituted thiadiazolyl, an optionally substituted isoquinolinyl, an optionally substituted indazolyl, an optionally substituted benzoxazolyl, an optionally substituted benzofuryl, an optionally substituted indolizinyl, an optionally substituted imidazopyridyl, an optionally substituted tetrazolyl, an optionally substituted benzimidazolyl, an optionally substituted benzothiazolyl, an optionally substituted benzothiadiazolyl, an optionally substituted benzoxadiazolyl, an optionally substituted indolyl, an optionally substituted carbazolyl, an optionally substituted 1,2,3,4-tetrahydro-carbazolyl, an optionally substituted tetrahydroindolyl, an optionally substituted azaindolyl, an optionally substituted indazolyl, an optionally substituted imidazopyridyl, an optionally substituted quinazolinyl, an optionally substituted purinyl, an optionally substituted pyrrolo[2,3]pyrimidinyl, an optionally substituted pyrazolo[3,4]pyrimidinyl, or an optionally substituted benzo(b)thienyl.

In some embodiments, when R₁ of formula (I) is group (XVIII), one of R^{a} or R^{b} is —H or a lower alkyl, and the other is an optionally substituted phenyl, an optionally substituted indolyl, an optionally substituted indanyl, an optionally substituted carbazolyl, or an optionally substituted 1,2,3,4-tetrahydro-carbazolyl.

In some embodiments, in the compounds represented by formula (I), (II), (III), (X) or (XIV), Y is O.

In some embodiments, in the compounds represented by formula (I), (II), (III), (X) or (XIV), Y is a covalent bond.

In some embodiments, in the compounds represented by formula (I), (II), (III), (X) or (XIV), R₃ is H.

In some embodiments, in the compounds represented by formula (I), (II), (III), (X) or (XIV), R₃ is an optionally substituted aryl or an optionally substituted heteroaryl.

In some embodiments, in the compounds represented by formula (I), (II), (III), (X) or (XIV), R₃ is an optionally substituted phenyl, an optionally substituted naphthyl, an optionally substituted anthracenyl, an optionally substituted fluorenyl, an optionally substituted indenyl, an optionally substituted azulenyl, an optionally substituted pyridyl, an optionally substituted 1-oxo-pyridyl, an optionally substituted furanyl, an optionally substituted benzo[1,3]dioxolyl, an optionally substituted benzo[1,4]dioxinyl, an optionally substituted thienyl, an optionally substituted pyrrolyl, an optionally substituted oxazolyl, an optionally substituted imidazolyl, an optionally substituted thiazolyl, an optionally substituted isoxazolyl, an optionally substituted quinolinyl, an optionally substituted pyrazolyl, an optionally substituted isothiazolyl, an optionally substituted pyridazinyl, an optionally substituted pyrimidinyl, an optionally substituted pyrazinyl, an optionally substituted triazinyl, an optionally substituted triazolyl, an optionally substituted thiadiazolyl, an optionally substituted isoquinolinyl, an optionally substituted indazolyl, an optionally substituted benzoxazolyl, an optionally substituted benzofuryl, an optionally substituted indolizinyl, an optionally substituted imidazopyridyl, an optionally substituted tetrazolyl, an optionally substituted benzimidazolyl, an optionally substituted benzothiazolyl, an optionally substituted benzothiadiazolyl, an optionally substituted benzoxadiazolyl, an optionally substituted indolyl, an optionally substituted tetrahydroindolyl, an optionally substituted azaindolyl, an optionally substituted indazolyl, an optionally substituted imidazopyridyl, an optionally substituted quinazolinyl, an optionally substituted purinyl, an optionally substituted pyrrolo[2,3]pyrimidinyl, an optionally substituted pyrazolo[3,4]pyrimidinyl, or an optionally substituted benzo(b)thienyl.

In some embodiments, in the compounds represented by formula (I), (II), (III), (X) or (XIV), R₃ is an optionally substituted piperidinyl, an optionally substituted piperazinyl, an optionally substituted 2-oxopiperazinyl, an optionally substituted 2-oxopiperidinyl, an optionally substituted 2-oxopyrrolidinyl, an optionally substituted 4-piperidonyl, an optionally substituted tetrahydropyranyl, an optionally substituted oxazolidinyl, an optionally substituted 2-oxo-oxazolidinyl, an optionally substituted tetrahydrothiopyranyl, an optionally substituted tetrahydrothiopyranyl sulfone, an optionally substituted morpholinyl, an optionally substituted thiomorpholinyl, an optionally substituted thiomorpholinyl sulfoxide, an optionally substituted thiomorpholinyl sulfone, an optionally substituted 1,3-dioxolanyl, an optionally substituted [1,4]dioxanyl, an optionally substituted 2-oxo-imidazolidinyl, tetrahydrofuranyl, or an optionally substituted tetrahydrothienyl.

In some embodiments, in the compounds represented by formula (I), (II), (III), (X) or (XIV), R₃ is a hydroxy, an optionally substituted heterocycloalkyl, an optionally substituted heterocyclyl, or an optionally substituted heteroaryl.

In some embodiments, in the compounds represented by formula (I), (II), (III), (X) or (XIV), R₃ is a hydroxy, an optionally substituted pyridinyl, an optionally substituted morpholino, or an optionally substituted oxazolidin-2-one.

In some embodiments, in the compounds represented by formula (I), (II), (III), (X) or (XIV), R₃ is —OR^{k} or —NR^{h}R^{j}, and R^{f}, R^{h} and R^{j} are each, independently, H, an optionally substituted alkyl, an optionally substituted aryl, an optionally substituted heteroaryl, an optionally substituted cycloalkyl, an optionally substituted heterocycloalkyl, or —C(O)R^{c}.

In some embodiments, in the compounds represented by formula (I), (II), (III), (X) or (XIV), R₃ is —C(O)OR^{k}, —OC(O)R^{k}, —C(O)NR^{h}R^{j}, —NR^{k}C(O)R^{k}, —C(S)OR^{k}, —OC(S)R^{k}, —NR^{k}C(O)NR^{h}R^{j}, —NR^{k}C(S)NR^{h}R^{j}, —C(O)NR^{h}R^{j}, —S(O)₂R^{k}, —S(O)₂NR^{h}R^{j}, —OC(O)NR^{h}R^{j}, or —NR^{k}C(O)OR^{k}.

In some embodiments, in the compounds represented by formula (I), (II), (III), (X) or (XIV), each of R₂ and R₄ is, independently, H, an optionally substituted alkyl, an optionally substituted alkylcarbonyl, an optionally substituted aryl, an optionally substituted heteroaryl, an optionally substituted cycloalkyl, an optionally substituted cyclyl, an optionally substituted heterocycloalkyl, or an optionally substituted heterocyclyl.

In some embodiments, in the compounds represented by formula (I), (II), (III), (X) or (XIV), n is 1, 2, or 3, and R₂ and R₄, for each occurrence are, independently, H or a lower alkyl.

In some embodiments, in the compounds represented by formula (I), (II), (III), (X) or (XIV), G is absent.

In some embodiments, in the compounds represented by formula (I), (II), (III), (X) or (XIV), G is an optionally substituted heteroaryl or an optionally substituted heterocyclyl.

In some embodiments, in the compounds represented by formula (I), (II), (III), (X) or (XIV), G is —C(O)NHNH—, —NHNHC(O)—, —CH═N—NH—, —NH—N═CH—, —NHNH—, —NHO—, —O—NH—, —NR^{k}—O—, —CH═N—O—, —O—N═CH—, —O—C(S)—NH—, or —NH—C(S)—O—.

In some embodiments, in the compounds represented by formula (I), (II), (III), (X) or (XIV), G is —O—C(O)—NH—, —NH—C(NH)—NH—, —NR^{k}—C(NH)—NH—, —NR^{k}—C(NR^{k})—NH—, —NH—C(N(CN))—NH—, —NH—C(NSO₂R^{c})—NH—, —NR^{k}—C(NSO₂R^{c})—NH—, —NH—C(NNO₂)—NH—, NH—C(NC(O)R^{c})—NH—, —NH—C(O)—NH—, or —NH—C(S)—NH—.

In some embodiments, in the compounds represented by formula (I), (II), (III), (X) or (XIV), G is —NH—S(O)₂—NH—, —NR^{k}—S(O)₂—O—, —P(O)(R^{c})—, —P(O)(R^{c})—O—, or —P(O)(R^{c})—NR^{k}—.

In some embodiments, in the compounds represented by formula (I), (II), (III), (X) or (XIV), G is an optionally substituted cyclyl, an optionally substituted cycloalkyl, an optionally substituted heterocycloalkyl or an optionally substituted heterocyclyl.

In some embodiments, in the compounds represented by formula (I), (II), (III), (X) or (XIV), G is an optionally substituted cyclopropyl, an optionally substituted cyclobutyl, an optionally substituted cyclopentyl, an optionally substituted cyclohexyl, an optionally substituted cycloheptyl, an optionally substituted aziridinyl, an optionally substituted oxiranyl, an optionally substituted azetidinyl, an optionally substituted oxetanyl, an optionally substituted morpholinyl, an optionally substituted piperazinyl or an optionally substituted piperidinyl.

In some embodiments, in the compounds represented by formula (I), (II), (III), (X) or (XIV), G is an optionally substituted aryl, an optionally substituted heteroaryl, an optionally substituted heteroaralkyl, —C(N—CN)—NH—, —Si(OH)₂—, —C(NH)—NR^{k}—, or —NR^{k}—CH₂—C(O)—.

In some embodiments, in the compounds represented by formula (I), (II), (III), (X) or (XIV), G is an optionally substituted imidazolyl, an optionally substituted imidazolidinone, an optionally substituted imidazolidineamine, an optionally substituted pyrrolidinyl, an optionally substituted pyrrolyl, an optionally substituted furanyl, an optionally substituted thienyl, an optionally substituted thiazolyl, an optionally substituted triazolyl, an optionally substituted oxadiazolyl, an optionally substituted thiadiazolyl, an optionally substituted pyrazolyl, an optionally substituted tetrazolyl, an optionally substituted oxazolyl, an optionally substituted isoxazolyl, an optionally substituted phenyl, an optionally substituted pyridyl, an optionally substituted pyrimidyl, an optionally substituted indolyl, or an optionally substituted benzothiazolyl.

In some embodiments, in the compounds represented by formula (I), (II), (III), (X) or (XIV), Y is O or CH₂; G is absent; and n is 0, 1, 2, 3 or 4.

In some embodiments, in the compounds represented by formula (I), (II), (III), (X) or (XIV), Y is absent, O, S, NR^{k}, or CH₂; and n is 0, 1, 2, 3, or 4.

In some embodiments, in the compounds represented by formula (II), X₁ is one of the following formulas:

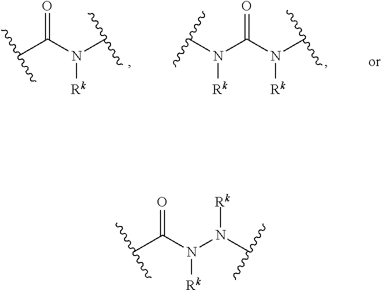

In some embodiments, in the compounds represented by formula (II), X₁ is represented by the following formula:

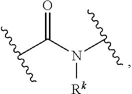

wherein R^{k} is —H or a lower alkyl.

In some embodiments, in the compounds represented by formula (II), X₁ is represented by the following formula:

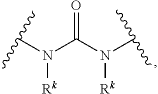

wherein R^{k} is —H or a lower alkyl.

In some embodiments, in the compounds represented by formula (II), X₁ is represented by the following formula:

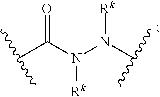

wherein R^{k} is —H or a lower alkyl.

In some embodiments, in the compounds represented by formula (III), X₃ is —C(R^{g})═N—NR^{k}—, wherein R^{g} and R^{k} of X₃ are each, independently, —H or a lower alkyl.

In some embodiments, in the compounds represented by formula (IV), the compound is represented by formula (V):

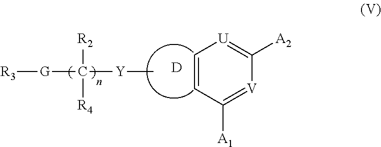

or a pharmaceutically acceptable salt thereof, wherein:

G, Y, R₂, R₃, R₄, and n are defined as for formula I; and

Ring D, A₁, A₂, U, and V are defined as for formula (IV).

In some embodiments, in the compounds represented by formula (IV) or (V), the compound is represented by one of the following structural formulas:

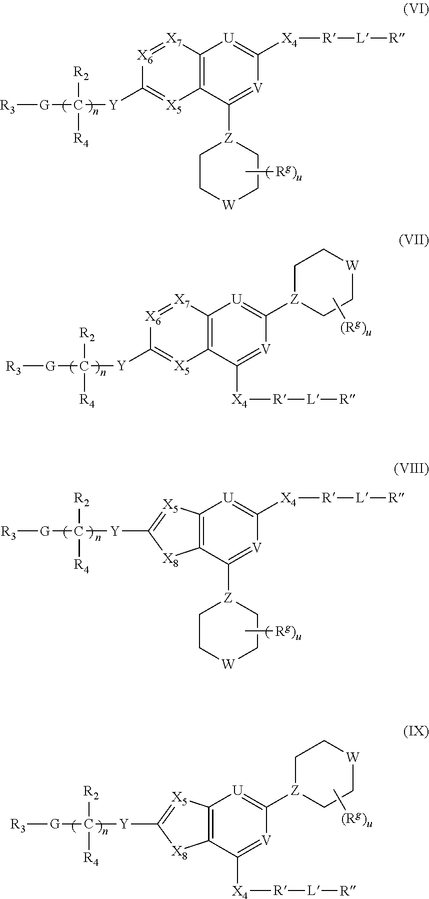

or a pharmaceutically acceptable salt thereof, wherein:

G, Y, R₂, R₃, R₄, R^{g}, and n are defined as for formula I;

U, V, L, X₄, W, Z, R′, R″, and u are defined as for formula (IV);

X₅, X₆ and X₇ are each, independently, N or CR^{g};

X₈ is CR^{g}R^{g}, O, S(O)ₚ, or NR^{k}, wherein R^{k} is defined as for formula (I).

In some embodiments, in the compounds represented by formula (VI) or formula (VII), U and V are N; and X₅, X₆ and X₇ are CR^{g}.

In some embodiments, in the compounds represented by formula (IV), (V), (VI), (VII), (VIII), or (IX), R′ and L′ are absent.

In some embodiments, in the compounds represented by formula (IV), (V), (VI), (VII), (VIII), or (IX), R″ is an optionally substituted cycloalkyl, an optionally substituted cyclyl, an optionally substituted aryl, an optionally substituted heterocycloalkyl, an optionally substituted heterocyclyl, or an optionally substituted heteroaryl.

In some embodiments, in the compounds represented by formula (IV), (V), (VI), (VII), (VIII), or (IX), R″ is an optionally substituted aryl or an optionally substituted heteroaryl.

In some embodiments, in the compounds represented by formula (IV), (V), (VI), (VII), (VIII), or (IX), R″ is substituted with one or more substituent selected from the group consisting of a lower alkyl, cyano, halo, nitro, —NH₂, a lower alkylamino, a lower dialkylamino, a lower alkoxy, a lower haloalkyl, —S(O)ₚR^{c}, and —C(O)R^{c}.

In some embodiments, in the compounds represented by formula (IV), (V), (VI), (VII), (VIII), or (IX), Z is N and W is O.

In some embodiments, in the compounds represented by formula (V), (VI), (VII), (VIII), or (IX), Y is a covalent bond, O, S, N(R^{k}), or CH₂, and n is 0, 1, 2, 3, or 4.

In some embodiments, in the compounds represented by formula (V), (VI), (VII), (VIII), or (IX), G is absent.

In some embodiments, in the compounds represented by formula (V), (VI), (VII), (VIII), or (IX), G is >C═N—R, —NR^{k}C(O)—, —C(O)NR^{k}—, —OC(O)—, —C(O)O—, —OC(O)O—, —NR^{k}C(O)O—, —OC(O)NR^{k}—, —NR^{k}C(S)O—, —OC(S)NR^{k}—, —NR^{k}C(NR)NR^{k}—, —NR^{k}C(O)NR^{k}—, —NR^{k}C(S)NR^{k}—, —NR^{k}S(O)₂NR^{k}—, —C(NR)NR^{k}—, or —NR^{k}CR^{g}R^{g}C(O)—.

In some embodiments, in the compounds represented by formula (V), (VI), (VII), (VIII), or (IX), R₃ is an optionally substituted alkyl, an optionally substituted aryl, an optionally substituted heteroaryl, an optionally substituted cycloalkyl, an optionally substituted cyclyl, an optionally substituted heterocycloalkyl, an optionally substituted heterocyclyl, nitro, cyano, halo, OR^{k}, SR^{k}, or NR^{h}R^{j}.

In some embodiments, in the compounds represented by formula (V), (VI), (VII), (VIII), or (IX), R₃ is optionally substituted aryl or optionally substituted heteroaryl.

In some embodiments, in the compounds represented by formula (V), (VI), (VII), (VIII), or (IX), R₃ is an optionally substituted phenyl, an optionally substituted naphthyl, an optionally substituted anthracenyl, an optionally substituted fluorenyl, an optionally substituted indenyl, an optionally substituted azulenyl, an optionally substituted pyridyl, an optionally substituted 1-oxo-pyridyl, an optionally substituted furanyl, an optionally substituted benzo[1,3]dioxolyl, an optionally substituted benzo[1,4]dioxinyl, an optionally substituted thienyl, an optionally substituted pyrrolyl, an optionally substituted oxazolyl, an optionally substituted imidazolyl, an optionally substituted thiazolyl, an optionally substituted isoxazolyl, an optionally substituted quinolinyl, an optionally substituted pyrazolyl, an optionally substituted isothiazolyl, an optionally substituted pyridazinyl, an optionally substituted pyrimidinyl, an optionally substituted pyrazinyl, an optionally substituted triazinyl, an optionally substituted triazolyl, an optionally substituted thiadiazolyl, an optionally substituted isoquinolinyl, an optionally substituted indazolyl, an optionally substituted benzoxazolyl, an optionally substituted benzofuryl, an optionally substituted indolizinyl, an optionally substituted imidazopyridyl, an optionally substituted tetrazolyl, an optionally substituted benzimidazolyl, an optionally substituted benzothiazolyl, an optionally substituted benzothiadiazolyl, an optionally substituted benzoxadiazolyl, an optionally substituted indolyl, an optionally substituted tetrahydroindolyl, an optionally substituted azaindolyl, an optionally substituted indazolyl, an optionally substituted imidazopyridyl, an optionally substituted quinazolinyl, an optionally substituted purinyl, an optionally substituted pyrrolo[2,3]pyrimidinyl, an optionally substituted pyrazolo[3,4]pyrimidinyl, or an optionally substituted benzo(b)thienyl.

In some embodiments, in the compounds represented by formula (V), (VI), (VII), (VIII), or (IX), R₃ is an optionally substituted heterocycloalkyl.

In some embodiments, in the compounds represented by formula (V), (VI), (VII), (VIII), or (IX), R₃ is an optionally substituted piperidinyl, an optionally substituted piperazinyl, an optionally substituted 2-oxopiperazinyl, an optionally substituted 2-oxopiperidinyl, an optionally substituted 2-oxopyrrolidinyl, an optionally substituted 4-piperidonyl, an optionally substituted tetrahydropyranyl, an optionally substituted oxazolidinyl, an optionally substituted 2-oxo-oxazolidinyl, an optionally substituted tetrahydrothiopyranyl, an optionally substituted tetrahydrothiopyranyl sulfone, an optionally substituted morpholinyl, an optionally substituted thiomorpholinyl, an optionally substituted thiomorpholinyl sulfoxide, an optionally substituted thiomorpholinyl sulfone, an optionally substituted 1,3-dioxolanyl, an optionally substituted [1,4]dioxanyl, an optionally substituted 2-oxo-imidazolidinyl, tetrahydrofuranyl, or an optionally substituted tetrahydrothienyl.

In some embodiments, in the compounds represented by formula (V), (VI), (VII), (VIII), or (IX), R₃ is —OR^{k} or —NR^{h}R^{j}, and R^{f}, R^{h} and R^{j} are each, independently, H, an optionally substituted alkyl, an optionally substituted aryl, an optionally substituted heteroaryl, an optionally substituted cycloalkyl, an optionally substituted heterocycloalkyl, or —C(O)R^{c}.

In some embodiments, in the compounds represented by formula (V), (VI), (VII), (VIII), or (IX), R₃ is —C(O)OR^{k}, —OC(O)R^{k}, —C(O)NR^{h}R^{j}, —NR^{k}C(O)R^{k}, —C(S)OR^{k}, —OC(S)R^{k}, —NR^{k}C(O)NR^{h}R^{j}, —NR^{k}C(S)NR^{h}R^{j}, —C(O)NR^{h}R^{j}, —S(O)₂R^{k}, —S(O)₂NR^{h}R^{j}, —OC(O)NR^{h}R^{j}, or —NR^{k}C(O)OR^{k}.

In some embodiments, in the compounds represented by formula (IV), the compound is represented by one of the following structural formulas:

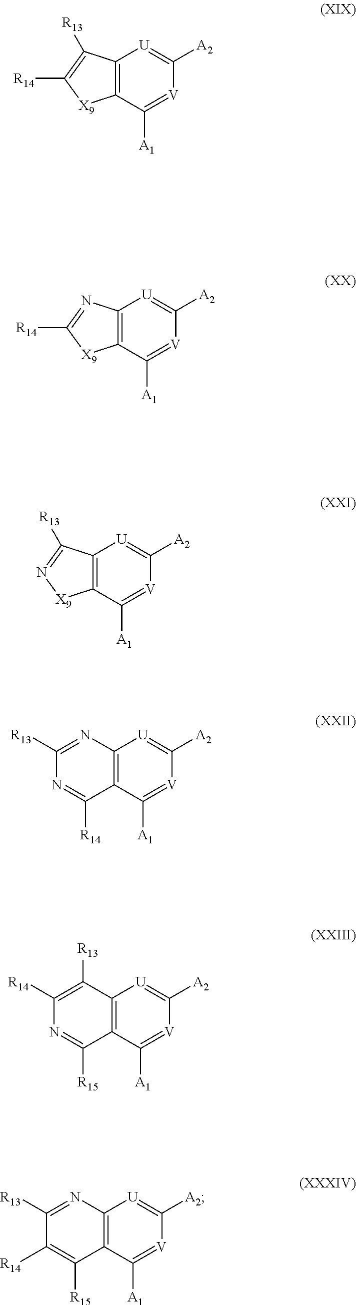

or a pharmaceutically acceptable salt thereof, wherein;

U, V, A₁, and A₂ are defined as for formula (IV);

X₉ is CR^{g}R^{g}, O, S(O)ₚ, or NR^{k};

one of R₁₃, R₁₄ and R₁₅ is a group represented by the following structural formula:

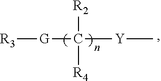

and the remainder of R₁₃, R₁₄ and R₁₅ are independently selected from H, R^{g}, or isothionitro; and

R₂, R₃, R₄, G, Y, R^{g}, R^{k} and n are defined as for formula (I).

In some embodiments, in the compounds represented by formula (XIX), (XX), (XXI), (XXII), (XXIII), or (XXIV), U and V are N.

In some embodiments, in the compounds represented by formula (XIX), (XX), (XXI), (XXII), (XXIII), or (XXIV), R′ and L′ are absent.

In some embodiments, in the compounds represented by formula (XIX), (XX), (XXI), (XXII), (XXIII), or (XXIV), R″ is an optionally substituted cycloalkyl, an optionally substituted cyclyl, an optionally substituted aryl, an optionally substituted heterocycloalkyl, an optionally substituted heterocyclyl, or an optionally substituted heteroaryl.

In some embodiments, in the compounds represented by formula (XIX), (XX), (XXI), (XXII), (XXIII), or (XXIV), R″ is an optionally substituted aryl or an optionally substituted heteroaryl.

In some embodiments, in the compounds represented by formula (XIX), (XX), (XXI), (XXII), (XXIII), or (XXIV), R″ is substituted with one or more substituent selected from the group consisting of a lower alkyl, cyano, halo, nitro, —NH₂, a lower alkylamino, a lower dialkylamino, a lower alkoxy, a lower haloalkyl, —S(O)ₚR^{c}, and —C(O)R^{c}.

In some embodiments, in the compounds represented by formula (XIX), (XX), (XXI), (XXII), (XXIII), or (XXIV), Z is N and W is O.

In some embodiments, in the compounds represented by formula (XIX), (XX), (XXI), (XXII), (XXIII), or (XXIV), Y is a covalent bond, O, S, N(R^{k}), or CH₂, and n is 0, 1, 2, 3, or 4.

In some embodiments, in the compounds represented by formula (XIX), (XX), (XXI), (XXII), (XXIII), or (XXIV), G is absent.

In some embodiments, in the compounds represented by formula (XIX), (XX), (XXI), (XXII), (XXIII), or (XXIV), G is >C═N—R, —NR^{k}C(O)—, —C(O)NR^{k}—, —OC(O)—, —C(O)O—, —OC(O)O—, —NR^{k}C(O)O—, —OC(O)NR^{k}—, —NR^{k}C(S)O—, —OC(S)NR^{k}—, —NR^{k}C(NR)NR^{k}—, —NR^{k}C(O)NR^{k}—, —NR^{k}C(S)NR^{k}—, —NR^{k}S(O)₂NR^{k}—, —C(NR)NR^{k}—, or —NR^{k}CR^{g}R^{g}C(O)—.

In some embodiments, in the compounds represented by formula (XIX), (XX), (XXI), (XXII), (XXIII), or (XXIV), R₃ is an optionally substituted alkyl, an optionally substituted aryl, an optionally substituted heteroaryl, an optionally substituted cycloalkyl, an optionally substituted cyclyl, an optionally substituted heterocycloalkyl, an optionally substituted heterocyclyl, nitro, cyano, halo, OR^{k}, SR^{k}, or NR^{h}R^{j}.

In some embodiments, in the compounds represented by formula (XIX), (XX), (XXI), (XXII), (XXIII), or (XXIV), R₃ is optionally substituted aryl or optionally substituted heteroaryl.

In some embodiments, in the compounds represented by formula (XIX), (XX), (XXI), (XXII), (XXIII), or (XXIV), R₃ is an optionally substituted phenyl, an optionally substituted naphthyl, an optionally substituted anthracenyl, an optionally substituted fluorenyl, an optionally substituted indenyl, an optionally substituted azulenyl, an optionally substituted pyridyl, an optionally substituted 1-oxo-pyridyl, an optionally substituted furanyl, an optionally substituted benzo[1,3]dioxolyl, an optionally substituted benzo[1,4]dioxinyl, an optionally substituted thienyl, an optionally substituted pyrrolyl, an optionally substituted oxazolyl, an optionally substituted imidazolyl, an optionally substituted thiazolyl, an optionally substituted isoxazolyl, an optionally substituted quinolinyl, an optionally substituted pyrazolyl, an optionally substituted isothiazolyl, an optionally substituted pyridazinyl, an optionally substituted pyrimidinyl, an optionally substituted pyrazinyl, an optionally substituted triazinyl, an optionally substituted triazolyl, an optionally substituted thiadiazolyl, an optionally substituted isoquinolinyl, an optionally substituted indazolyl, an optionally substituted benzoxazolyl, an optionally substituted benzofuryl, an optionally substituted indolizinyl, an optionally substituted imidazopyridyl, an optionally substituted tetrazolyl, an optionally substituted benzimidazolyl, an optionally substituted benzothiazolyl, an optionally substituted benzothiadiazolyl, an optionally substituted benzoxadiazolyl, an optionally substituted indolyl, an optionally substituted tetrahydroindolyl, an optionally substituted azaindolyl, an optionally substituted indazolyl, an optionally substituted imidazopyridyl, an optionally substituted quinazolinyl, an optionally substituted purinyl, an optionally substituted pyrrolo[2,3]pyrimidinyl, an optionally substituted pyrazolo[3,4]pyrimidinyl, or an optionally substituted benzo(b)thienyl.

In some embodiments, in the compounds represented by formula (XIX), (XX), (XXI), (XXII), (XXIII), or (XXIV), R₃ is an optionally substituted heterocycloalkyl.

In some embodiments, in the compounds represented by formula (XIX), (XX), (XXI), (XXII), (XXIII), or (XXIV), R₃ is an optionally substituted piperidinyl, an optionally substituted piperazinyl, an optionally substituted 2-oxopiperazinyl, an optionally substituted 2-oxopiperidinyl, an optionally substituted 2-oxopyrrolidinyl, an optionally substituted 4-piperidonyl, an optionally substituted tetrahydropyranyl, an optionally substituted oxazolidinyl, an optionally substituted 2-oxo-oxazolidinyl, an optionally substituted tetrahydrothiopyranyl, an optionally substituted tetrahydrothiopyranyl sulfone, an optionally substituted morpholinyl, an optionally substituted thiomorpholinyl, an optionally substituted thiomorpholinyl sulfoxide, an optionally substituted thiomorpholinyl sulfone, an optionally substituted 1,3-dioxolanyl, an optionally substituted [1,4]dioxanyl, an optionally substituted 2-oxo-imidazolidinyl, tetrahydrofuranyl, or an optionally substituted tetrahydrothienyl.

In some embodiments, in the compounds represented by formula (XIX), (XX), (XXI), (XXII), (XXIII), or (XXIV), R₃ is —OR^{k} or —NR^{h}R^{j}, and R^{f}, R^{h} and R^{j} are each, independently, H, an optionally substituted alkyl, an optionally substituted aryl, an optionally substituted heteroaryl, an optionally substituted cycloalkyl, an optionally substituted heterocycloalkyl, or —C(O)R^{c}.

In some embodiments, in the compounds represented by formula (XIX), (XX), (XXI), (XXII), (XXIII), or (XXIV), R₃ is —C(O)OR^{k}, —OC(O)R^{k}, —C(O)NR^{h}R^{j}, —NR^{k}C(O)R^{k}, —C(S)OR^{k}, —OC(S)R^{k}, —NR^{k}C(O)NR^{h}R^{j}, —NR^{k}C(S)NR^{h}R^{j}, —C(O)NR^{h}R^{j}, —S(O)₂R^{k}, —S(O)₂NR^{h}R^{j}, —OC(O)NR^{h}R^{j}, or —NR^{k}C(O)OR^{k}.

In some embodiments, in the compounds represented by formula (X), the compound is represented by one of the following structural formulas:

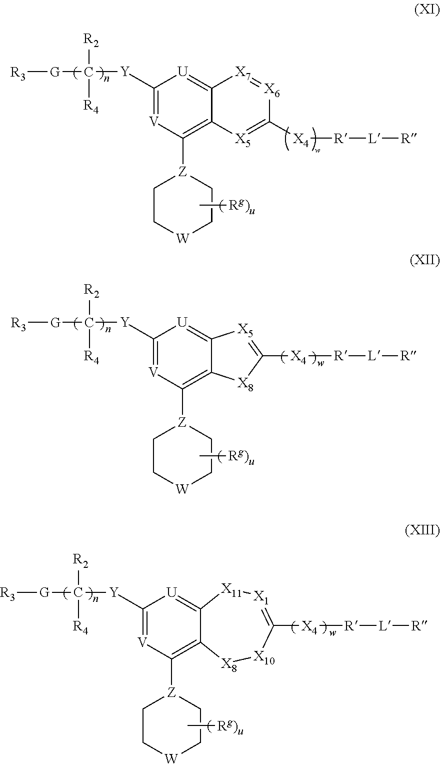

or a pharmaceutically acceptable salt thereof, wherein:

G, Y, R₂, R₃, R₄, R^{g} and n are defined as for formula (I);

R′, R″, L′, X₄, U, V, W, Z, and u are defined as for formula (IV);

w is defined as for formula (X);

X₅, X₆ and X₇ are each, independently, N or CR^{g}; and

X₈, X₁₀, and X₁₁ are each, independently, CR^{g}R^{g}, O, S(O)ₚ, or NR^{k}, wherein R^{k} is defined as for formula (I).

In some embodiments, in the compounds represented by formula (XI), U and V are N; and X₅ and X₆ are CR^{g}.

In some embodiments, in the compounds represented by formula (XI), U and V are N; X₅ and X₆ are CR^{g}; and X₇ is N.

In some embodiments, in the compounds represented by formula (XI), U and V are N; X₅ and X₆ are CR^{g}; and X₇ is CR^{g}.

In some embodiments, in the compounds represented by formula (XI), (XII), or (XIII), w is 0, and R′ and L′ are absent.

In some embodiments, in the compounds represented by formula (XI), (XII), or (XIII), R″ is an optionally substituted cycloalkyl, an optionally substituted cyclyl, an optionally substituted aryl, an optionally substituted heterocycloalkyl, an optionally substituted heterocyclyl, or an optionally substituted heteroaryl.

In some embodiments, in the compounds represented by formula (XI), (XII), or (XIII), R″ is an optionally substituted aryl or an optionally substituted heteroaryl.

In some embodiments, in the compounds represented by formula (XI), (XII), or (XIII), R″ is substituted with one or more substituent selected from the group consisting of a lower alkyl, cyano, halo, nitro, —NH₂, a lower alkylamino, a lower dialkylamino, a lower alkoxy, a lower haloalkyl, —S(O)ₚR^{c}, and —C(O)R^{c}.

In some embodiments, in the compounds represented by formula (XI), (XII), or (XIII), Z is N and W is O.

In some embodiments, in the compounds represented by formula (XI), (XII), or (XIII), Y is a covalent bond, O, S, N(R^{k}), or CH₂, and n is 0, 1, 2, 3, or 4.

In some embodiments, in the compounds represented by formula (XI), (XII), or (XIII), G is absent.

In some embodiments, in the compounds represented by formula (XI), (XII), or (XIII), G is >C═N—R, —NR^{k}C(O)—, —C(O)NR^{k}—, —OC(O)—, —C(O)O—, —OC(O)O—, —NR^{k}C(O)O—, —OC(O)NR^{k}—, —NR^{k}C(S)O—, —OC(S)NR^{k}—, —NR^{k}C(NR)NR^{k}—, —NR^{k}C(O)NR^{k}—, —NR^{k}C(S)NR^{k}—, —NR^{k}S(O)₂NR^{k}—, —C(NR)NR^{k}—, or —NR^{k}CR^{g}R^{g}C(O)—.

In some embodiments, in the compounds represented by formula (XI), (XII), or (XIII), R₃ is an optionally substituted alkyl, an optionally substituted aryl, an optionally substituted heteroaryl, an optionally substituted cycloalkyl, an optionally substituted cyclyl, an optionally substituted heterocycloalkyl, an optionally substituted heterocyclyl, nitro, cyano, halo, OR^{k}, SR^{k}, or NR^{h}R^{j}.

In some embodiments, in the compounds represented by formula (XI), (XII), or (XIII), R₃ is optionally substituted aryl or optionally substituted heteroaryl.

In some embodiments, in the compounds represented by formula (XI), (XII), or (XIII), R₃ is an optionally substituted phenyl, an optionally substituted naphthyl, an optionally substituted anthracenyl, an optionally substituted fluorenyl, an optionally substituted indenyl, an optionally substituted azulenyl, an optionally substituted pyridyl, an optionally substituted 1-oxo-pyridyl, an optionally substituted furanyl, an optionally substituted benzo[1,3]dioxolyl, an optionally substituted benzo[1,4]dioxinyl, an optionally substituted thienyl, an optionally substituted pyrrolyl, an optionally substituted oxazolyl, an optionally substituted imidazolyl, an optionally substituted thiazolyl, an optionally substituted isoxazolyl, an optionally substituted quinolinyl, an optionally substituted pyrazolyl, an optionally substituted isothiazolyl, an optionally substituted pyridazinyl, an optionally substituted pyrimidinyl, an optionally substituted pyrazinyl, an optionally substituted triazinyl, an optionally substituted triazolyl, an optionally substituted thiadiazolyl, an optionally substituted isoquinolinyl, an optionally substituted indazolyl, an optionally substituted benzoxazolyl, an optionally substituted benzofuryl, an optionally substituted indolizinyl, an optionally substituted imidazopyridyl, an optionally substituted tetrazolyl, an optionally substituted benzimidazolyl, an optionally substituted benzothiazolyl, an optionally substituted benzothiadiazolyl, an optionally substituted benzoxadiazolyl, an optionally substituted indolyl, an optionally substituted tetrahydroindolyl, an optionally substituted azaindolyl, an optionally substituted indazolyl, an optionally substituted imidazopyridyl, an optionally substituted quinazolinyl, an optionally substituted purinyl, an optionally substituted pyrrolo[2,3]pyrimidinyl, an optionally substituted pyrazolo[3,4]pyrimidinyl, or an optionally substituted benzo(b)thienyl.

In some embodiments, in the compounds represented by formula (XI), (XII), or (XIII), R₃ is an optionally substituted heterocycloalkyl.

In some embodiments, in the compounds represented by formula (XI), (XII), or (XIII), R₃ is an optionally substituted piperidinyl, an optionally substituted piperazinyl, an optionally substituted 2-oxopiperazinyl, an optionally substituted 2-oxopiperidinyl, an optionally substituted 2-oxopyrrolidinyl, an optionally substituted 4-piperidonyl, an optionally substituted tetrahydropyranyl, an optionally substituted oxazolidinyl, an optionally substituted 2-oxo-oxazolidinyl, an optionally substituted tetrahydrothiopyranyl, an optionally substituted tetrahydrothiopyranyl sulfone, an optionally substituted morpholinyl, an optionally substituted thiomorpholinyl, an optionally substituted thiomorpholinyl sulfoxide, an optionally substituted thiomorpholinyl sulfone, an optionally substituted 1,3-dioxolanyl, an optionally substituted [1,4]dioxanyl, an optionally substituted 2-oxo-imidazolidinyl, tetrahydrofuranyl, or an optionally substituted tetrahydrothienyl.

In some embodiments, in the compounds represented by formula (XI), (XII), or (XIII), R₃ is —OR^{k} or —NR^{h}R^{j}, and R^{f}, R^{h} and R^{j} are each, independently, H, an optionally substituted alkyl, an optionally substituted aryl, an optionally substituted heteroaryl, an optionally substituted cycloalkyl, an optionally substituted heterocycloalkyl, or —C(O)R^{c}.

In some embodiments, in the compounds represented by formula (XI), (XII), or (XIII), R₃ is —C(O)OR^{k}, —OC(O)R^{k}, —C(O)NR^{h}R^{j}, —NR^{k}C(O)R^{k}, —C(S)OR^{k}, —OC(S)R^{k}, —NR^{k}C(O)NR^{h}R^{j}, —NR^{k}C(S)NR^{h}R^{j}, —C(O)NR^{h}R^{j}, —S(O)₂R^{k}, —S(O)₂NR^{h}R^{j}, —OC(O)NR^{h}R^{j}, or —NR^{k}C(O)OR^{k}.

In some embodiments, in the compounds represented by formula (XI), (XII), or (XIII), w is 1; X₄ is O, S, or NRₖ; and R′ and L′ are absent.

In some embodiments, in the compounds represented by formula (XIV), the compound is represented by formula (XV):

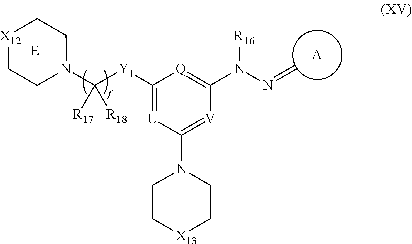

or a pharmaceutically acceptable salt thereof, wherein:

Q, U, and V are defined as for formula (I);

R₁₆ is defined as for formula (XIV);

ring E is optionally substituted with one to four substituents selected from a lower alkyl, a halo, an amino, a lower alkyl amino, a lower dialkyl amino, a cyano, a nitro, a lower haloalkyl, a hydroxyl, and a lower hydroxyalkyl;

X₁₂ is O, S, S(O), S(O)₂, or CR^{g}R^{g};

X₁₃ is O, S, S(O), S(O)₂, or CH₂;

Y₁ is O, S, NR^{k}, or CH₂;

R₁₇ and R₁₈, for each occurrence, are independently, H or a lower alkyl; or R₁₇ and R₁₈ taken together with the carbon to which they are attached form a cycloalkyl; and

f is 0, 1, 2, or 3.

In some embodiments, in the compounds represented by formula (XIV), the compound is represented by formula (XVI):

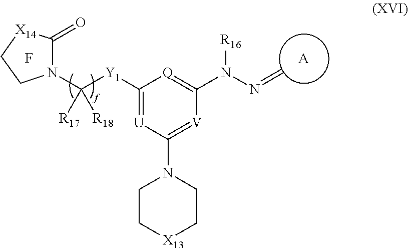

or a pharmaceutically acceptable salt thereof, wherein:

Q, U, and V are defined as for formula (I);

R₁₆ is defined as for formula (XIV);

Y₁, R₁₇, R₁₈, X₁₃, and f are defined as for formula (XV);

ring F is optionally substituted with one or two substituents selected from a lower alkyl, a halo, an amino, a lower alkyl amino, a lower dialkyl amino, a cyano, a nitro, a lower haloalkyl, a hydroxyl, and a lower hydroxyalkyl; and

X₁₄ is O, NR^{k}, or CR^{g}R^{g}.

In some embodiments, in the compounds represented by formula (XIV), the compound is represented by formula (XVII):

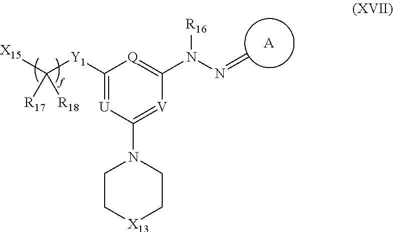

or a pharmaceutically acceptable salt thereof, wherein:

Q, U, and V are defined as for formula (I);

R₁₆ is defined as for formula (XIV);

Y₁, R₁₇, R₁₈, X₁₃, and f are defined as for formula (XV); and

X₁₅ is —OH, —NH₂ or —SH.

In some embodiments, in the compounds represented by formula (XV), (XVI), or (XVII), Q, U, and V are N.

In some embodiments, in the compounds represented by formula (XV), (XVI), or (XVII), one of Q, U, or V is CR^{g}, and the other two are N.

In some embodiments, in the compounds represented by formula (XV), (XVI), or (XVII), V is CR^{g}, Q and U are N.

In some embodiments, in the compounds represented by formula (XV), (XVI), or (XVII), Q is CR^{g}, V and U are N.

In some embodiments, in the compounds represented by formula (XV), (XVI), or (XVII), U is CR^{g}, V and Q are N.

In some embodiments, in the compounds represented by formula (XV), (XVI), or (XVII), one of Q, U, or V is N, and the other two are CR^{g}.

In some embodiments, in the compounds represented by formula (XV), (XVI), or (XVII), V is N, and Q and U are CR^{g}.

In some embodiments, in the compounds represented by formula (XV), (XVI), or (XVII), Q is N, and V and U are CR^{g}.

In some embodiments, in the compounds represented by formula (XV), (XVI), or (XVII), U is N and Q, and V are CR^{g}.

In some embodiments, in the compounds represented by formula (XV), (XVI), or (XVII), —NR₅R₆ is an optionally substituted morpholino, an optionally substituted thiomorpholino, an optionally substituted 1-oxo-thiomorpholino, an optionally substituted 1,1-dioxo-thiomorpholino, an optionally substituted piperidinyl, or an optionally substituted piperazinyl.

In some embodiments, in the compounds represented by formula (XIV), (XV), (XVI), or (XVII), ring A is a ring system selected from the group consisting of:

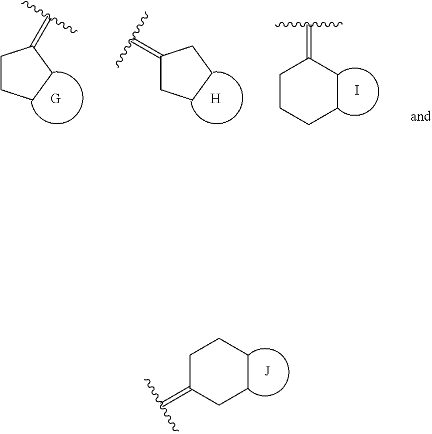

wherein:

[symbol] represents the point of attachment;

rings G, H, I, and J are each, independently, an aryl or a heteroaryl; and

each ring system is optionally substituted with one or more substituents.

In some embodiments, in the compounds represented by formula (XIV), (XV), (XVI), or (XVII), ring A is a ring system selected from the group consisting of:

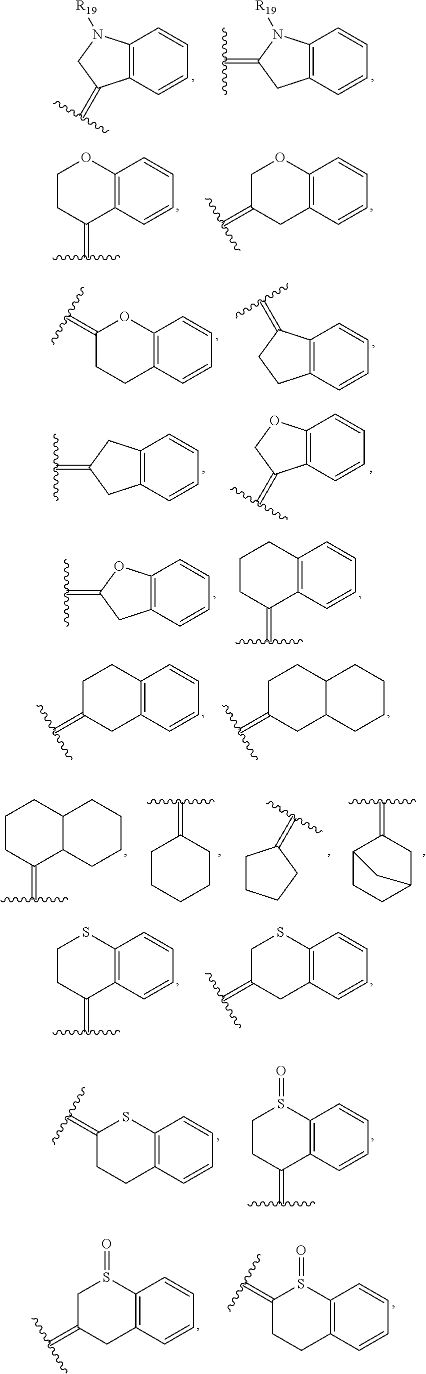

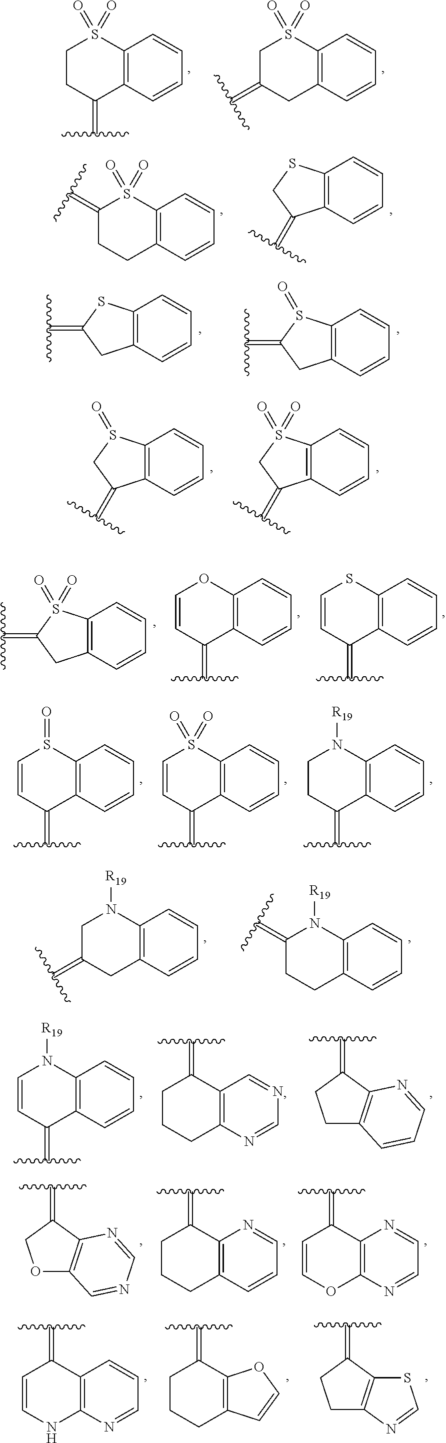

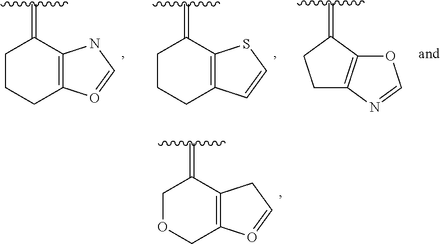

wherein:
each ring system is optionally substituted with one or more substituents;
[symbol] represents the point of attachment; and
R₁₉ is H, an alkyl, an aralkyl, or an alkylcarbonyl.

In some embodiments, in the compounds represented by formula (XIV), (XV), (XVI), or (XVII), ring A is a ring system selected from the group consisting of:

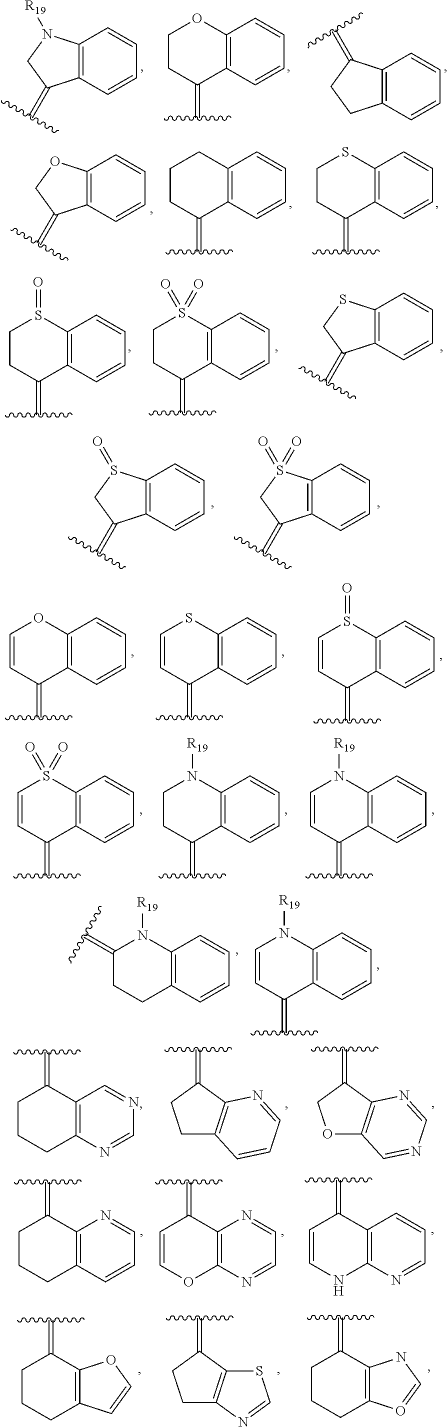

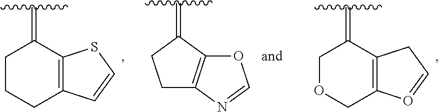

wherein:

each ring system is optionally substituted with one or more substituents.

In some embodiments, in the compounds represented by formula (XIV), (XV), (XVI), or (XVII), ring A is optionally substituted with one or more substituents selected from the group consisting of an optionally substituted alkyl, an optionally substituted alkoxy, an optionally substituted alkyl sulfanyl, an optionally substituted alkenyl, an optionally substituted alkynyl, an optionally substituted cycloalkyl, an optionally substituted cyclyl, an optionally substituted heterocyclyl, an optionally substituted heterocycloalkyl, an optionally substituted aryl, an optionally substituted heteroaryl, an optionally substituted aralkyl, an optionally substituted heteroaralkyl, a haloalkyl, halo, cyano, nitro, haloalkoxy, ═O, ═S, ═NR, —OR^{k}, —NR^{h}R^{j}, —SR^{k}, —C(O)R^{k}, —C(O)NR^{h}R^{j}, —NR^{k}C(O)R^{k}, —C(O)OR^{k}, —OC(O)R^{k}, —NR^{k}C(O)NR^{h}R^{j}, —OC(O)NR^{h}R^{j}, —NR^{k}C(O)OR^{k}, —C(NR)R^{k}, —C(NR)NR^{h}R^{j}, —NR^{k}C(NR)R^{k}, —C(NR)OR^{k}, —OC(NR)R^{k}, —NR^{k}C(NR)NR^{h}R^{j}, —OC(NR)NR^{h}R^{j}, —NR^{k}C(NR)OR^{k}, —C(S)R′, —C(S)NR^{h}R^{j}, —NR^{k}C(S)R^{k}, —C(S)OR^{k}, —OC(S)R^{k}, —NR^{k}C(S)NR^{h}R^{j}, —OC(S)NR^{h}R^{j}, —NR^{k}C(S)OR^{k}, —C(O)SR^{k}, —SC(O)R^{k}, —S(O)ₚR^{k}, —S(O)ₚNR^{h}R^{j}, —OS(O)ₚR^{k}, —S(O)ₚOR^{k}, —OS(O)ₚOR^{k}, —P(O)(OR^{k})₂, —OP(O)(OR^{k})₂, —P(S)(OR^{k})₂, —SP(O)(OR^{k})₂, —P(O)(SR^{k})(OR^{k}), —OP(O)(SR^{k})(OR^{k}), —P(O)(SR^{k})₂, or —OP(O)(SR^{k})₂, wherein p is 1 or 2.

In some embodiments, in the compounds represented by formula (XIV), (XV), (XVI), or (XVII), ring A is optionally substituted with from one to three substituents selected from the group consisting of a lower alkyl, a lower alkoxy, ═O, nitro, cyano, hydroxy, amino, lower alkyl amino, lower dialkyl amino, mercapto, lower alkyl sulfanyl, halo, or haloalkyl.

In some embodiments, in the compounds represented by formula (XV), (XVI), or (XVII), Y₁ is O.

In some embodiments, in the compounds represented by formula (XV), (XVI), or (XVII), Y₁ is a covalent bond.

In some embodiments, in the compounds represented by formula (XIV), Y is O or CH₂; G is absent; and n is 0, 1, 2, 3 or 4.

In some embodiments, in the compounds represented by formula (XIV), Y is absent, O, S, NR^{k}, or CH₂; and n is 0, 1, 2, 3, or 4.

In some embodiments, in the compounds represented by formula (XV), X₁₂, X₁₃, Y₁ is O; and R₁₇ and R₁₈ are each, independently, H or a lower alkyl.

In some embodiments, in the compounds represented by formula (XVI), X₁₃, X₁₄, and Y₁ are O; and R₁₇ and R₁₈ are each, independently, H or a lower alkyl.

In some embodiments, in the compounds represented by formula (XVII), X₁₃ and Y₁ are O; X₁₅ is —OH; and R₁₇ and R₁₈ are each, independently, H or a lower alkyl.

Specific examples of compounds that can be used in the compostions of the invention are set forth below in Table 1:

TABLE 1

| No. | Structure | Name |
|---|---|---|
| 1 | 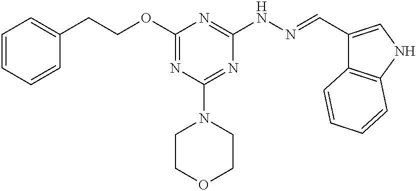 | N-(1H-Indol-3-ylmethylene)- N′-(4-morpholin-4-yl-6- phenethyloxy-[1,3,5]triazin-2- yl)-hydrazine |
| 2 | 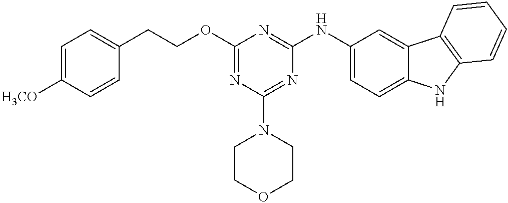 | N-(9H-carbazol-3-yl)-{4-[2-(4- methoxy-phenyl)-ethoxy]-6- morpholin-4-yl-[1,3,5]triazin- 2-yl}-amine |
| 3 | 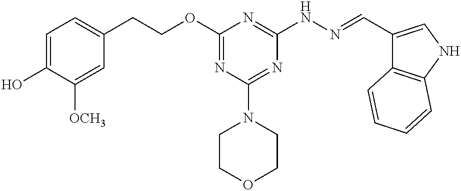 | N-(1H-Indol-3-ylmethylene)- N′-(4-morpholin-4-yl-6-(3- methoxy-4-hydroxy- phenethyloxy-[1,3,5]triazin-2- yl)-hydrazine |
| 4 | 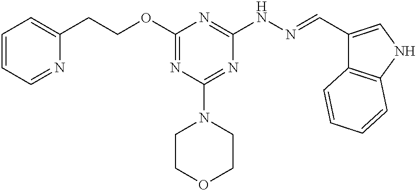 | N-(1H-Indol-3-ylmethylene)- N′-(4-morpholin-4-yl-6-(2- pyridine-2-yl-ethyloxy- [1,3,5]triazin-2-yl)-hydrazine |
| 5 | 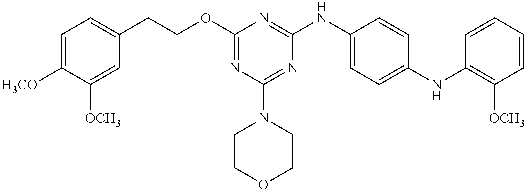 | N-[4-(2-methoxy- phenylamino)-phenyl]-{4-[2- (3,4-dimethoxy-phenethyloxy]- 6-morpholin-4-yl- [1,3,5]triazin-2-yl}-amine |
| 6 | 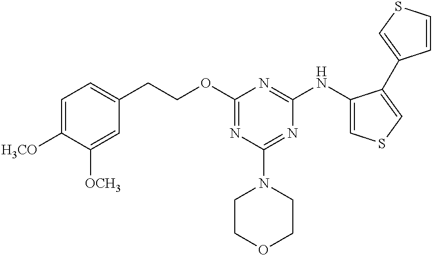 | [3,3′]Bithiophen-4-yl-{4-[2- (3,4-dimethoxy-phenethyloxy]- 6-morpholin-4-yl- [1,3,5]triazin-2-yl}-amine |
| 7 | 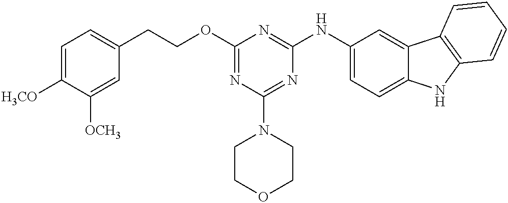 | N-(9H-carbazol-3-yl)-{4-[2- (3,4-dimethoxy-phenyl)- ethoxy]-6-morpholin-4-yl- [1,3,5]triazin-2-yl}-amine |
| 8 | 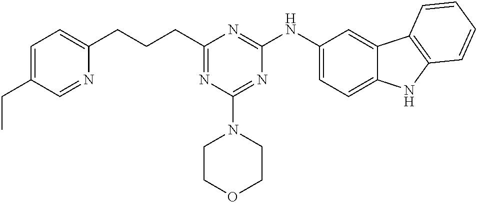 | N-(9H-carbazol-3-yl)-{4-[3-(5- ethyl-phenyl)-propyl]-6- morpholin-4-yl-[1,3,5]triazin- 2-yl}-amine |
| 9 | 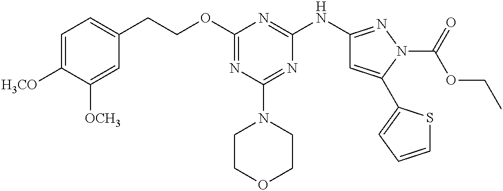 | 3-{4-[2-(3,4-Dimethoxy- phenyl)-ethoxy]-6-morpholin-4-yl- [1,3,5]triazin-2-ylamino}- 5-thiophen-2-yl-pyrazole- 1-carboxylic acid ethyl ester |
| 10 | 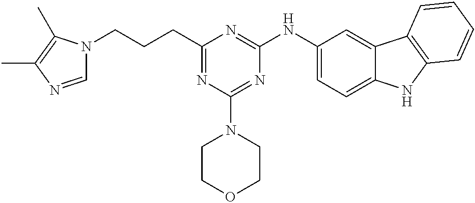 | (9H-Carbazol-3-yl)-{4-[3- (4,5-dimethyl-imidazol- 1-yl)-propyl]-6-morpholin- 4-yl-[1,3,5]triazin-2-yl}- amine |
| 11 | 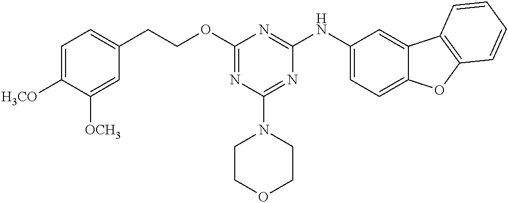 | Dibenzofuran-2-yl-{4-[2- (3,4-dimethoxy-phenyl)- ethoxy]-6-morpholin- 4-yl-[1,3,5]triazin-2-yl}- amine |
| 12 | 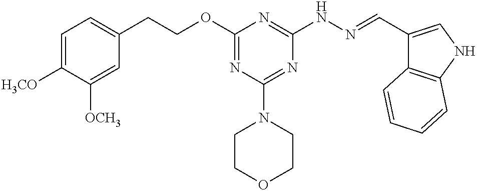 | N-{4-[2-(3,4-Dimethoxy- phenyl)-ethoxy]-6-morpholin- 4-yl-[1,3,5]triazin-2-yl}- N′-(1H-indol-3-ylmethylene)- hydrazine |
| 13 | 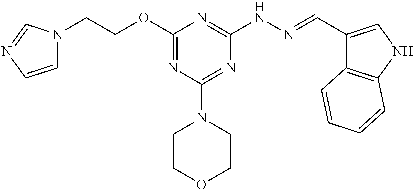 | N-[4-(2-Imidazol-1-yl-ethoxy)- 6-morpholin-4-yl- [1,3,5]triazin-2-yl]-N′-(1H- indol-3-ylmethylene)-hydrazine |
| 14 | 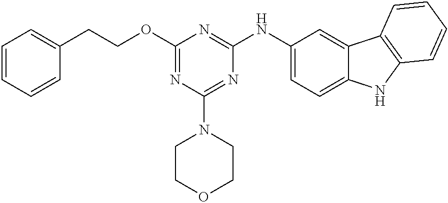 | (9H-Carbazol-3-yl)-(4-morpholin- 4-yl-6-phenethyloxy- [1,3,5]triazin-2-yl)-amine |
| 15 | 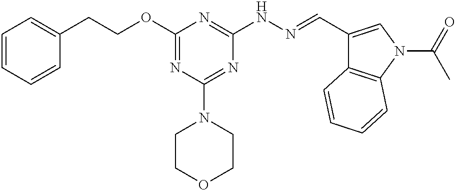 | 1-{3-[(4-Morpholin-4- yl-6-phenethyloxy- [1,3,5]triazin-2-yl)- hydrazonomethyl]-indol- 1-yl}-ethanone |
| 16 | 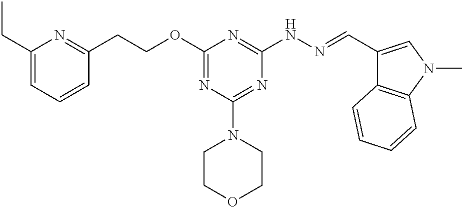 | N-{4-[2-(6-Ethyl-pyridin-2-yl)- ethoxy]-6-morpholin-4-yl- [1,3,5]triazin-2-yl}-N′-(1-methyl- 1H-indol-3-ylmethylene)-hydrazine |
| 17 | 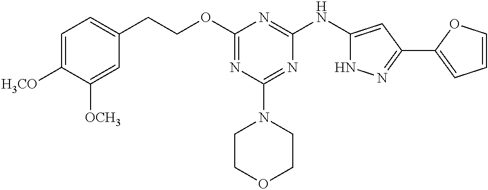 | {4-[2-(3,4-Dimethoxy-phenyl)- ethoxy]-6-morpholin-4-yl- [1,3,5]triazin-2-yl}-(5- furan-2-yl-2H-pyrazol-3- yl)-amine |
| 18 | 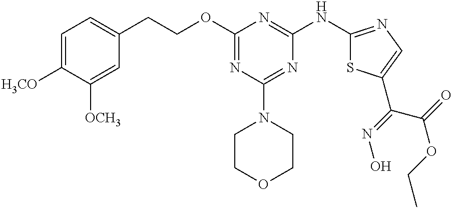 | (2-{4-[2-(3,4-Dimethoxy-phenyl)- ethoxy]-6-morpholin-4-yl- [1,3,5]triazin-2-ylamino}-thiazol- 5-yl)-hydroxyimino-acetic acid ethyl ester |
| 19 | 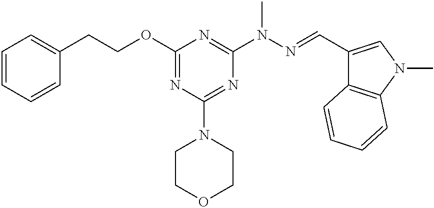 | N-Methyl-N′-(1-methyl-1H- indol-3-ylmethylene)-N- (4-morpholin-4-yl-6- phenethyloxy-[1,3,5]triazin- 2-yl)-hydrazine |
| 20 | 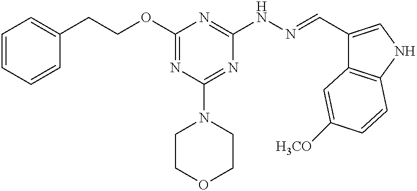 | N-(5-Methoxy-1H-indol-3- ylmethylene)-N′-(4-morpholin- 4-yl-6-phenethyloxy- [1,3,5]triazin-2-yl)-hydrazine |
| 21 | 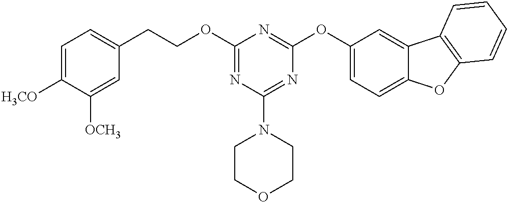 | 2-(Dibenzofuran-2-yloxy)-4- [2-(3,4-dimethoxy-phenyl)- ethoxy]-6-morpholin-4-yl- [1,3,5]triazine |
| 22 | 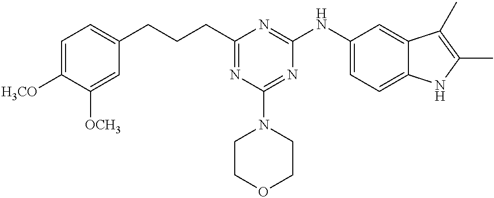 | {4-[3-(3,4-Dimethoxy-phenyl)- propyl]-6-morpholin-4-yl- [1,3,5]triazin-2-yl}-(2,3- dimethyl-1H-indol-5-yl)-amine |
| 23 | 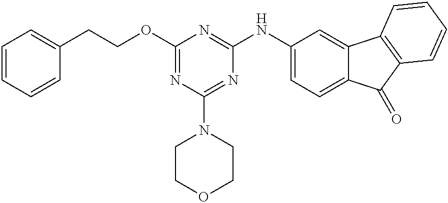 | 3-(4-Morpholin-4-yl-6- phenethyloxy-[1,3,5]triazin- 2-ylamino)-fluoren-9-one |
| 24 | 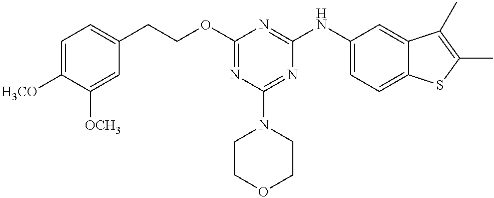 | {4-[2-(3,4-Dimethoxy-phenyl)- ethoxy]-6-morpholin-4-yl- [1,3,5]triazin-2-yl}-(2,3- dimethyl-benzo[b]thiophen-5- yl)-amine |
| 25 | 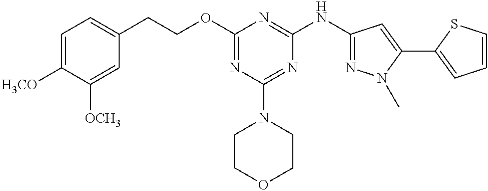 | {4-[2-(3,4-Dimethoxy-phenyl)- ethoxy]-6-morpholin-4-yl- [1,3,5]triazin-2-yl}-(1- methyl-5-thiophen-2-yl- 1H-pyrazol-3-yl)-amine |
| 26 | 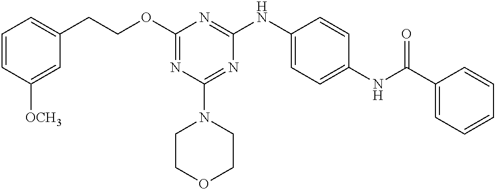 | N-(4-{4-[2-(3-Methoxy-phenyl)- ethoxy]-6-morpholin-4-yl- [1,3,5]triazin-2-ylamino}- phenyl)-benzamide |
| 27 | 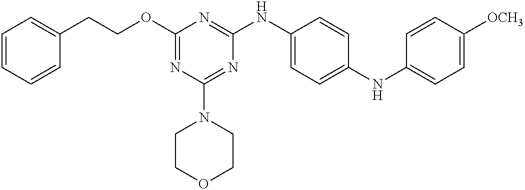 | N-(4-Methoxy-phenyl)-N′-(4- morpholin-4-yl-6-phenethyloxy- [1,3,5]triazin-2-yl)-benzene- 1,4-diamine |
| 28 | 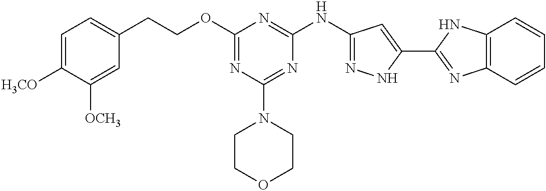 | [5-(1H-Benzoimidazol-2-yl)- 1H-pyrazol-3-yl]-{4-[2-(3,4- dimethoxy-phenyl)-ethoxy]-6- morpholin-4-yl-[1,3,5]triazin- 2-yl}-amine |
| 29 | 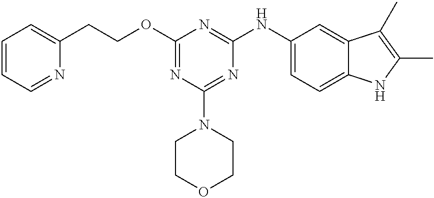 | (2,3-Dimethyl-1H-indol-5-yl)- [4-morpholin-4-yl-6-(2-pyridin- 2-yl-ethoxy)-[1,3,5]triazin-2-yl]- amine |
| 30 | 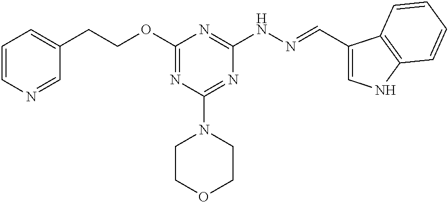 | N-(1H-Indol-3-ylmethylene)-N′- [4-morpholin-4-yl-6-(2-pyridin- 3-yl-ethoxy)-[1,3,5]triazin-2- yl]-hydrazine |
| 31 | 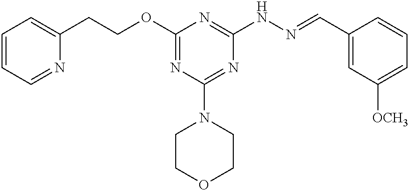 | N-(3-Methoxy-benzylidene)-N′- [4-morpholin-4-yl-6-(2- pyridin-2-yl-ethoxy)- [1,3,5]triazin-2-yl]-hydrazine |
| 32 | 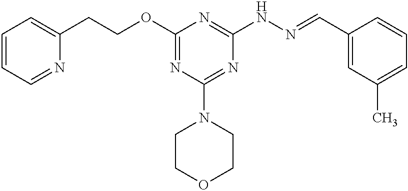 | N-(3-Methyl-benzylidene)-N′- [4-morpholin-4-yl-6-(2- pyridin-2-yl-ethoxy)- [1,3,5]triazin-2-yl]-hydrazine |
| 33 | 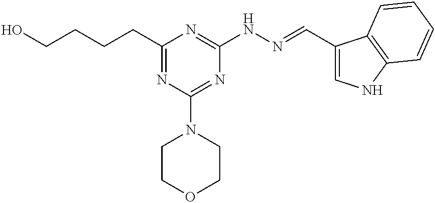 | 4-{4-[N′-(1H-Indol-3- ylmethylene)-hydrazino]-6- morpholin-4-yl-[1,3,5]triazin- 2-yl}-butan-1-ol |
| 34 | 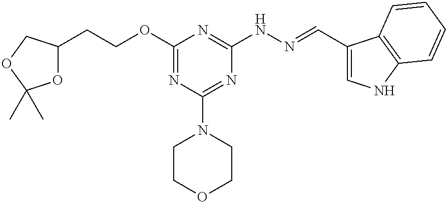 | N-{4-[2-(2,2-Dimethyl- [1,3]dioxolan-4-yl)-ethoxy]- 6-morpholin-4-yl- [1,3,5]triazin-2-yl}-N′-(1H-indol- 3-ylmethylene)-hydrazine |
| 35 | 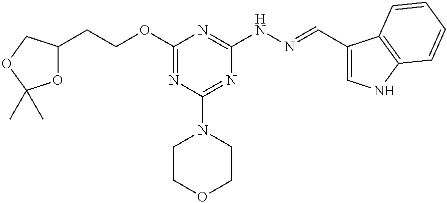 | N-{4-[2-(2,2-Dimethyl- [1,3]dioxolan-4-yl)-ethoxy]- 6-morpholin-4-yl-[1,3,5]triazin- 2-yl}-N′-(1H-indol-3- ylmethylene)-hydrazine |
| 36 | 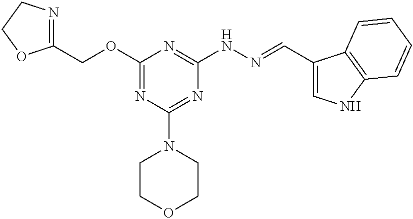 | N-[4-(4,5-Dihydro-oxazol-2- ylmethoxy)-6-morpholin-4-yl- [1,3,5]triazin-2-yl]-N′-(1H-indol- 3-ylmethylene)-hydrazine |
| 37 | 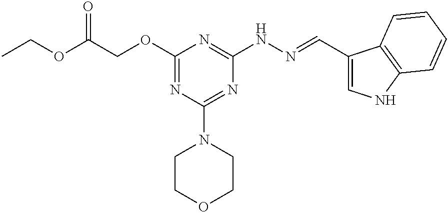 | {4-[N′-(1H-Indol- 3-ylmethylene)-hydrazino]- 6-morpholin-4-yl-[1,3,5]triazin- 2-yloxy}-acetic acid ethyl ester |
| 38 | 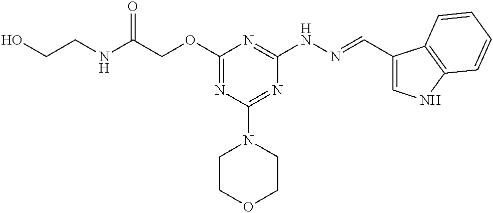 | N-(2-Hydroxy-ethyl)-2-{4-[N′- (1H-indol-3-ylmethylene)- hydrazino]-6-morpholin-4-yl- [1,3,5]triazin-2-yloxy}-acetamide |
| 39 | 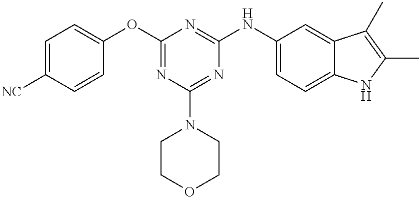 | 4-[4-(2,3-Dimethyl-1H- indol-5-ylamino)-6- morpholin-4-yl-[1,3,5]triazin- 2-yloxy]-benzonitrile |
| 40 | 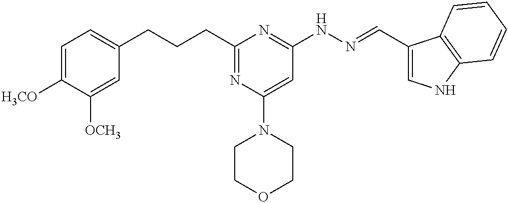 | N-{2-[3-(3,4-Dimethoxy- phenyl)-propyl]-6- morpholin-4-yl-pyrimidin-4-yl}- N′-(1H-indol-3-ylmethylene)- hydrazine |
| 41 | 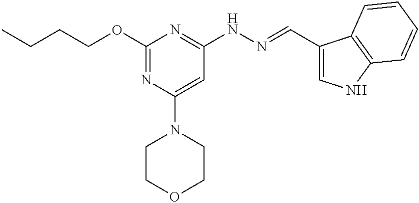 | N-(2-Butoxy-6-morpholin- 4-yl-pyrimidin-4-yl)- N′-(1H-indol-3-ylmethylene)- hydrazine |
| 42 | 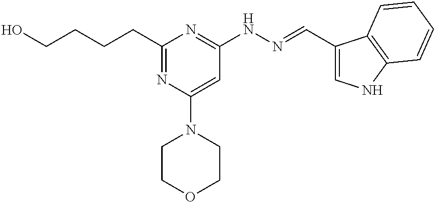 | 4-{4-[N′-(1H-Indol- 3-ylmethylene)-hydrazino]- 6-morpholin-4-yl-pyrimidin- 2-yl}-butan-1-ol |
| 43 | 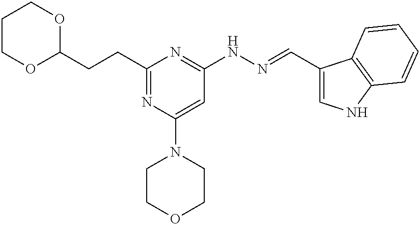 | N-[2-(2-[1,3]Dioxan-2-yl-ethyl)- 6-morpholin-4-yl-pyrimidin-4-yl]- N′-(1H-indol-3-ylmethylene)- hydrazine |
| 44 | 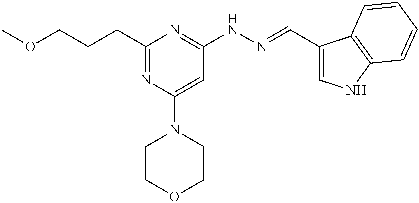 | N-(1H-Indol-3-ylmethylene)- N′-[2-(3-methoxy-propyl)- 6-morpholin-4-yl-pyrimidin- 4-yl]-hydrazine |
| 45 | 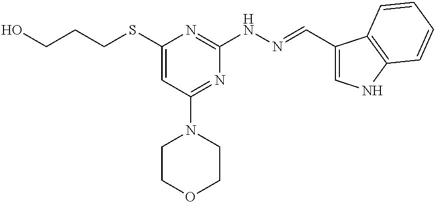 | 3-{2-[N′-(1H-Indol-3- ylmethylene)-hydrazino]-6- morpholin-4-yl-pyrimidin-4- ylsulfanyl}-propan-1-ol |
| 46 | 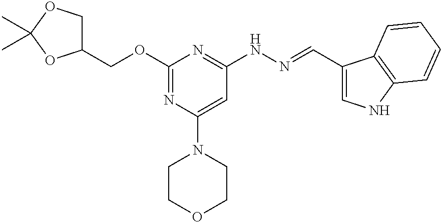 | N-[2-(2,2-Dimethyl- [1,3]dioxolan-4-ylmethoxy)- 6-morpholin-4-yl-pyrimidin- 4-yl]-N′-(1H-indol-3- ylmethylene)-hydrazine |
| 47 | 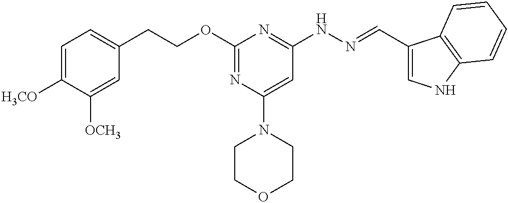 | N-[2-[2-(3,4-Dimethoxy- phenyl)-ethoxy]-6- morpholin-4-yl-pyrimidin-4-yl}- N′-(1H-indol-3-ylmethylene)- hydrazine |
| 48 | 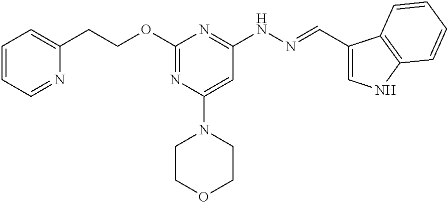 | N-(1H-Indol-3-ylmethylene)- N′-[6-morpholin-4-yl-2-(2- pyridin-2-yl-ethoxy)- pyrimidin-4-yl]-hydrazine |
| 49 | 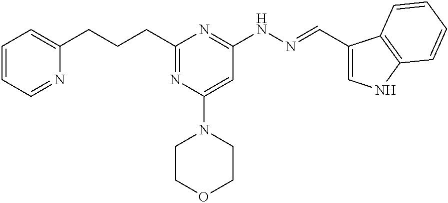 | N-(1H-Indol-3-ylmelhylene)- N′-[6-morpholin-4-yl-2-(3- pyridin-2-yl-propyl)- pyrimidin-4-yl]-hydrazine |
| 50 | 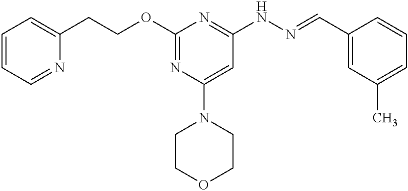 | N-(3-Methyl-benzylidene)- N′-[6-morpholin-4-yl-2-(2- pyridin-2-yl-ethoxy)- pyrimidin-4-yl]-hydrazine |
| 60 | 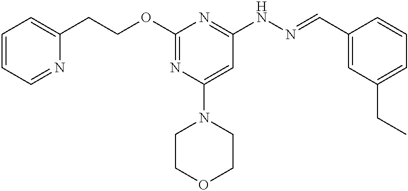 | N-(3-Ethyl-benzylidene)- N′-[6-morpholin-4-yl-2-(2- pyridin-2-yl-ethoxy)- pyrimidin-4-yl]-hydrazine |
| 61 | 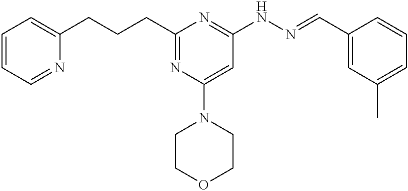 | N-(3-Methyl-benzylidene)- N′-[6-morpholin-4-yl-2-(3- pyridin-2-yl-propyl)- pyrimidin-4-yl]-hydrazine |
| 62 | 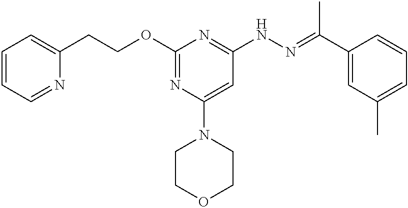 | N-[6-Morpholin-4-yl-2-(2- pyridin-2-yl-ethoxy)- pyrimidin-4-yl]-N′-(1-m- tolyl-ethylidene)-hydrazine |
| 63 | 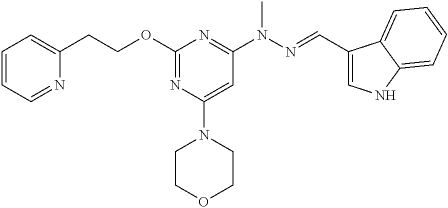 | N′-(1H-Indol-3-ylmethylene)- N-methyl-N-[6-morpholin-4- yl-2-(2-pyridin-2-yl-ethoxy)- pyrimidin-4-yl]-hydrazine |
| 64 | 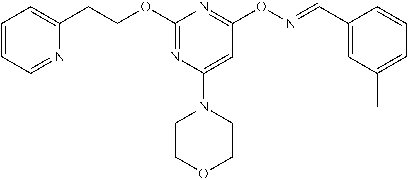 | 3-Methyl-benzaldehyde O-[6-morpholin-4-yl-2-(2- pyridin-2-yl-ethoxy)- pyrimidin-4-yl]-oxime |
| 65 | 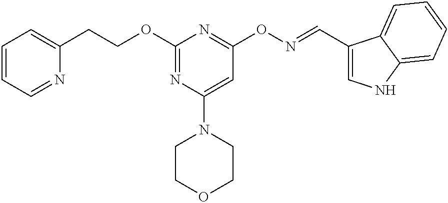 | 1H-Indole-3-carbaldehyde O-[6-morpholin-4-yl-2-(2- pyridin-2-yl-ethoxy)- pyrimidin-4-yl]-oxime |
| 66 | 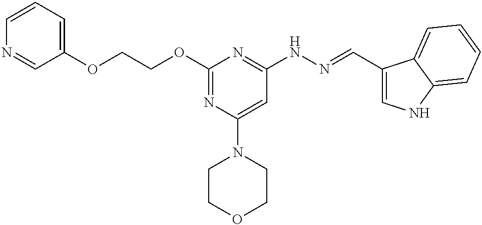 | N-(1H-Indol-3-ylmethylene)- N′-{6-morpholin-4-yl-2-[2- (pyridin-3-yloxy)-ethoxy]- pyrimidin-4-yl}-hydrazine |
| 67 | 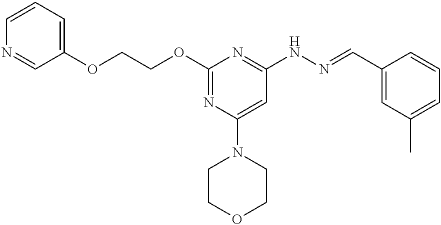 | N-(3-Methyl-benzylidene)- N′-{6-morpholin-4-yl-2-[2- (pyridin-3-yloxy)-ethoxy]- pyrimidin-4-yl}-hydrazine |
| 68 | 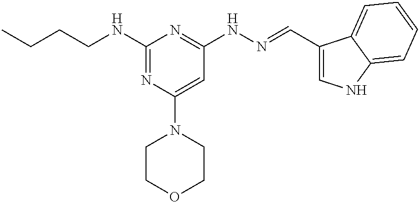 | Butyl-{4-[N′-(1H-indol-3- ylmethylene)-hydrazino]- 6-morpholin-4-yl-pyrimidin- 2-yl}-amine |
| 69 | 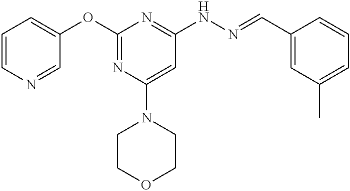 | N-(3-Methyl-benzylidene)- N′-[6-morpholin-4-yl-2- (pyridin-3-yloxy)- pyrimidin-4-yl]-hydrazine |
| 70 | 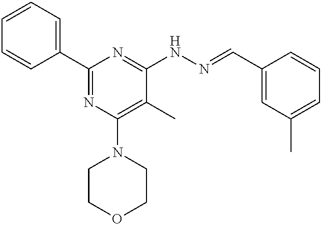 | N-(3-Methyl-benzylidene)- N′-(5-methyl-6-morpholin- 4-yl-2-phenyl-pyrimidin- 4-yl)-hydrazine |
| 71 | 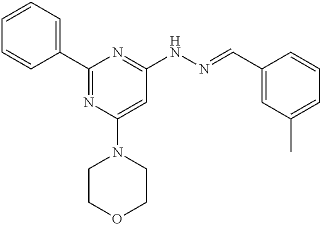 | N-(3-Methyl-benzylidene)- N′-(6-morpholin-4-yl-2- phenyl-pyrimidin-4-yl)- hydrazine |
| 72 | 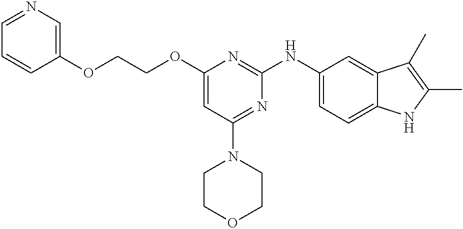 | (2,3-Dimethyl-1H-indol- 5-yl)-{4-morpholin-4-yl-6- [2-(pyridin-3-yloxy)-ethoxy]- pyrimidin-2-yl}-amine |
| 73 | 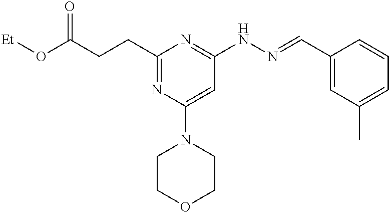 | 3-{4-[N′-(3-Methyl- benzylidene)-hydrazino]-6- morpholin-4-yl-pyrimidin-2- yl}-propionic acid ethyl ester |
| 74 | 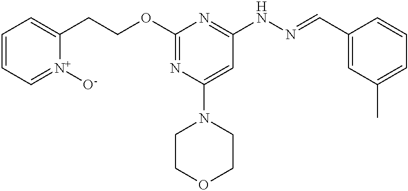 | N-(3-Methyl-benzylidene)- N′-{6-morpholin-4-yl-2-[2- (1-oxy-pyridin-2-yl)-ethoxy]- pyrimidin-4-yl}-hydrazine |
| 75 | 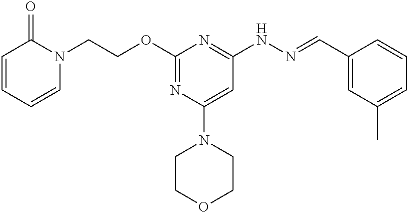 | 1-(2-{4-[N′-(3-Methyl- benzylidene)-hydrazino]-6- morpholin-4-yl-pyrimidin- 2-yloxy}-ethyl)-1H- pyridin-2-one |
| 76 | 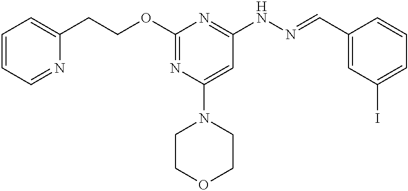 | N-(3-Iodo-benzylidene)- N′-[6-morpholin-4-yl-2- (2-pyridin-2-yl-ethoxy)- pyrimidin-4-yl]-hydrazine |
| 77 | 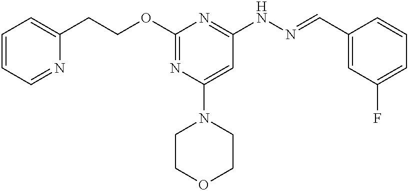 | N-(3-Fluoro-benzylidene)- N′-[6-morpholin-4-yl-2-(2- pyridin-2-yl-ethoxy)- pyrimidin-4-yl]-hydrazine |
| 78 | 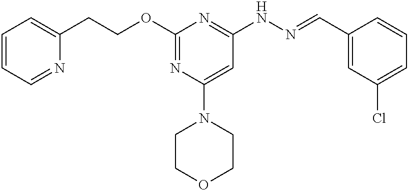 | N-(3-Chloro-benzylidene)- N′-[6-morpholin-4-yl-2-(2- pyridin-2-yl-ethoxy)- pyrimidin-4-yl]-hydrazine |
| 79 | 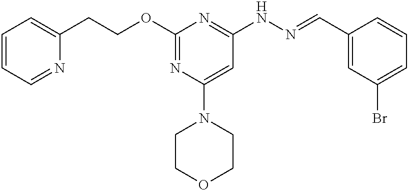 | N-(3-Bromo-benzylidene)- N′-[6-morpholin-4-yl-2-(2- pyridin-2-yl-ethoxy)- pyrimidin-4-yl]-hydrazine |
| 80 | 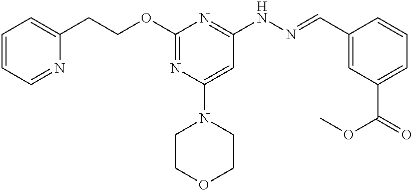 | 3-{[6-Morpholin-4-yl-2- (2-pyridin-2-yl-ethoxy)- pyrimidin-4-yl]- hydrazonomethyl}-benzoic acid methyl ester |
| 81 | 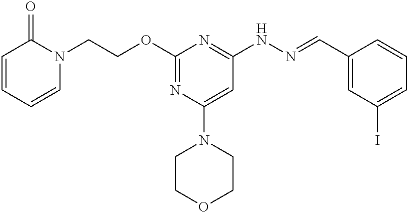 | l-(2-{4-[N′-(3-Iodo- benzylidene)-hydrazino]- 6-morpholin-4-yl- pyrimidin-2-yloxy}-ethyl)- 1H-pyridin-2-one |
| 82 | 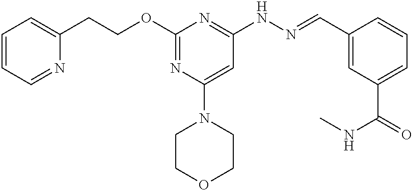 | N-Methyl-3-{[6-morpholin- 4-yl-2-(2-pyridin-2-yl- ethoxy)-pyrimidin-4-yl]- hydrazonomethyl}-benzamide |
| 83 | 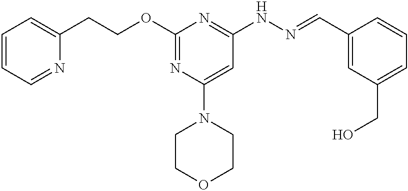 | (3-{[6-Morpholin-4-yl-2- (2-pyridin-2-yl-ethoxy)- pyrimidin-4-yl]- hydrazonomethyl}-phenyl)- methanol |
| 84 | 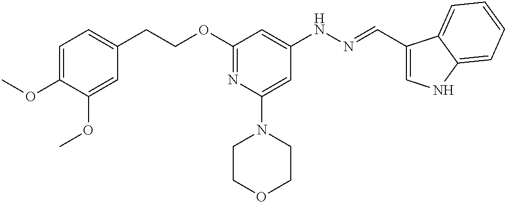 | N-{2-[2-(3,4-Dimethoxy- phenyl)-ethoxy]-6- morpholin-4-yl-pyridin-4- yl}-N′-(1H-indol-3- ylmethylene)-hydrazine |
| 85 | 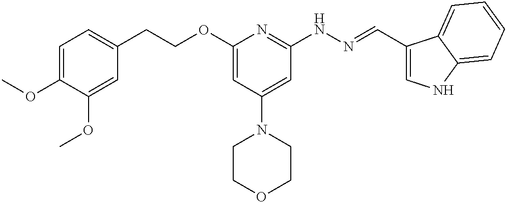 | N-{6-[2-(3,4-Dimethoxy- phenyl)-ethoxy]-4- morpholin-4-yl-pyridin- 2-yl}-N′-(1H-indol-3- ylmethylene)-hydrazine |
| 86 | 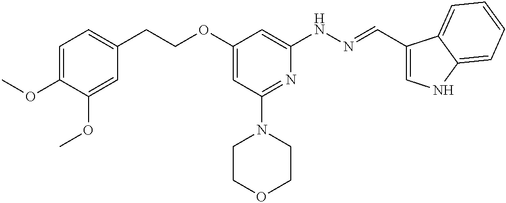 | N-{4-[2-(3,4-Dimethoxy- phenyl)-ethoxy]-6- morpholin-4-yl-pyridin- 2-yl}-N′-(1H-indol-3- ylmethylene)-hydrazine |
| 87 | 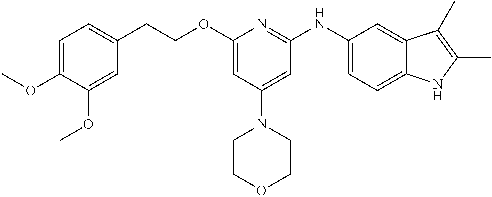 | {6-[2-(3,4-Dimethoxy- phenyl)-ethoxy]-4- morpholin-4-yl-pyridin- 2-yl}-(2,3-dimethyl- 1H-indol-5-yl)-amine |
| 88 | 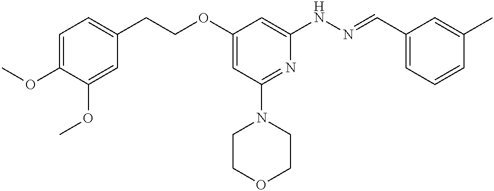 | N-{4-[2-(3,4-Dimethoxy- phenyl)-ethoxy]-6- morpholin-4-yl-pyridin- 2-yl}-N′-(3-methyl- benzylidene)-hydrazine |
| 89 | 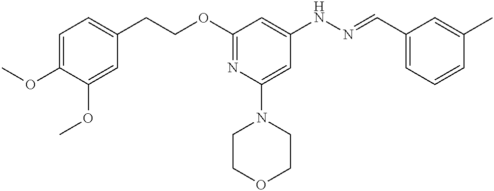 | N-{2-[2-(3,4-Dimcthoxy- phenyl)-ethoxy]-6- morpholin-4-yl-pyridin- 4-yl}-N′-(3-methyl- benzylidene)-hydrazine |
| 90 | 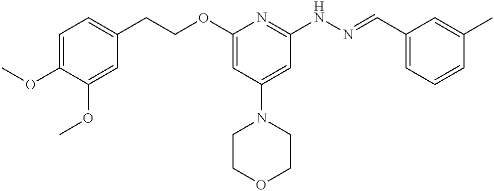 | N-{6-[2-(3,4-Dimethoxy- phenyl)-ethoxy]-4- morpholin-4-yl-pyridin- 2-yl}-N′-(3-methyl- benzylidene)-hydrazine |
| 91 | 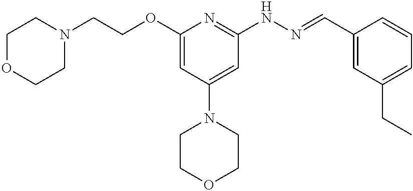 | N-(3-Ethyl-benzylidene)-N′-[4- morpholin-4-yl-6-(2- morpholin-4-yl-ethoxy)- pyridin-2-yl]-hydrazine |
| 92 | 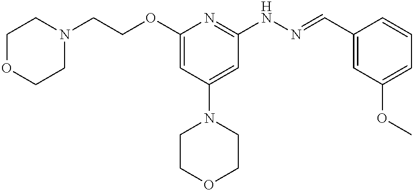 | N-(3-Methoxy-benzylidene)- N′-[4-morpholin-4-yl-6-(2- morpholin-4-yl-ethoxy)- pyridin-2-yl]-hydrazine |
| 93 | 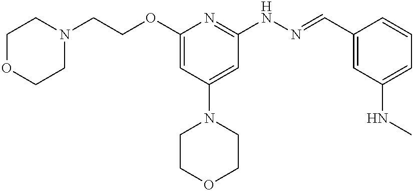 | Methyl-(3-{[4-morpholin-4-yl- 6-(2-morpholin-4-yl-ethoxy)- pyridin-2-yl]- hydrazonomethyl}-phenyl)- amine |
| 94 | 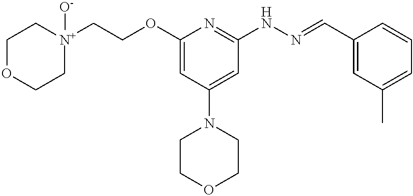 | N-(3-Methyl-benzylidene)-N′- {4-morpholin-4-yl-6-[2-(4-oxy- morpholin-4-yl)-ethoxy]- pyridin-2-yl}-hydrazine |
| 95 | 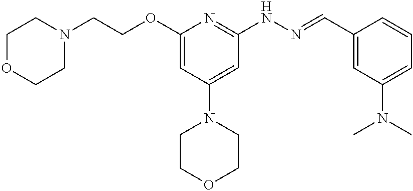 | Dimethyl-(3-{[4-morpholin-4- yl-6-(2-morpholin-4-yl- ethoxy)-pyridin-2-yl]- hydrazonomethyl}-phenyl)- amine |
| 96 | 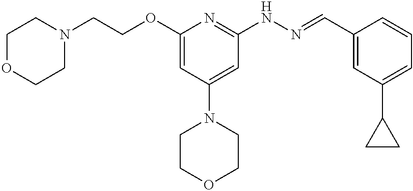 | N-(3-Cyclopropyl- benzylidene)-N′-[4-morpholin- 4-yl-6-(2-morpholin-4-yl- ethoxy)-pyridin-2-yl]- hydrazine |
| 97 | 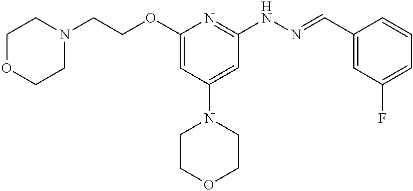 | N-(3-Fluoro-benzylidene)-N′- [4-morpholin-4-yl-6-(2- morpholin-4-yl-ethoxy)- pyridin-2-yl]-hydrazine |
| 98 | 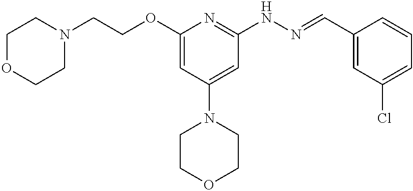 | N-(3-Chloro-benzylidene)-N′- [4-morpholin-4-yl-6-(2- morpholin-4-yl-ethoxy)- pyridin-2-yl]-hydrazine |
| 99 | 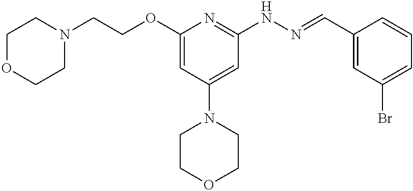 | N-(3-Bromo-benzylidene)-N′- [4-morpholin-4-yl-6-(2- morpholin-4-yl-ethoxy)- pyridin-2-yl]-hydrazine |
| 100 | 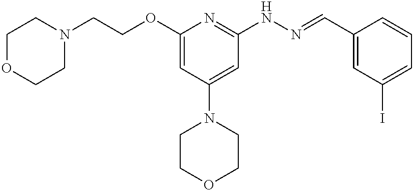 | N-(3-Iodo-benzylidene)-N′-[4- morpholin-4-yl-6-(2- morpholin-4-yl-ethoxy)- pyridin-2-yl]-hydrazine |
| 101 | 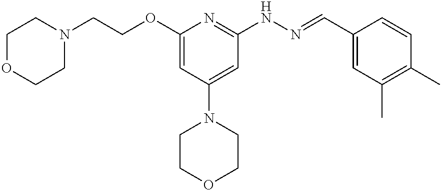 | N-(3,4-Dimethyl-benzylidene)- N′-[4-morpholin-4-yl-6-(2- morpholin-4-yl-ethoxy)- pyridin-2-yl]-hydrazine |
| 102 | 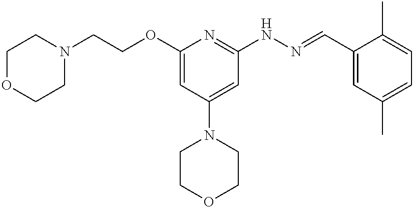 | N-(2,5-Dimethyl-benzylidene)- N′-[4-morpholin-4-yl-6-(2- morpholin-4-yl-ethoxy)- pyridin-2-yl]-hydrazine |
| 103 | 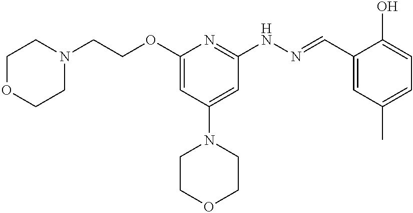 | 4-Methyl-2-{[4-morpholin-4- yl-6-(2-morpholin-4-yl- ethoxy)-pyridin-2-yl]- hydrazonomethyl}-phenol |
| 104 | 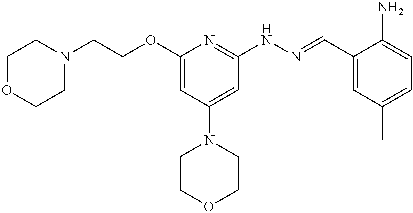 | 4-Methyl-2-{[4-morpholin-4- yl-6-(2-morpholin-4-yl- ethoxy)-pyridin-2-yl]- hydrazonomethyl}- phenylamine |
| 105 | 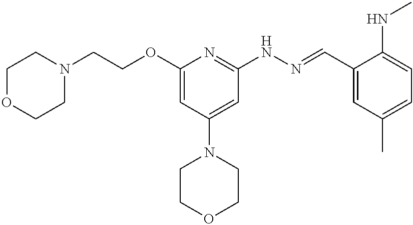 | Methyl-(4-methyl-2-{[4- morpholin-4-yl-6-(2- morpholin-4-yl-ethoxy)- pyridin-2-yl]- hydrazonomethyl}-phenyl)- amine |
| 106 | 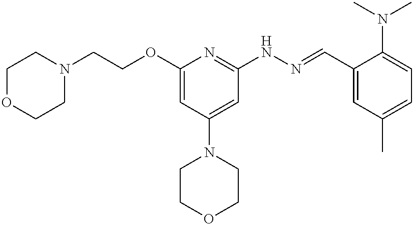 | Dimethyl-(4-methyl-2-{[4- morpholin-4-yl-6-(2- morpholin-4-yl-ethoxy)- pyridin-2-yl]- hydrazonomethyl}-phenyl)- amine |
| 107 | 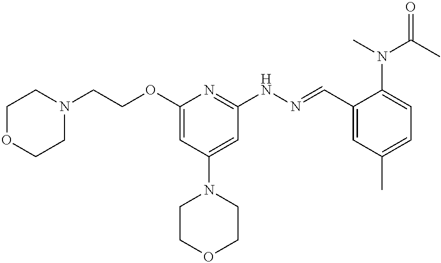 | N-Methyl-N-(4-methyl-2-{[4- morpholin-4-yl-6-(2- morpholin-4-yl-ethoxy)- pyridin-2-yl]- hydrazonomethyl}-phenyl)- acetamide |
| 108 | 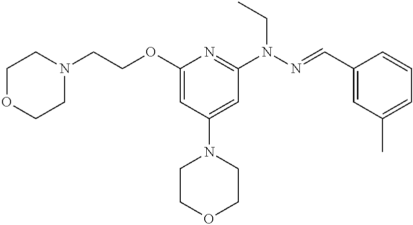 | N-Ethyl-N′-(3-methyl- benzylidene)-N-[4-morpholin- 4-yl-6-(2-morpholin-4-yl- ethoxy)-pyridin-2-yl]- hydrazine |
| 109 | 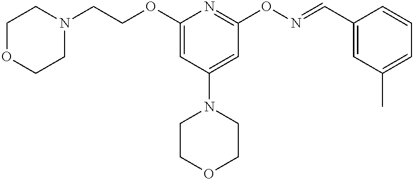 | 3-Methyl-benzaldehyde O-[4- morpholin-4-yl-6-(2- morpholin-4-yl-ethoxy)- pyridin-2-yl]-oxime |
| 110 | 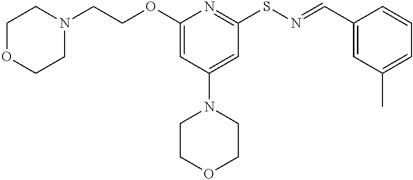 | 3-Methyl-benzaldehyde O-[4- morpholin-4-yl-6-(2- morpholin-4-yl-ethoxy)- pyridin-2-yl]-thiooxime |
| 111 | 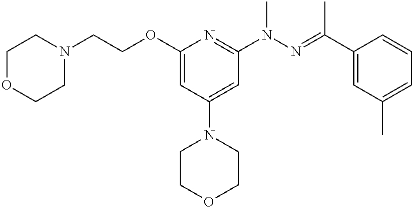 | N-Methyl-N-[4-morpholin-4- yl-6-(2-morpholin-4-yl- ethoxy)-pyridin-2-yl]-N′-(1-m- tolyl-ethylidene)-hydrazine |
| 112 | 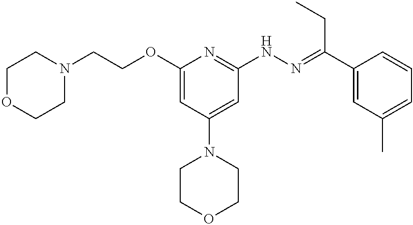 | N-[4-Morpholin-4-yl-6-(2- morpholin-4-yl-ethoxy)- pyridin-2-yl]-N′-(1-m-tolyl- propylidene)-hydrazine |
| 113 | 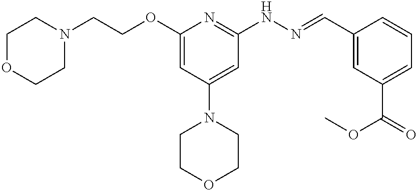 | 3-{[4-Morpholin-4-yl-6-(2- morpholin-4-yl-ethoxy)- pyridin-2-yl]- hydrazonomethyl}-benzoic acid methyl ester |
| 114 | 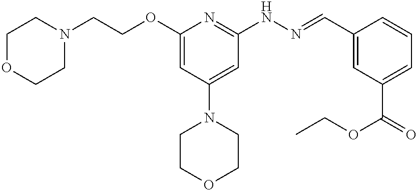 | 3-{[4-Morpholin-4-yl-6-(2- morpholin-4-yl-ethoxy)- pyridin-2-yl]- hydrazonomethyl}-benzoic acid ethyl ester |
| 115 | 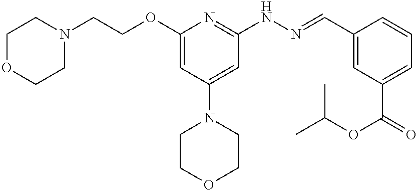 | 3-{[4-Morpholin-4-yl-6-(2- morpholin-4-yl-ethoxy)- pyridin-2-yl]- hydrazonomethyl}-benzoic acid isopropyl ester |
| 116 | 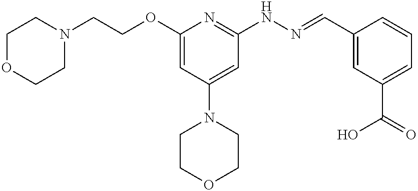 | 3-{[4-Morpholin-4-yl-6-(2- morpholin-4-yl-ethoxy)- pyridin-2-yl]- hydrazonomethyl}-benzoic acid |
| 117 | 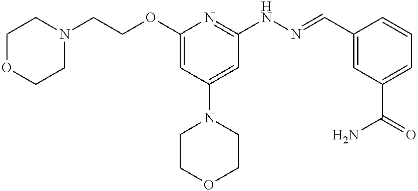 | 3-{[4-Morpholin-4-yl-6-(2- morpholin-4-yl-ethoxy)- pyridin-2-yl]- hydrazonomethyl}-benzamide |
| 118 | 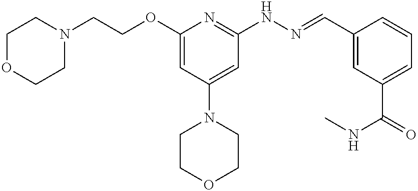 | N-Methyl-3-{[4-morpholin-4- yl-6-(2-morpholin-4-yl- ethoxy)-pyridin-2-yl]- hydrazonomethyl}-benzamide |
| 119 | 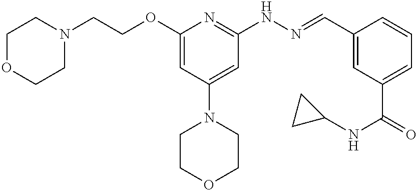 | N-Cyclopropyl-3-{[4- morpholin-4-yl-6-(2- morpholin-4-yl-ethoxy)- pyridin-2-yl]- hydrazonomethyl}-benzamide |
| 120 | 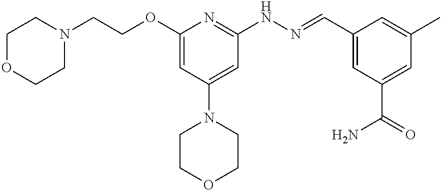 | 3-Methyl-5-{[4-morpholin-4- yl-6-(2-morpholin-4-yl- ethoxy)-pyridin-2-yl]- hydrazonomethyl}-benzamide |
| 121 | 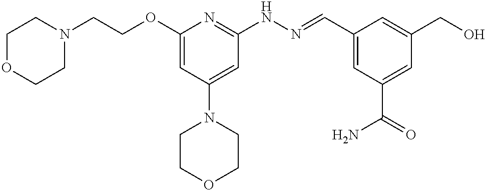 | 3-Hydroxymethyl-5-{[4- morpholin-4-yl-6-(2- morpholin-4-yl-ethoxy)- pyridin-2-yl]- hydrazonomethyl}-benzamide |
| 122 | 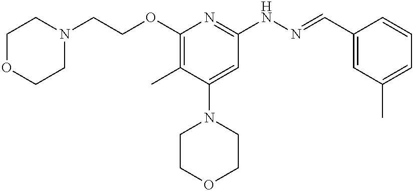 | N-(3-Methyl-benzylidene)-N′- [5-methyl-4-morpholin-4-yl-6- (2-morpholin-4-yl-ethoxy)- pyridin-2-yl]-hydrazine |
| 123 | 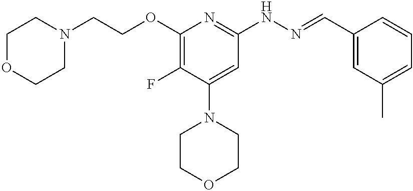 | N-[5-Fluoro-4-morpholin-4-yl- 6-(2-morpholin-4-yl-ethoxy)- pyridin-2-yl]-N′-(3-methyl- benzylidene)-hydrazine |
| 124 | 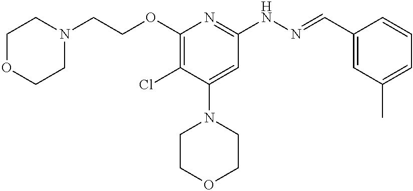 | N-[5-Chloro-4-morpholin-4-yl- 6-(2-morpholin-4-yl-ethoxy)- pyridin-2-yl]-N′-(3-methyl- benzylidene)-hydrazine |
| 125 | 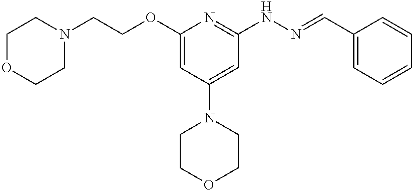 | N-Bcnzylidene-N′-[4- morpholin-4-yl-6-(2- morpholin-4-yl-ethoxy)- pyridin-2-yl]-hydrazine |
| 126 | 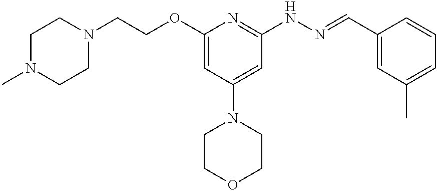 | N-(3-Methyl-benzylidene)-N′- {6-[2-(4-methyl-piperazin-1- yl)-ethoxy]-4-morpholin-4-yl- pyridin-2-yl}-hydrazine |
| 127 | 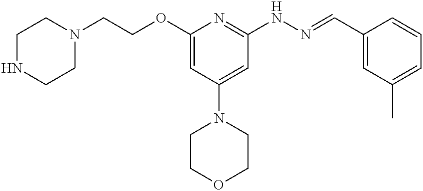 | N-(3-Methyl-benzylidene)-N′- [4-morpholin-4-yl-6-(2- piperazin-1-yl-ethoxy)-pyridin- 2-yl]-hydrazine |
| 128 | 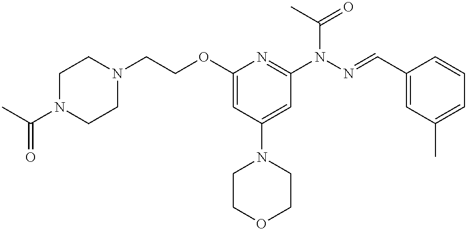 | Acetic acid N-{6-[2-(4-acetyl- piperazin-1-yl)-ethoxy]-4- morpholin-4-yl-pyridin-2-yl}- N′-(3-methyl-benzylidene)- hydrazide |
| 129 | 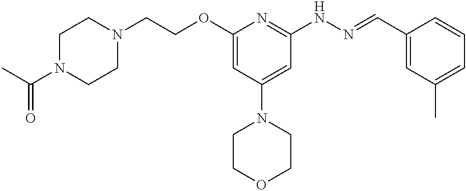 | 1-[4-(2-{6-[N′-(3-Methyl- benzylidene)-hydrazino]-4- morpholin-4-yl-pyridin-2- yloxy}-ethyl)-piperazin-1-yl]- ethanone |
| 130 | 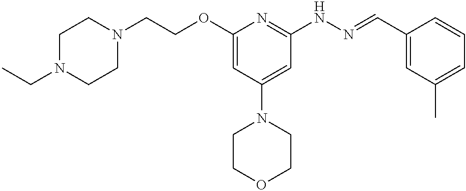 | N-{6-[2-(4-Ethyl-piperazin-1- yl)-ethoxy]-4-morpholin-4-yl- pyridin-2-yl}-N′-(3-methyl- benzylidene)-hydrazine |
| 131 | 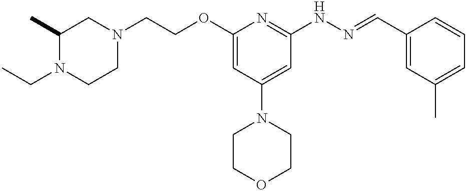 | N-{6-[2-(4-Ethyl-3-methyl- piperazin-1-yl)-ethoxy]-4- morpholin-4-yl-pyridin-2-yl}- N′-(3-methyl-benzylidene)- hydrazine |
| 132 | 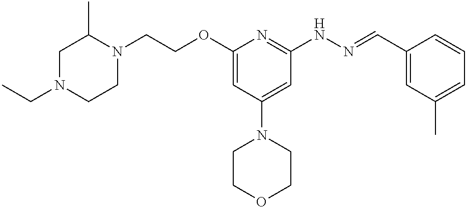 | N-{6-[2-(4-Ethyl-2-methyl- piperazin-1-yl)-ethoxy]-4- morpholin-4-yl-pyridin-2-yl}- N′-(3-methyl-benzylidene)- hydrazine |
| 133 | 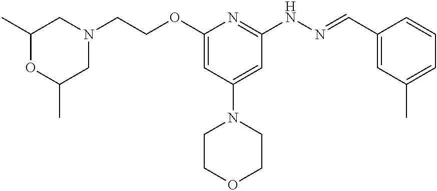 | N-{6-[2-(2,6-Dimethyl- morpholin-4-yl)-ethoxy]-4- morpholin-4-yl-pyridin-2-yl}- N′-(3-methyl-benzylidene)- hydrazine |
| 134 | 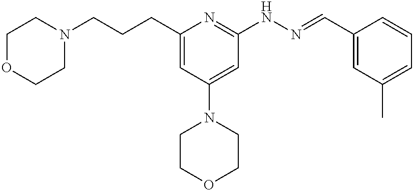 | N-(3-Methyl-benzylidene)-N′- [4-morpholin-4-yl-6-(3- morpholin-4-yl-propyl)- pyridin-2-yl]-hydrazine |
| 135 | 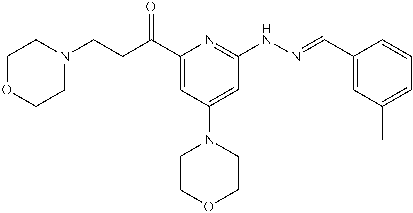 | 1-{6-[N′-(3-Methyl- benzylidene)-hydrazino]-4- morpholin-4-yl-pyridin-2-yl}- 3-morpholin-4-yl-propan-1-one |
| 136 | 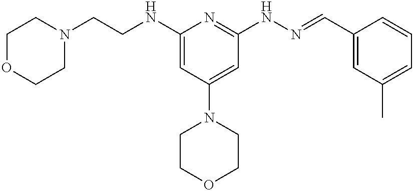 | {6-[N′-(3-Methyl-benzylidene)- hydrazino]-4-morpholin-4-yl- pyridin-2-yl}-(2-morpholin-4- yl-ethyl)-amine |
| 137 | 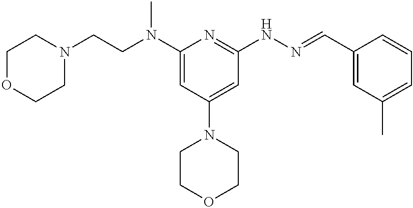 | Methyl-{6-[N′-(3-methyl- benzylidene)-hydrazino]-4- morpholin-4-yl-pyridin-2-yl}- (2-morpholin-4-yl-ethyl)-amine |
| 138 | 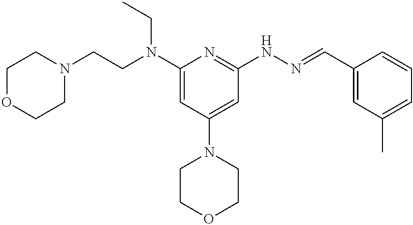 | Ethyl-{6-[N′-(3-methyl- benzylidene)-hydrazino]-4- morpholin-4-yl-pyridin-2-yl}- (2-morpholin-4-yl-ethyl)-amine |
| 139 | 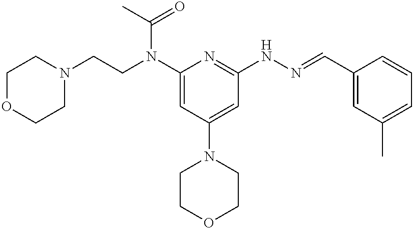 | N-{6-[N′-(3-Methyl- benzylidene)-hydrazino]-4- morpholin-4-yl-pyridin-2-yl}- N-(2-morpholin-4-yl-ethyl)- acetamide |
| 140 | 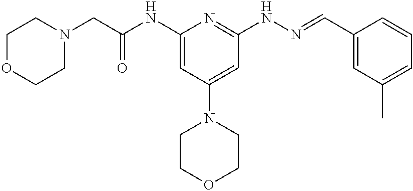 | N-{6-[N′-(3-Methyl- benzylidene)-hydrazino]-4- morpholin-4-yl-pyridin-2-yl}- 2-morpholin-4-yl-acetamide |
| 141 | 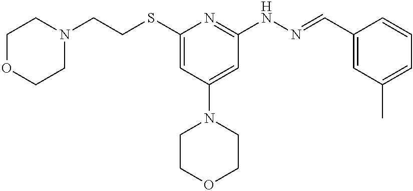 | N-(3-Methyl-benzylidene)-N′- [4-morpholin-4-yl-6-(2- morpholin-4-yl-ethylsulfanyl)- pyridin-2-yl]-hydrazine |
| 142 | 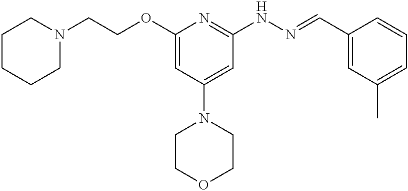 | N-(3-Methyl-benzylidene)-N′- [4-morpholin-4-yl-6-(2- piperidin-1-yl-ethoxy)-pyridin- 2-yl]-hydrazine |
| 143 | 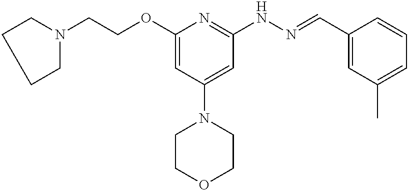 | N-(3-Methyl-benzylidene)-N′- [4-morpholin-4-yl-6-(2- pyrrolidin-1-yl-ethoxy)- pyridin-2-yl]-hydrazine |
| 144 | 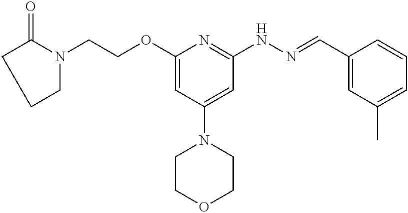 | 1-(2-{6-[N′-(3-Methyl- benzylidene)-hydrazino]-4- morpholin-4-yl-pyridin-2- yloxy}-ethyl)-pyrrolidin-2-one |
| 145 | 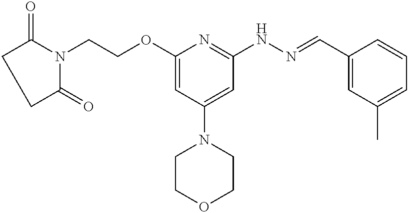 | 1-(2-{6-[N′-(3-Methyl- benzylidene)-hydrazino]-4- morpholin-4-yl-pyridin-2- yloxy}-ethyl)-pyrrolidine-2,5- dione |
| 146 | 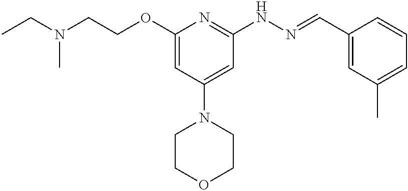 | Ethyl-methyl-(2-{6-[N′-(3- methyl-benzylidene)- hydrazino]-4-morpholin-4-yl- pyridin-2-yloxy}-ethyl)-amine |
| 147 | 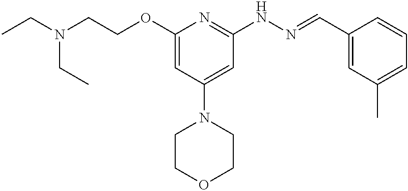 | Diethyl-(2-{6-[N′-(3-methyl- benzylidene)-hydrazino]-4- morpholin-4-yl-pyridin-2- yloxy}-ethyl)-amine |
| 148 | 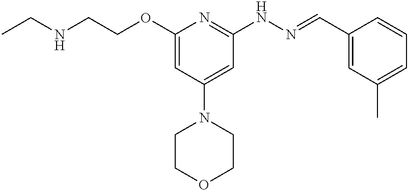 | Ethyl-(2-{6-[N′-(3-methyl- benzylidene)-hydrazino]-4- morpholin-4-yl-pyridin-2- yloxy}-ethyl)-amine |
| 149 | 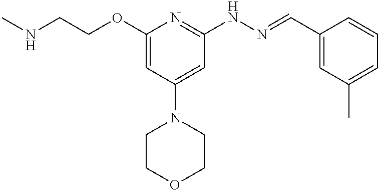 | Methyl-(2-{6-[N′-(3-methyl- benzylidene)-hydrazino]-4- morpholin-4-yl-pyridin-2- yloxy}-ethyl)-amine |
| 150 | 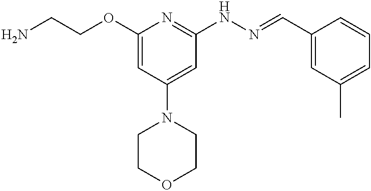 | 2-{6-[N′-(3-Methyl- benzylidene)-hydrazino]-4- morpholin-4-yl-pyridin-2- yloxy}-ethylamine |
| 151 | 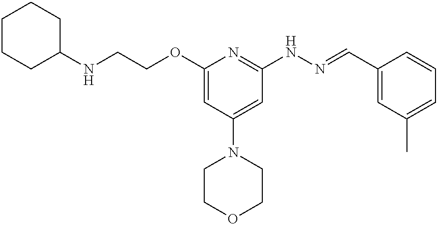 | Cyclohexyl-(2-{6-[N′-(3- methyl-benzylidene)- hydrazino]-4-morpholin-4-yl- pyridin-2-yloxy}-ethyl)-amine |
| 152 | 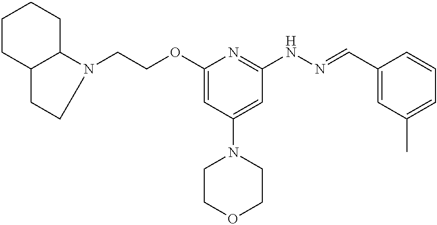 | N-(3-Methyl-benzylidene)-N′- {4-morpholin-4-yl-6-[2- (octahydro-indol-1-yl)-ethoxy]- pyridin-2-yl}-hydrazine |
| 153 | 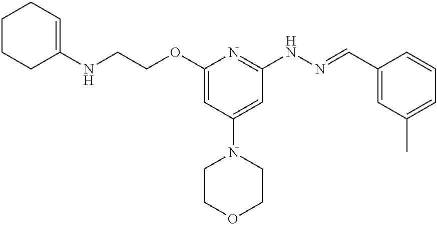 | Cyclohex-1-enyl-(2-{6-[N′-(3- methyl-benzylidene)- hydrazino]-4-morpholin-4-yl- pyridin-2-yloxy}-ethyl)-amine |
| 154 | 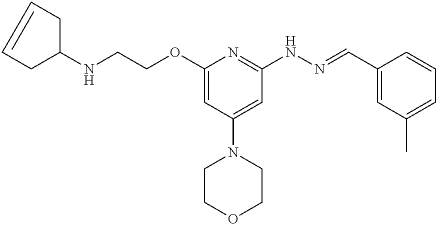 | Cyclopent-3-enyl-(2-{6-[N′-(3- methyl-benzylidene)- hydrazino]-4-morpholin-4-yl- pyridin-2-yloxy}-ethyl)-amine |
| 155 | 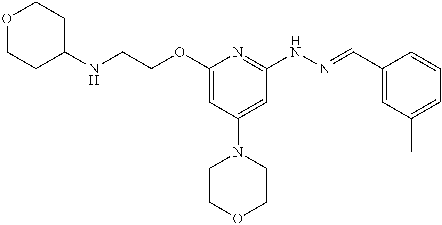 | (2-{6-[N′-(3-Methyl- benzylidene)-hydrazino]-4- morpholin-4-yl-pyridin-2- yloxy}-ethyl)-(tetrahydro- pyran-4-yl)-amine |
| 156 | 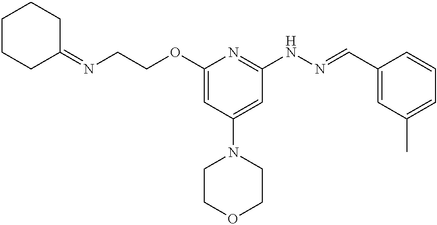 | Cyclohexylidene-(2-{6-[N′-(3- methyl-benzylidene)- hydrazino]-4-morpholin-4-yl- pyridin-2-yloxy}-ethyl)-amine |
| 157 | 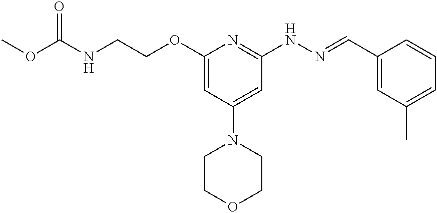 | (2-{6-[N′-(3-Methyl- benzylidene)-hydrazino]-4- morpholin-4-yl-pyridin-2- yloxy}-ethyl)-carbamic acid methyl ester |
| 158 | 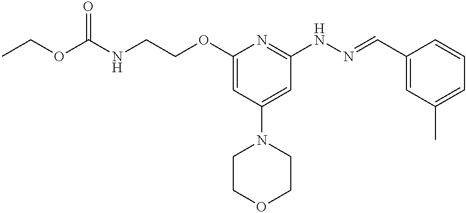 | (2-{6-[N′-(3-Methyl- benzylidene)-hydrazino]-4- morpholin-4-yl-pyridin-2- yloxy}-ethyl)-carbamic acid ethyl ester |
| 159 | 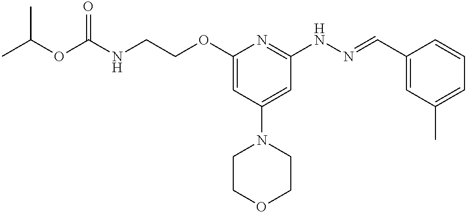 | (2-{6-[N′-(3-Methyl- benzylidene)-hydrazino]-4- morpholin-4-yl-pyridin-2- yloxy}-ethyl)-carbamic acid isopropyl ester |
| 160 | 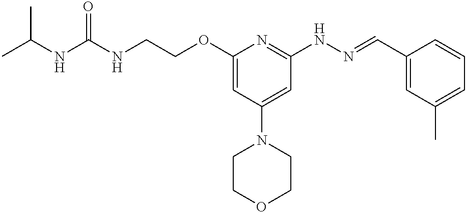 | 1-Isopropyl-3-(2-{6-[N′-(3- methyl-benzylidene)- hydrazino]-4-morpholin-4-yl- pyridin-2-yloxy}-ethyl)-urea |
| 161 | 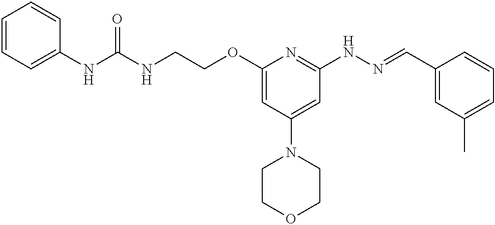 | 1-(2-{6-[N′-(3-Methyl- benzylidene)-hydrazino]-4- morpholin-4-yl-pyridin-2- yloxy}-ethyl)-3-phenyl-urea |
| 162 | 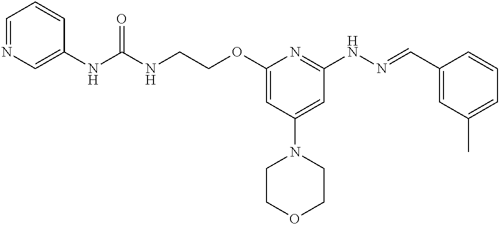 | 1-(2-{6-[N′-(3-Methyl- benzylidene)-hydrazino]-4- morpholin-4-yl-pyridin-2- yloxy}-ethyl)-3-pyridin-3-yl- urea |
| 163 | 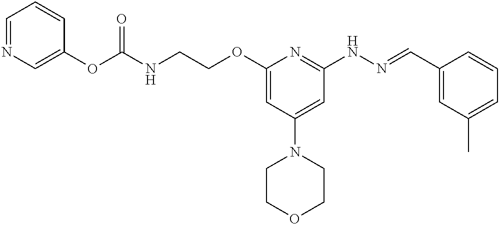 | (2-{6-[-N′-(3-Methyl- benzylidene)-hydrazino]-4- morpholin-4-yl-pyridin-2- yloxy}-ethyl)-carbamic acid pyridin-3-yl ester |
| 164 | 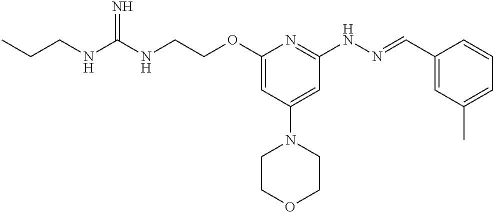 | N-(2-{6-[N′-(3-Methyl- benzylidene)-hydrazino]-4- morpholin-4-yl-pyridin-2- yloxy}-ethyl)-N′-propyl- guanidine |
| 165 | 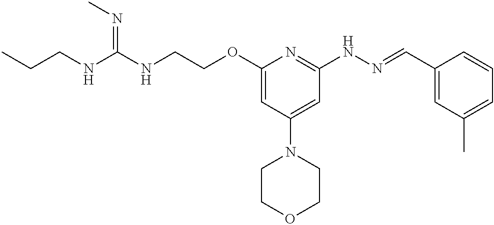 | N-Methyl-N′-(2-{6-[N'-(3- methyl-benzylidene)- hydrazino]-4-morpholin-4-yl- pyridin-2-yloxy}-ethyl)-N″- propyl-guanidine |
| 166 | 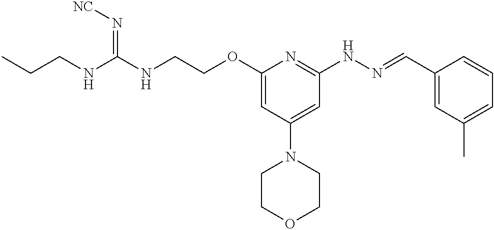 | N-Cyano-N′-(2-{6-[N′-(3- methyl-benzylidene)- hydrazino]-4-morpholin-4-yl- pyridin-2-yloxy}-ethyl)-N″- propyl-guanidine |
| 167 | 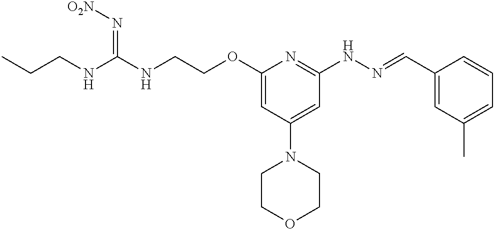 | N-Nitro-N′-(2-{6-[N′-(3- methyl-benzylidene)- hydrazino]-4-morpholin-4-yl- pyridin-2-yloxy}-ethyl)-N″- propyl-guanidine |
| 168 | 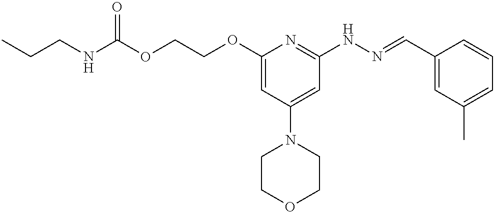 | Propyl-carbamic acid 2-{6-[N′- (3-methyl-benzylidene)- hydrazino]-4-morpholin-4-yl- pyridin-2-yloxy}-ethyl ester |
| 169 | 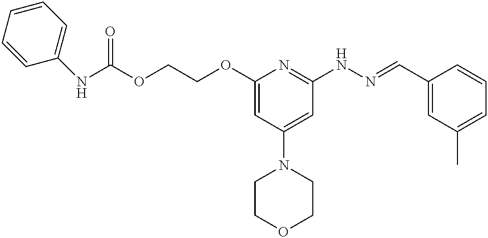 | Phenyl-carbamic acid 2-{6-[N′- (3-methyl-benzylidene)- hydrazino]-4-morpholin-4-yl- pyridin-2-yloxy}-ethyl ester |
| 170 | 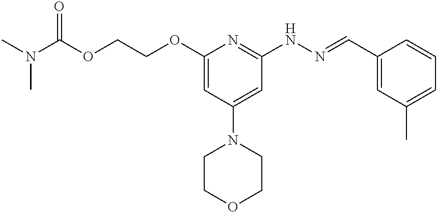 | Dimethyl-carbamic acid 2-{6- [N′-(3-methyl-benzylidene)- hydrazino]-4-morpholin-4-yl- pyridin-2-yloxy}-ethyl ester |
| 171 | 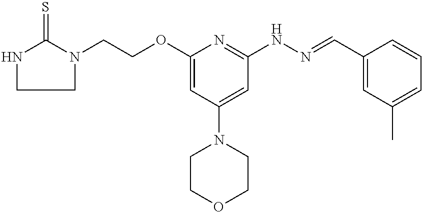 | 1-(2-{6-[N′-(3-Methyl- benzylidene)-hydrazino]-4- morpholin-4-yl-pyridin-2- yloxy}-ethyl)-imidazolidine-2- thione |
| 172 | 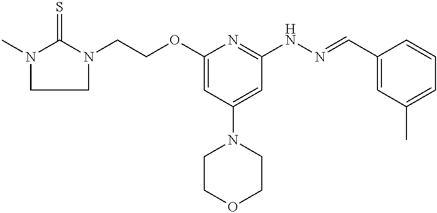 | 1-Methyl-3-(2-{6-[N′-(3- methyl-benzylidene)- hydrazino]-4-morpholin-4-yl- pyridin-2-yloxy}-ethyl)- imidazolidine-2-thione |
| 173 | 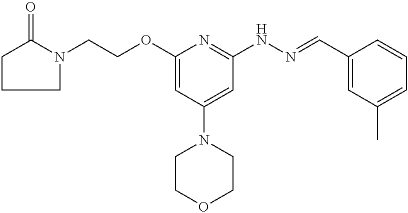 | 1-(2-{6-[N′-(3-Methyl- benzylidene)-hydrazino]-4- morpholin-4-yl-pyridin-2- yloxy}-ethyl)-pyrrolidin-2-one |
| 174 | 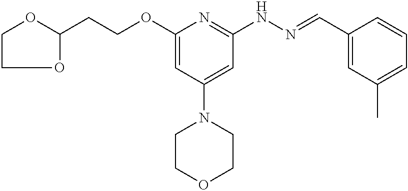 | N-[6-(2-[1,3]Dioxolan-2-yl- ethoxy)-4-morpholin-4-yl- pyridin-2-yl]-N′-(3-methyl- benzylidene)-hydrazine |
| 175 | 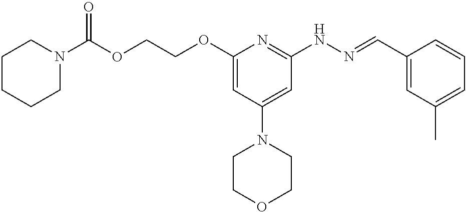 | Piperidine-1-carboxylic acid 2- {6-[N′-(3-methyl-benzylidene)- hydrazino]-4-morpholin-4-yl- pyridin-2-yloxy}-ethyl ester |
| 176 | 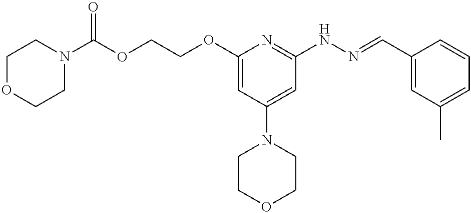 | Morpholine-4-carboxylic acid 2-{6-[N′-(3-methyl- benzylidene)-hydrazino]-4- morpholin-4-yl-pyridin-2- yloxy}-ethyl ester |
| 177 | 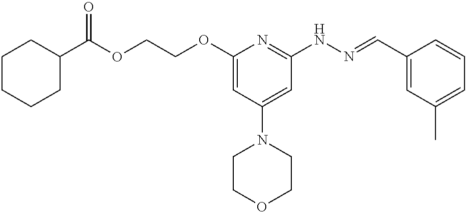 | Cyclohexanecarboxylic acid 2- {6-[N′-(3-methyl-benzylidene)- hydrazino]-4-morpholin-4-yl- pyridin-2-yloxy}-ethyl ester |
| 178 | 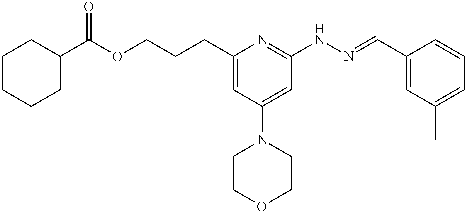 | Cyclohexanecarboxylic acid 3- {6-[N′-(3-methyl-benzylidene)- hydrazino]-4-morpholin-4-yl- pyridin-2-yl}-propyl ester |
| 179 | 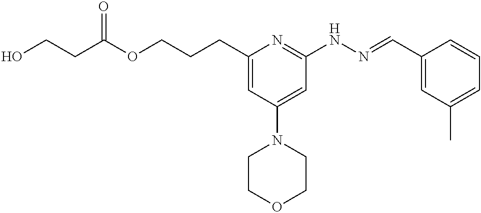 | 3-Hydroxy-propionic acid 3- {6-[N′-(3-methyl-benzylidene)- hydrazino]-4-morpholin-4-yl- pyridin-2-yl}-propyl ester |
| 180 | 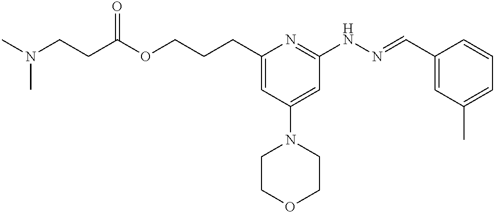 | 3-Dimethylamino-propionic acid 3-{6-[N′-(3-methyl- benzylidene)-hydrazino]-4- morpholin-4-yl-pyridin-2-yl}- propyl ester |
| 181 | 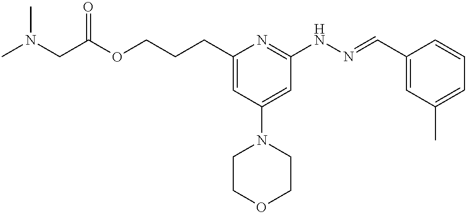 | Dimethylamino-acetic acid 3- {6-[N′-(3-methyl-benzylidene)- hydrazino]-4-morpholin-4-yl- pyridin-2-yl}-propyl ester |
| 182 | 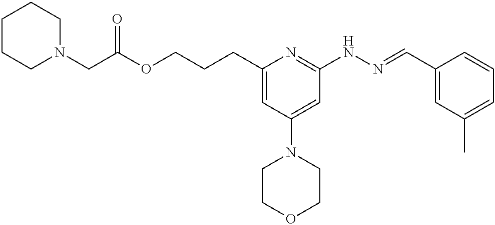 | Piperidin-1-yl-acetic acid 3-{6- [N′-(3-methyl-benzylidene)- hydrazino]-4-morpholin-4-yl- pyridin-2-yl}-propyl ester |
| 183 | 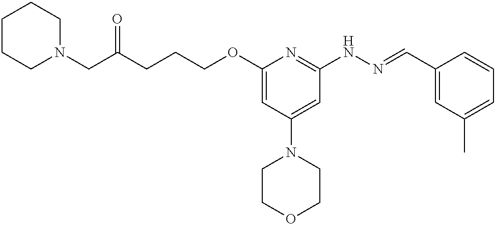 | 5-{6-[N′-(3-Methyl- benzylidene)-hydrazino]-4- morpholin-4-yl-pyridin-2- yloxy}-1-piperidin-1-yl- pentan-2-one |
| 184 | 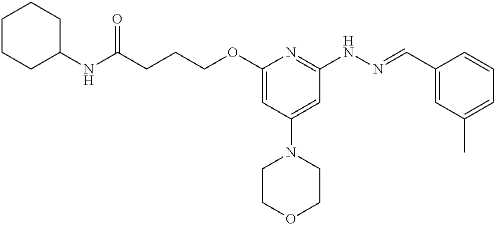 | N-Cyclohexyl-4-{6-[N′-(3- methyl-benzylidene)- hydrazino]-4-morpholin-4-yl- pyridin-2-yloxy}-butyramide |
| 185 | 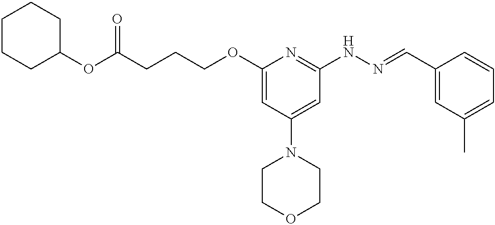 | 4-{6-[N′-(3-Methyl- benzylidene)-hydrazino]-4- morpholin-4-yl-pyridin-2- yloxy}-butyric acid cyclohexyl ester |
| 186 | 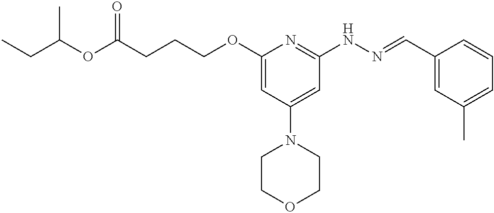 | 4-{6-[N′-(3-Methyl- benzylidene)-hydrazino]-4- morpholin-4-yl-pyridin-2- yloxy}-butyric acid sec-butyl ester |
| 187 | 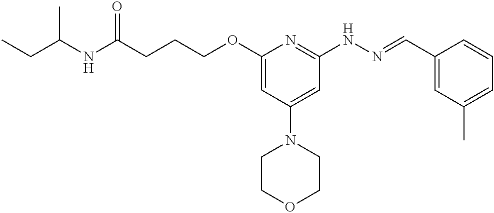 | N-sec-Butyl-4-{6-[N′-(3- methyl-benzylidene)- hydrazino]-4-morpholin-4-yl- pyridin-2-yloxy}-butyramide |
| 188 | 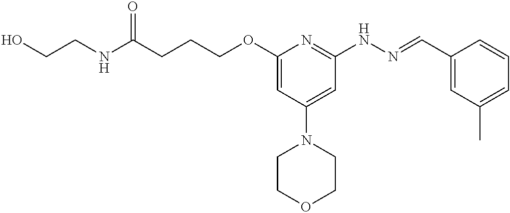 | N-(2-Hydroxy-ethyl)-4-{6-[N′- (3-methyl-benzylidene)- hydrazino]-4-morpholin-4-yl- pyridin-2-yloxy}-butyramide |
| 189 | 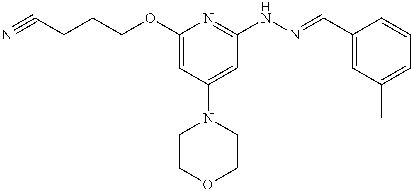 | 4-{6-[N′-(3-Methyl- benzylidene)-hydrazino]-4- morpholin-4-yl-pyridin-2- yloxy}-butyronitrile |
| 190 | 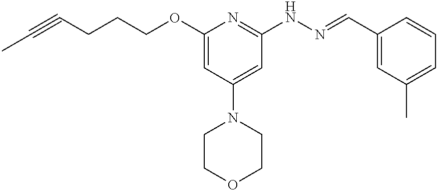 | N-(6-Hex-4-ynyloxy-4- morpholin-4-yl-pyridin-2-yl)- N′-(3-methyl-benzylidene)- hydrazine |
| 191 | 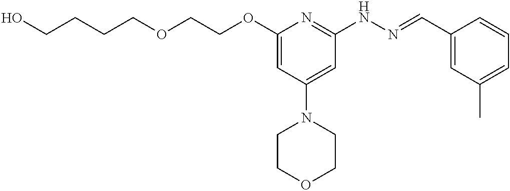 | 4-(2-{6-[N′-(3-Methyl- benzylidene)-hydrazino]-4- morpholin-4-yl-pyridin-2- yloxy}-ethoxy)-butan-1-ol |
| 192 | 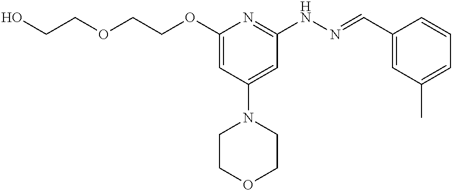 | 2-(2-{6-[N′-(3-Methyl- benzylidene)-hydrazino]-4- morpholin-4-yl-pyridin-2- yloxy}-ethoxy)-ethanol |
| 193 | 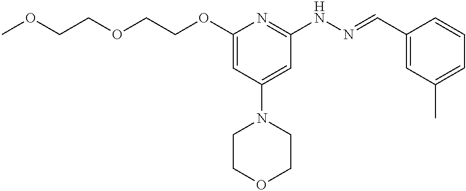 | N-{6-[2-(2-Methoxy-ethoxy)- ethoxy]-4-morpholin-4-yl- pyridin-2-yl}-N′-(3-methyl- benzylidene)-hydrazine |
| 194 | 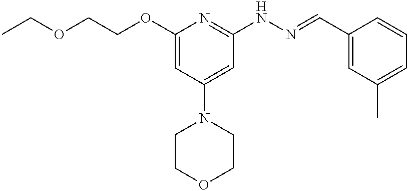 | N-[6-(2-Ethoxy-ethoxy)-4- morpholin-4-yl-pyridin-2-yl]- N′-(3-methyl-benzylidene)- hydrazine |
| 195 | 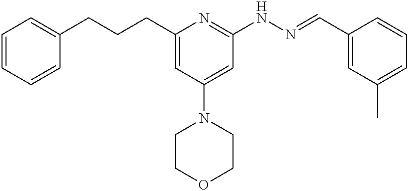 | N-(3-Methyl-benzylidene)-N′- [4-morpholin-4-yl-6-(3-phenyl- propyl)-pyridin-2-yl]-hydrazine |
| 196 | 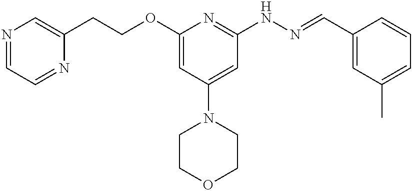 | N-(3-Methyl-benzylidene)-N′- [4-morpholin-4-yl-6-(2- pyrazin-2-yl-ethoxy)-pyridin-2- yl]-hydrazine |
| 197 | 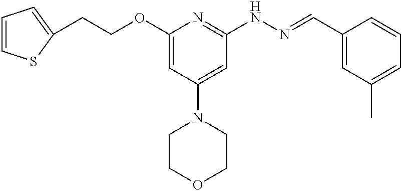 | N-(3-Methyl-benzylidene)-N′- [4-morpholin-4-yl-6-(2- thiophen-2-yl-ethoxy)-pyridin- 2-yl]-hydrazine |
| 198 | 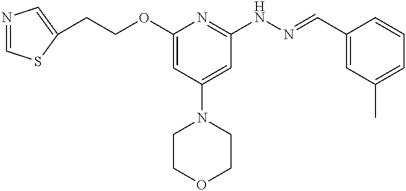 | N-(3-Methyl-benzylidene)-N′- [4-morpholin-4-yl-6-(2-thiazol- 5-yl-ethoxy)-pyridin-2-yl]- hydrazine |
| 199 | 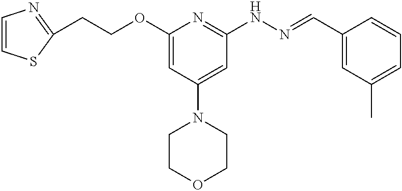 | N-(3-Methyl-benzylidene)-N′- [4-morpholin-4-yl-6-(2-thiazol- 2-yl-ethoxy)-pyridin-2-yl]- hydrazine |
| 200 | 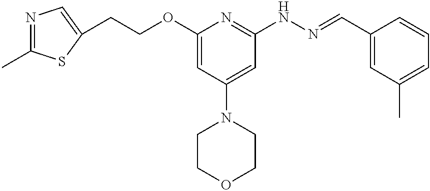 | N-(3-Methyl-benzylidene)-N′- {6-[2-(2-methyl-thiazol-5-yl)- ethoxy]-4-morpholin-4-yl- pyridin-2-yl}-hydrazine |
| 201 | 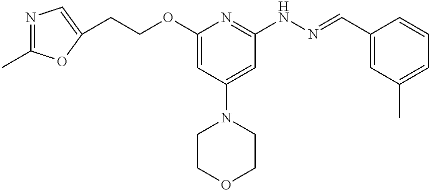 | N-(3-Methyl-benzylidene)-N′- {6-[2-(2-methyl-oxazol-5-yl)- ethoxy]-4-morpholin-4-yl- pyridin-2-yl}-hydrazine |
| 202 | 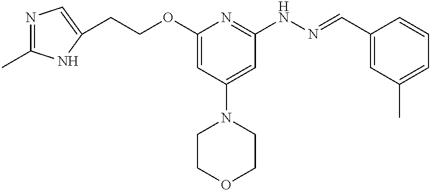 | N-(3-Methyl-benzylidene)-N′- {6-[2-(2-methyl-3H-imidazol- 4-yl)-ethoxy]-4-morpholin-4- yl-pyridin-2-yl}-hydrazine |
| 203 | 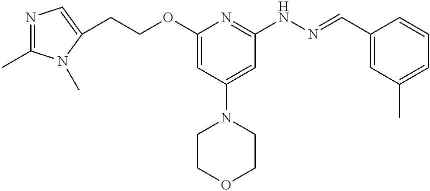 | N-{6-[2-(2,3-Dimethyl-3H- imidazol-4-yl)-ethoxy]-4- morpholin-4-yl-pyridin-2-yl}- N′-(3-methyl-benzylidene)- hydrazine |
| 204 | 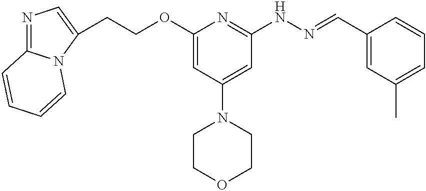 | N-[6-(2-Imidazo[1,2-a]pyridin- 3-yl-ethoxy)-4-morpholin-4-yl- pyridin-2-yl]-N′-(3-methyl- benzylidene)-hydrazine |
| 205 | 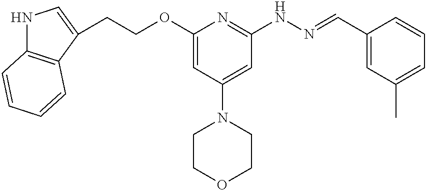 | N-{6-[2-(1H-Indol-3-yl)- ethoxy]-4-morpholin-4-yl- pyridin-2-yl}-N′-(3-methyl- benzylidene)-hydrazine |
| 206 | 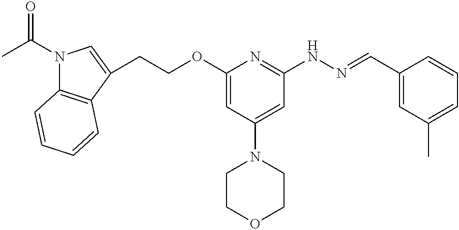 | 1-[3-(2-{6-[N′-(3-Methyl- benzylidene)-hydrazino]-4- morpholin-4-yl-pyridin-2- yloxy}-ethyl)-indol-1-yl]- ethanone |
| 207 | 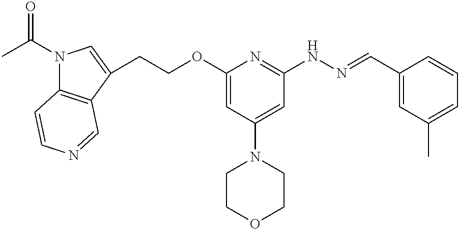 | 1-[3-(2-{6-[N′-(3-Methyl- benzylidene)-hydrazino]-4- morpholin-4-yl-pyridin-2- yloxy}-ethyl)-pyrrolo[3,2- c]pyridin-1-yl]-ethanone |
| 208 | 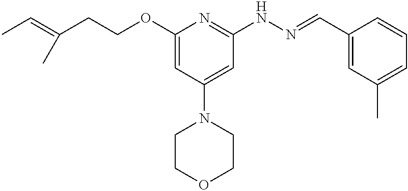 | N-(3-Methyl-benzylidene)-N′- [6-(3-methyl-pent-3-enyloxy)- 4-morpholin-4-yl-pyridin-2-yl]- hydrazine |
| 209 | 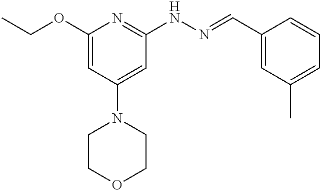 | N-(6-Ethoxy-4-morpholin-4-yl- pyridin-2-yl)-N′-(3-methyl- benzylidene)-hydrazine |
| 210 | 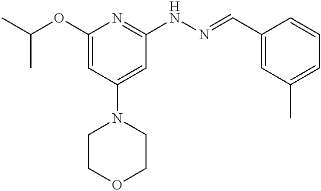 | N-(6-Isopropoxy-4-morpholin- 4-yl-pyridin-2-yl)-N′-(3- methyl-benzylidene)-hydrazine |
| 211 | 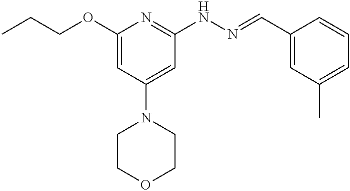 | N-(3-Methyl-benzylidene)-N′- (4-morpholin-4-yl-6-propoxy- pyridin-2-yl)-hydrazine |
| 212 | 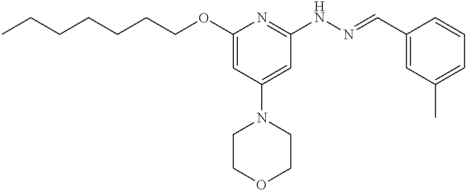 | N-(6-Heptyloxy-4-morpholin- 4-yl-pyridin-2-yl)-N′-(3- methyl-benzylidene)-hydrazine |
| 213 | 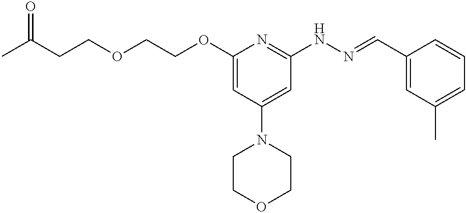 | 4-(2-{6-[N′-(3-Methyl- benzylidene)-hydrazino]-4- morpholin-4-yl-pyridin-2- yloxy}-ethoxy)-butan-2-one |
| 214 | 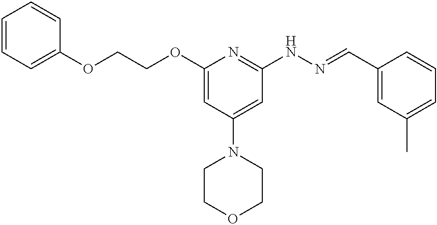 | N-(3-Methyl-benzylidene)-N′- [4-morpholin-4-yl-6-(2- phenoxy-ethoxy)-pyridin-2-yl]- hydrazine |
| 215 | 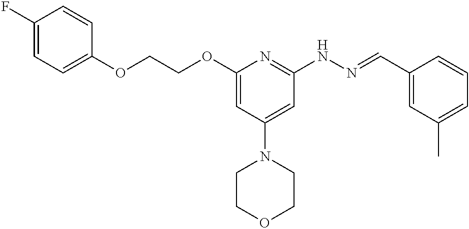 | N-{6-[2-(4-Fluoro-phenoxy)- ethoxy]-4-morpholin-4-yl- pyridin-2-yl}-N′-(3-methyl- benzylidene)-hydrazine |
| 216 | 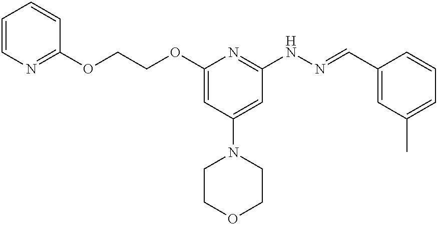 | N-(3-Methyl-benzylidene)-N′- {4-morpholin-4-yl-6-[2- (pyridin-2-yloxy)-ethoxy]- pyridin-2-yl}-hydrazine |
| 217 | 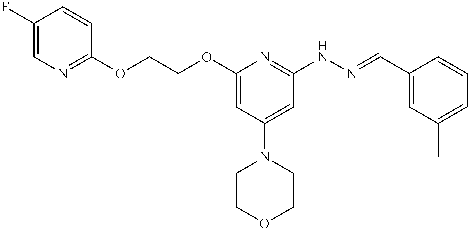 | N-{6-[2-(5-Fluoro-pyridin-2- yloxy)-ethoxy]-4-morpholin-4- yl-pyridin-2-yl}-N′-(3-methyl- benzylidene)-hydrazine |
| 218 | 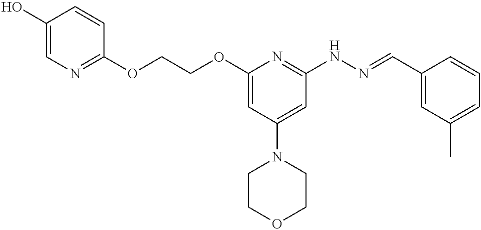 | 6-(2-{6-[N′-(3-Methyl- benzylidene)-hydrazino]-4- morpholin-4-yl-pyridin-2- yloxy}-ethoxy)-pyridin-3-ol |
| 219 | 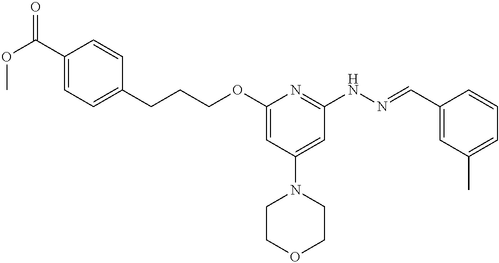 | 4-(3-{6-[N′-(3-Methyl- benzylidene)-hydrazino]-4- morpholin-4-yl-pyridin-2- yloxy}-propyl)-benzoic acid methyl ester |
| 220 | 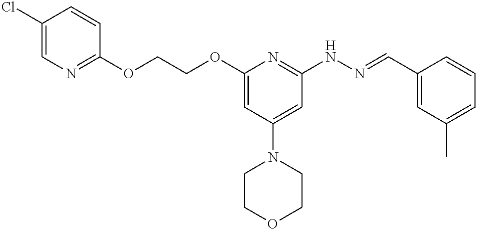 | N-{6-[2-(5-Chloro-pyridin-2- yloxy)-ethoxy]-4-morpholin-4- yl-pyridin-2-yl}-N′-(3-methyl- benzylidene)-hydrazine |
| 221 | 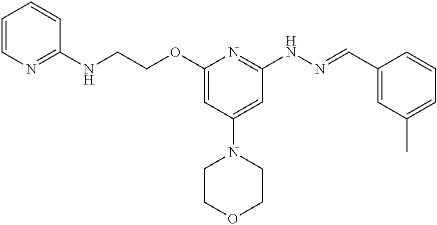 | (2-{6-[N′-(3-Methyl- benzylidene)-hydrazino]-4- morpholin-4-yl-pyridin-2- yloxy}-ethyl)-pyridin-2-yl- amine |
| 222 | 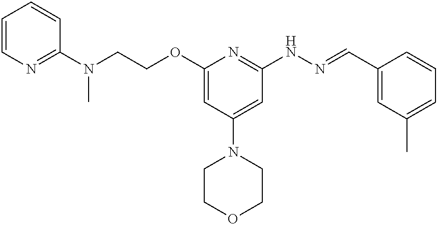 | Methyl-(2-{6-[N′-(3-methyl- benzylidene)-hydrazino]-4- morpholin-4-yl-pyridin-2- yloxy}-ethyl)-pyridin-2-yl- amine |
| 223 | 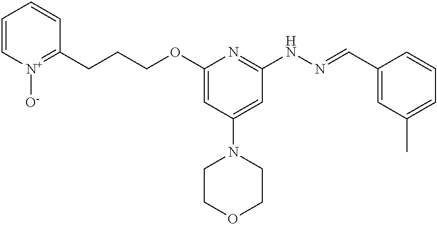 | N-(3-Methyl-benzylidene)-N′- {4-morpholin-4-yl-6-[3-(1-oxy- pyridin-2-yl)-propoxy]-pyridin- 2-yl)-hydrazine |
| 224 | 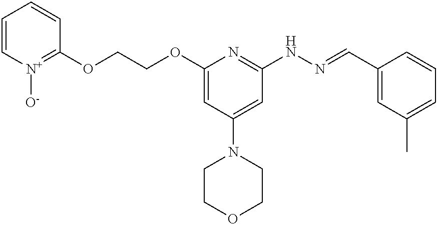 | N-(3-Methyl-benzylidene)-N′- {4-morpholin-4-yl-6-[2-(1-oxy- pyridin-2-yloxy)-ethoxy]- pyridin-2-yl}-hydrazine |
| 225 | 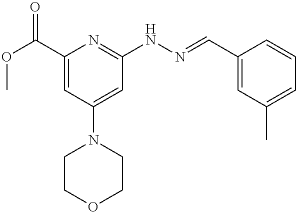 | 6-[N′-(3-Methyl-benzylidene)- hydrazino]-4-morpholin-4-yl- pyridine-2-carboxylic acid methyl ester |
| 226 | 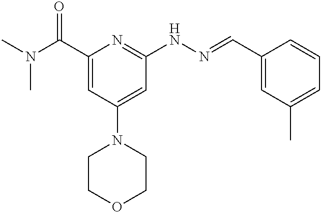 | 6-[N′-(3-Methyl-benzylidene)- hydrazino]-4-morpholin-4-yl- pyridine-2-carboxylic acid dimethylamide |
| 227 | 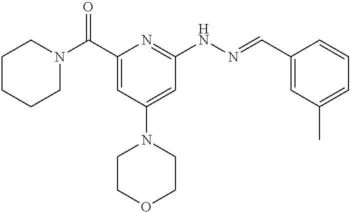 | {6-[N′-(3-Methyl-benzylidene)- hydrazino]-4-morpholin-4-yl- pyridin-2-yl}-piperidin-1-yl- methanone |
| 228 | 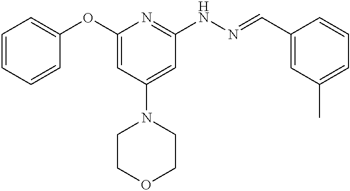 | N-(3-Methyl-benzylidene)-N′- (4-morpholin-4-yl-6-phenoxy- pyridin-2-yl)-hydrazine |
| 229 | 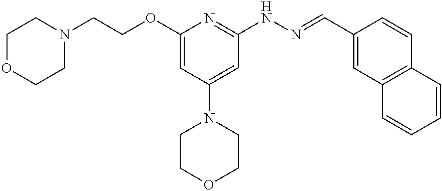 | N-[4-Morpholin-4-yl-6-(2- morpholin-4-yl-ethoxy)- pyridin-2-yl]-N′-naphthalen-2- ylmethylene-hydrazine |
| 230 | 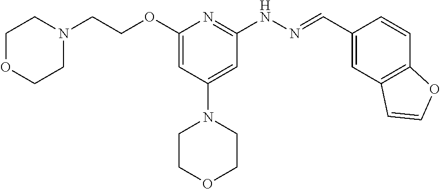 | N-Benzofuran-5-ylmethylene- N′-[4-morpholin-4-yl-6-(2- morpholin-4-yl-ethoxy)- pyridin-2-yl]-hydrazine |
| 231 | 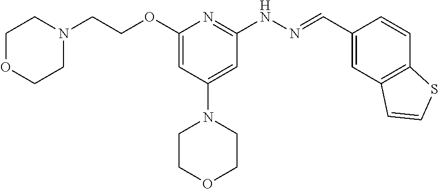 | N-Benzo[b]thiophen-5- ylmethylene-N′-[4-morpholin- 4-yl-6-(2-morpholin-4-yl- ethoxy)-pyridin-2-yl]- hydrazine |
| 232 | 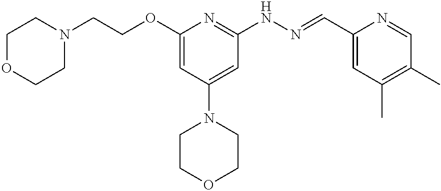 | N-(4,5-Dimethyl-pyridin-2- ylmethylene)-N′-[4-morpholin- 4-yl-6-(2-morpholin-4-yl- ethoxy)-pyridin-2-yl]- hydrazine |
| 233 | 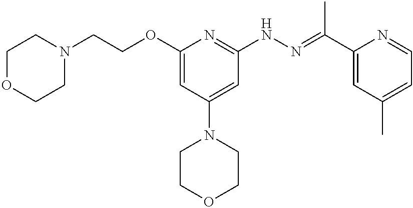 | N-[1-(4-Methyl-pyridin-2-yl)- ethylidene]-N′-[4-morpholin-4- yl-6-(2-morpholin-4-yl- ethoxy)-pyridin-2-yl]- hydrazine |
| 234 | 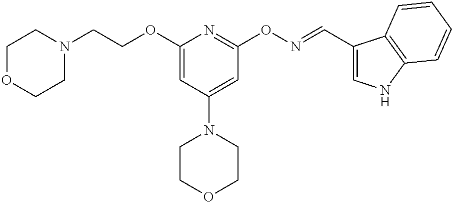 | 1H-Indole-3-carbaldehyde O- [4-morpholin-4-yl-6-(2- morpholin-4-yl-ethoxy)- pyridin-2-yl]-oxime |
| 235 | 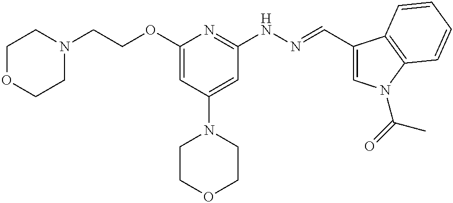 | 1-(3-{[4-Morpholin-4-yl-6-(2- morpholin-4-yl-ethoxy)- pyridin-2-yl]- hydrazonomethyl}-indol-1-yl)- ethanone |
| 236 | 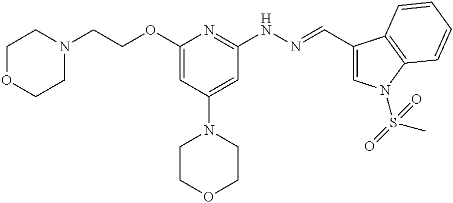 | N-(1-Methanesulfonyl-1H- indol-3-ylmethylene)-N′-[4- morpholin-4-yl-6-(2- morpholin-4-yl-ethoxy)- pyridin-2-yl]-hydrazine |
| 237 | 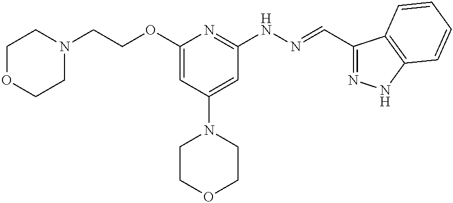 | N-(1H-Indazol-3-ylmethylene)- N′-[4-morpholin-4-yl-6-(2- morpholin-4-yl-ethoxy)- pyridin-2-yl]-hydrazine |
| 238 | 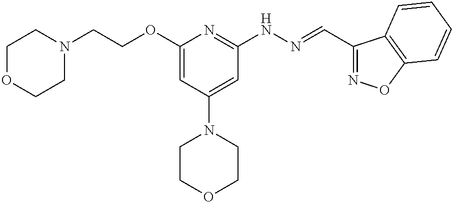 | N-Benzo[d]isoxazol-3- ylmethylene-N′-[4-morpholin- 4-yl-6-(2-morpholin-4-yl- ethoxy)-pyridin-2-yl]- hydrazine |
| 239 | 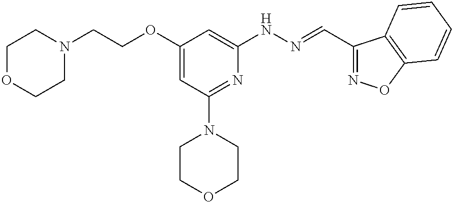 | N-Benzo[d]isoxazol-3- ylmethylene-N′-[6-morpholin- 4-yl-4-(2-morpholin-4-yl- ethoxy)-pyridin-2-yl]- hydrazine |
| 240 | 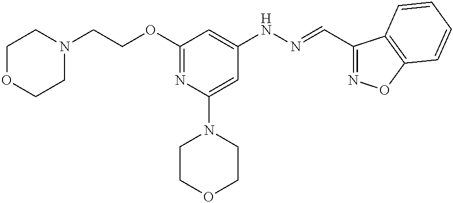 | N-Benzo[d]isoxazol-3- ylmethylene-N′-[2-morpholin- 4-yl-6-(2-morpholin-4-yl- ethoxy)-pyridin-4-yl]- hydrazine |
| 241 | 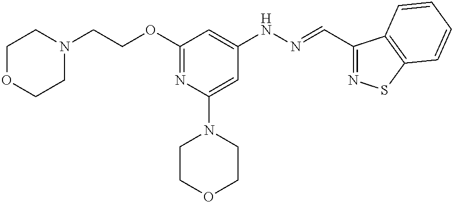 | N-Benzo[d]isothiazol-3- ylmethylene-N′-[2-morpholin- 4-yl-6-(2-morpholin-4-yl- ethoxy)-pyridin-4-yl]- hydrazine |
| 242 | 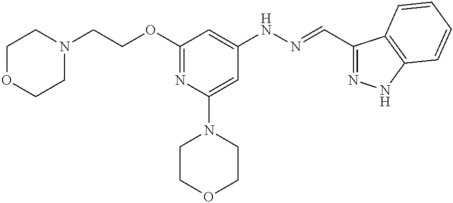 | N-(1H-Indazol-3-ylmethylene)- N′-[2-morpholin-4-yl-6-(2- morpholin-4-yl-ethoxy)- pyridin-4-yl]-hydrazine |
| 243 | 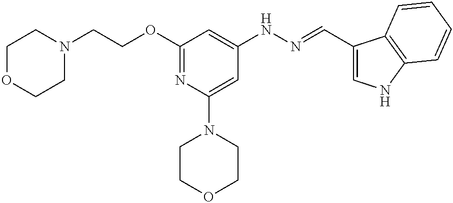 | N-(1H-Indol-3-ylmethylene)- N′-[2-morpholin-4-yl-6-(2- morpholin-4-yl-ethoxy)- pyridin-4-yl]-hydrazine |
| 244 | 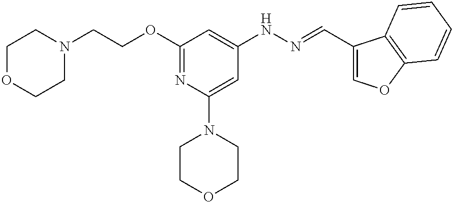 | N-Benzofuran-3-ylmethylene- N′-[2-morpholin-4-yl-6-(2- morpholin-4-yl-ethoxy)- pyridin-4-yl]-hydrazine |
| 245 | 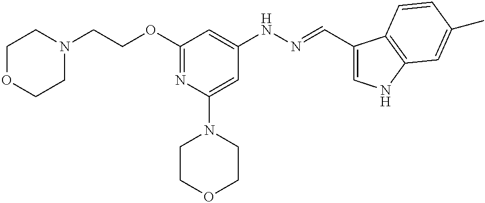 | N-(6-Methyl-1H-indol-3- ylmethylene)-N′-[2-morpholin- 4-yl-6-(2-morpholin-4-yl- ethoxy)-pyridin-4-yl]- hydrazine |
| 246 | 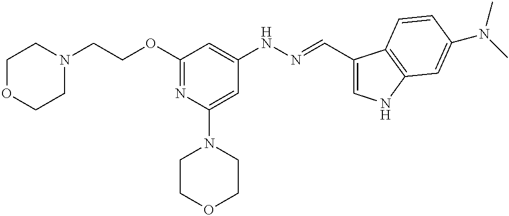 | Dimethyl-(3-{[2-morpholin-4- yl-6-(2-morpholin-4-yl- ethoxy)-pyridin-4-yl]- hydrazonomethyl}-1H-indol-6- yl)-amine |
| 247 | 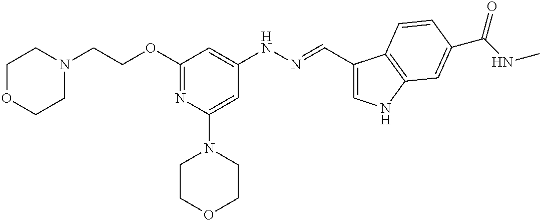 | 3-{[2-Morpholin-4-yl-6-(2- morpholin-4-yl-ethoxy)- pyridin-4-yl]- hydrazonomethyl}-1H-indole- 6-carboxylic acid methylamide |
| 248 | 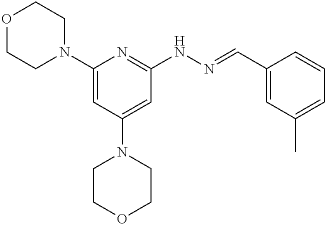 | N-(4,6-Di-morpholin-4-yl- pyridin-2-yl)-N′-(3-methyl- benzylidene)-hydrazine |
| 249 | 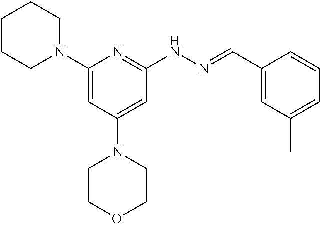 | N-(3-Methyl-benzylidene)-N′- (4′-morpholin-4-yl-3,4,5,6- tetrahydro-2H- [1,2′]bipyridinyl-6′-yl)- hydrazine |
| 250 | 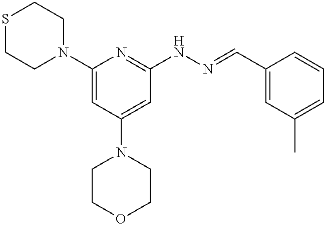 | N-(3-Methyl-benzylidene)-N′- (4-morpholin-4-yl-6- thiomorpholin-4-yl-pyridin-2- yl)-hydrazine |
| 251 | 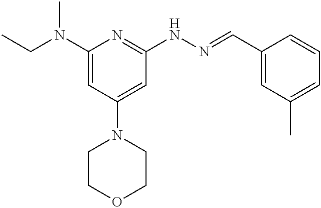 | Ethyl-methyl-{6-[N′-(3-methyl- benzylidene)-hydrazino]-4- morpholin-4-yl-pyridin-2-yl}- amine |
| 252 | 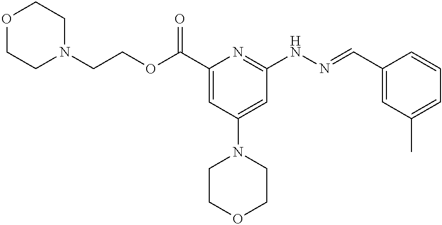 | 6-[N′-(3-Methyl-benzylidene)- hydrazino]-4-morpholin-4-yl- pyridine-2-carboxylic acid 2- morpholin-4-yl-ethyl ester |
| 253 | 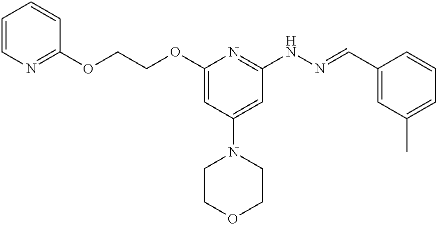 | N-(3-Methyl-benzylidene)-N′- {4-morpholin-4-yl-6-[2- (pyridin-2-yloxy)-ethoxy]- pyridin-2-yl}-hydrazine |
| 254 | 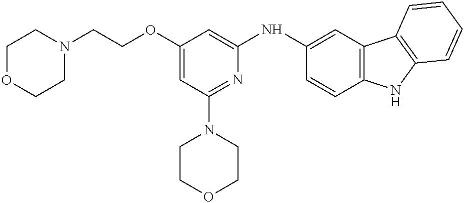 | (9H-Carbazol-3-yl)-[6- morpholin-4-yl-4-(2- morpholin-4-yl-ethoxy)- pyridin-2-yl]-amine |
| 255 | 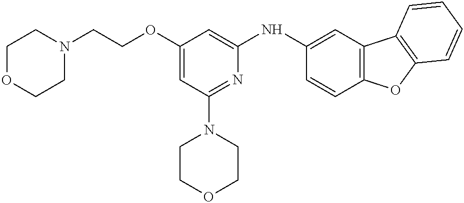 | Dibenzofuran-2-yl-[6- morpholin-4-yl-4-(2- morpholin-4-yl-ethoxy)- pyridin-2-yl]-amine |
| 256 | 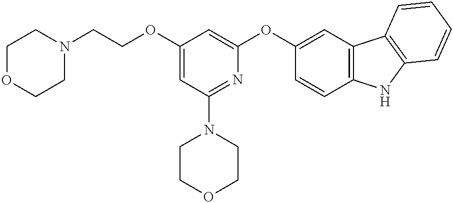 | 3-[6-Morpholin-4-yl-4-(2- morpholin-4-yl-ethoxy)- pyridin-2-yloxy]-9H-carbazole |
| 257 | 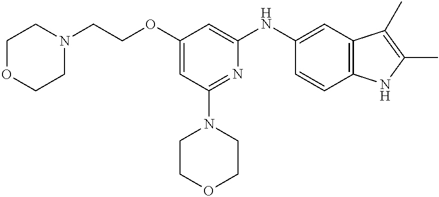 | (2,3-Dimethyl-1H-indol-5-yl)- [6-morpholin-4-yl-4-(2- morpholin-4-yl-ethoxy)- pyridin-2-yl]-amine |
| 258 | 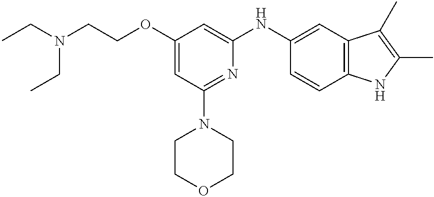 | [4-(2-Diethylamino-ethoxy)-6- morpholin-4-yl-pyridin-2-yl]- (2,3-dimethyl-1H-indol-5-yl)- amine |
| 259 | 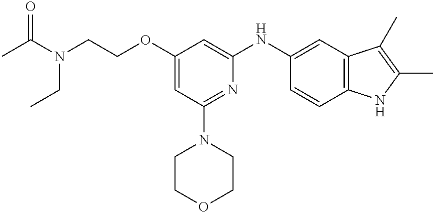 | N-{2-[2-(2,3-Dimethyl-1H- indol-5-ylamino)-6-morpholin- 4-yl-pyridin-4-yloxy]-ethyl}- N-ethyl-acetamide |
| 260 | 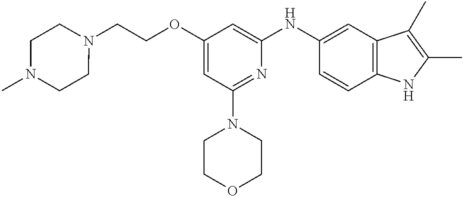 | (2,3-Dimethyl-1H-indol-5-yl)- {4-[2-(4-methyl-piperazin-1- yl)-ethoxy]-6-morpholin-4-yl- pyridin-2-yl}-amine |
| 261 | 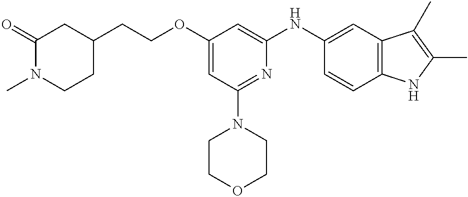 | 4-{2-[2-(2,3-Dimethyl-1H- indol-5-ylamino)-6-morpholin- 4-yl-pyridin-4-yloxy]-ethyl}-1- methyl-piperidin-2-one |
| 262 | 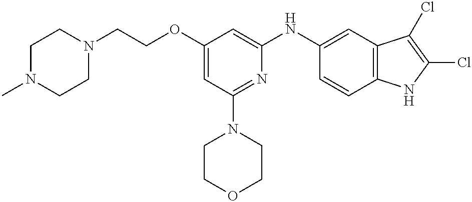 | (2,3-Dichloro-1H-indol-5-yl)- {4-[2-(4-methyl-piperazin-1- yl)-ethoxy]-6-morpholin-4-yl- pyridin-2-yl}-amine |
| 263 | 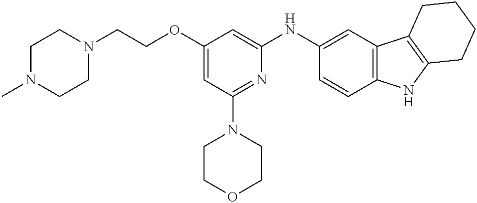 | {4-[2-(4-Methyl-piperazin-1- yl)-ethoxy]-6-morpholin-4-yl- pyridin-2-yl}-(6,7,8,9- tetrahydro-5H-carbazol-3-yl)- amine |
| 264 | 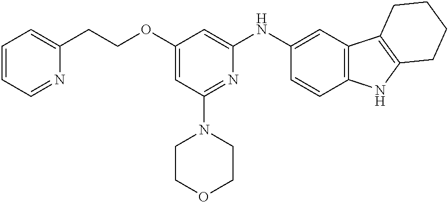 | [6-Morpholin-4-yl-4-(2- pyridin-2-yl-ethoxy)-pyridin-2- yl]-(6,7,8,9-tetrahydro-5H- carbazol-3-yl)-amine |
| 265 | 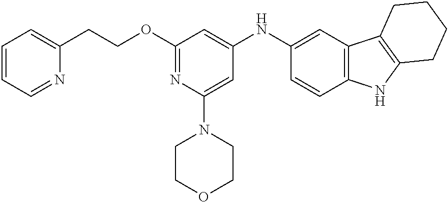 | [2-Morpholin-4-yl-6-(2- pyridin-2-yl-ethoxy)-pyridin-4- yl]-(6,7,8,9-tetrahydro-5H- carbazol-3-yl)-amine |
| 266 | 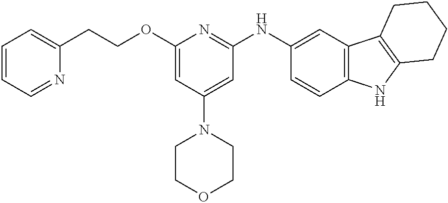 | [4-Morpholin-4-yl-6-(2- pyridin-2-yl-ethoxy)-pyridin-2- yl]-(6,7,8,9-tetrahydro-5H- carbazol-3-yl)-amine |
| 267 | 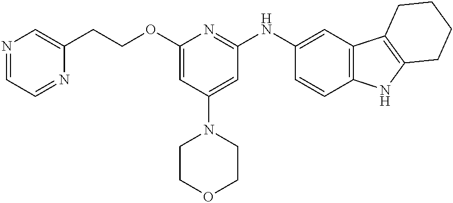 | [4-Morpholin-4-yl-6-(2- pyrazin-2-yl-ethoxy)-pyridin-2- yl]-(6,7,8,9-tetrahydro-5H- carbazol-3-yl)-amine |
| 268 | 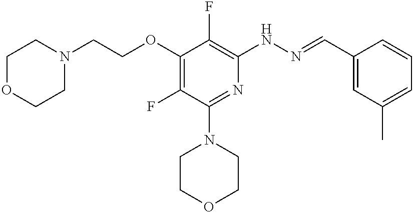 | N-[3,5-Difluoro-6-morpholin- 4-yl-4-(2-morpholin-4-yl- ethoxy)-pyridin-2-yl]-N′-(3- methyl-benzylidene)-hydrazine |
| 269 | 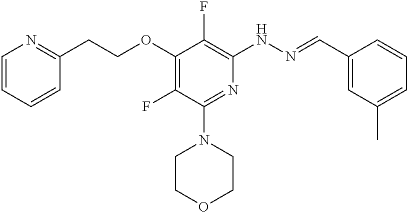 | N-[3,5-Difluoro-6-morpholin- 4-yl-4-(2-pyridin-2-yl-ethoxy)- pyridin-2-yl]-N′-(3-methyl- benzylidene)-hydrazine |
| 270 | 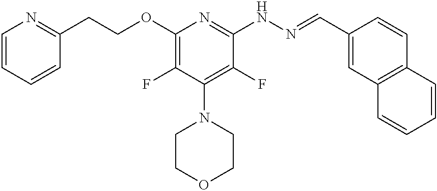 | N-[3,5-Difluoro-4-morpholin- 4-yl-6-(2-pyridin-2-yl-ethoxy)- pyridin-2-yl]-N′-naphthalen-2- ylmethylene-hydrazine |
| 271 | 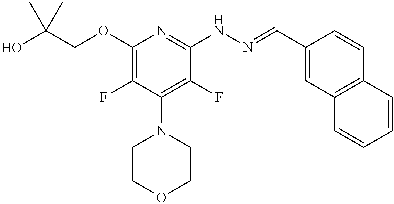 | 1-[3,5-Difluoro-4-morpholin-4- yl-6-(N′-naphthalen-2- ylmethylene-hydrazino)- pyridin-2-yloxy]-2-methyl- propan-2-ol |
| 272 | 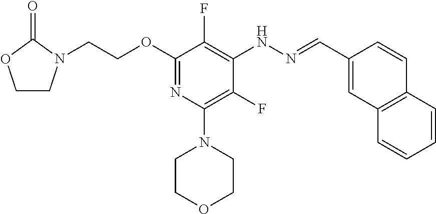 | 3-{2-[3,5-Diiluoro-6- morpholin-4-yl-4-(N′- naphthalen-2-ylmethylene- hydrazino)-pyridin-2-yloxy]- ethyl}-oxazolidin-2-one |
| 273 | 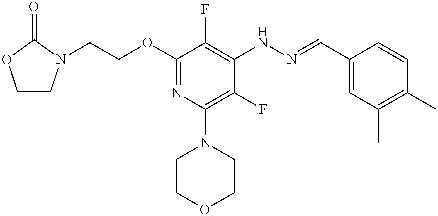 | 3-(2-{4-[N′-(3,4-Dimethyl- benzylidene)-hydrazino]-3,5- difluoro-6-morpholin-4-yl- pyridin-2-yloxy}-ethyl)- oxazolidin-2-one |
| 274 | 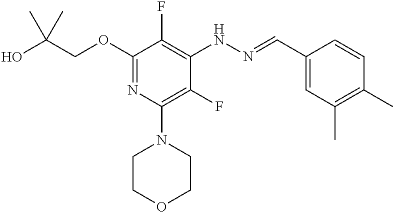 | 4-{4-[N′-(3,4-Dimethyl- benzylidene)-hydrazino]-3,5- difluoro-6-morpholin-4-yl- pyridin-2-yl}-2-methyl-butan- 2-ol |
| 275 | 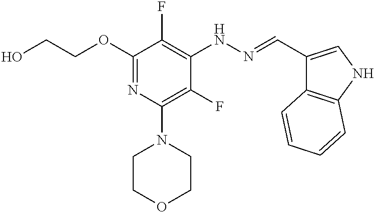 | 2-{3,5-Difluoro-4-[N′-(1H- indol-3-ylmethylene)- hydrazino]-6-morpholin-4-yl- pyridin-2-yloxy}-ethanol |
| 276 | 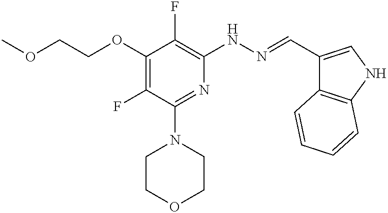 | N-[3,5-Difluoro-4-(2-methoxy- ethoxy)-6-morpholin-4-yl- pyridin-2-yl]-N′-(1H-indol-3- ylmethylene)-hydrazine |
| 277 | 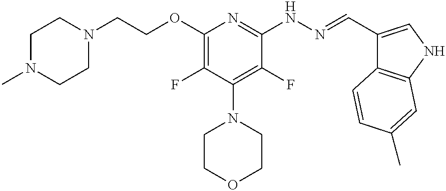 | N-{3,5-Difluoro-6-[2-(4- methyl-piperazin-1-yl)- ethoxy]-4-morpholin-4-yl- pyridin-2-yl}-N′-(6-methyl-1H- indol-3-ylmethylene)-hydrazine |
| 278 | 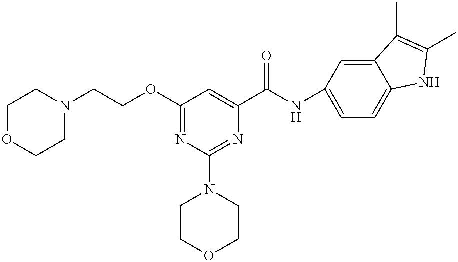 | 2-Morpholin-4-yl-6-(2- morpholin-4-yl-ethoxy)- pyrimidine-4-carboxylic acid (2,3-dimethyl-1H-indol-5-yl)- amide |
| 279 | 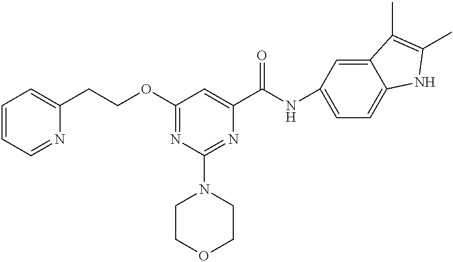 | 2-Morpholin-4-yl-6-(2-pyridin- 2-yl-ethoxy)-pyrimidine-4- carboxylic acid (2,3-dimethyl- 1H-indol-5-yl)-amide |
| 280 | 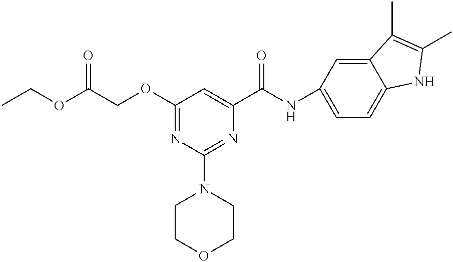 | [6-(2,3-Dimethyl-1H-indol-5- ylcabamoyl)-2-morpholin-4-yl- pyrimidin-4-yloxy]-acetic acid ethyl ester |
| 281 | 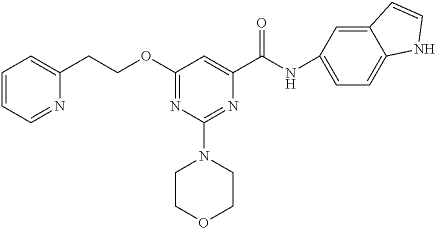 | 2-Morpholin-4-yl-6-(2-pyridin- 2-yl-ethoxy)-pyrimidine-4- carboxylic acid (1H-indol-5- yl)-amide |
| 282 | 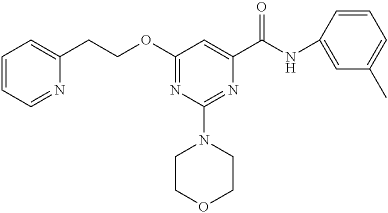 | 2-Morpholin-4-yl-6-(2-pyridin- 2-yl-ethoxy)-pyrimidine-4- carboxylic acid m-tolylamide |
| 283 | 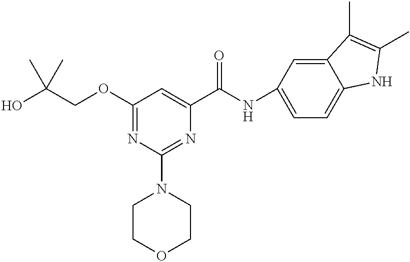 | 6-(2-hydroxy-2-methyl- propoxy)-2-morpholin-4-yl- pyrimidine-4-carboxylic acid (2,3-dimethyl-1H-indol-5-yl)- amide |
| 284 | 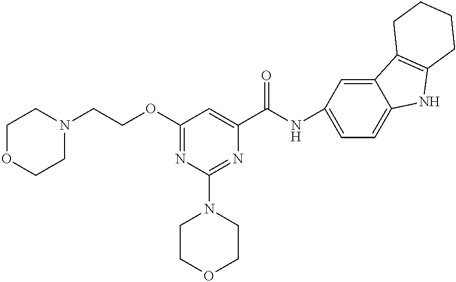 | 2-Morpholin-4-yl-6-(2- morpholin-4-yl-ethoxy)- pyrimidine-4-carboxylic acid (6,7,8,9-tetrahydro-5H- carbazol-3-yl)-amide |
| 285 | 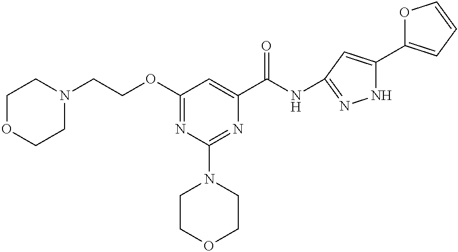 | 2-Morpholin-4-yl-6-(2- morpholin-4-yl-ethoxy)- pyrimidine-4-carboxylic acid (5-furan-2-yl-1H-pyrazol-3-yl)- amide |
| 286 | 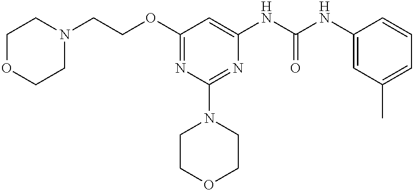 | 1-[2-Morpholin-4-yl-6-(2- morpholin-4-yl-ethoxy)- pyrimidin-4-yl]-3-m-tolyl-urea |
| 287 | 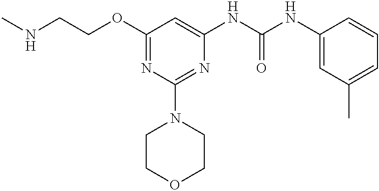 | 1-[6-(2-Methylamino-ethoxy)- 2-morpholin-4-yl-pyrimidin-4- yl]-3-m-tolyl-urea |
| 288 | 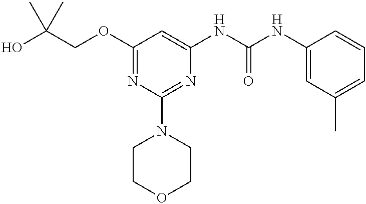 | 1-[6-(2-Hydroxy-2-methyl- propoxy)-2-morpholin-4-yl- pyrimidin-4-yl]-3-m-tolyl-urea |
| 289 | 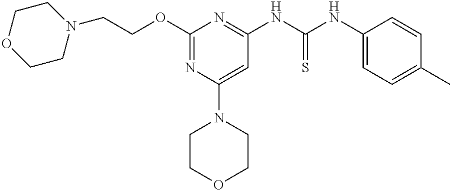 | 1-[6-Morpholin-4-yl-2-(2- morpholin-4-yl-ethoxy)- pyrimidin-4-yl]-3-p-tolyl- thiourea |
| 290 | 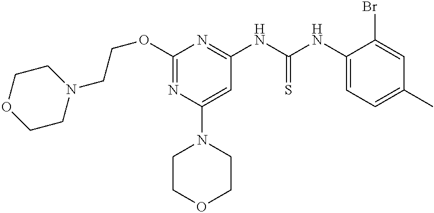 | 1-(2-Bromo-4-methyl-phenyl)- 3-[6-morpholin-4-yl-2-(2- morpholin-4-yl-ethoxy)- pyrimidin-4-yl]-thiourea |
| 291 | 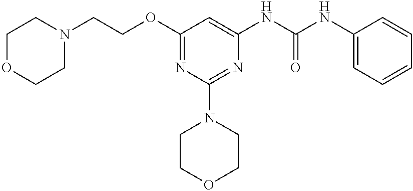 | 1-[2-Morpholin-4-yl-6-(2- morpholin-4-yl-ethoxy)- pyrimidin-4-yl]-3-phenyl-urea |
| 293 | 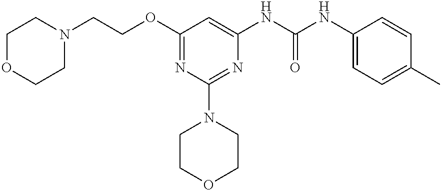 | 1-[2-Morpholin-4-yl-6-(2- morpholin-4-yl-ethoxy)- pyrimidin-4-yl]-3-p-tolyl-urea |
| 294 | 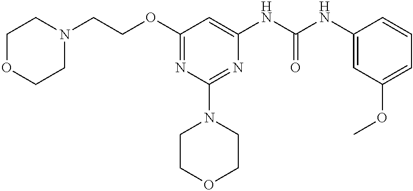 | 1-(3-Methoxy-phenyl)-3-[2- morpholin-4-yl-6-(2- morpholin-4-yl-ethoxy)- pyrimidin-4-yl]-urea |
| 295 | 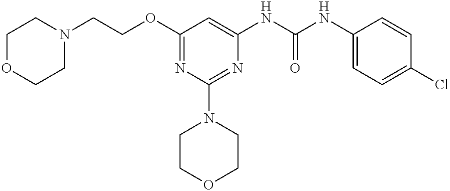 | 1-(4-Chloro-phenyl)-3-[2- morpholin-4-yl-6-(2- morpholin-4-yl-ethoxy)- pyrimidin-4-yl]-urea |
| 296 | 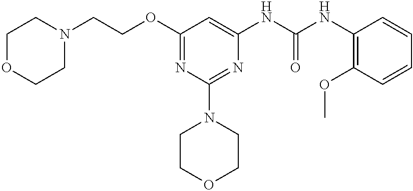 | 1-(2-Methoxy-phenyl)-3-[2- morpholin-4-yl-6-(2- morpholin-4-yl-ethoxy)- pyrimidin-4-yl]-urea |
| 297 | 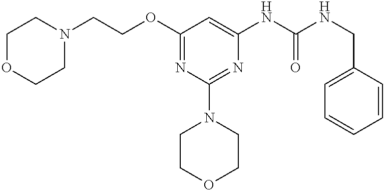 | 1-Benzyl-3-[2-morpholin-4-yl- 6-(2-morpholin-4-yl-ethoxy)- pyrimidin-4-yl]-urea |
| 298 | 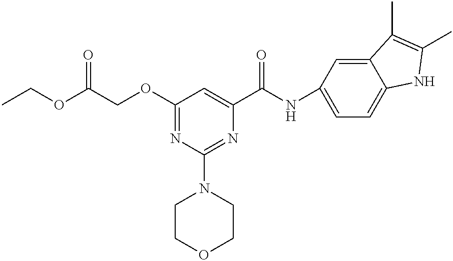 | [6-(2,3-Dimethyl-1H-indol-5- ylcarbamoyl)-2-morpholin-4- yl-pyrimidin-4-yloxy]-acetic acid ethyl ester |
| 299 | 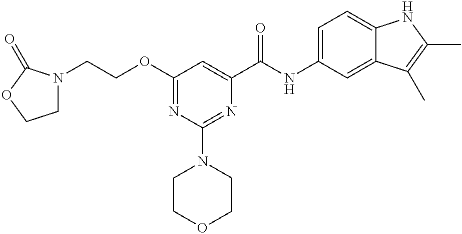 | 2-Morpholin-4-yl-6-[2-(2-oxo- oxazolidin-3-yl)-ethoxy]- pyrimidine-4-carboxylic acid (2,3-dimethyl-1H-indol-5-yl)- amide |
| 300 | 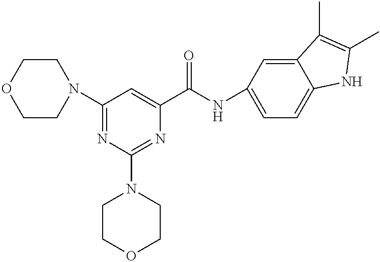 | 2,6-Di-morpholin-4-yl- pyrimidine-4-carboxylic acid (2,3-dimethyl-1H-indol-5-yl)- amide |
| 301 | 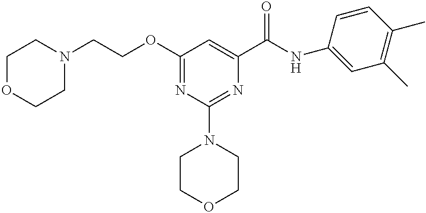 | 2-Morpholin-4-yl-6-(2- morpholin-4-yl-ethoxy)- pyrimidine-4-carboxylic acid (3,4-dimethyl-phenyl)-amide |
| 302 | 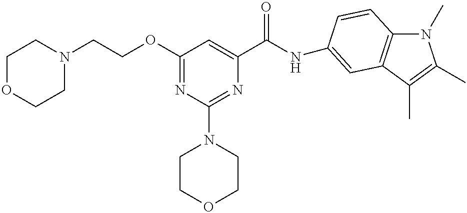 | 2-Morpholin-4-yl-6-(2- morpholin-4-yl-ethoxy)- pyrimidine-4-carboxylic acid (1,2,3-trimethyl-1H-indol-5- yl)-amide |
| 303 | 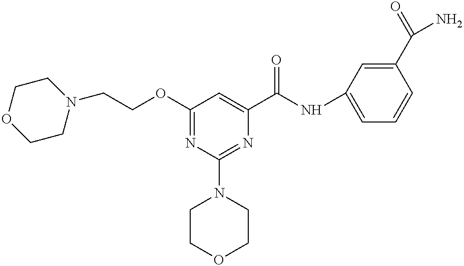 | 2-Morpholin-4-yl-6- (2-morpholin-4-yl-ethoxy)- pyrimidine-4-carboxylic acid (3-carbamoyl-phenyl)-amide |
| 304 | 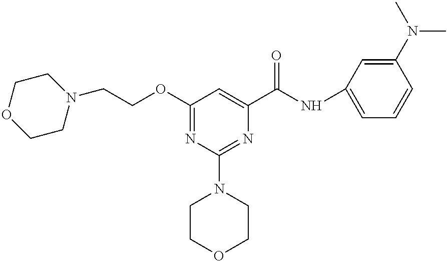 | 2-Morpholin-4-yl-6-(2- morpholin-4-yl-ethoxy)- pyrimidine-4-carboxylic acid (3-dimethylamino-phenyl)- amide |
| 305 | 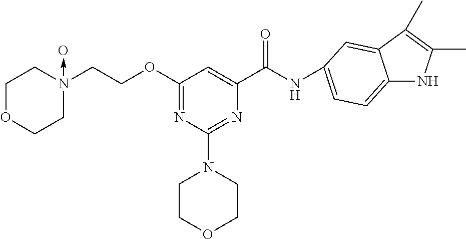 | 2-Morpholin-4-yl-6-[2-(4-oxy- morpholin-4-yl)-ethoxy]-pyrimidine- 4-carboxylic acid (2,3-dimethyl-1H- indol-5-yl)-amide |
| 306 | 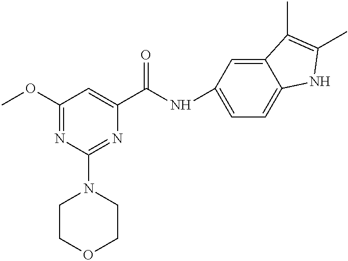 | 6-Methoxy-2-morpholin-4-yl- pyrimidine-4-carboxylic acid (2,3-dimethyl-1H-indol-5-yl)- amide |
| 307 | 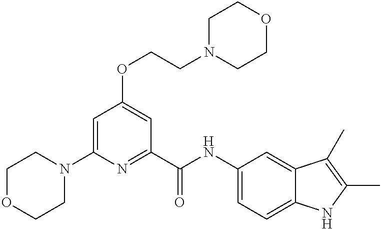 | 6-Morpholin-4-yl-4-(2- morpholin-4-yl-ethoxy)- pyridine-2-carboxylic acid (2,3-dimethyl-1H-indol-5-yl)- amide |
| 307 | 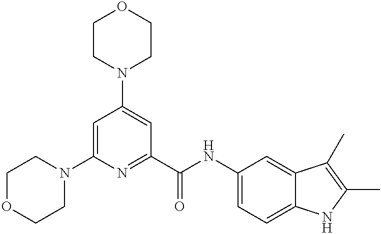 | 4,6-Di-morpholin-4-yl- pyridine-2-carboxylic aci (2,3- dimethyl-1H-indol-5-yl)-amide |
| 308 | 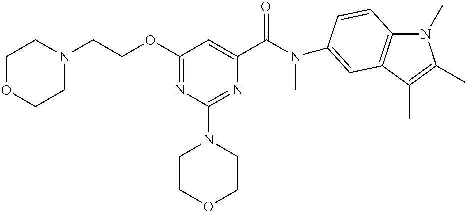 | 2-Morpholin-4-yl-6-(2- morpholin-4-yl-ethoxy)- pyrimidine-4-carboxylic acid methyl-(1,2,3-trimethyl-1H- indol-5-yl)-amide |
| 309 | 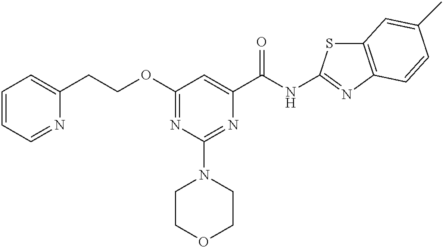 | 2-Morpholin-4-yl-6-(2-pyridin- 2-yl-ethoxy)-pyrimidine-4- carboxylic acid (6-methyl- benzothiazol-2-yl)-amide |
| 310 | 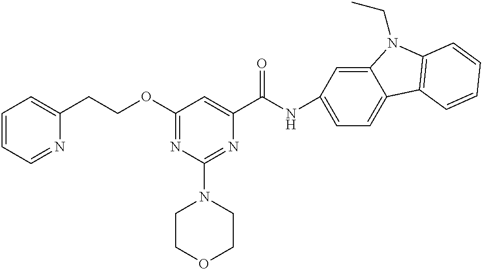 | 2-Morpholin-4-yl-6-(2-pyridin- 2-yl-ethoxy)-pyrimidine-4- carboxylic acid (9-ethyl-9H- carbazol-2-yl)-amide |
| 311 | 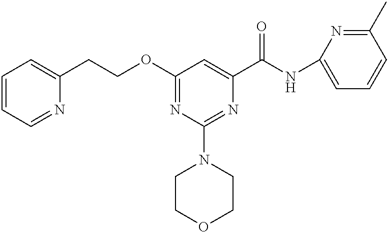 | 2-Morpholin-4-yl-6-(2-pyridin- 2-yl-ethoxy)-pyrimidine-4- carboxylic acid (6-methyl- pyridin-2-yl)-amide |
| 312 | 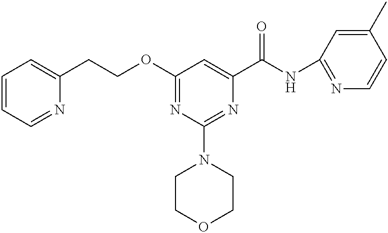 | 2-Morpholin-4-yl-6-(2-pyridin- 2-yl-ethoxy)-pyrimidine-4- carboxylic acid (4-methyl- pyridin-2-yl)-amide |
| 313 | 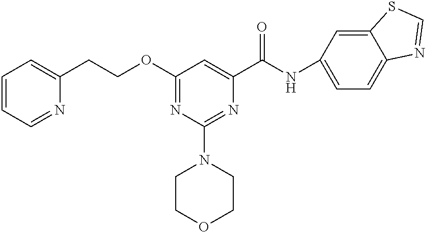 | 2-Morpholin-4-yl-6-(2-pyridin- 2-yl-ethoxy)-pyrimidine-4- carboxylic acid benzothiazol-6- ylamide |
| 314 | 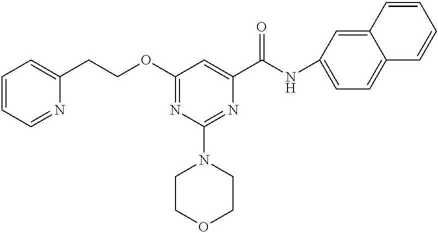 | 2-Morpholin-4-yl-6-(2-pyridin- 2-yl-ethoxy)-pyrimidine-4- carboxylic acid naphthalen-2- ylamide |
| 315 | 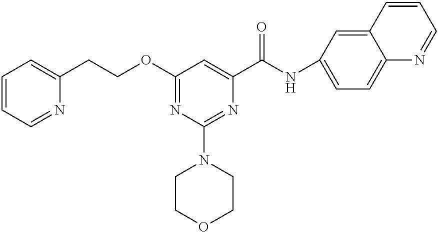 | 2-Morpholin-4-yl-6-(2-pyridin- 2-yl-ethoxy)-pyrimidine-4- carboxylic acid quinolin-6- ylamide |
| 316 | 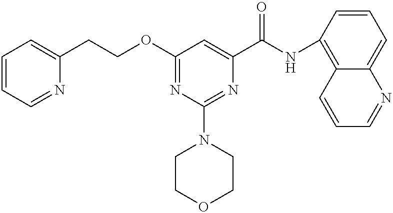 | 2-Morpholin-4-yl-6-(2-pyridin- 2-yl-ethoxy)-pyrimidine-4- carboxylic acid quinolin-5- ylamide |
| 317 | 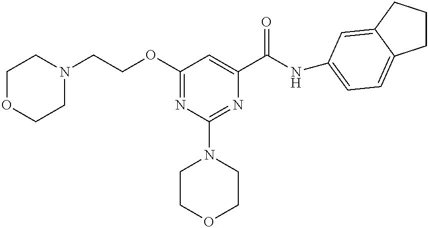 | 2-Morpholin-4-yl-6-(2- morpholin-4-yl-ethoxy)- pyrimidine-4-carboxylic acid indan-5-ylamide |
| 318 | 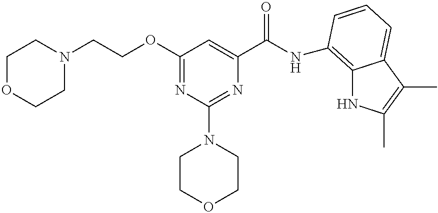 | 2-Morpholin-4-yl-6-(2- morpholin-4-yl-ethoxy)- pyrimidine-4-carboxylic acid (2,3-dimethyl-1H-indol-7-yl)- amide |
| 319 | 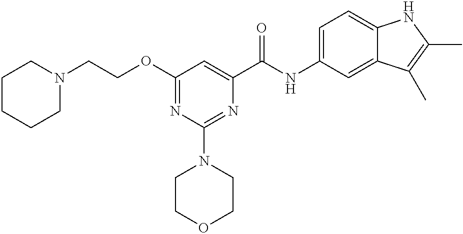 | 2-Morpholin-4-yl-6-(2- piperidin-1-yl-ethoxy)- pyrimidine-4-carboxylic acid (2,3-dimethyl-1H-indol-5-yl)- amide |
| 320 | 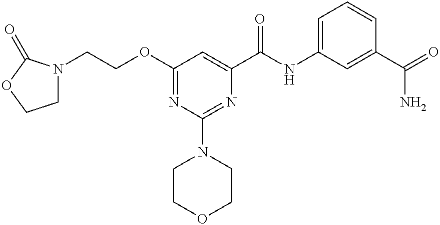 | 2-Morpholin-4-yl-6-[2-(2-oxo- oxazolidin-3-yl)-ethoxy]- pyrimidine-4-carboxylic acid (3-carbamoyl-phenyl)-amide |
| 321 | 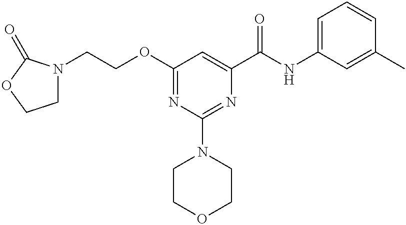 | 2-Morpholin-4-yl-6-[2-(2-oxo- oxazolidin-3-yl)-ethoxy]- pyrimidine-4-carboxylic acid m-tolylamide |
| 322 | 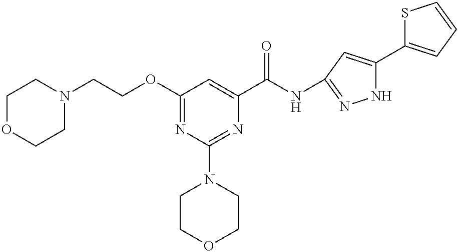 | 2-Morpholin-4-yl-6-(2- morpholin-4-yl-ethoxy)- pyrimidine-4-carboxylic acid (5-thiophen-2-yl-1H-pyrazol-3- yl)-amide |
| 323 | 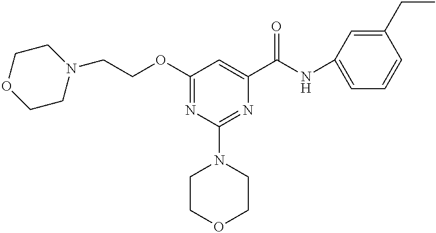 | 2-Morpholin-4-yl-6-(2- morpholin-4-yl-ethoxy)- pyrimidine-4-carboxylic acid (3-ethyl-phenyl)-amide |
| 324 | 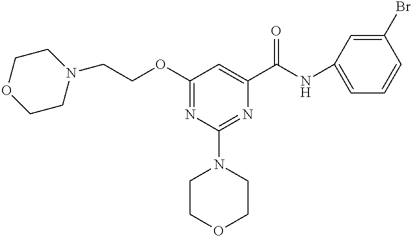 | 2-Morpholin-4-yl-6-(2- morpholin-4-yl-ethoxy)- pyrimidine-4-carboxylic acid (3-bromo-phenyl)-amide |
| 325 | 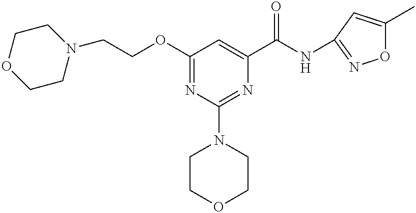 | 2-Morpholin-4-yl-6-(2- morpholin-4-yl-ethoxy)- pyrimidine-4-carboxylic acid (5-methyl-isoxazol-3-yl)-amide |
| 326 | 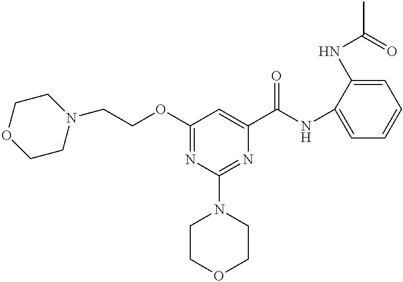 | 2-Morpholin-4-yl-6-(2- morpholin-4-yl-ethoxy)- pyrimidine-4-carboxylic acid (2-acetylamino-phenyl)-amide |
| 327 | 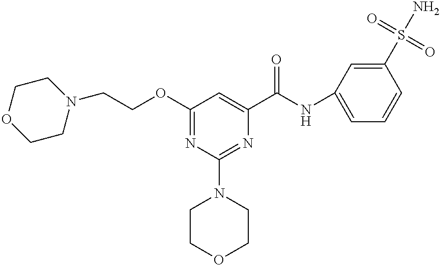 | 2-Morpholin-4-yl-6-(2- morpholin-4-yl-ethoxy)- pyrimidine-4-carboxylic acid (3-sulfamoyl-phenyl)-amide |
| 328 | 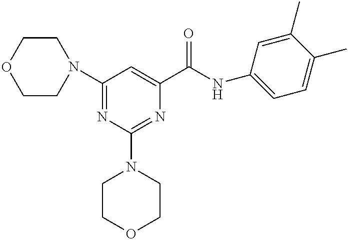 | 2,6-Di-morpholin-4-yl- pyrimidine-4-carboxylic acid (3,4-dimethyl-phenyl)-amide |
| 329 | 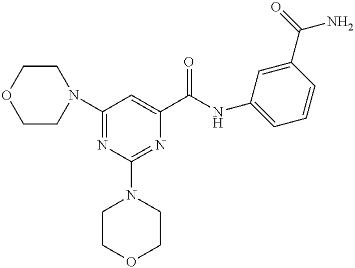 | 2,6-Di-morpholin-4-yl- pyrimidine-4-carboxylic acid (3-carbamoyl-phenyl)-amide |
| 330 | 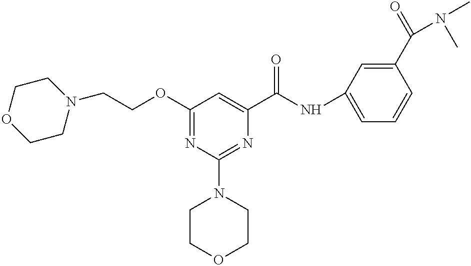 | 2-Morpholin-4-yl-6-(2- morpholin-4-yl-ethoxy)- pyrimidine-4-carboxylic acid (3-dimethylcarbamoyl-phenyl)- amide |
| 331 | 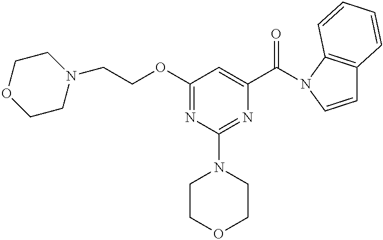 | Indol-1-yl-[2-morpholin-4-yl- 6-(2-morpholin-4-yl-ethoxy)- pyrimidin-4-yl]-methanone |
| 332 | 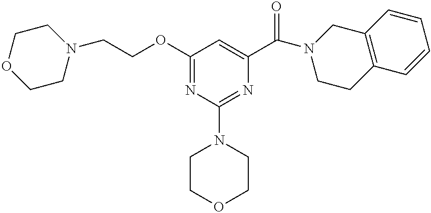 | (3,4-Dihydro-1H-isoquinolin- 2-yl)-[2-morpholin-4-yl-6-(2- morpholin-4-yl-ethoxy)- pyrimidin-4-yl]-methanone |
| 333 | 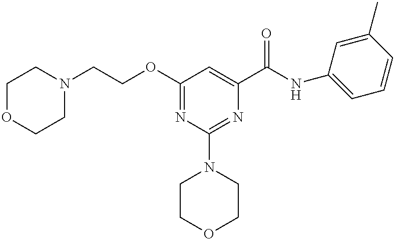 | 2-Morpholin-4-yl-6-(2- morpholin-4-yl-ethoxy)- pyrimidine-4-carboxylic acid m-tolylamide |
| 334 | 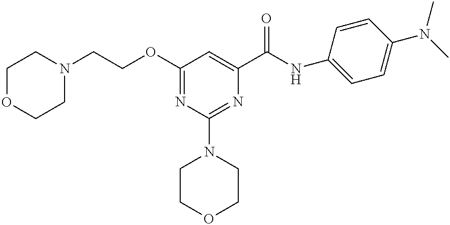 | 2-Morpholin-4-yl-6-(2- morpholin-4-yl-ethoxy)- pyrimidine-4-carboxylic acid (4-dimethylamino-phenyl)- amide |
| 335 | 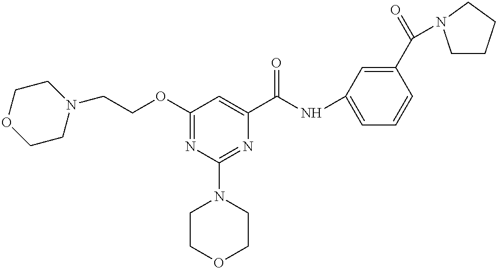 | 2-Morpholin-4-yl-6-(2- morpholin-4-yl-ethoxy)- pyrimidine-4-carboxylic acid [3-(pyrrolidine-1-carbonyl)- phenyl]-amide |
| 336 | 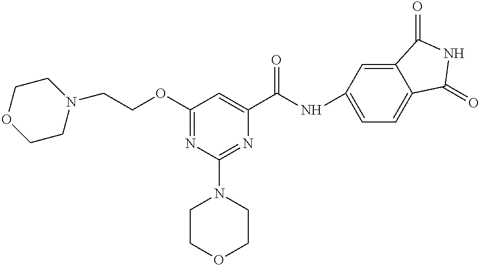 | 2-Morpholin-4-yl-6-(2- morpholin-4-yl-ethoxy)- pyrimidine-4-carboxylic acid (1,3-dioxo-2,3-dihydro-1H- isoindol-5-yl)-amide |
| 337 | 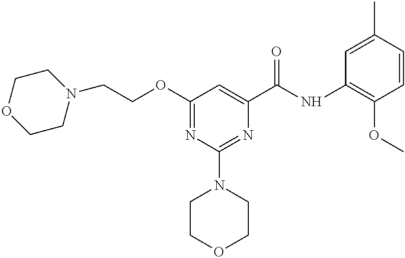 | 2-Morpholin-4-yl-6-(2- morpholin-4-yl-ethoxy)- pyrimidine-4-carboxylic acid (2-methoxy-5-methyl-phenyl)- amide |
| 338 | 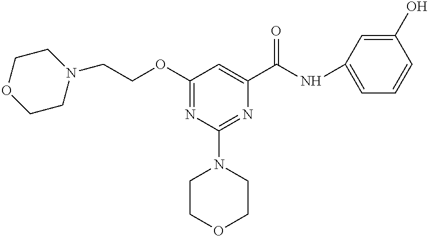 | 2-Morpholin-4-yl-6-(2- morpholin-4-yl-ethoxy)- pyrimidine-4-carboxylic acid (3-hydroxy-phenyl)-amide |
| 339 | 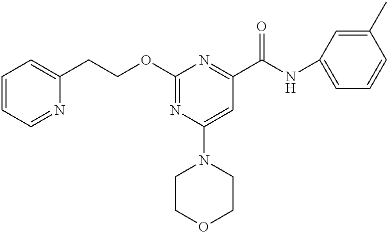 | 6-Morpholin-4-yl-2-(2-pyridin- 2-yl-ethoxy)-pyrimidine-4- carboxylic acid m-tolylamide |
| 340 | 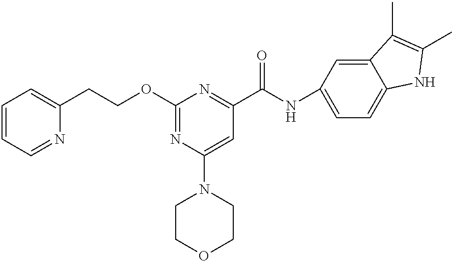 | 6-Morpholin-4-yl-2-(2-pyridin- 2-yl-ethoxy)-pyrimidine-4- carboxylic acid (2,3-dimethyl- 1H-indol-5-yl)-amide |
| 341 | 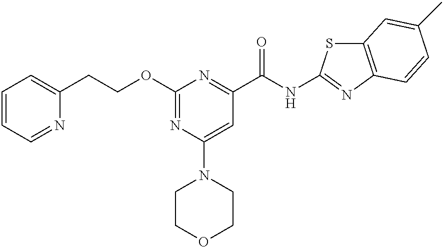 | 6-Morpholin-4-yl-2-(2-pyridin- 2-yl-ethoxy)-pyrimidine-4- carboxylic acid (6-methyl- benzothiazol-2-yl)-amide |
| 342 | 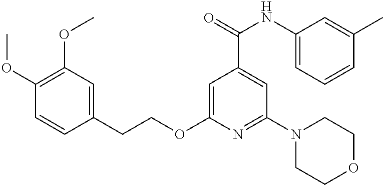 | 2-[2-(3,4-Dimethoxy-phenyl)- ethoxy]-6-morpholin-4-yl-N-m- tolyl-isonicotinamide |
| 343 | 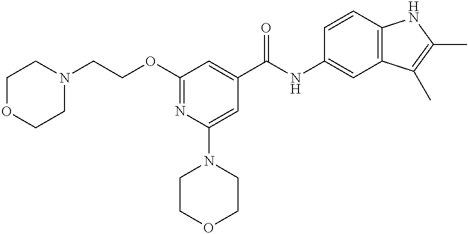 | N-(2,3-Dimelhyl-1H-indol-5- yl)-2-morpholin-4-yl-6-(2- morpholin-4-yl-ethoxy)- isonicotinamide |
| 344 | 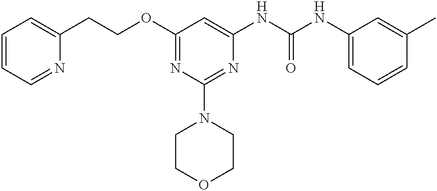 | 1-[2-Morpholin-4-yl-6-(2- pyridin-2-yl-ethoxy)- pyrimidin-4-yl]-3-m-tolyl-urea |
| 345 | 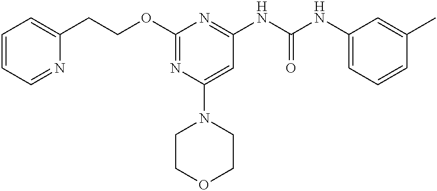 | 1-[6-Morpholin-4-yl-2-(2- pyridin-2-yl-ethoxy)- pyrimidin-4-yl]-3-m-tolyl-urea |
| 346 | 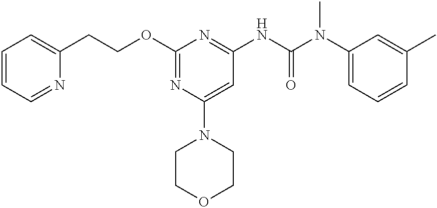 | 1-Methyl-3-[6-morpholin-4-yl- 2-(2-pyridin-2-yl-ethoxy)- pyrimidin-4-yl]-1-m-tolyl-urea |
| 347 | 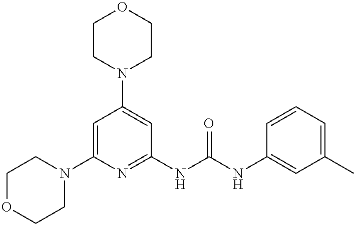 | 1-(4,6-Di-morpholin-4-yl- pyridin-2-yl)-3-m-tolyl-urea |
| 348 | 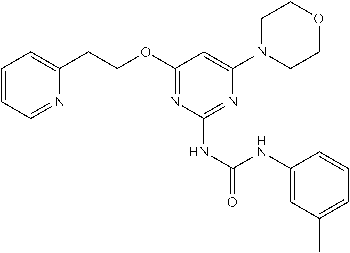 | 1-[(4-Morpholin-4-yl-6-(2- pyridin-2-yl-ethoxy)- pyrimidin-2-yl]-3-m-tolyl-urea |
| 349 | 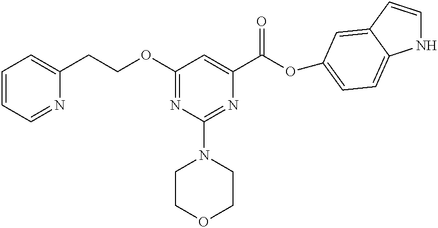 | 2-Morpholin-4-yl-6-(2-pyridin- 2-yl-ethoxy)-pyrimidine-4- carboxylic acid 1H-indol-5-yl ester |
| 350 | 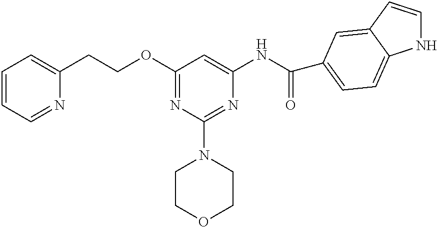 | 1H-Indole-5-carboxylic acid [2-morpholin-4-yl-6-(2- pyridin-2-yl-ethoxy)- pyrimidin-4-yl]-amide |
| 351 | 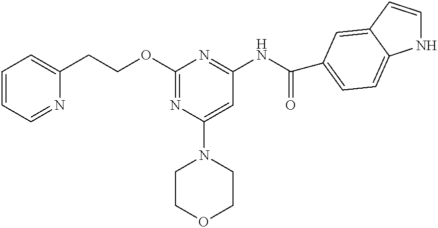 | 1H-Indole-5-carboxylic acid [6-morpholin-4-yl-2-(2- pyridin-2-yl-ethoxy)- pyrimidin-4-yl]-amide |
| 352 | 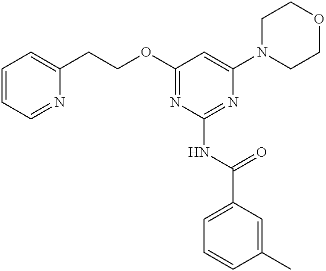 | 3-Methyl-N-[4-morpholin-4-yl- 6-(2-pyridin-2-yl-ethoxy)- pyrimidin-2-yl]-benzamide |
| 353 | 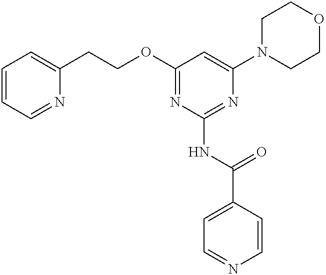 | N-[4-Morpholin-4-yl-6-(2- pyridin-2-yl-ethoxy)- pyrimidin-2-yl]- isonicotinamide |
| 354 | 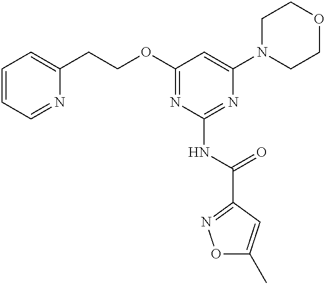 | 5-Methyl-isoxazole-3- carboxylic acid-[4-morpholin- 4-yl-6-(2-pyridin-2-yl-ethoxy)- pyrimidin-2-yl]-amide |
| 355 | 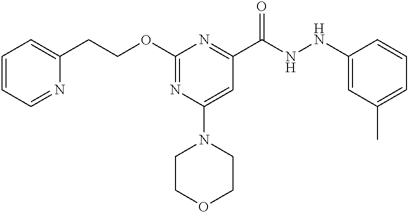 | 6-Morpholin-4-yl-2-(2-pyridin- 2-yl-ethoxy)-pyrimidine-4- carboxylic acid N′-m-tolyl- hydrazide |
| 356 | 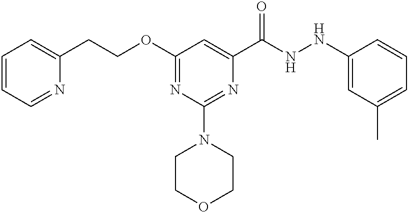 | 2-Morpholin-4-yl-6-(2-pyridin- 2-yl-ethoxy)-pyrimidine-4- carboxylic acid N′-m-tolyl- hydrazide |
| 357 | 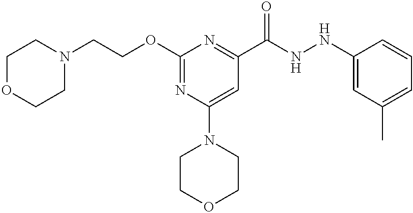 | 6-Morpholin-4-yl-2-(2- morpholin-4-yl-ethoxy)- pyrimidine-4-carboxylic acid N′-m-tolyl-hydrazide |
| 358 | 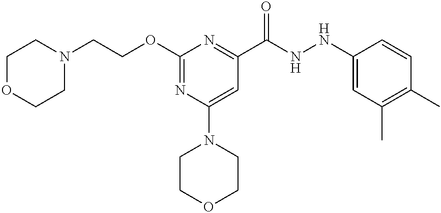 | 6-Morpholin-4-yl-2-(2- morpholin-4-yl-ethoxy)- pyrimidine-4-carboxylic acid N′-(3,4-dimethyl-phenyl)- hydrazide |
| 359 | 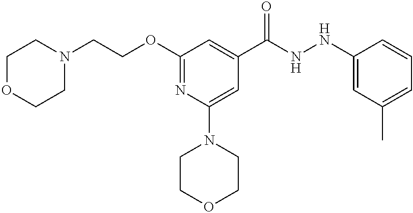 | 2-Morpholin-4-yl-6-(2- morpholin-4-yl-ethoxy)- isonicotinic acid N′-m-tolyl- hydrazide |
| 360 | 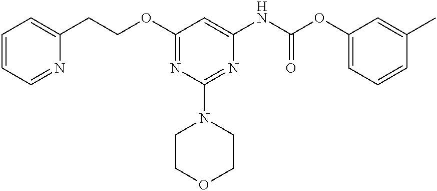 | [2-Morpholin-4-yl-6-(2- pyridin-2-yl-ethoxy)- pyrimidin-4-yl]-carbamic acid m-tolyl ester |
| 361 | 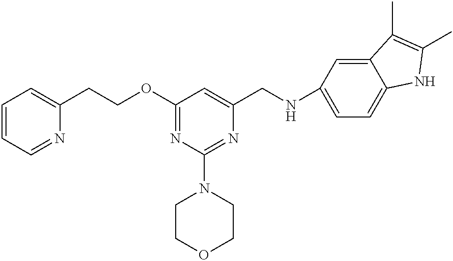 | (2,3-Dimethyl-1H-indol-5-yl)- [2-morpholin-4-yl-6-(2- pyridin-2-yl-ethoxy)- pyrimidin-4-ylmethyl]-amine |
| 362 | 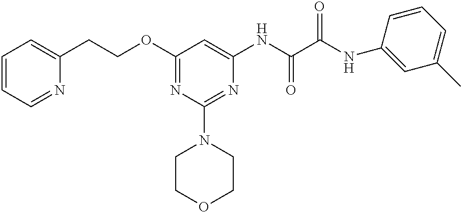 | N-[2-Morpholin-4-yl-6-(2- pyridin-2-yl-ethoxy)- pyrimidin-4-yl]-N′-m-tolyl- oxalamide |
| 363 | 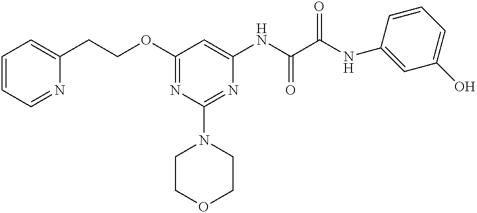 | N-(3-Hydroxy-phenyl)-N′-[2- morpholin-4-yl-6-(2-pyridin-2- yl-ethoxy)-pyrimidin-4-yl]- oxalamide |
| 364 | 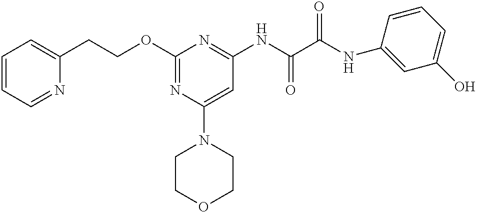 | N-(3-Hydroxy-phenyl)-N′-[6- morpholin-4-yl-2-(2-pyridin-2- yl-ethoxy)-pyrimidin-4-yl]- oxalamide |
| 365 | 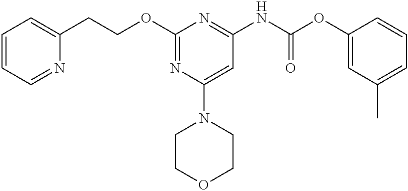 | [6-Morpholin-4-yl-2-(2- pyridin-2-yl-ethoxy)- pyrimidin-4-yl]-carbamic acid m-tolyl ester |
| 366 | 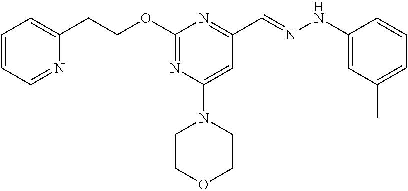 | N-[6-Morpholin-4-yl-2-(2- pyridin-2-yl-ethoxy)- pyrimidin-4-ylmethylene]-N′- m-tolyl-hydrazine |
| 367 | 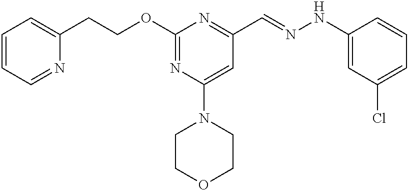 | N-(3-Chloro-phenyl)-N′-[6- morpholin-4-yl-2-(2-pyridin-2- yl-ethoxy)-pyrimidin-4- ylmethylene]-hydrazine |
| 368 | 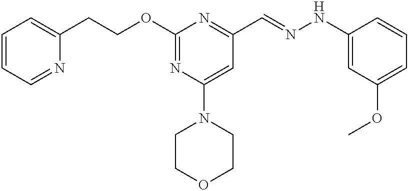 | N-(3-Methoxy-phenyl)-N′-[6- morpholin-4-yl-2-(2-pyridin-2- yl-ethoxy)-pyrimidin-4- ylmethylene]-hydrazine |
| 369 | 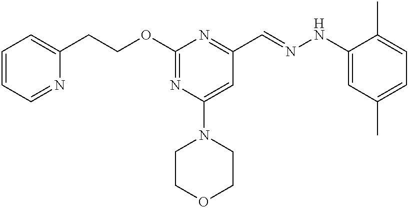 | N-(2,5-Dimethyl-phenyl)-N′- [6-morpholin-4-yl-2-(2- pyridin-2-yl-ethoxy)- pyrimidin-4-ylmethylene]- hydrazine |
| 370 | 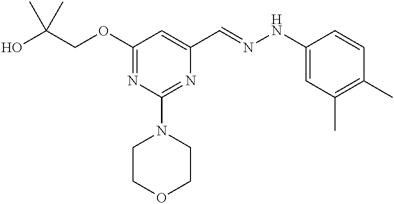 | 1-{6-[(3,4-Dimethyl-phenyl)- hydrazonomethyl]-2- morpholin-4-yl-pyrimidin-4- yloxy}-2-methyl-propan-2-ol |
| 371 | 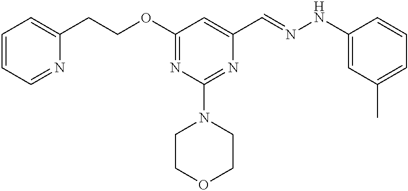 | N-[2-Morpholin-4-yl-6-(2- pyridin-2-yl-ethoxy)- pyrimidin-4-ylmethylene]-N′- m-tolyl-hydrazine |
| 372 | 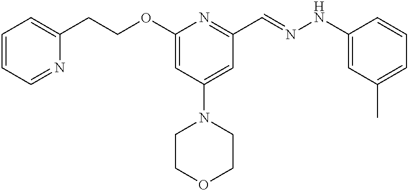 | N-[4-Morpholin-4-yl-6-(2- pyridin-2-yl-ethoxy)-pyridin-2- ylmethylene]-N′-m-tolyl- hydrazine |
| 373 | 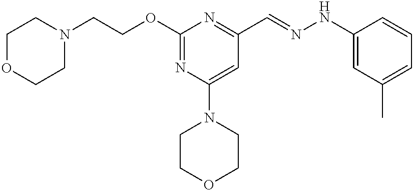 | N-[6-Morpholin-4-yl-2-(2- morpholin-4-yl-ethoxy)- pyrimidin-4-ylmethylene]-N′- m-tolyl-hydrazine |
| 374 | 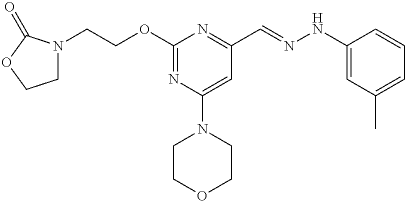 | 3-{2-[4-Morpholin-4-yl-6-(m- tolyl-hydrazonomethyl)- pyrimidin-2-yloxy]-ethyl}- oxazolidin-2-one |
| 375 | 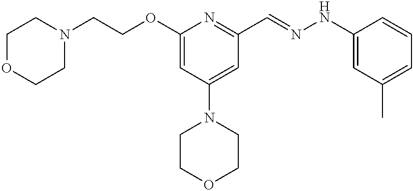 | N-[4-Morpholin-4-yl-6-(2- morpholin-4-yl-ethoxy)- pyridin-2-ylmethylene]-N′-m- tolyl-hydrazine |
| 376 | 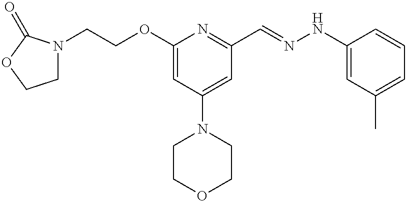 | 3-{2-[4-Morpholin-4-yl-6-(m- tolyl-hydrazonomethyl)- pyridin-2-yloxy]-ethyl}- oxazolidin-2-one |
| 377 | 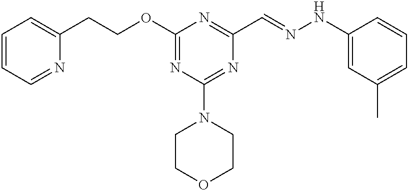 | N-[4-Morpholin-4-yl-6-(2- pyridin-2-yl-ethoxy)- [1,3,5]triazin-2-ylmethylene]- N′-m-tolyl-hydrazine |
| 378 | 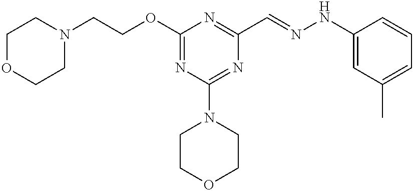 | N-[4-Morpholin-4-yl-6-(2- morpholin-4-yl-ethoxy)- [1,3,5]triazin-2-ylmethylene]- N′-m-tolyl-hydrazine |
| 379 | 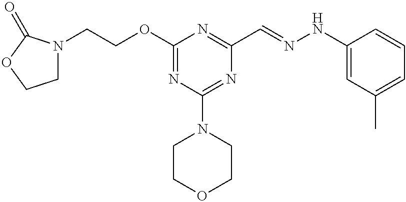 | 3-{2-[4-Morpholin-4-yl-6-(m- tolyl-hydrazonomethyl)- [1,3,5]triazin-2-yloxy]-ethyl}- oxazolidin-2-one |
| 380 | 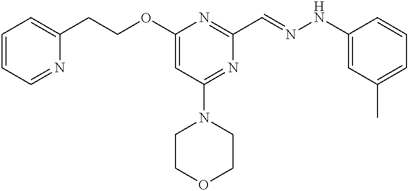 | N-[4-Morpholin-4-yl-6-(2- pyridin-2-yl-ethoxy)- pyrimidin-2-ylmethylene]-N′- m-tolyl-hydrazine |
| 381 | 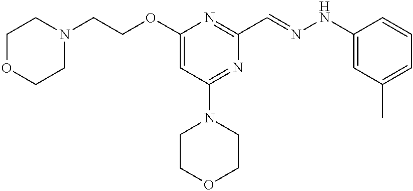 | N-[4-Morpholin-4-yl-6-(2- morpholin-4-yl-ethoxy)- pyrimidin-2-ylmethylene]-N′- m-tolyl-hydrazine |
| 382 | 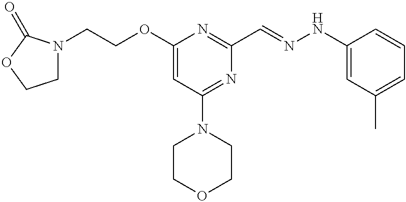 | 3-{2-[6-Morpholin-4-yl-2-(m- tolyl-hydrazonomethyl)- pyrimidin-4-yloxy]-ethyl}- oxazolidin-2-one |
| 383 | 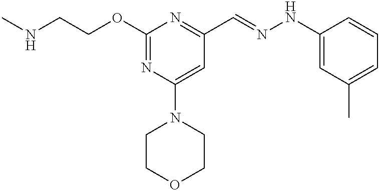 | Methyl-{2-[4-morpholin-4-yl- 6-(m-tolyl-hydrazonomethyl)- pyrimidin-2-yloxy]-ethyl}- amine |
| 384 | 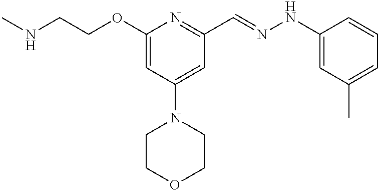 | Methyl-{2-[4-morpholin-4-yl- 6-(m-tolyl-hydrazonomethyl)- pyridin-2-yloxy]-ethyl}- amine |
| 385 | 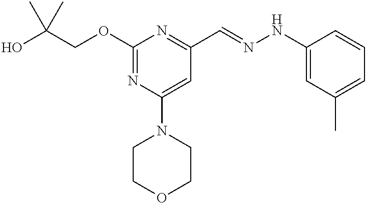 | 2-Methyl-1-[4-morpholin-4-yl- 6-(m-tolyl-hydrazonomethyl)- pyrimidin-2-yloxy]-propan-2-ol |
| 386 | 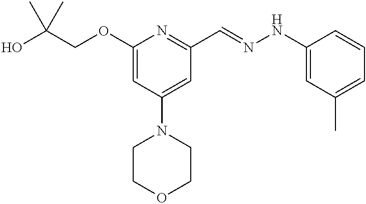 | 2-Methyl-1-[4-morpholin-4-yl- 6-(m-tolyl-hydrazonomethyl)- pyridin-2-yloxy]-propan-2-ol |
| 387 | 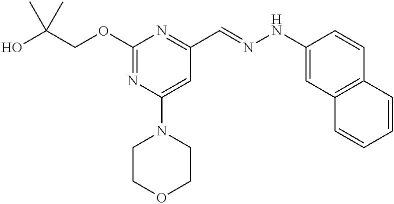 | 2-Methyl-1-[4-morpholin-4-yl- 6-(naphthalen-2-yl- hydrazonomethyl)-pyrimidin-2- yloxy]-propan-2-ol |
| 388 | 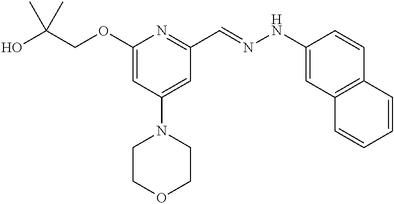 | 2-Methyl-1-[4-morpholin-4-yl- 6-(naphthalen-2-yl- hydrazonomethyl)-pyridin-2- yloxy]-propan-2-ol |
| 389 | 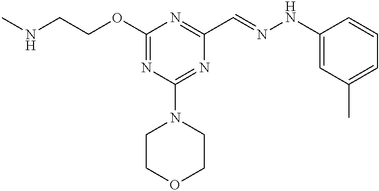 | Methyl-{2-[4-morpholin-4-yl- 6-(m-tolyl-hydrazonomethyl)- [1,3,5]triazin-2-yloxy]-ethyl}- amine |
| 390 | 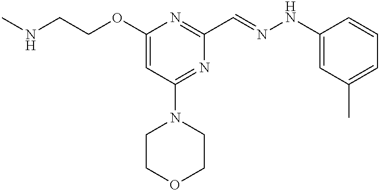 | Methyl-{2-[6-morpholin-4-yl- 2-(m-tolyl-hydrazonomethyl)- pyrimidin-4-yloxy]-ethyl}- amine |
| 391 | 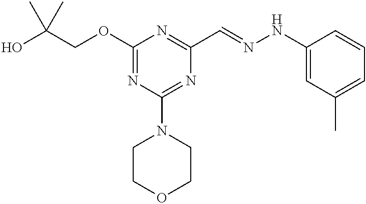 | 2-Methyl-1-[4-morpholin-4-yl- 6-(m-tolyl-hydrazonomethyl)- [1,3,5]triazin-2-yloxy]-propan- 2-ol |
| 392 | 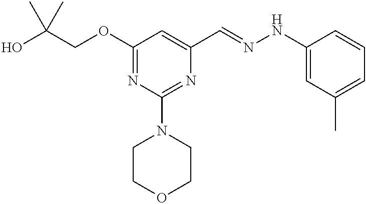 | 2-Methyl-1-[2-morpholin-4-yl- 6-(m-tolyl-hydrazonomethyl)- pyrimidin-4-yloxy]-propan-2-ol |
| 393 | 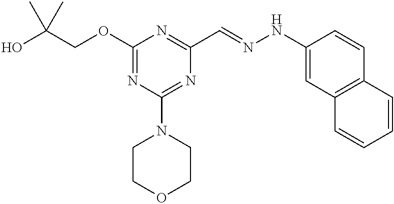 | 2-Methyl-1-[4-morpholin-4-yl- 6-(naphthalen-2-yl- hydrazonomethyl)- [1,3,5]triazin-2-yloxy]-propan- 2-ol |
| 394 | 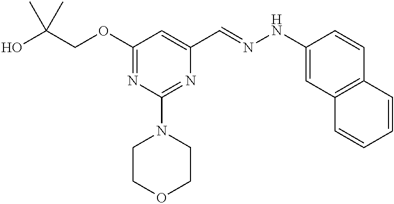 | 2-Methyl-1-[2-morpholin-4-yl- 6-(naphthalen-2-yl- hydrazonomethyl)-pyrimidin-4- yloxy]-propan-2-ol |
| 395 | 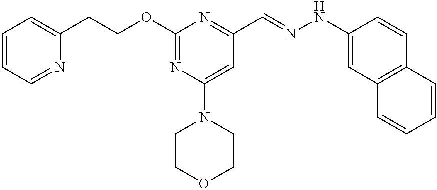 | N-[6-Morpholin-4-yl-2-(2- pyridin-2-yl-ethoxy)- pyrimidin-4-ylmethylene]-N′- naphthalen-2-yl-hydrazine |
| 396 | 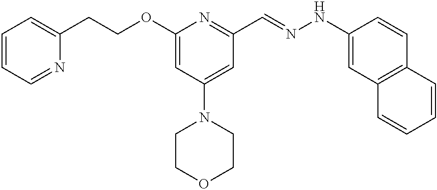 | N-[4-Morpholin-4-yl-6-(2- pyridin-2-yl-ethoxy)-pyridin-2- ylmethylene]-N′-naphthalen-2- yl-hydrazine |
| 397 | 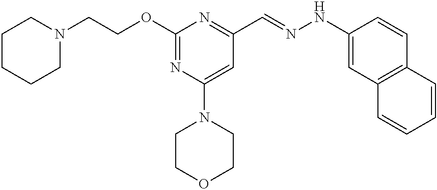 | N-[6-Morpholin-4-yl-2-(2- piperidin-1-yl-ethoxy)- pyrimidin-4-ylmethylene]-N′- naphthalen-2-yl-hydrazine |
| 398 | 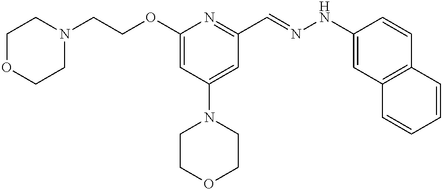 | N-[4-Morpholin-4-yl-6-(2- morpholin-4-yl-ethoxy)- pyridin-2-ylmethylene]-N′- naphthalen-2-yl-hydrazine |
| 399 | 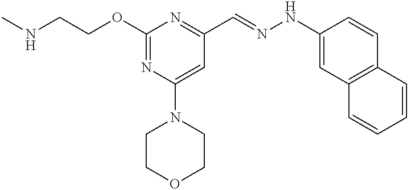 | Methyl-{2-[4-morpholin- 4-yl-6-(naphthalen-2-yl- hydrazonomethyl)-pyrimidin-2- yloxy]-ethyl}-amine |
| 400 | 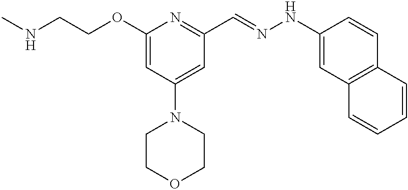 | Methyl-{2-[4-morpholin-4-yl- 6-(naphthalen-2-yl- hydrazonomethyl)-pyridin-2- yloxy]-ethyl}-amine |
| 401 | 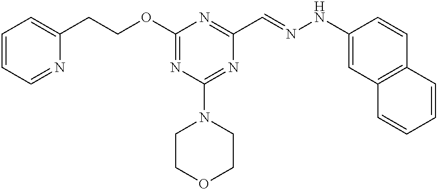 | N-[4-Morpholin-4-yl-6-(2- pyridin-2-yl-ethoxy)- [1,3,5]triazin-2-ylmethylene]- N′-naphthalen-2-yl-hydrazine |
| 402 | 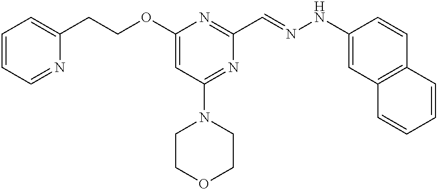 | N-[4-Morpholin-4-yl-6-(2- pyridin-2-yl-ethoxy)- pyrimidin-2-ylmethylene]-N′- naphthalen-2-yl-hydrazine |
| 403 | 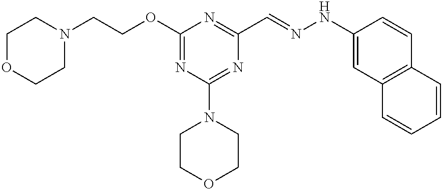 | N-[4-Morpholin-4-yl-6-(2- morpholin-4-yl-ethoxy)- [1,3,5]triazin-2-ylmethylene]- N′-naphthalen-2-yl-hydrazine |
| 404 | 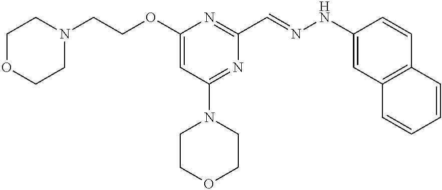 | N-[4-Morpholin-4-yl-6-(2- morpholin-4-yl-ethoxy)- pyrimidin-2-ylmethylene]-N′- naphthalen-2-yl-hydrazine |
| 405 | 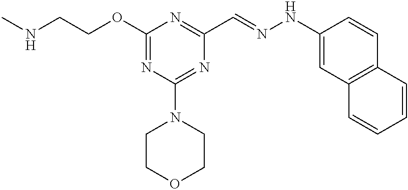 | Methyl-{2-[4-morpholin-4-yl- 6-(naphthalen-2-yl- hydrazonomethyl)- [1,3,5]triazin-2-yloxy]-ethyl}- amine |
| 406 | 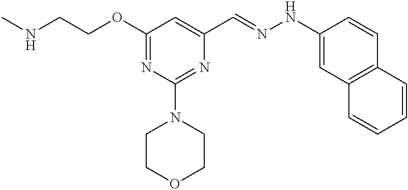 | Methyl-{2-[2-morpholin-4-yl- 6-(naphthalen-2-yl- hydrazonomethyl)-pyrimidin-4- yloxy]-ethyl}-amine |
| 407 | 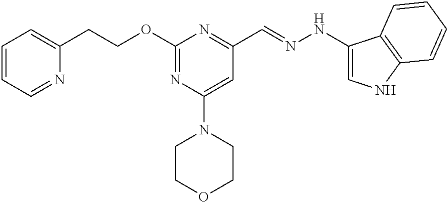 | N-(1H-Indol-3-yl)-N′-[6- morpholin-4-yl-2-(2-pyridin-2- yl-ethoxy)-pyrimidin-4- ylmethylene]-hydrazine |
| 408 | 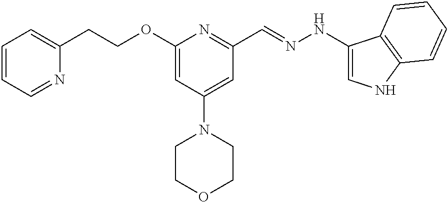 | N-(1H-Indol-3-yl)-N′-[4- morpholin-4-yl-6-(2-pyridin-2- yl-ethoxy)-pyridin-2- ylmethylene]-hydrazine |
| 409 | 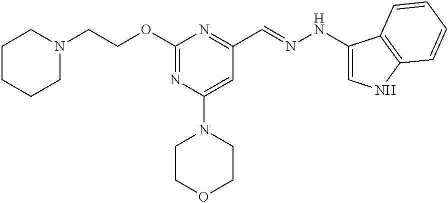 | N-(1H-Indol-3-yl)-N′-[6- morpholin-4-yl-2-(2-piperidin- 1-yl-ethoxy)-pyrimidin-4- ylmethylene]-hydrazine |
| 410 | 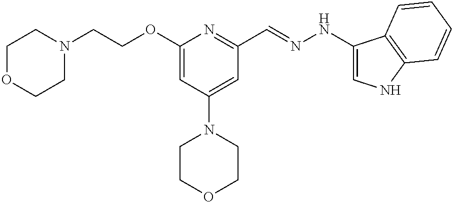 | N-(1H-Indol-3-yl)-N′-[4- morpholin-4-yl-6-(2- morpholin-4-yl-ethoxy)- pyridin-2-ylmethylene]- hydrazine |
| 411 | 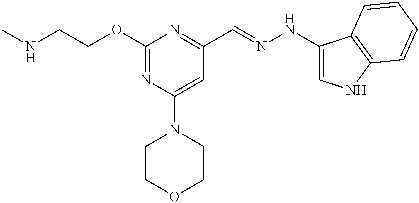 | (2-{4-[(1H-Indol-3-yl)- hydrazonomethyl]-6- morpholin-4-yl-pyrimidin-2- yloxy}-ethyl)-methyl-amine |
| 412 | 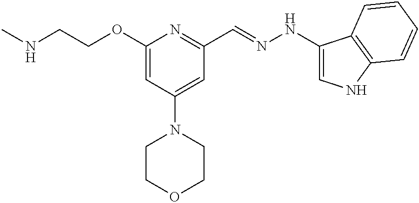 | (2-{6-[(1H-Indol-3-yl)- hydrazonomethyl]-4- morpholin-4-yl-pyridin-2- yloxy}-ethyl)-methyl-amine |
| 413 | 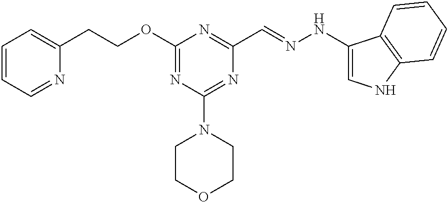 | N-(1H-Indol-3-yl)-N′-[4- morpholin-4-yl-6-(2-pyridin-2- yl-ethoxy)-[1,3,5]triazin-2- ylmethylene]-hydrazine |
| 414 | 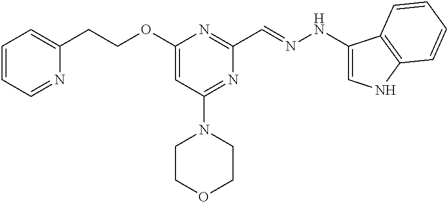 | N-(1H-Indol-3-yl)-N′-[4- morpholin-4-yl-6-(2-pyridin-2- yl-ethoxy)-pyrimidin-2- ylmethylene]-hydrazine |
| 415 | 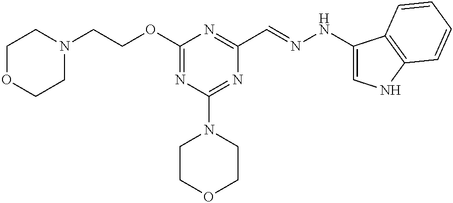 | N-(1H-Indol-3-yl)-N′-[4- morpholin-4-yl-6-(2- morpholin-4-yl-ethoxy)- [1,3,5]triazin-2-ylmethylene]- hydrazine |
| 416 | 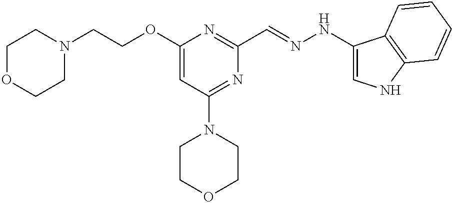 | N-(1H-Indol-3-yl)-N′-[4- morpholin-4-yl-6-(2- morpholin-4-yl-ethoxy)- pyrimidin-2-ylmethylene]- hydrazine |
| 417 | 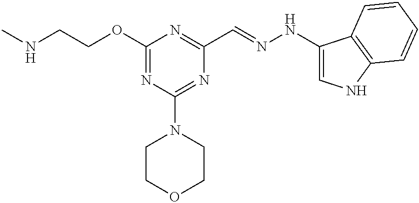 | (2-{4-[(1H-Indol-3-yl)- hydrazonomethyl]-6- morpholin-4-yl-[1,3,5]triazin- 2-yloxy}-ethyl)-methyl-amine |
| 418 | 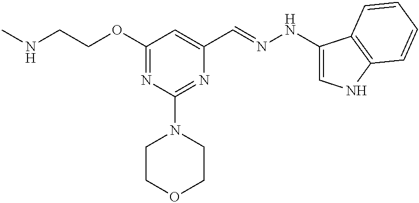 | (2-{6-[(1H-Indol-3-yl)- hydrazonomethyl]-2- morpholin-4-yl-pyrimidin-4- yloxy}-ethyl)-methyl-amine |
| 419 | 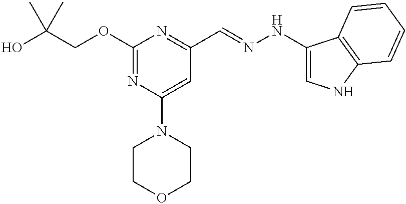 | 1-{4-[(1H-Indol-3-yl)- hydrazonomethyl]-6- morpholin-4-yl-pyrimidin-2- yloxy}-2-methyl-propan-2-ol |
| 420 | 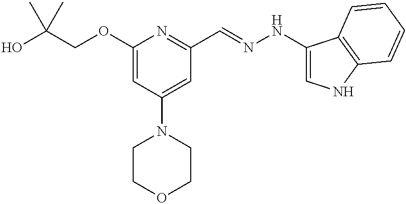 | 1-{6-[(1H-Indol-3-yl)- hydrazonomethyl]-4- morpholin-4-yl-pyridin-2- yloxy}-2-methyl-propan-2-ol |
| 421 | 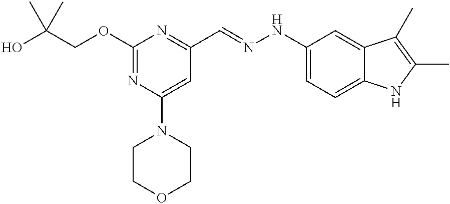 | 1-{4-[(2,3-Dimethyl-1H-indol- 5-yl)-hydrazonomethyl]-6- morpholin-4-yl-pyrimidin-2- yloxy}-2-methyl-propan-2-ol |
| 422 | 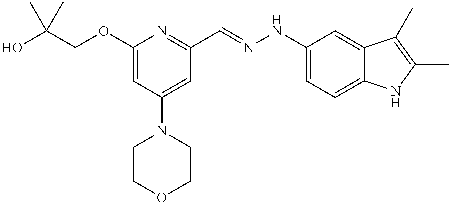 | 1-{6-[(2,3-Dimethyl-1H-indol- 5-yl)-hydrazonomethyl]-4- morpholin-4-yl-pyridin-2- yloxy}-2-methyl-propan-2-ol |
| 423 | 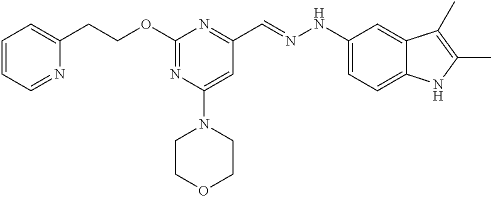 | N-(2,3-Dimethyl-1H-indol-5- yl)-N′-[6-morpholin-4-yl-2-(2- pyridin-2-yl-ethoxy)- pyrimidin-4-ylmethylene]- hydrazine |
| 424 | 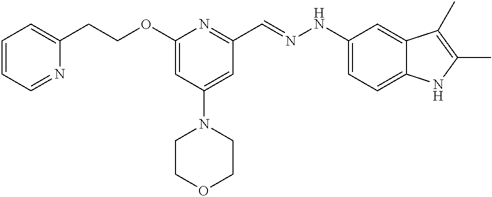 | N-(2,3-Dimethyl-1H-indol-5- yl)-N′-[4-morpholin-4-yl-6-(2- pyridin-2-yl-ethoxy)-pyridin-2- ylmethylene]-hydrazine |
| 425 | 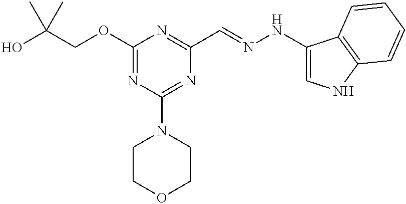 | 1-{4-[(1H-Indol-3-yl)- hydrazonomethyl]-6- morpholin-4-yl-[1,3,5]triazin- 2-yloxy}-2-methyl-propan-2-ol |
| 426 | 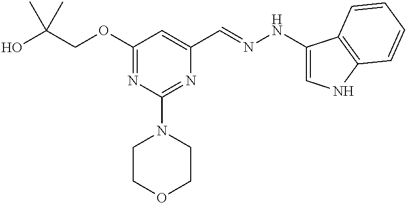 | 1-{6-[(1H-Indol-3-yl)- hydrazonomethyl]-2- morpholin-4-yl-pyrimidin-4- yloxy}-2-methyl-propan-2-ol |
| 427 | 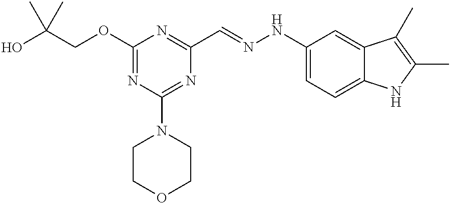 | 1-{4-[(2,3-Dimethyl-1H-indol- 5-yl)-hydrazonomethyl]-6- morpholin-4-yl-[1,3,5]triazin- 2-yloxy}-2-methyl-propan-2-ol |
| 428 | 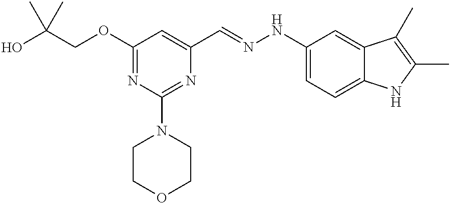 | 1-{6-[(2,3-Dimethyl-1H-indol- 5-yl)-hydrazonomethyl]-2- morpholin-4-yl-pyrimidin-4- yloxy}-2-methyl-propan-2-ol |
| 429 | 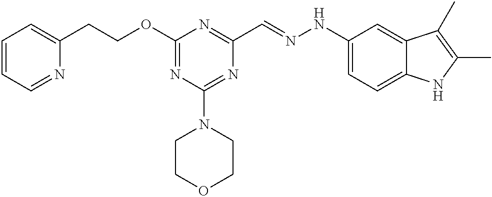 | N-(2,3-Dimethyl-1H-indol-5- yl)-N′-[4-morpholin-4-yl-6-(2- pyridin-2-yl-ethoxy)- [1,3,5]triazin-2-ylmethylene]- hydrazine |
| 430 | 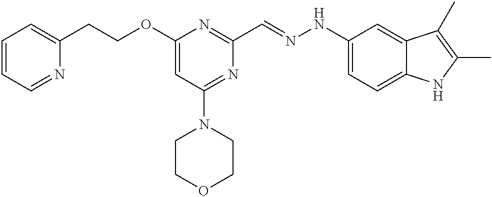 | N-(2,3-Dimethyl-1H-indol-5- yl)-N′-[4-morpholin-4-yl-6-(2- pyridin-2-yl-ethoxy)- pyrimidin-2-ylmethylene]- hydrazine |
| 431 | 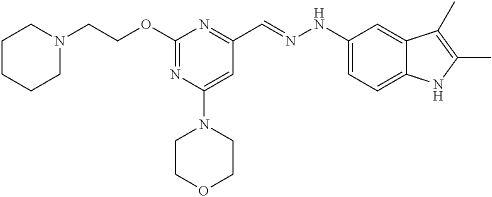 | N-(2,3-Dimethyl-1H-indol-5- yl)-N′-[6-morpholin-4-yl-2-(2- piperidin-1-yl-ethoxy)- pyrimidin-4-ylmethylene]- hydrazine |
| 432 | 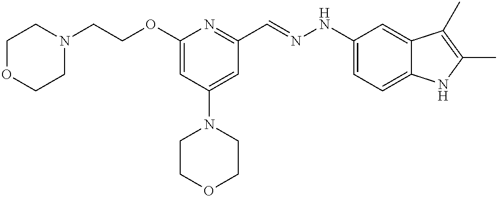 | N-(2,3-Dimethyl-1H-indol-5- yl)-N′-[4-morpholin-4-yl-6-(2- morpholin-4-yl-ethoxy)- pyridin-2-ylmethylene]- hydrazine |
| 433 | 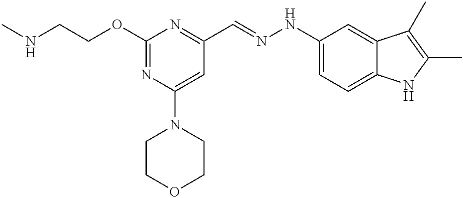 | (2-{4-[(2,3-Dimethyl-1H- indol-5-yl)-hydrazonomethyl]- 6-morpholin-4-yl-pyrimidin-2- yloxy}-ethyl)-methyl-amine |
| 434 | 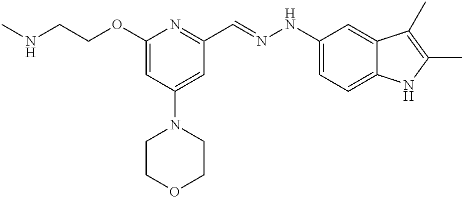 | (2-{6-[(2,3-Dimethyl-1H- indol-5-yl)-hydrazonomethyl]- 4-morpholin-4-yl-pyridin-2- yloxy}-ethyl)-methyl-amine |
| 435 | 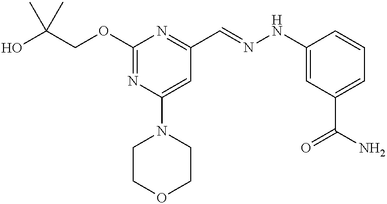 | 3-{N′-[2-(2-Hydroxy-2-methyl- propoxy)-6-morpholin-4-yl- pyrimidin-4-ylmethylene]- hydrazino}-benzamide |
| 436 | 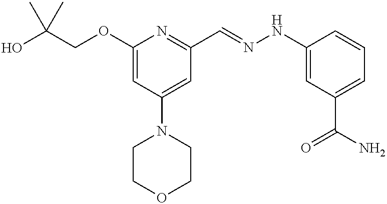 | 3-{N′-[6-(2-Hydroxy-2-methyl- propoxy)-4-morpholin-4-yl- pyridin-2-ylmethylene]- hydrazino}-benzamide |
| 437 | 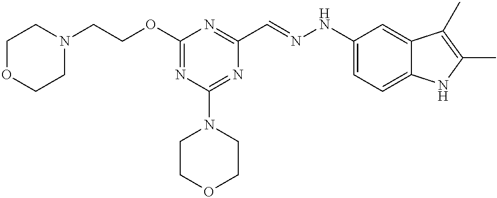 | N-(2,3-Dimethyl-1H-indol-5- yl)-N′-[4-morpholin-4-yl-6-(2- morpholin-4-yl-ethoxy)- [1,3,5]triazin-2-ylmethylene]- hydrazine |
| 438 | 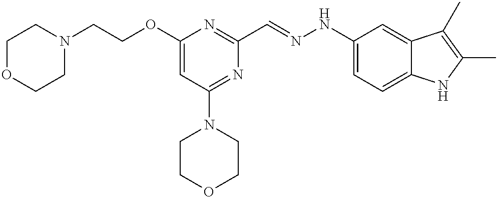 | N-(2,3-Dimethyl-1H-indol-5- yl)-N′-[4-morpholin-4-yl-6-(2- morpholin-4-yl-ethoxy)- pyrimidin-2-ylmethylene]- hydrazine |
| 439 | 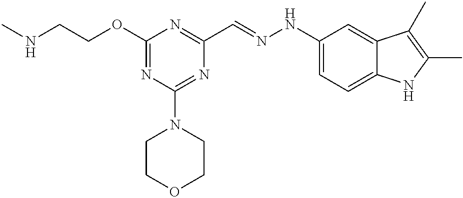 | (2-{4-[(2,3-Dimethyl-1H- indol-5-yl)-hydrazonomethyl]- 6-morpholin-4-yl- [1,3,5]triazin-2-yloxy}-ethyl)- methyl-amine |
| 440 | 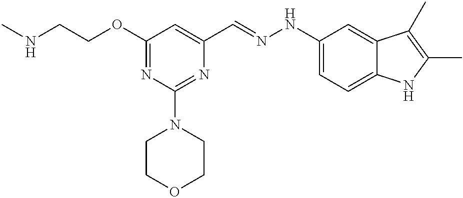 | (2-{6-[(2,3-Dimethyl-1H- indol-5-yl)-hydrazonomethyl]- 2-morpholin-4-yl-pyrimidin-4- yloxy}-ethyl)-methyl-amine |
| 441 | 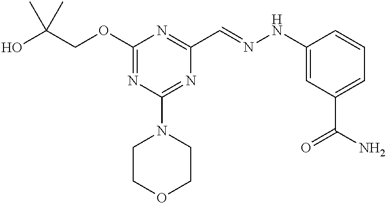 | 3-{N′-[4-(2-Hydroxy-2-methyl- propoxy)-6-morpholin-4-yl- [1,3,5]triazin-2-ylmethylene]- hydrazino}-benzamide |
| 442 | 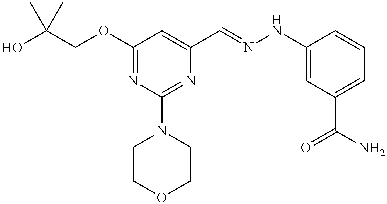 | 3-{N′-[6-(2-Hydroxy-2-methyl- propoxy)-2-morpholin-4-yl- pyrimidin-4-ylmethylene]- hydrazino}-benzamide |
| 443 | 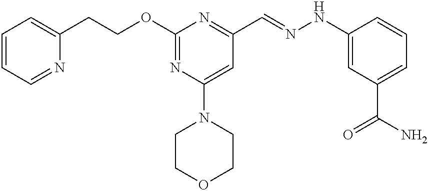 | 3-{N′-[6-Morpholin-4-yl-2-(2- pyridin-2-yl-ethoxy)- pyrimidin-4-ylmethylene]- hydrazino}-benzamide |
| 444 | 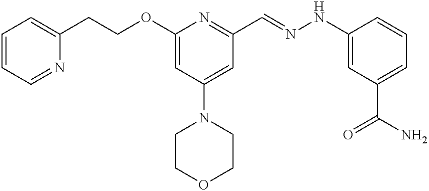 | 3-{N′-[4-Morpholin-4-yl-6-(2- pyridin-2-yl-ethoxy)-pyridin-2- ylmethylene]-hydrazino}- benzamide |
| 445 | 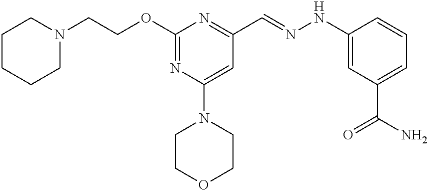 | 3-{N′-[6-Morpholin-4-yl-2-(2- piperidin-1-yl-ethoxy)- pyrimidin-4-ylmethylene]- hydrazino}-benzamide |
| 446 | 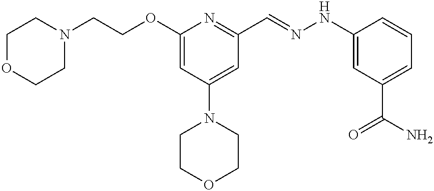 | 3-{N′-[4-Morpholin-4-yl-6-(2- morpholin-4-yl-ethoxy)- pyridin-2-ylmethylene]- hydrazino}-benzamide |
| 447 | 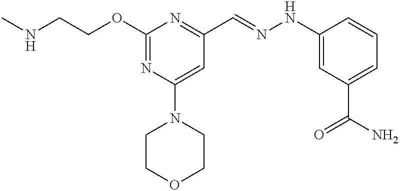 | 3-{N′-[2-(2-Methylamino- ethoxy)-6-morpholin-4-yl- pyrimidin-4-ylmethylene]- hydrazino}-benzamide |
| 448 | 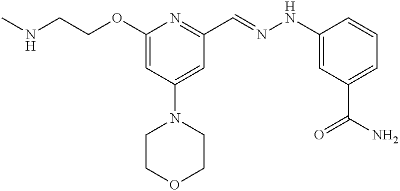 | 3-{N′-[6-(2-Methylamino- ethoxy)-4-morpholin-4-yl- pyridin-2-ylmethylene]- hydrazino}-benzamide |
| 449 | 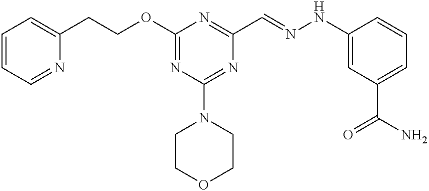 | 3-{N′-[4-Morpholin-4-yl-6-(2- pyridin-2-yl-ethoxy)- [1,3,5]triazin-2-ylmethylene]- hydrazino}-benzamide |
| 450 | 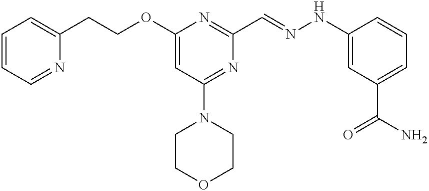 | 3-{N′-[4-Morpholin-4-yl-6-(2- pyridin-2-yl-ethoxy)- pyrimidin-2-ylmethylene]- hydrazino}-benzamide |
| 451 | 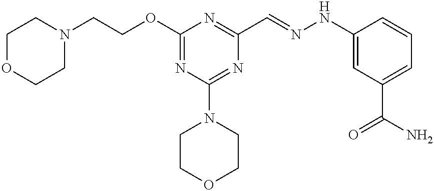 | 3-{N′-[4-Morpholin-4-yl-6-(2- morpholin-4-yl-ethoxy)- [1,3,5]triazin-2-ylmethylene]- hydrazino}-benzamide |
| 452 | 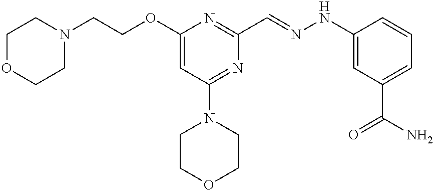 | 3-{N′-[4-Morpholin-4-yl-6-(2- morpholin-4-yl-ethoxy)- pyrimidin-2-ylmethylene]- hydrazino}-benzamide |
| 453 | 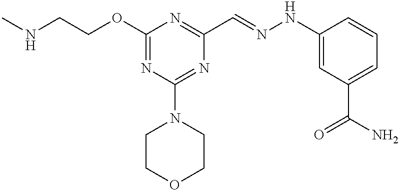 | 3-{N′-[4-(2-Methylamino- ethoxy)-6-morpholin-4-yl- [1,3,5]triazin-2-ylmethylene]- hydrazino}-benzamide |
| 454 | 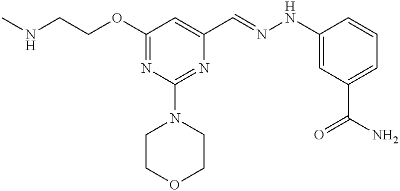 | 3-{N′-[6-(2-Methylamino- ethoxy)-2-morpholin-4-yl- pyrimidin-4-ylmethylene]- hydrazino}-benzamide |
| 455 | 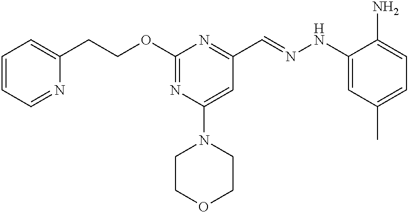 | 4-Methyl-2-{N′-[6-morpholin- 4-yl-2-(2-pyridin-2-yl-ethoxy)- pyrimidin-4-ylmethylene]- hydrazino}-phenylamine |
| 456 | 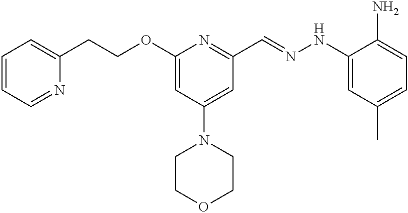 | 4-Methyl-2-{N′-[4-morpholin- 4-yl-6-(2-pyridin-2-yl-ethoxy)- pyridin-2-ylmethylene]- hydrazino}-phenylamine |
| 457 | 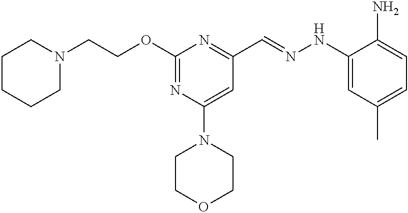 | 4-Methyl-2-{N′-[6-morpholin- 4-yl-2-(2-piperidin-1-yl- ethoxy)-pyrimidin-4- ylmethylene]-hydrazino}- phenylamine |
| 458 | 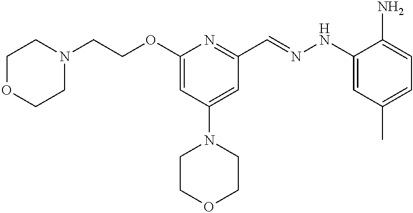 | 4-Methyl-2-{N′-[4-morpholin- 4-yl-6-(2-morpholin-4-yl- ethoxy)-pyridin-2- ylmethylene]-hydrazino}- phenylamine |
| 459 | 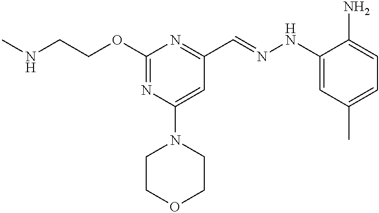 | 4-Methyl-2-{N′-[2-(2- methylamino-ethoxy)-6- morpholin-4-yl-pyrimidin-4- ylmethylene]-hydrazino}- phenylamine |
| 460 | 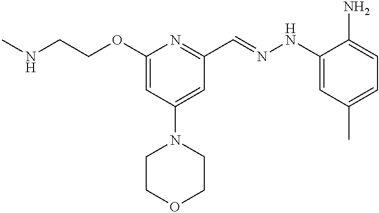 | 4-Methyl-2-{N′-[6-(2- methylamino-ethoxy)-4- morpholin-4-yl-pyridin-2- ylmethylene]-hydrazino}- phenylamine |
| 461 | 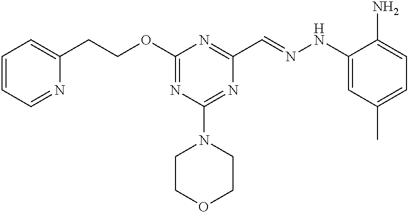 | 4-Methyl-2-{N′-[4-morpholin- 4-yl-6-(2-pyridin-2-yl-ethoxy)- [1,3,5]triazin-2-ylmethylene]- hydrazino}-phenylamine |
| 462 | 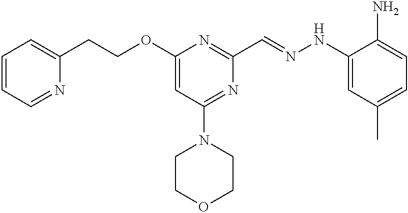 | 4-Methyl-2-{N′-[4-morpholin- 4-yl-6-(2-pyridin-2-yl-ethoxy)- pyrimidin-2-ylmethylene]- hydrazino}-phenylamine |
| 463 | 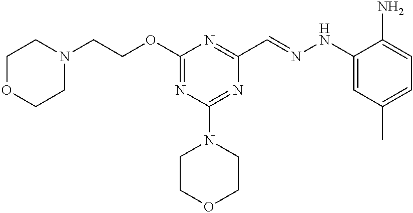 | 4-Methyl-2-{N′-[4-morpholin- 4-yl-6-(2-morpholin-4-yl- ethoxy)-[1,3,5]triazin-2- ylmethylene]-hydrazino}- phenylamine |
| 464 | 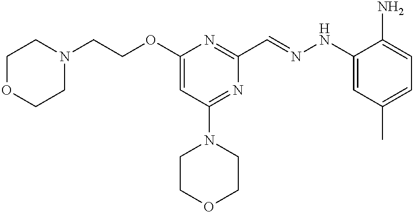 | 4-Methyl-2-{N′-[4-morpholin- 4-yl-6-(2-morpholin-4-yl- ethoxy)-pyrimidin-2- ylmethylene]-hydrazino}- phenylamine |
| 465 | 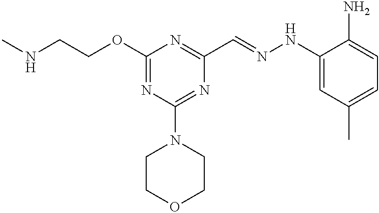 | 4-Methyl-2-{N′-[4-(2- methylamino-ethoxy)-6- morpholin-4-yl-[1,3,5]triazin- 2-ylmethylene]-hydrazino}- phenylamine |
| 466 | 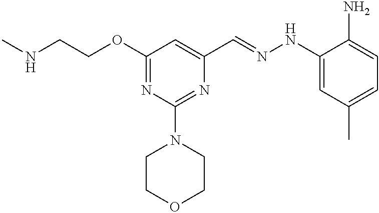 | 4-Methyl-2-{N′-[6-(2- methylamino-ethoxy)-2- morpholin-4-yl-pyrimidin-4- ylmethylene]-hydrazino}- phenylamine |
| 467 | 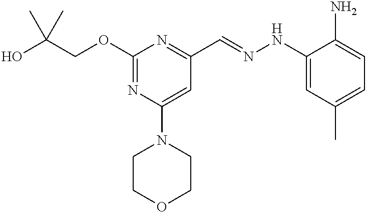 | 1-{4-[(2-Amino-5-methyl- phenyl)-hydrazonomethyl]-6- morpholin-4-yl-pyrimidin-2- yloxy}-2-methyl-propan-2-ol |
| 468 | 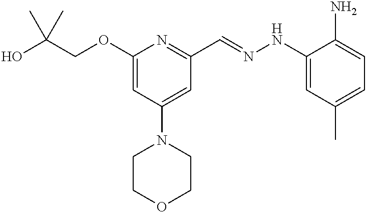 | 1-{6-[(2-Amino-5-methyl- phenyl)-hydrazonomethyl]-4- morpholin-4-yl-pyridin-2- yloxy}-2-methyl-propan-2-ol |
| 469 | 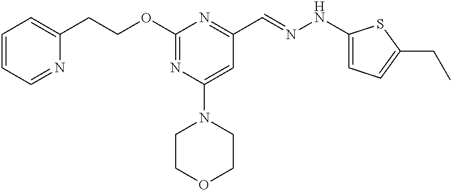 | N-(5-Ethyl-thiophen-2-yl)-N′- [6-morpholin-4-yl-2-(2- pyridin-2-yl-ethoxy)- pyrimidin-4-ylmethylene]- hydrazine |
| 470 | 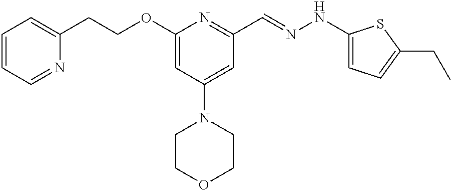 | N-(5-Ethyl-thiophen-2-yl)-N′- [4-morpholin-4-yl-6-(2- pyridin-2-yl-ethoxy)-pyridin-2- ylmethylene]-hydrazine |
| 471 | 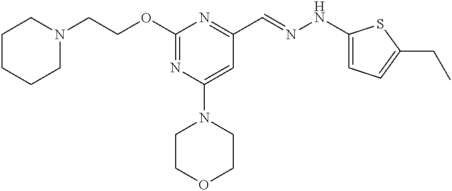 | N-(5-Ethyl-thiophen-2-yl)-N′- [6-morpholin-4-yl-2-(2- piperidin-1-yl-ethoxy)- pyrimidin-4-ylmethylene]- hydrazine |
| 472 | 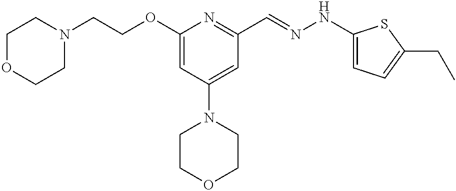 | N-(5-Ethyl-thiophen-2-yl)-N′- [4-morpholin-4-yl-6-(2- morpholin-4-yl-ethoxy)- pyridin-2-ylmethylene]- hydrazine |
| 473 | 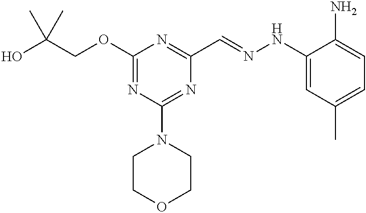 | 1-{4-[(2-Amino-5-methyl- phenyl)-hydrazonomethyl]-6- morpholin-4-yl-[1,3,5]triazin- 2-yloxy}-2-methyl-propan-2-ol |
| 474 | 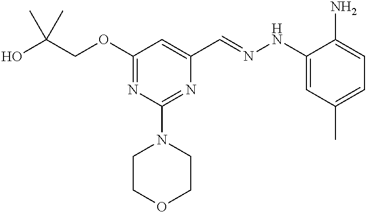 | 1-{6-[(2-Amino-5-methyl- phenyl)-hydrazonomethyl]-2- morpholin-4-yl-pyrimidin-4- yloxy}-2-methyl-propan-2-ol |
| 475 | 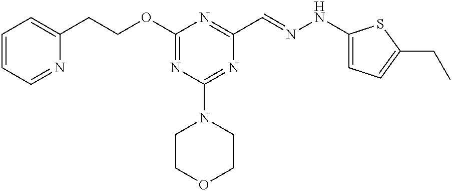 | N-(5-Ethyl-thiophen-2-yl)-N′- [4-morpholin-4-yl-6-(2- pyridin-2-yl-ethoxy)- [1,3,5]triazin-2-ylmethylene]- hydrazine |
| 476 | 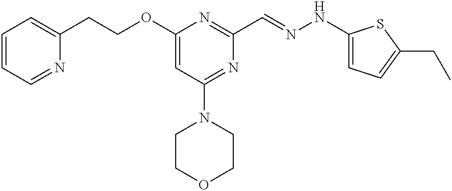 | N-(5-Ethyl-thiophen-2-yl)-N′- [4-morpholin-4-yl-6-(2- pyridin-2-yl-ethoxy)- pyrimidin-2-ylmethylene]- hydrazine |
| 477 | 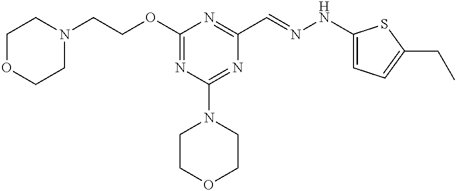 | N-(5-Ethyl-thiophen-2-yl)-N′- [4-morpholin-4-yl-6-(2- morpholin-4-yl-ethoxy)- [1,3,5]triazin-2-ylmethylene]- hydrazine |
| 478 | 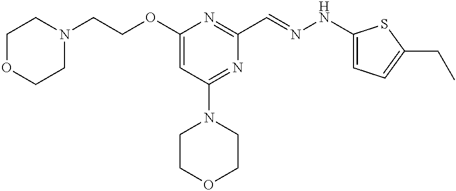 | N-(5-Ethyl-thiophen-2-yl)-N′- [4-morpholin-4-yl-6-(2- morpholin-4-yl-ethoxy)- pyrimidin-2-ylmethylene]- hydrazine |
| 479 | 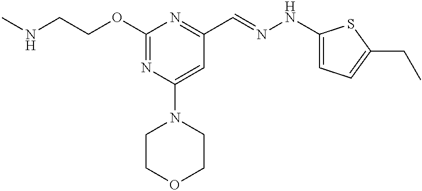 | (2-{4-[(5-Ethyl-thiophen-2-yl)- hydrazonomethyl]-6- morpholin-4-yl-pyrimidin-2- yloxy}-ethyl)-methyl-amine |
| 480 | 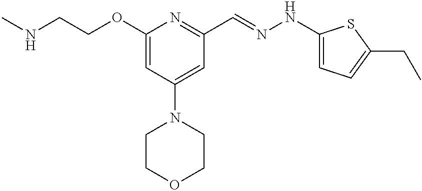 | (2-{6-[(5-Ethyl-thiophen-2-yl)- hydrazonomethyl]-4- morpholin-4-yl-pyridin-2- yloxy}-ethyl)-methyl-amine |
| 481 | 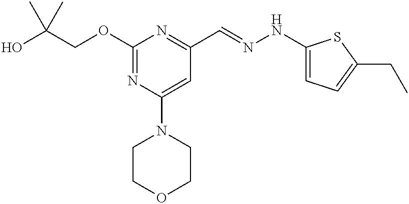 | 1-{4-[(5-Ethyl-thiophen-2-yl)- hydrazonomethyl]-6- morpholin-4-yl-pyrimidin-2- yloxy}-2-methyl-propan-2-ol |
| 482 | 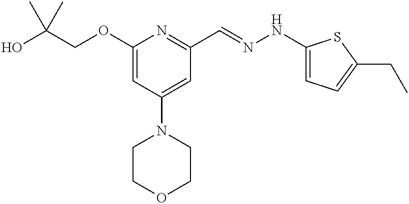 | 1-{6-[(5-Ethyl-thiophen-2-yl)- hydrazonomethyl]-4- morpholin-4-yl-pyridin-2- yloxy}-2-methyl-propan-2-ol |
| 483 | 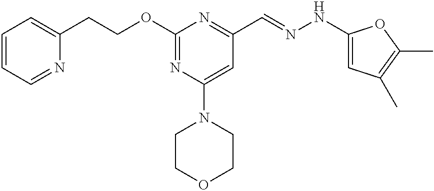 | N-(4,5-Dimethyl-furan-2-yl)- N′-[6-morpholin-4-yl-2-(2- pyridin-2-yl-ethoxy)- pyrimidin-4-ylmethylene]- hydrazine |
| 484 | 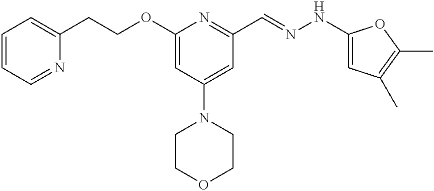 | N-(4,5-Dimethyl-furan-2-yl)- N′-[4-morpholin-4-yl-6-(2- pyridin-2-yl-ethoxy)-pyridin-2- ylmethylene]-hydrazine |
| 485 | 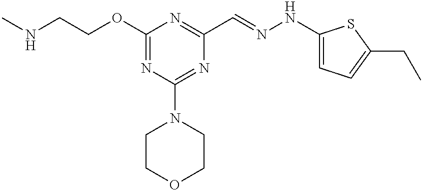 | (2-{4-[(5-Ethyl-thiophen-2-yl)- hydrazonomethyl]-6- morpholin-4-yl-[1,3,5]triazin- 2-yloxy}-ethyl)-methyl-amine |
| 486 | 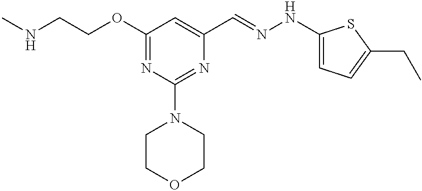 | (2-{6-[(5-Ethyl-thiophen-2-yl)- hydrazonomethyl]-2- morpholin-4-yl-pyrimidin-4- yloxy}-ethyl)-methyl-amine |
| 487 | 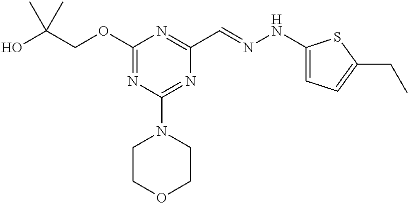 | 1-{4-[(5-Ethyl-thiophen-2-yl)- hydrazonomethyl]-6- morpholin-4-yl-[1,3,5]triazin- 2-yloxy}-2-methyl-propan-2-ol |
| 488 | 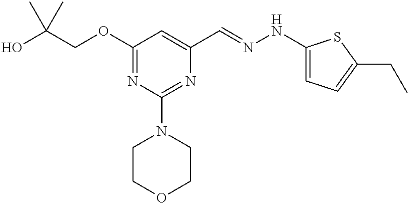 | 1-{6-[(5-Ethyl-thiophen-2-yl)- hydrazonomethyl]-2- morpholin-4-yl-pyrimidin-4- yloxy}-2-methyl-propan-2-ol |
| 489 | 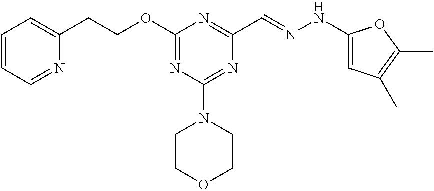 | N-(4,5-Dimethyl-furan-2-yl)- N′-[4-morpholin-4-yl-6-(2- pyridin-2-yl-ethoxy)- [1,3,5]triazin-2-ylmethylene]- hydrazine |
| 490 | 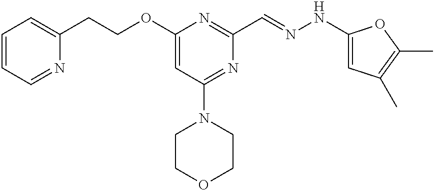 | N-(4,5-Dimethyl-furan-2-yl)- N′-[4-morpholin-4-yl-6-(2- pyridin-2-yl-ethoxy)- pyrimidin-2-ylmethylene]- hydrazine |
| 491 | 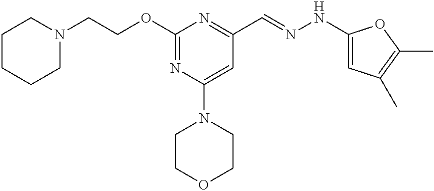 | N-(4,5-Dimethyl-furan-2-yl)- N′-[6-morpholin-4-yl-2-(2- piperidin-1-yl-ethoxy)- pyrimidin-4-ylmethylene]- hydrazine |
| 492 | 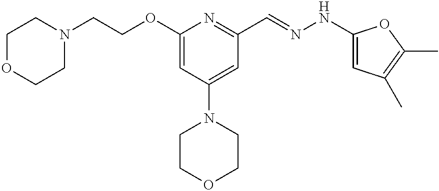 | N-(4,5-Dimethyl-furan-2-yl)- N′-[4-morpholin-4-yl-6-(2- morpholin-4-yl-ethoxy)- pyridin-2-ylmethylene]- hydrazine |
| 493 | 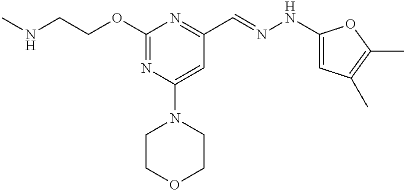 | (2-{4-[(4,5-Dimethyl-furan-2- yl)-hydrazonomethyl]-6- morpholin-4-yl-pyrimidin-2- yloxy}-ethyl)-methyl-amine |
| 494 | 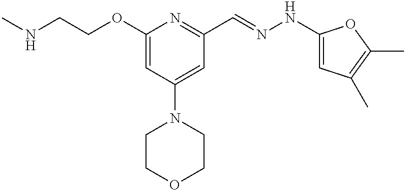 | (2-{6-[(4,5-Dimethyl-furan-2- yl)-hydrazonomethyl]-4- morpholin-4-yl-pyridin-2- yloxy}-ethyl)-methyl-amine |
| 495 | 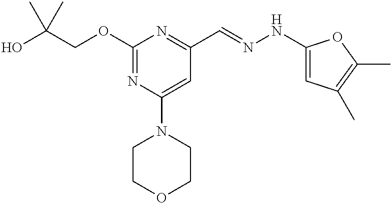 | 1-{4-[(4,5-Dimethyl-furan-2- yl)-hydrazonomethyl]-6- morpholin-4-yl-pyrimidin-2- yloxy}-2-methyl-propan-2-ol |
| 496 | 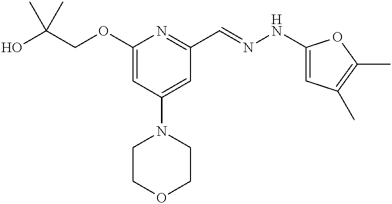 | 1-{6-[(4,5-Dimethyl-furan-2- yl)-hydrazonomethyl]-4- morpholin-4-yl-pyridin-2- yloxy}-2-methyl-propan-2-ol |
| 497 | 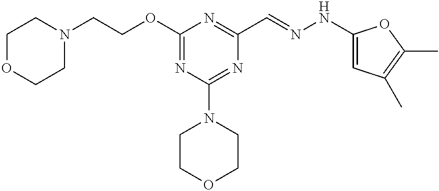 | N-(4,5-Dimethyl-furan-2-yl)- N′-[4-morpholin-4-yl-6-(2- morpholin-4-yl-ethoxy)- [1,3,5]triazin-2-ylmethylene]- hydrazine |
| 498 | 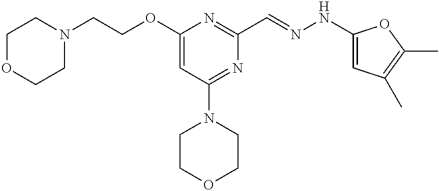 | N-(4,5-Dimethyl-furan-2-yl)- N′-[4-morpholin-4-yl-6-(2- morpholin-4-yl-ethoxy)- pyrimidin-2-ylmethylene]- hydrazine |
| 499 | 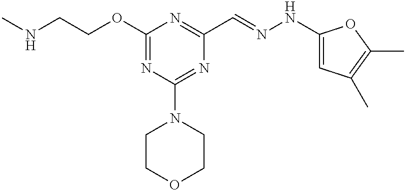 | (2-{4-[(4,5-Dimethyl-furan-2- yl)-hydrazonomethyl]-6- morpholin-4-yl-[1,3,5]triazin- 2-yloxy}-ethyl)-methyl-amine |
| 500 | 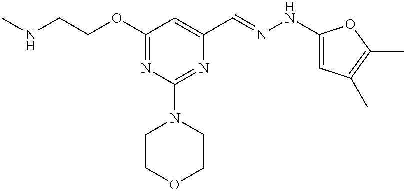 | (2-{6-[(4,5-Dimethyl-furan-2- yl)-hydrazonomethyl]-2- morpholin-4-yl-pyrimidin-4- yloxy}-ethyl)-methyl-amine |
| 501 | 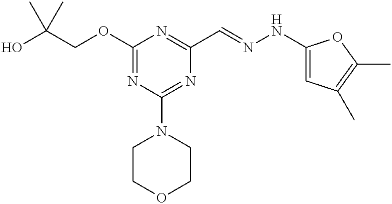 | {4-[(4,5-Dimethyl-furan-2-yl)- hydrazonomethyl]-6- morpholin-4-yl-[1,3,5]triazin- 2-yloxy}-2-methyl-propan-2-ol |
| 502 | 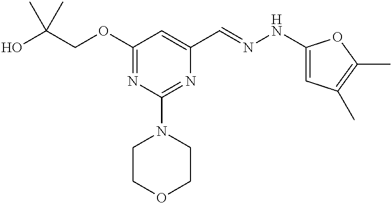 | 1-{6-[(4,5-Dimethyl-furan-2- yl)-hydrazonomethyl]-2- morpholin-4-yl-pyrimidin-4- yloxy}-2-methyl-propan-2-ol |
| 503 | 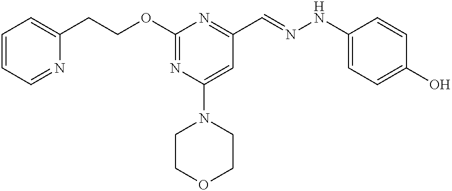 | 4-{N′-[6-Morpholin-4-yl-2-(2- pyridin-2-yl-ethoxy)- pyrimidin-4-ylmethylene]- hydrazino}-phenol |
| 504 | 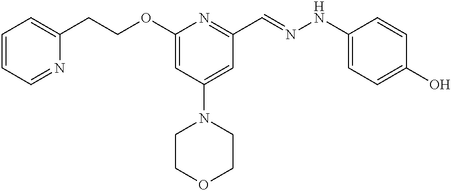 | 4-{N′-[4-Morpholin-4-yl-6-(2- pyridin-2-yl-ethoxy)-pyridin-2- ylmethylene]-hydrazino}- phenol |
| 505 | 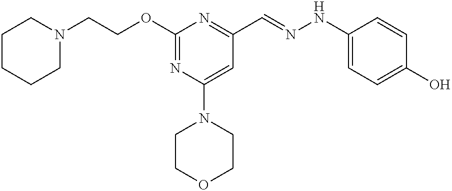 | 4-{N′-[6-Morpholin-4-yl-2-(2- piperidin-1-yl-ethoxy)- pyrimidin-4-ylmethylene]- hydrazino}-phenol |
| 506 | 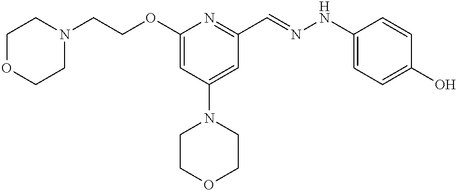 | 4-{N′-[4-Morpholin-4-yl-6-(2- morpholin-4-yl-ethoxy)- pyridin-2-ylmethylene]- hydrazino}-phenol |
| 507 | 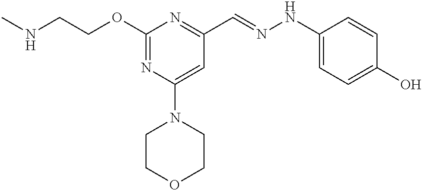 | 4-{N′-[2-(2-Methylamino- ethoxy)-6-morpholin-4-yl- pyrimidin-4-ylmethylene]- hydrazino}-phenol |
| 508 | 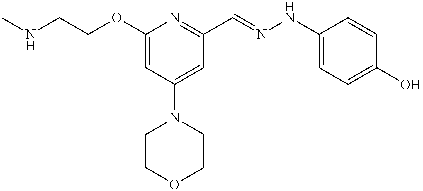 | 4-{N′-[6-(2-Methylamino- ethoxy)-4-morpholin-4-yl- pyridin-2-ylmethylene]- hydrazino}-phenol |
| 509 | 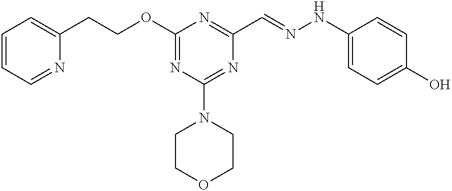 | 4-{N′-[4-Morpholin-4-yl-6-(2- pyridin-2-yl-ethoxy)- [1,3,5]triazin-2-ylmethylene]- hydrazino}-phenol |
| 510 | 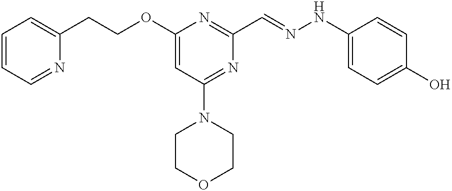 | 4-{N′-[4-Morpholin-4-yl-6-(2- pyridin-2-yl-ethoxy)- pyrimidin-2-ylmethylene]- hydrazino}-phenol |
| 511 | 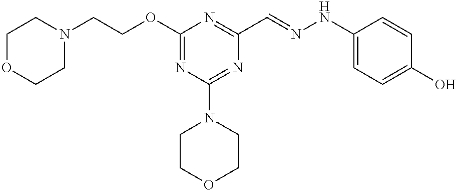 | 4-{N′-[4-Morpholin-4-yl-6-(2- morpholin-4-yl-ethoxy)- [1,3,5]triazin-2-ylmethylene]- hydrazino}-phenol |
| 512 | 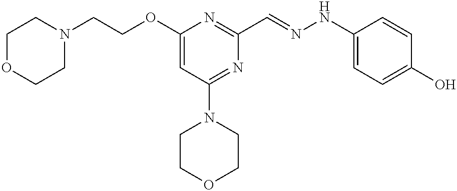 | 4-{N′-[4-Morpholin-4-yl-6-(2- morpholin-4-yl-ethoxy)- pyrimidin-2-ylmethylene]- hydrazino}-phenol |
| 513 | 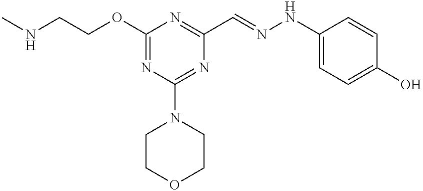 | 4-{N′-[4-(2-Methylamino- ethoxy)-6-morpholin-4-yl- [1,3,5]triazin-2-ylmethylene]- hydrazino}-phenol |
| 514 | 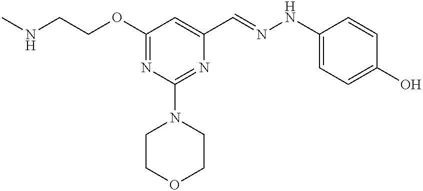 | 4-{N′-[6-(2-Methylamino- ethoxy)-2-morpholin-4-yl- pyrimidin-4-ylmethylene]- hydrazino}-phenol |
| 515 | 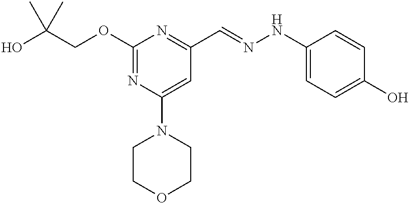 | 4-{N′-[2-(2-Hydroxy-2-methyl- propoxy)-6-morpholin-4-yl- pyrimidin-4-ylmethylene]- hydrazino}-phenol |
| 516 | 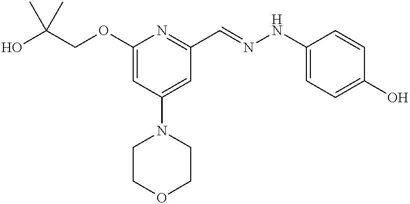 | 4-{N′-[6-(2-Hydroxy-2-methyl- propoxy)-4-morpholin-4-yl- pyridin-2-ylmethylene]- hydrazino}-phenol |
| 517 | 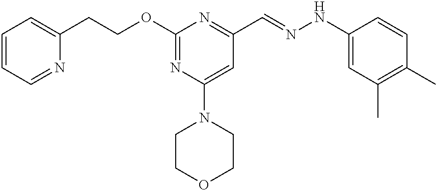 | N-(3,4-Dimethyl-phenyl)-N′- [6-morpholin-4-yl-2-(2- pyridin-2-yl-ethoxy)- pyrimidin-4-ylmethylene]- hydrazine |
| 518 | 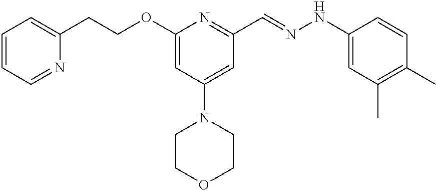 | N-(3,4-Dimethyl-phenyl)-N′- [4-morpholin-4-yl-6-(2- pyridin-2-yl-ethoxy)-pyridin-2- ylmethylene]-hydrazine |
| 519 | 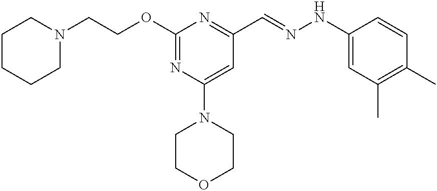 | N-(3,4-Dimethyl-phenyl)-N′- [6-morpholin-4-yl-2-(2- piperidin-1-yl-ethoxy)- pyrimidin-4-ylmethylene]- hydrazine |
| 520 | 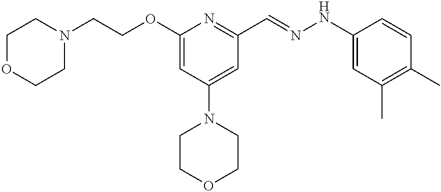 | N-(3,4-Dimethyl-phenyl)-N′- [4-morpholin-4-yl-6-(2- morpholin-4-yl-ethoxy)- pyridin-2-ylmethylene]- hydrazine |
| 521 | 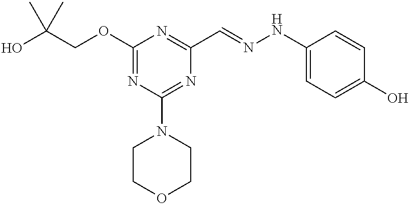 | 4-{N′-[4-(2-Hydroxy-2-methyl- propoxy)-6-morpholin-4-yl- [1,3,5]triazin-2-ylmethylene]- hydrazino}-phenol |
| 522 | 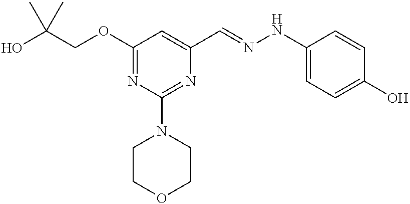 | 4-{N′-[6-(2-Hydroxy-2-methyl- propoxy)-2-morpholin-4-yl- pyrimidin-4-ylmethylene]- hydrazino}-phenol |
| 523 | 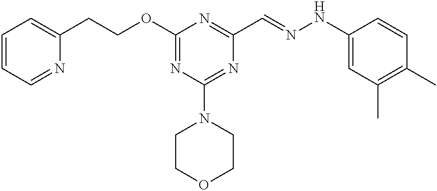 | N-(3,4-Dimethyl-phenyl)-N′- [4-morpholin-4-yl-6-(2- pyridin-2-yl-ethoxy)- [1,3,5]triazin-2-ylmethylene]- hydrazine |
| 524 | 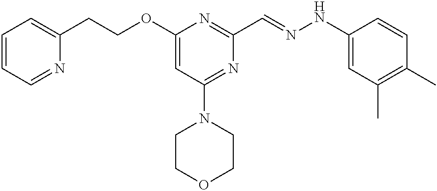 | N-(3,4-Dimethyl-phenyl)-N′- [4-morpholin-4-yl-6-(2- pyridin-2-yl-ethoxy)- pyrimidin-2-ylmethylene]- hydrazine |
| 525 | 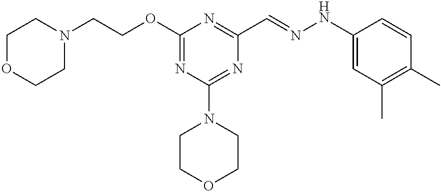 | N-(3,4-Dimethyl-phenyl)-N′- [4-morpholin-4-yl-6-(2- morpholin-4-yl-ethoxy)- [1,3,5]triazin-2-ylmethylene]- hydrazine |
| 526 | 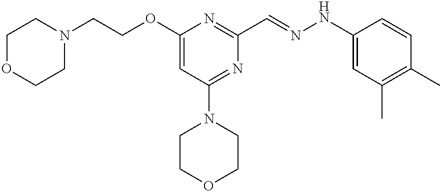 | N-(3,4-Dimethyl-phenyl)-N′- [4-morpholin-4-yl-6-(2- morpholin-4-yl-ethoxy)- pyrimidin-2-ylmethylene]- hydrazine |
| 527 | 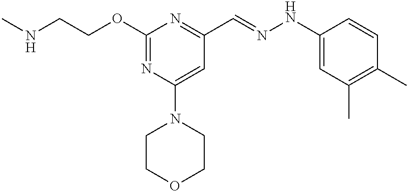 | (2-{4-[(3,4-Dimethyl-phenyl)- hydrazonomethyl]-6- morpholin-4-yl-pyrimidin-2- yloxy}-ethyl)-methyl-amine |
| 528 | 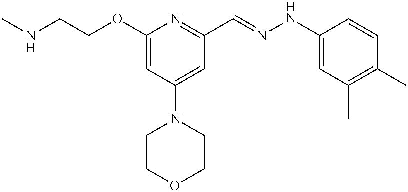 | (2-{6-[(3,4-Dimethyl-phenyl)- hydrazonomethyl]-4- morpholin-4-yl-pyridin-2- yloxy}-ethyl)-methyl-amine |
| 529 | 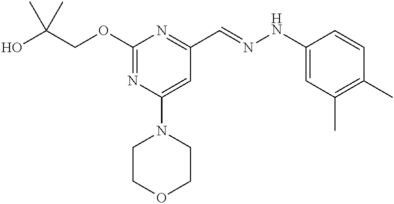 | 1-{4-[(3,4-Dimethyl-phenyl)- hydrazonomethyl]-6- morpholin-4-yl-pyrimidin-2- yloxy}-2-methyl-propan-2-ol |
| 530 | 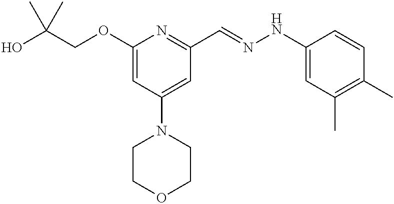 | 1-{6-[(3,4-Dimethyl-phenyl)- hydrazonomethyl]-4- morpholin-4-yl-pyridin-2- yloxy}-2-methyl-propan-2-ol |
| 531 | 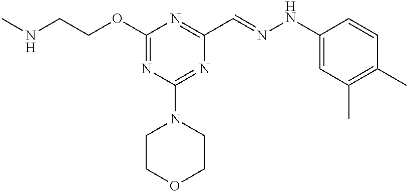 | (2-{4-[(3,4-Dimethyl-phenyl)- hydrazonomethyl]-6- morpholin-4-yl-[1,3,5]triazin- 2-yloxy}-ethyl)-methyl-amine |
| 532 | 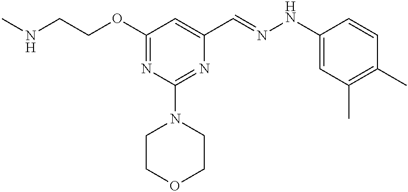 | (2-{6-[(3,4-Dimethyl-phenyl)- hydrazonomethyl]-2- morpholin-4-yl-pyrimidin-4- yloxy}-ethyl)-methyl-amine |
| 533 | 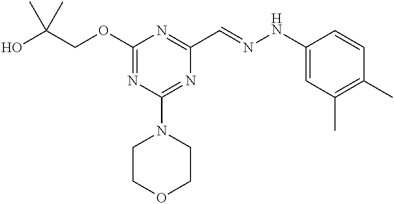 | 1-{4-[(3,4-Dimethyl-phenyl)- hydrazonomethyl]-6- morpholin-4-yl-[1,3,5]triazin- 2-yloxy}-2-methyl-propan-2-ol |
| 534 | 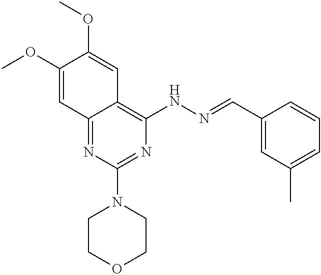 | N-(6,7-dimethoxy-2- morpholin-4-yl-quinazolin-4- yl)-N′-(3-methyl-benzylidene)- hydrazine |
| 535 | 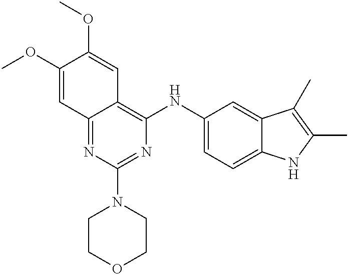 | (6,7-dimethoxy-2-morpholin-4- yl-quinazolin-4-yl)-(2,3- dimethyl-1H-indol-5-yl)-amine |
| 536 | 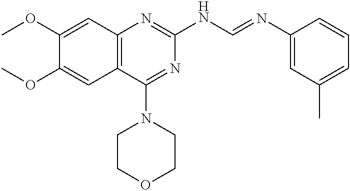 | N-(6,7-dimethoxy-4- morpholin-4-yl-quinazolin-2- yl)-N′-(3-methyl-benzylidene)- hydrazine |
| 537 | 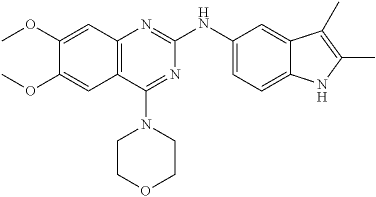 | (6,7-dimethoxy-4-morpholin-4- yl-quinazolin-2-yl)-(2,3- dimethyl-1H-indol-5-yl)-amine |
| 538 | 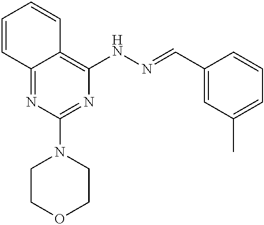 | N-(3-methyl-benzylidene)-N′- (2-morpholin-4-yl-quinazolin- 4-yl)-hydrazine |
| 539 | 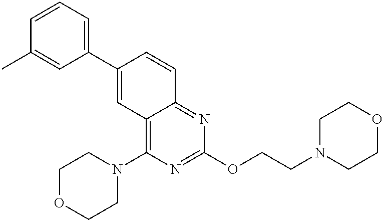 | 4-Morpholin-4-yl-2-(2- morpholin-4-yl-ethoxy)-6-m- tolyl-quinazoline |
| 540 | 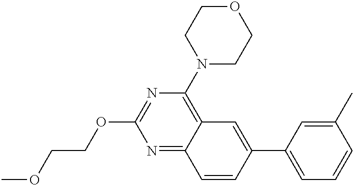 | 2-(2-Methoxy-ethoxy)-4- morpholin-4-yl-6-m-tolyl- quinazoline |
| 541 | 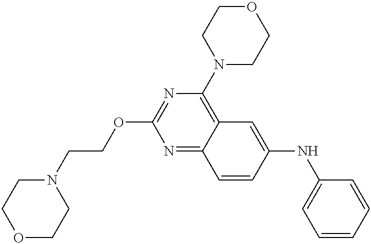 | [4-Morpholin-4-yl-2-(2- morpholin-4-yl-ethoxy)- quinazolin-6-yl]-phenyl-amine |
| 542 | 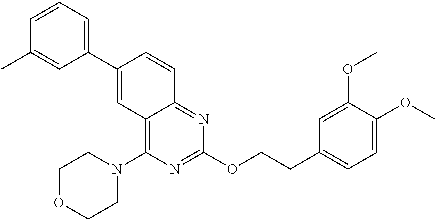 | 2-[2-(3,4-Dimethoxy-phenyl)- ethoxy]-4-morpholin-4-yl-6-m- tolyl-quinazoline |
| 543 | 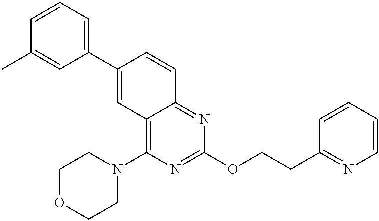 | 4-Morpholin-4-yl-2-(2-pyridin- 2-yl-ethoxy)-6-m-tolyl- quinazoline |
| 544 | 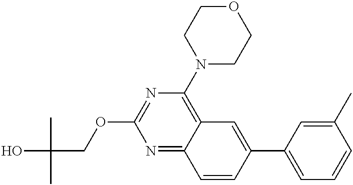 | 2-Methyl-1-(4-morpholin-4-yl- 6-m-tolyl-quinazolin-2-yloxy)- propan-2-ol |
| 545 | 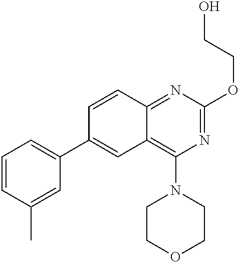 | 2-(4-Morpholin-4-yl-6-m-tolyl- quinazolin-2-yloxy)-ethanol |
| 546 | 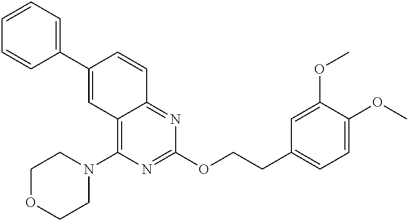 | 2-[2-(3,4-Dimethoxy-phenyl)- ethoxy]-4-morpholin-4-yl-6- phenyl-quinazoline |
| 547 | 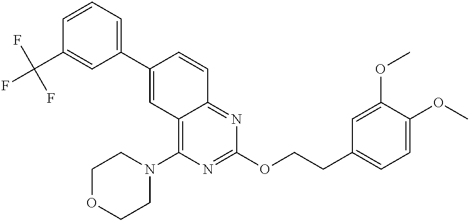 | 2-[2-(3,4-Dimethoxy-phenyl)- ethoxy]-4-morpholin-4-yl-6-(3- trifluoromethyl-phenyl)- quinazoline |
| 548 | 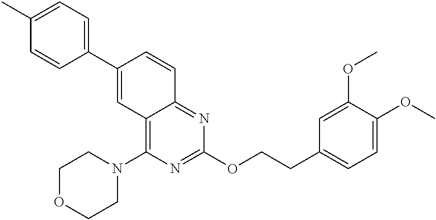 | 2-[2-(3,4-Dimethoxy-phenyl)- ethoxy]-4-morpholin-4-yl-6-p- tolyl-quinazoline |
| 549 | 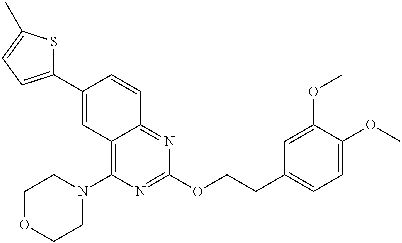 | 2-[2-(3,4-Dimethoxy-phenyl)- ethoxy]-6-(5-methyl-thiophen- 2-yl)-4-morpholin-4-yl- quinazoline |
| 550 | 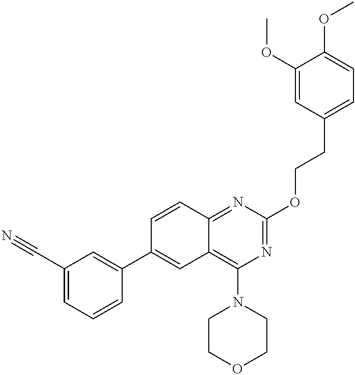 | 3-{2-[2-(3,4-Dimethoxy- phenyl)-ethoxy]-4-morpholin- 4-yl-quinazolin-6-yl}- benzonitrile |
| 551 | 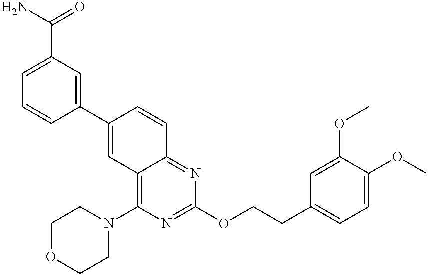 | 3-{2-[2-(3,4-Dimethoxy- phenyl)-ethoxy]-4-morpholin- 4-yl-quinazolin-6-yl}- benzamide |
| 552 | 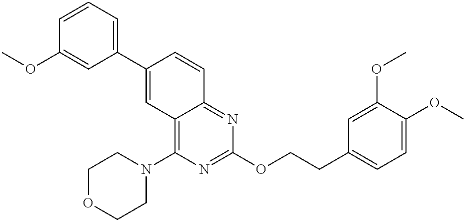 | 2-[2-(3,4-Dimethoxy-phenyl)- ethoxy]-6-(3-methoxy-phenyl)- 4-morpholin-4-yl-quinazoline |
| 553 | 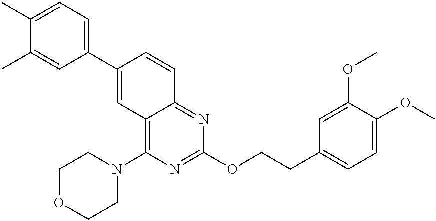 | 2-[2-(3,4-Dimethoxy-phenyl)- ethoxy]-6-(3,4-dimethyl- phenyl)-4-morpholin-4-yl- quinazoline |
| 554 | 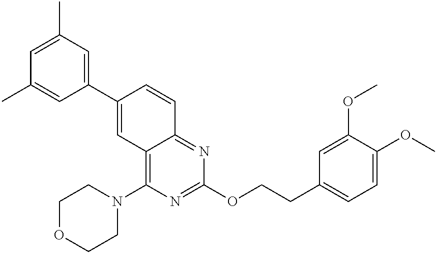 | 2-[2-(3,4-Dimethoxy-phenyl)- ethoxy]-6-(3,5-dimethyl- phenyl)-4-morpholin-4-yl- quinazoline |
| 555 | 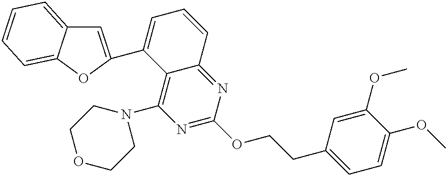 | 6-Benzofuran-2-yl-2-[2-(3,4- dimethoxy-phenyl)-ethoxy]-4- morpholin-4-yl-quinazoline |
| 556 | 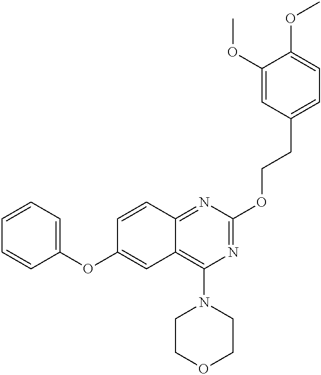 | 2-[2-(3,4-Dimethoxy-phenyl)- ethoxy]-4-morpholin-4-yl-6- phenoxy-quinazoline |
| 557 | 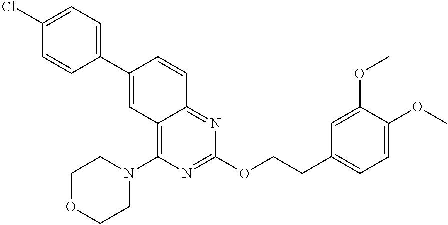 | 6-(4-Chloro-phenyl)-2-[2-(3,4- dimethoxy-phenyl)-ethoxy]-4- morpholin-4-yl-quinazoline |
| 558 | 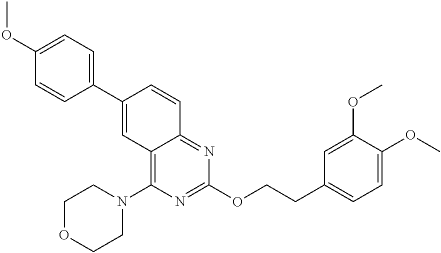 | 2-[2-(3,4-Dimethoxy-phenyl)- ethoxy]-6-(4-methoxy-phenyl)- 4-morpholin-4-yl-quinazoline |
| 559 | 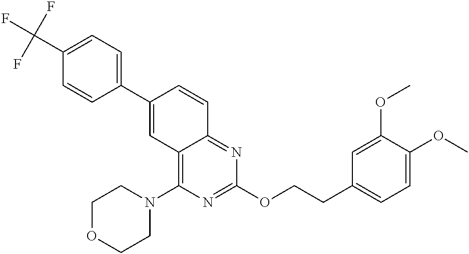 | 2-[2-(3,4-Dimethoxy-phenyl)- ethoxy]-4-morpholin-4-yl-6-(4- trifluoromethyl-phenyl)- quinazoline |
| 560 | 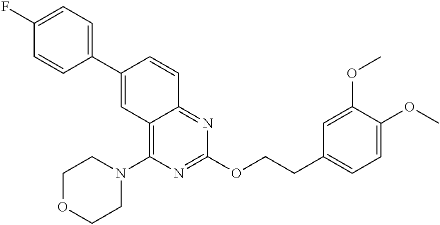 | 6-(4-Fluoro-phenyl)-2-[2-(3,4- dimethoxy-phenyl)-ethoxy]-4- morpholin-4-yl-quinazoline |
| 561 | 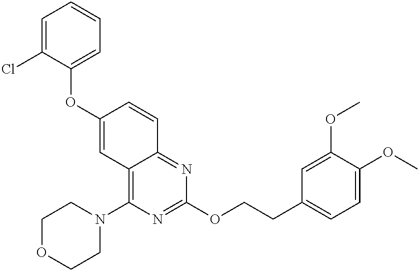 | 6-(2-Chloro-phenoxy)-2-[2- (3,4-dimethoxy-phenyl)- ethoxy]-4-morpholin-4-yl- quinazoline |
| 562 | 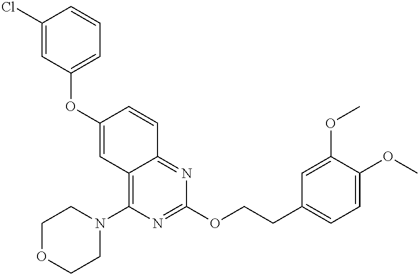 | 6-(3-Chloro-phenoxy)-2-[2- (3,4-dimethoxy-phenyl)- ethoxy]-4-morpholin-4-yl- quinazoline |
| 563 | 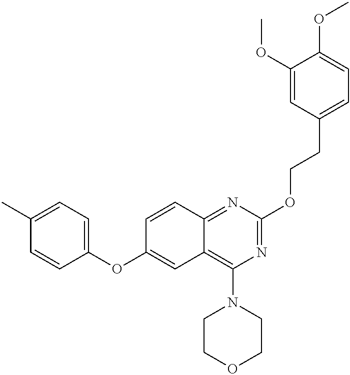 | 2-[2-(3,4-Dimethoxy-phenyl)- ethoxy]-4-morpholin-4-yl-6-p- tolyloxy-quinazoline |
| 564 | 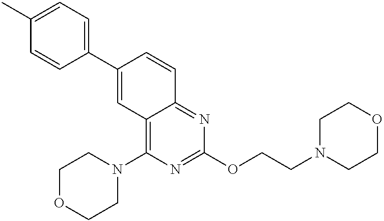 | 4-Morpholin-4-yl-2-(2- morpholin-4-yl-ethoxy)-6-p- tolyl-quinazoline |
| 565 | 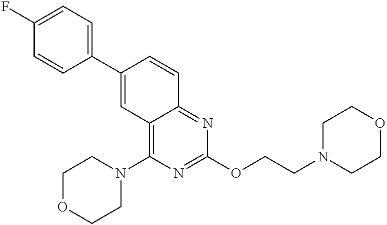 | 6-(4-Fluoro-phenyl)-4- morpholin-4-yl-2-(2- morpholin-4-yl-ethoxy)- quinazoline |
| 566 | 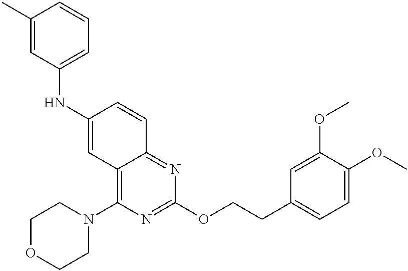 | {2-[2-(3,4-Dimethoxy-phenyl)- ethoxy]-4-morpholin-4-yl- quinazolin-6-yl}-m-tolyl-amine |
| 567 | 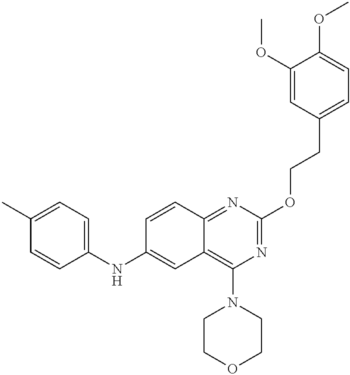 | {2-[2-(3,4-Dimethoxy-phenyl)- ethoxy]-4-morpholin-4-yl- quinazolin-6-yl}-p-tolyl-amine |
| 568 | 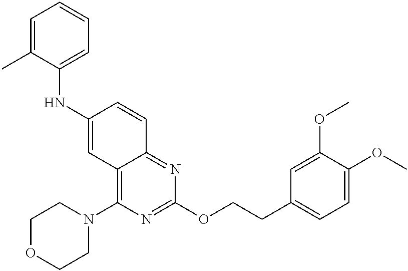 | {2-[2-(3,4-Dimethoxy-phenyl)- ethoxy]-4-morpholin-4-yl- quinazolin-6-yl}-o-tolyl-amine |
| 569 | 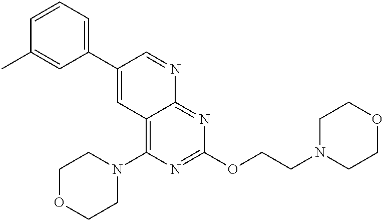 | 4-Morpholin-4-yl-2-(2- morpholin-4-yl-ethoxy)-6-m- tolyl-pyrido[2,3-d]pyrimidine |
| 570 | 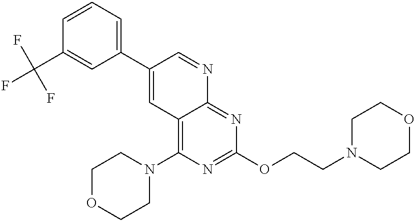 | 4-Morpholin-4-yl-2-(2- morpholin-4-yl-ethoxy)-6-(3- trifluoromethyl-phenyl)- pyrido[2,3-d]pyrimidine |
| 571 | 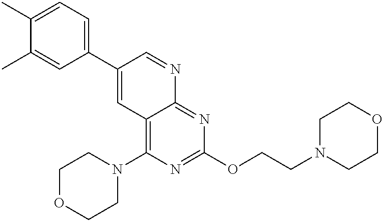 | 6-(3,4-Dimethyl-phenyl)-4- morpholin-4-yl-2-(2- morpholin-4-yl-ethoxy)- pyrido[2,3-d]pyrimidine |
| 572 | 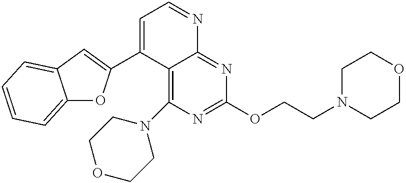 | 6-Benzofuran-2-yl-4- morpholin-4-yl-2-(2- morpholin-4-yl-ethoxy)- pyrido[2,3-d]pyrimidine |
| 573 | 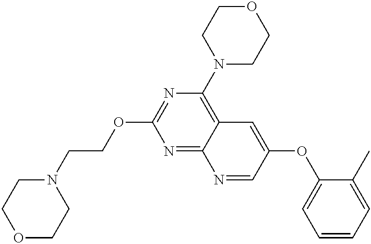 | 4-Morpholin-4-yl-2-(2- morpholin-4-yl-ethoxy)-6-o- tolyloxy-pyrido[2,3- d]pyrimidine |
| 574 | 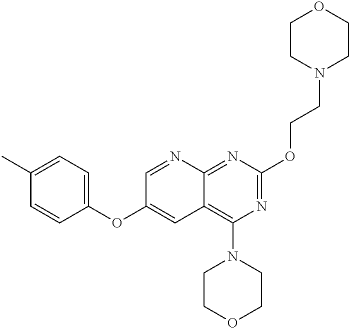 | 4-Morpholin-4-yl-2-(2- morpholin-4-yl-ethoxy)-6-p- tolyloxy-pyrido[2,3- d]pyrimidine |
| 575 | 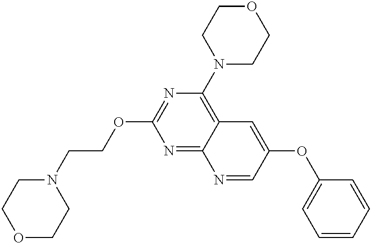 | 4-Morpholin-4-yl-2-(2- morpholin-4-yl-ethoxy)-6- phenoxy-pyrido[2,3- d]pyrimidine |
| 576 | 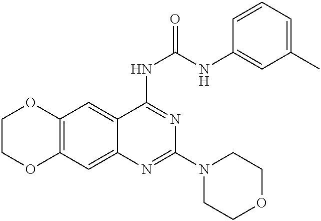 | 1-(2-Morpholin-4-yl-7,8- dihydro-[1,4]dioxino[2,3- g]quinazolin-4-yl)-3-m-tolyl- urea |
| 577 | 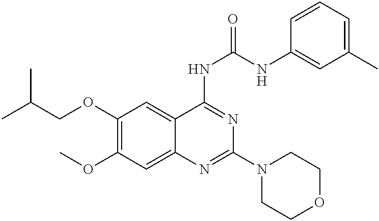 | 1-(6-Isobutoxy-7-methoxy-2- morpholin-4-yl-quinazolin-4- yl)-3-m-tolyl-urea |
| 578 | 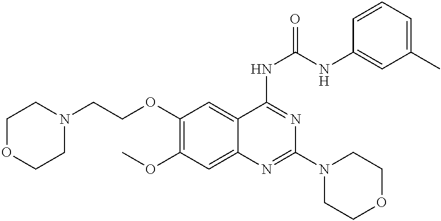 | 1-[7-Methoxy-2-morpholin-4- yl-6-(2-morpholin-4-yl- ethoxy)-quinazolin-4-yl]-3-m- tolyl-urea |
| 579 | 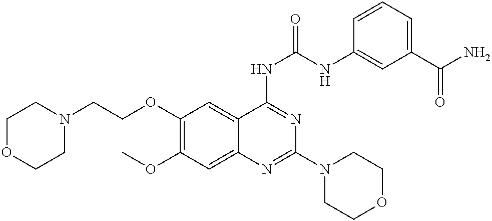 | 3-{3-[7-Methoxy-2-morpholin- 4-yl-6-(2-morpholin-4-yl- ethoxy)-quinazolin-4-yl]- ureido}-benzamide |
| 580 | 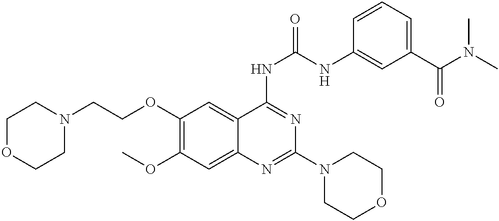 | 3-{3-[7-Methoxy-2-morpholin- 4-yl-6-(2-morpholin-4-yl- ethoxy)-quinazolin-4-yl]- ureido}-N,N-dimethyl- benzamide |
| 581 | 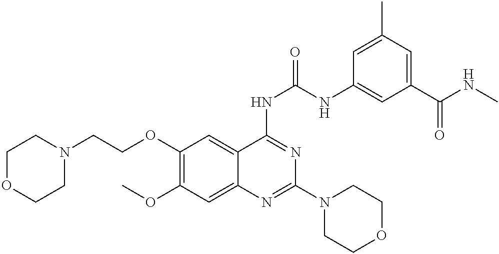 | 3-{3-[7-Methoxy-2-morpholin- 4-yl-6-(2-morpholin-4-yl- ethoxy)-quinazolin-4-yl]- ureido}-5,N-dimethyl- benzamide |
| 582 | 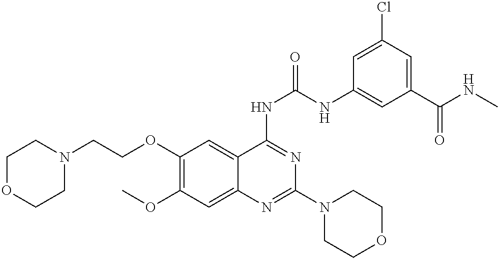 | 3-Chloro-5-{3-[7-methoxy-2- morpholin-4-yl-6-(2- morpholin-4-yl-ethoxy)- quinazolin-4-yl]-ureido}-N- methyl-benzamide |
| 583 | 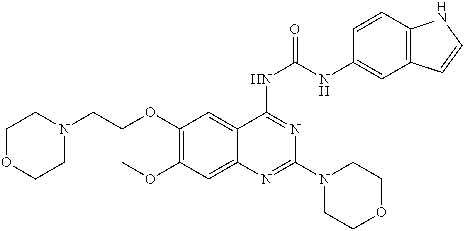 | 1-(1H-Indol-5-yl)-3-[7- methoxy-2-morpholin-4-yl-6- (2-morpholin-4-yl-ethoxy)- quinazolin-4-yl]-urea |
| 584 | 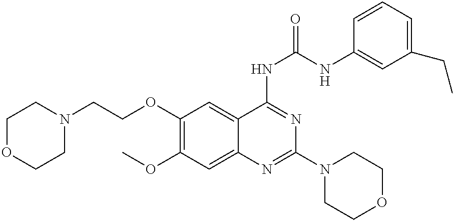 | 1-(3-Ethyl-phenyl)-3-[7- methoxy-2-morpholin-4-yl-6- (2-morpholin-4-yl-ethoxy)- quinazolin-4-yl]-urea |
| 585 | 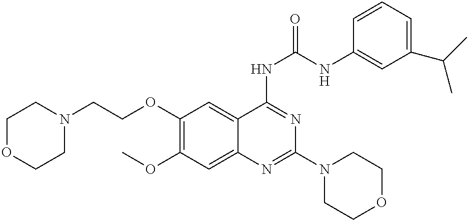 | 1-(3-Isopropyl-phenyl)-3-[7- methoxy-2-morpholin-4-yl-6- (2-morpholin-4-yl-ethoxy)- quinazolin-4-yl]-urea |
| 586 | 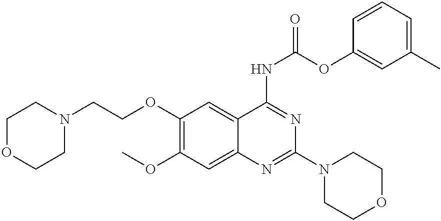 | [7-Methoxy-2-morpholin-4-yl- 6-(2-morpholin-4-yl-ethoxy)- quinazolin-4-yl]-carbamic acid m-tolyl ester |
| 587 | 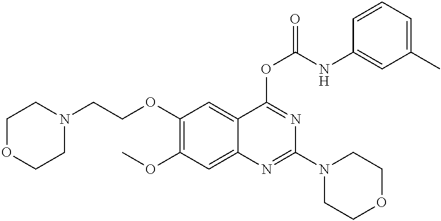 | m-Tolyl-carbamic acid 7- methoxy-2-morpholin-4-yl-6- (2-morpholin-4-yl-ethoxy)- quinazolin-4-yl ester |
| 588 | 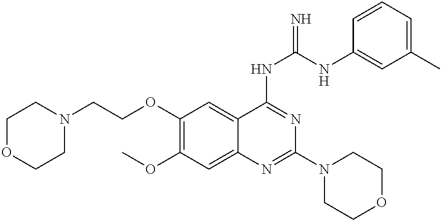 | N-[7-Methoxy-2-morpholin-4- yl-6-(2-morpholin-4-yl- ethoxy)-quinazolin-4-yl]-N′-m- tolyl-guanidine |
| 589 | 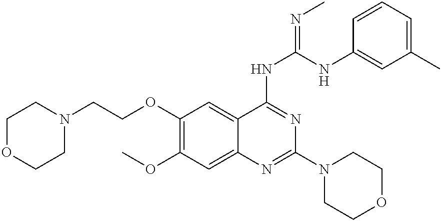 | N-[7-Methoxy-2-morpholin-4- yl-6-(2-morpholin-4-yl- ethoxy)-quinazolin-4-yl-N′- methyl-N″-m-tolyl-guanidine |
| 590 | 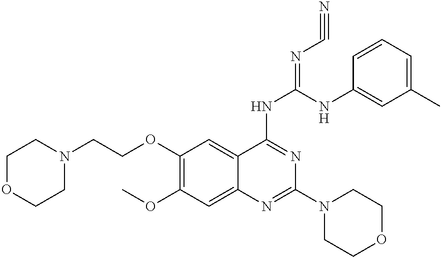 | N-[7-Methoxy-2-morpholin-4- yl-6-(2-morpholin-4-yl- ethoxy)-quinazolin-4-yl]-N′- cyano-N″-m-tolyl-guanidine |
| 591 | 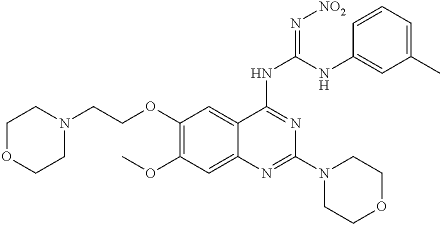 | N-[7-Methoxy-2-morpholin-4- yl-6-(2-morpholin-4-yl- ethoxy)-quinazolin-4-yl]-N′- nitro-N″-m-tolyl-guanidine |
| 592 | 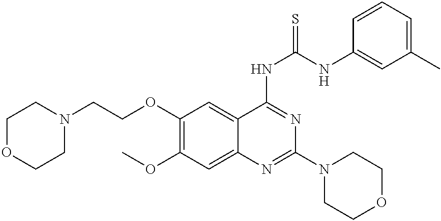 | 1-[7-Methoxy-2-morpholin-4- yl-6-(2-morpholin-4-yl- ethoxy)-quinazolin-4-yl]-3-m- tolyl-thioure |
| 593 | 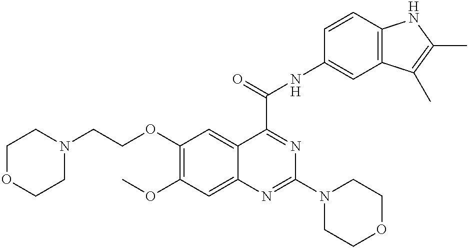 | 7-Methoxy-2-morpholin-4-yl- 6-(2-morpholin-4-yl-ethoxy)- quinazoline-4-carboxylic acid (2,3-dimethyl-1H-indol-5-yl)- amide |
| 594 | 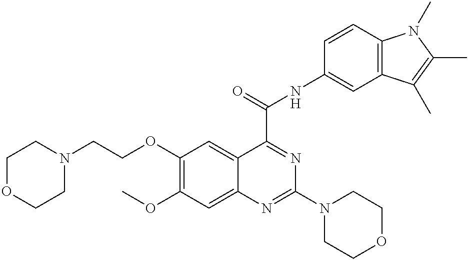 | 7-Methoxy-2-morpholin-4-yl- 6-(2-morpholin-4-yl-ethoxy)- quinazoline-4-carboxylic acid (1,2,3-trimethyl-1H-indol-5- yl)-amide |
| 595 | 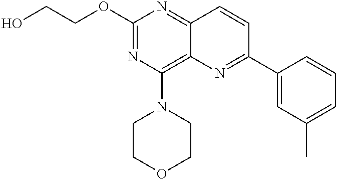 | 2-(4-Morpholin-4-yl-6-m-tolyl- pyrido[3,2-d]pyrimidin-2- yloxy)-ethanol |
| 596 | 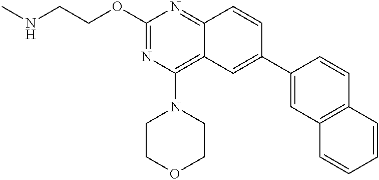 | Methyl-[2-(4-morpholin-4-yl- 6-naphthalen-2-yl-quinazolin- 2-yloxy)-ethyl]-amine |
| 597 | 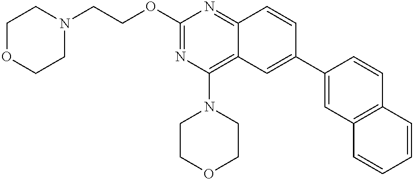 | 4-Morpholin-4-yl-2-(2- morpholin-4-yl-ethoxy)-6- naphthalen-2-yl-quinazoline |
| 598 | 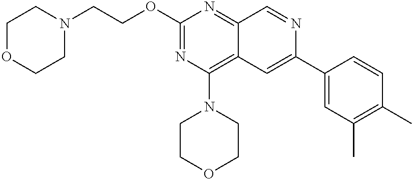 | 6-(3,4-Dimethyl-phenyl)-4- morpholin-4-yl-2-(2- morpholin-4-yl-ethoxy)- pyrido[3,4-d]pyrimidine |
| 599 | 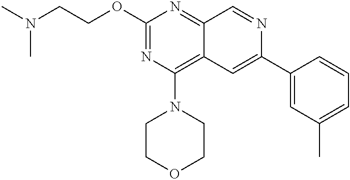 | Dimethyl-[2-(4-morpholin-4- yl-6-m-tolyl-pyrido[3,4- d]pyrimidin-2-yloxy)-ethyl]- amine |
| 600 | 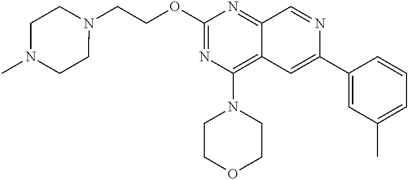 | 2-[2-(4-Methyl-piperazin-1-yl)- ethoxy]-4-morpholin-4-yl-6-m- tolyl-pyrido[3,4-d]pyrimidine |
| 601 | 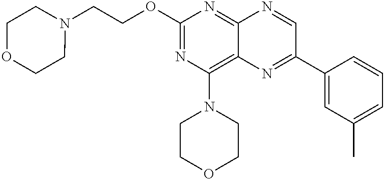 | 4-Morpholin-4-yl-2-(2- morpholin-4-yl-ethoxy)-6-m- tolyl-pteridine |
| 602 | 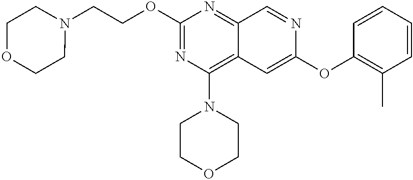 | 4-Morpholin-4-yl-2-(2- morpholin-4-yl-ethoxy)-6-o- tolyloxy-pyrido[3,4- d]pyrimidine |
| 603 | 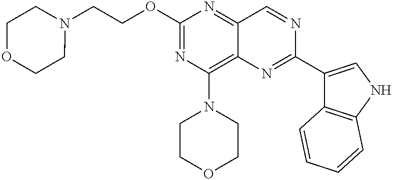 | 6-(1H-Indol-3-yl)-4-morpholin- 4-yl-2-(2-morpholin-4-yl- ethoxy)-pyrimido[5,4- d]pyrimidine |
| 604 | 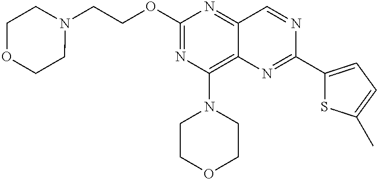 | 6-(5-Methyl-thiophen-2-yl)-4- morpholin-4-yl-2-(2- morpholin-4-yl-ethoxy)- pyrimido[5,4-d]pyrimidine |
| 605 | 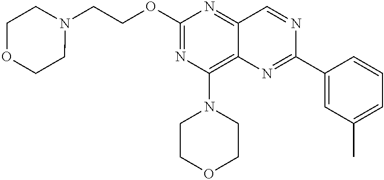 | 4-Morpholin-4-yl-2-(2- morpholin-4-yl-ethoxy)-6-m- tolyl-pyrimido[5,4- d]pyrimidine |
| 606 | 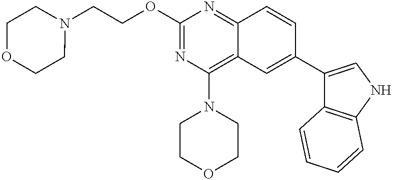 | 6-(1H-Indol-3-yl)-4-morpholin- 4-yl-2-(2-morpholin-4-yl- ethoxy)-quinazoline |
| 607 | 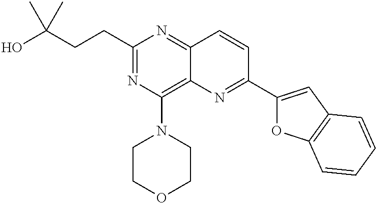 | 4-(6-Benzofuran-2-yl-4- morpholin-4-yl-pyrido[3,2- d]pyrimidin-2-yl)-2-methyl- butan-2-ol |
| 608 | 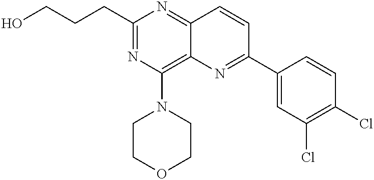 | 3-[6-(3,4-Dichloro-phenyl)-4- morpholin-4-yl-pyrido[3,2- d]pyrimidin-2-yl]-propan-1-ol |
| 609 | 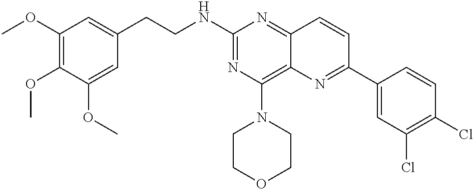 | [6-(3,4-Dichloro-phenyl)-4- morpholin-4-yl-pyrido[3,2- d]pyrimidin-2-yl]-[2-(3,4,5- trimethoxy-phenyl)-ethyl]- amine |
| 610 | 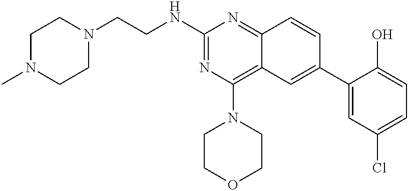 | 4-Chloro-2-{2-[2-(4-methyl- piperazin-1-yl)-ethylamino]-4- morpholin-4-yl-quinazolin-6- yl}-phenol |
| 611 | 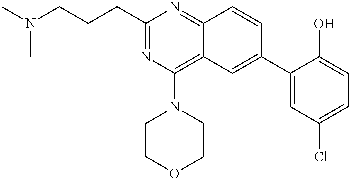 | 4-Chloro-2-{2-[2-(4-methyl- piperazin-1-yl)-ethylamino]-4- morpholin-4-yl-quinazolin-6- yl}-phenol |
| 612 | 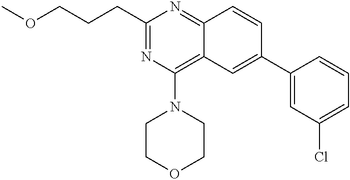 | 6-(3-Chloro-phenyl)-2-(3- methoxy-propyl)-4-morpholin- 4-yl-quinazoline |
| 623 | 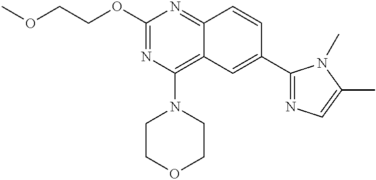 | 6-(1,5-Dimethyl-1H-imidazol- 2-yl)-2-(2-methoxy-ethoxy)-4- morpholin-4-yl-quinazoline |
| 614 | 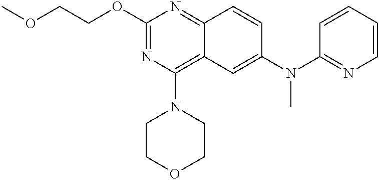 | [2-(2-Methoxy-ethoxy)-4- morpholin-4-yl-quinazolin-6- yl]-methyl-pyridin-2-yl-amine |
| 615 | 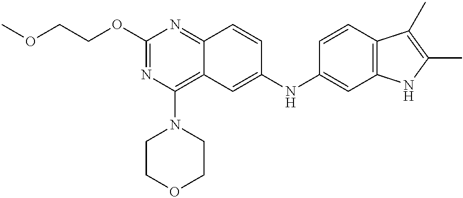 | (2,3-Dimethyl-1H-indol-6-yl)- [2-(2-methoxy-ethoxy)-4- morpholin-4-yl-quinazolin-6- yl]-amine |
| 616 | 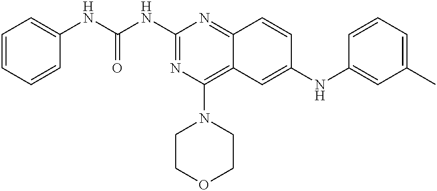 | 1-(4-Morpholin-4-yl-6-m- tolylamino-quinazolin-2-yl)-3- phenyl-urea |
| 617 | 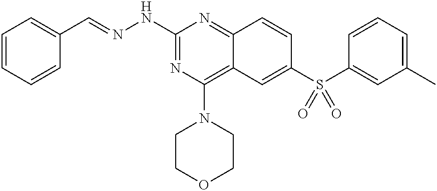 | N-Benzylidene-N′-[4- morpholin-4-yl-6-(toluene-3- sulfonyl)-quinazolin-2-yl]- hydrazine |
| 618 | 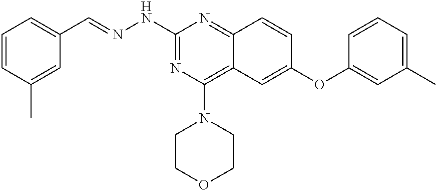 | N-(3-Methyl-benzylidene)-N′- (4-morpholin-4-yl-6-m- tolyloxy-quinazolin-2-yl)- hydrazine |
| 619 | 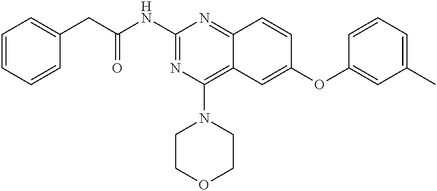 | N-(4-Morpholin-4-yl-6-m- tolyloxy-quinazolin-2-yl)-2- phenyl-acetamide |
| 620 | 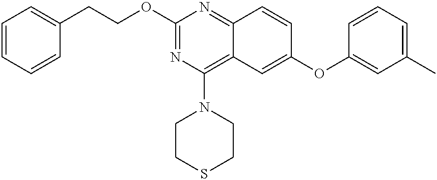 | 2-Phenethyloxy-4- thiomorpholin-4-yl-6-m- tolyloxy-quinazoline |
| 621 | 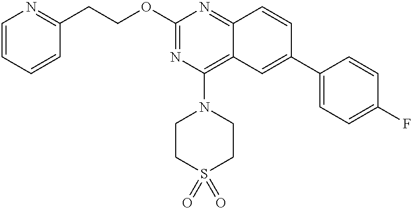 | 4-(1,1-Dioxo-1l6- thiomorpholin-4-yl)-6-(4- fluoro-phenyl)-2-(2-pyridin-2- yl-ethoxy)-quinazoline |
| 622 | 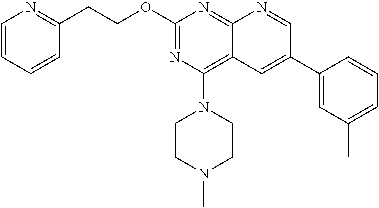 | 4-(4-Methyl-piperazin-1-yl)-2- (2-pyridin-2-yl-ethoxy)-6-m- tolyl-pyrido[2,3-d]pyrimidine |
| 623 | 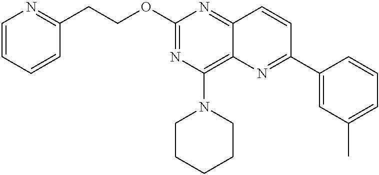 | 4-Piperidin-1-yl-2-(2-pyridin- 2-yl-ethoxy)-6-m-tolyl- pyrido[3,2-d]pyrimidine |
| 624 | 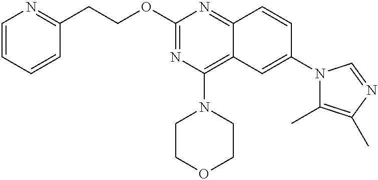 | 6-(4,5-Dimethyl-imidazol-1- yl)-4-morpholin-4-yl-2-(2- pyridin-2-yl-ethoxy)- quinazoline |
| 625 | 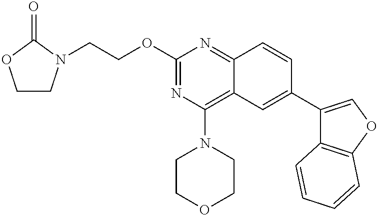 | 3-[2-(6-Benzofuran-3-yl-4- morpholin-4-yl-quinazolin-2- yloxy)-ethyl]-oxazolidin-2-one |
| 626 | 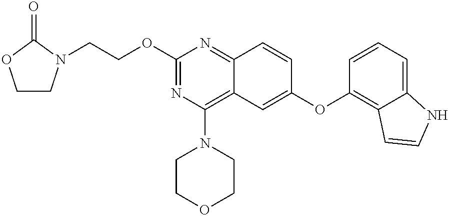 | 3-{2-[6-(1H-Indol-4-yloxy)-4- morpholin-4-yl-quinazolin-2- yloxy]-ethyl}-oxazolidin-2-one |
| 627 | 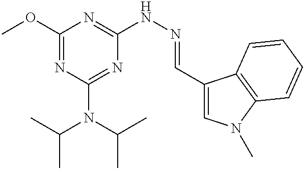 | Diisopropyl-{4-methoxy-6-[N′- (1-methyl-1H-indol-3- ylmethylene)-hydrazino]- [1,3,5]triazin-2-yl}-amine |
| 628 | 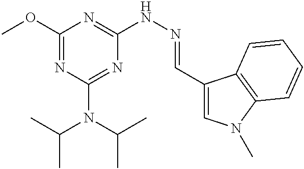 | {4-[N′-(1H-Indol-3- ylmethylene)-hydrazino]-6- methoxy-[1,3,5]triazin-2-yl}- diisopropyl-amine |
| 629 | 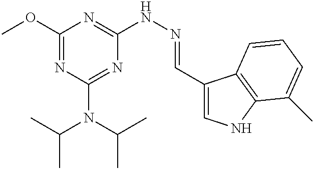 | Diisopropyl-{4-methoxy-6-[N′- (7-methyl-1H-indol-3- ylmethylene)-hydrazino]- [1,3,5]triazin-2-yl}-amine |
| 630 | 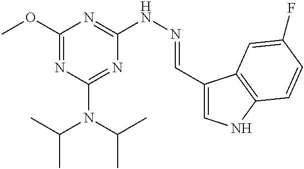 | {4-[N′-(5-Fluoro-1H-indol-3- ylmethylene)-hydrazino]-6- methoxy-[1,3,5]triazin-2-yl}- diisopropyl-amine |
| 631 | 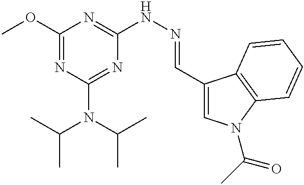 | 1-{3-[(4-Diisopropylamino-6- methoxy-[1,3,5]triazin-2-yl)- hydrazonomethyl]-indol-1-yl}- ethanone |
| 632 | 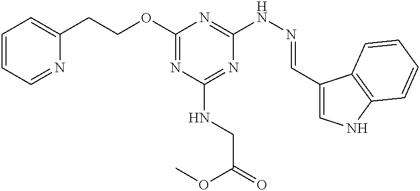 | [4-[N′-(1H-Indol-3- ylmethylene)-hydrazino]-6-(2- pyridin-2-yl-ethoxy)- [1,3,5]triazin-2-ylamino]-acetic acid methyl ester |
| 633 | 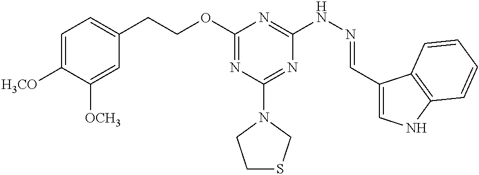 | N-{4-[2-(3,4-Dimethoxy- phenyl)-ethoxy]-6-thiazolidin- 3-yl-[1,3,5]triazin-2-yl}-N′- (1H-indol-3-ylmethylene)- hydrazine |
| 634 | 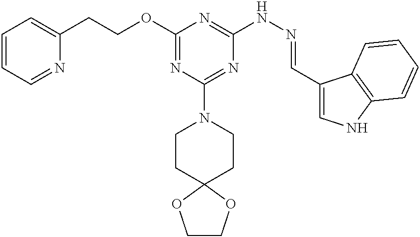 | N-[4-(1,4-Dioxa-8-aza- spiro[4.5]dec-8-yl)-6-(2- pyridin-2-yl-ethoxy)- [1,3,5]triazin-2-yl]-N′-(1H- indol-3-ylmethylene)-hydrazine |
| 635 | 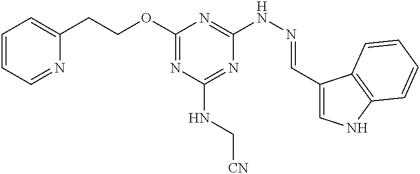 | [4-[N′-(1H-Indol-3- ylmethylene)-hydrazino]-6-(2- pyridin-2-yl-ethoxy)- [1,3,5]triazin-2-ylamino]- acetonitrile |
| 636 | 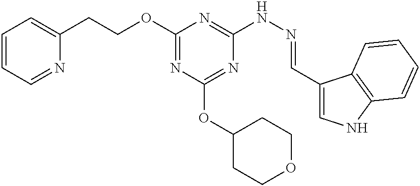 | N-(1H-Indol-3-ylmethylene)- N′-[4-(2-pyridin-2-yl-ethoxy)- 6-(tetrahydro-pyran-4-yloxy)- [1,3,5]triazin-2-yl]-hydrazine |
| 637 | 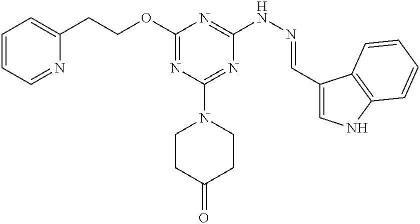 | 1-[4-[N′-(1H-Indol-3- ylmethylene)-hydrazino]-6-(2- pyridin-2-yl-ethoxy)- [1,3,5]triazin-2-yl]-piperidin-4- one |
| 638 | 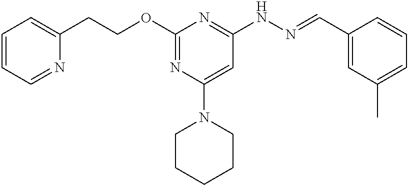 | N-(3-Methyl-benzylidene)N′- [6-piperidin-1-yl-2-(2-pyridin- 2-yl-ethoxy)-pyrimidin-4-yl]- hydrazine |
| 639 | 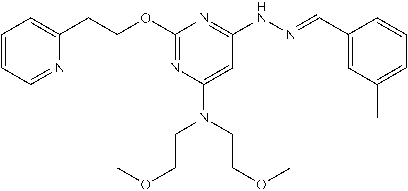 | Bis-(2-methoxy-ethyl)-[6-[N′- (3-methyl-benzylidene)- hydrazino]-2-(2-pyridin-2-yl- ethoxy)-pyrimidin-4-yl]-amine |
| 640 | 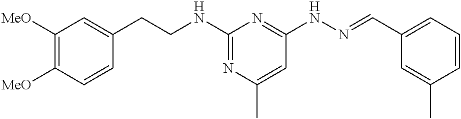 | [2-(3,4-Dimethoxy-phenyl)- ethyl]-{4-methyl-6-[N′-(3- methyl-benzylidene)- hydrazino]-pyrimidin-2-yl}- amine |
| 641 | 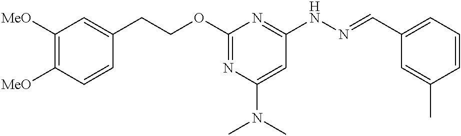 | {2-[2-(3,4-Dimethoxy-phenyl)- ethoxy]-6-[N′-(3-methyl- benzylidene)-hydrazino]- pyrimidin-4-yl}-dimethyl- amine |
| 642 | 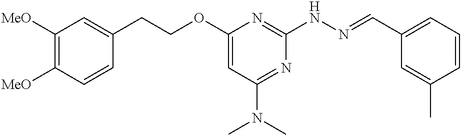 | {6-[2-(3,4-Dimethoxy-phenyl)- ethoxy]-2-[N′-(3-methyl- benzylidene)-hydrazino]- pyrimidin-4-yl}-dimethyl- amine |
| 643 | 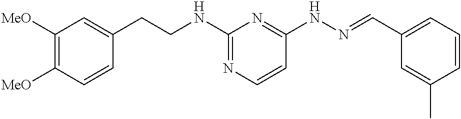 | [2-(3,4-Dimethoxy-phenyl)- ethyl]-{4-[N′-(3-methyl- benzylidene)-hydrazino]- pyrimidin-2-yl}-amine |
| 644 | 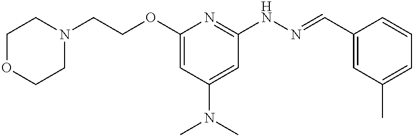 | Dimethyl-[2-[N′-(3-methyl- benzylidene)-hydrazino]-6-(2- morpholin-4-yl-ethoxy)- pyridin-4-yl]-amine |
| 645 | 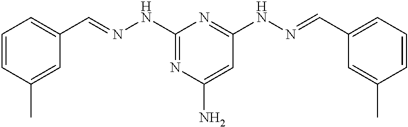 | 2,6-Bis-[N′-(3-methyl- benzylidene)-hydrazino]- pyrimidin-4-ylamine |
| 646 | 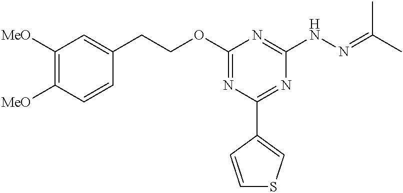 | N-{4-[2-(3,4-Dimethoxy- phenyl)-ethoxy]-6-thiophen-3- yl-[1,3,5]triazin-2-yl}-N′- isopropylidene-hydrazine |
| 647 | 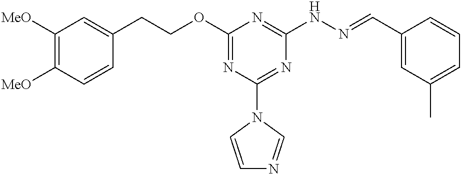 | N-{4-[2-(3,4-Dimethoxy- phenyl)-ethoxy]-6-imidazol-1- yl-[1,3,5]triazin-2-yl}-N′-(3- methyl-benzylidene)- hydrazine |
| 648 | 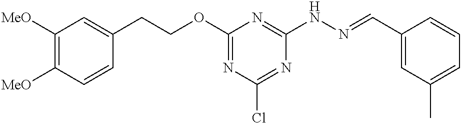 | N-{4-Chloro-6-[2-(3,4- dimethoxy-phenyl)-ethoxy]- [1,3,5]triazin-2-yl}-N′-(3- methyl-benzylidene)- hydrazine |
| 649 | 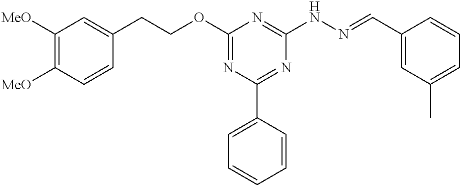 | N-{4-[2-(3,4-Dimethoxy- phenyl)-ethoxy]-6-phenyl- [1,3,5]triazin-2-yl}-N′-(3- methyl-benzylidene)- hydrazine |
| 650 | 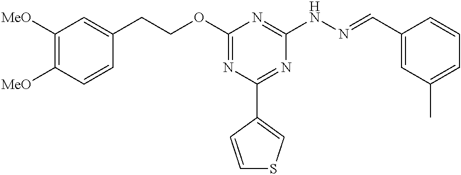 | N-{4-[2-(3,4-Dimethoxy- phenyl)-ethoxy]-6-thiophen-3- yl-[1,3,5]triazin-2-yl}-N′-(3- methyl-benzylidene)- hydrazine |
| 651 | 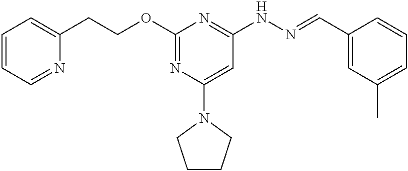 | N-(3-Methyl-benzylidene)-N′- [2-(2-pyridin-2-yl-ethoxy)-6- pyrrolidin-1-yl-pyrimidin-4- yl]-hydrazine |
| 652 | 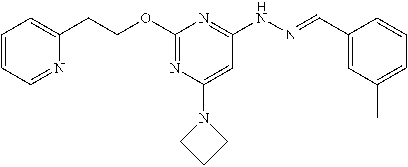 | N-[6-Azetidin-1-yl-2-(2- pyridin-2-yl-ethoxy)- pyrimidin-4-yl]-N′-(3-methyl- benzylidene)-hydrazine |
| 653 | 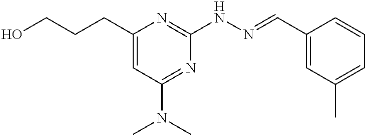 | 3-{6-Dimethylamino-2-[N′-(3- methyl-benzylidene)- hydrazino]-pyrimidin-4-yl}- propan-1-ol |
| 654 | 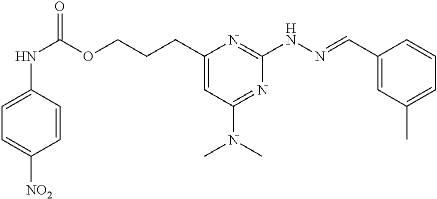 | (4-Nitro-phenyl)-carbamic acid 3-{6-dimethylamino-2-[N′-(3- methyl-benzylidene)- hydrazino]-pyrimidin-4-yl}- propyl ester |
| 655 | 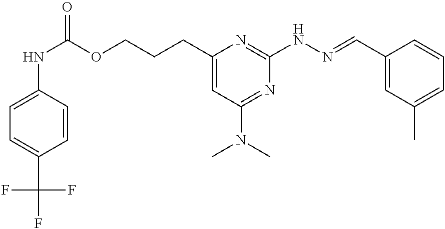 | (4-Trifluoromethyl-phenyl)- carbamic acid 3-{6- dimethylamino-2-[N′-(3- methyl-benzylidene)- hydrazino]-pyrimidin-4-yl}- propyl ester |
| 656 | 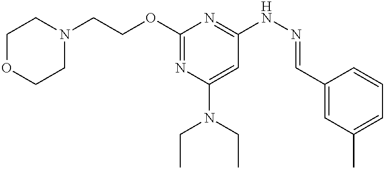 | Diethyl-[6-[N′-(3-methyl- benzylidene)-hydrazino]-2-(2- morpholin-4-yl-ethoxy)- pyrimidin-4-yl]-amine |
| 657 | 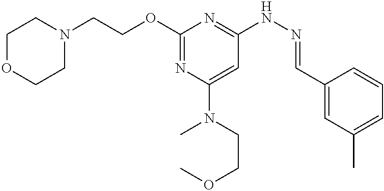 | (2-Methoxy-ethyl)-methyl-[6- [N′-(3-methyl-benzylidene)- hydrazino]-2-(2-morpholin-4- yl-ethoxy)-pyrimidin-4-yl]- amine |
| 658 | 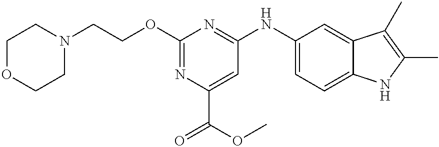 | 6-(2,3-Dimethyl-1H-indol-5- ylamino)-2-(2-morpholin-4-yl- ethoxy)-pyrimidine-4- carboxylic acid methyl ester |
| 659 | 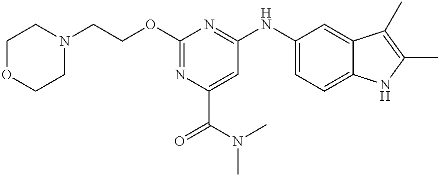 | 6-(2,3-Dimethyl-1H-indol-5- ylamino)-2-(2-morpholin-4-yl- ethoxy)-pyrimidine-4- carboxylic acid dimethylamide |
| 660 | 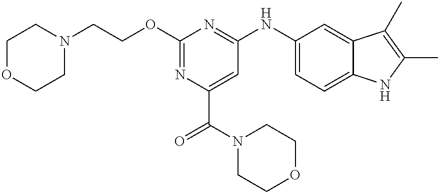 | [6-(2,3-Dimethyl-1H-indol-5- ylamino)-2-(2-morpholin-4-yl- ethoxy)-pyrimidin-4-yl]- morpholin-4-yl-methanone |
| 661 | 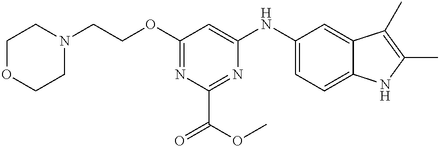 | 4-(2,3-Dimethyl-1H-indol-5- ylamino)-6-(2-morpholin-4-yl- ethoxy)-pyrimidine-2- carboxylic acid methyl ester |
| 662 | 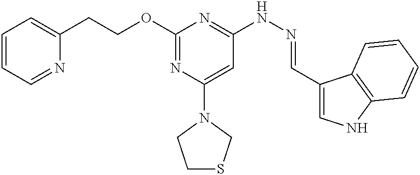 | N-(1H-Indol-3-ylmethylene)- N′-[2-(2-pyridin-2-yl-ethoxy)- 6-thiazolidin-3-yl- pyrimidin-4-yl]-hydrazine |
| 663 | 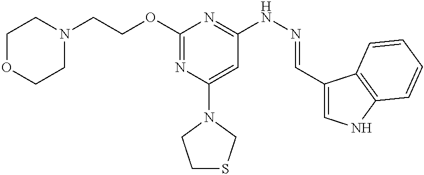 | N-(1H-Indol-3-ylmethylene)- N′-[2-(2-morpholin-4-yl- ethoxy)-6-thiazolidin-3- yl-pyrimidin-4-yl]-hydrazine |
| 664 | 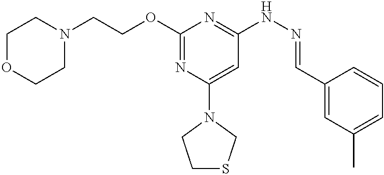 | N-(3-Methyl-benzylidene)-N′- [2-(2-morpholin-4-yl-ethoxy)- 6-thiazolidin- 3-yl-pyrimidin-4-yl]-hydrazine |
| 665 | 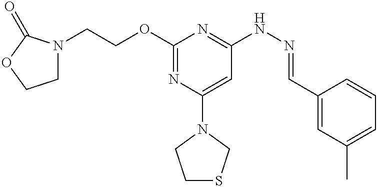 | 3-(2-{4-[N′-(3-Methyl- benzylidene)-hydrazino]-6- thiazolidin-3-yl-pyrimidin- 2-yloxy}-ethyl)-oxazolidin-2- one |
| 666 | 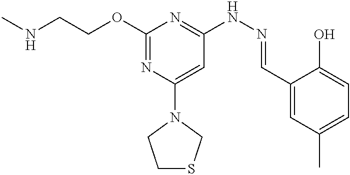 | 4-Methyl-2-{[2-(2- methylamino-ethoxy)-6- thiazolidin-3-yl-pyrimidin-4- yl]-hydrazonomethyl}-phenol |
| 667 | 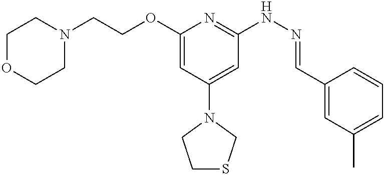 | N-(3-Methyl-benzylidene)-N′- [6-(2-morpholin-4-yl-ethoxy)- 4-thiazolidin-3-yl-pyridin-2- yl]-hydrazine |
| 668 | 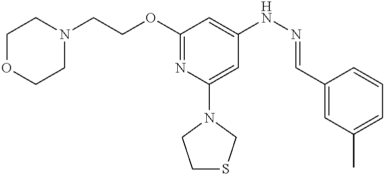 | N-(3-Methyl-benzylidene)-N′- [2-(2-morpholin-4-yl-ethoxy)- 6-thiazolidin-3-yl-pyridin-4- yl]-hydrazine |
| 669 | 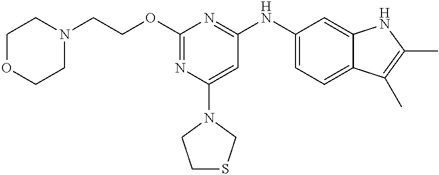 | (2,3-Dimethyl-1H-indol-6-yl)- [2-(2-morpholin-4-yl-ethoxy)- 6-thiazolidin-3-yl-pyrimidin-4- yl]-amine |
| 670 | 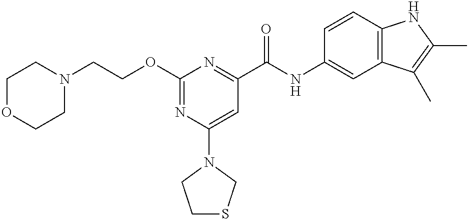 | 2-(2-Morpholin-4-yl-ethoxy)-6- thiazolidin-3-yl-pyrimidine-4- carboxylic acid (2,3-dimethyl- 1H-indol-5-yl)-amide |
| 671 | 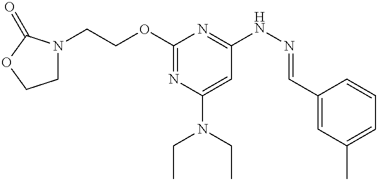 | 3-(2-{4-Diethylamino-6-[N′-(3- methyl-benzylidene)- hydrazino]-pyrimidin-2- yloxy}-ethyl)-oxazolidin-2-one |
| 672 | 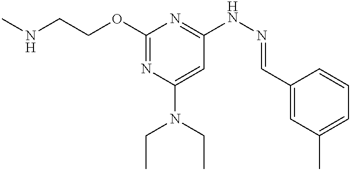 | Diethyl-{2-(2-methylamino- ethoxy)-6-[N′-(3-methyl- benzylidene)-hydrazino]- pyrimidin-4-yl}-amine |
| 673 | 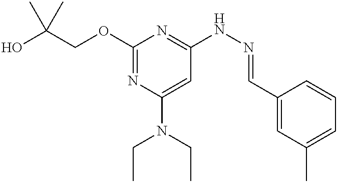 | 1-{4-Diethylamino-6-[N′-(3- methyl-benzylidene)- hydrazino]-pyrimidin-2- yloxy}-2-methyl-propan-2-ol |
| 674 | 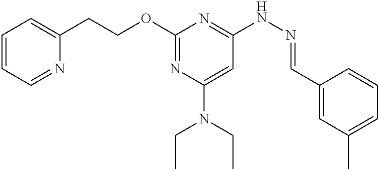 | Diethyl-[6-[N′-(3-methyl- benzylidene)-hydrazino]-2-(2- pyridin-2-yl-ethoxy)- pyrimidin-4-yl]-amine |
| 675 | 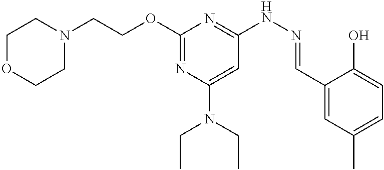 | 2-{[6-Diethylamino-2-(2- morpholin-4-yl-ethoxy)- pyrimidin-4-yl]- hydrazonomethyl}-4-methyl- phenol |
| 676 | 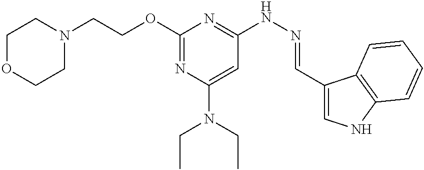 | Diethyl-[6-[N′-(1H-indol-3- ylmethylene)-hydrazino]-2-(2- morpholin-4-yl-ethoxy)- pyrimidin-4-yl]-amine |
| 677 | 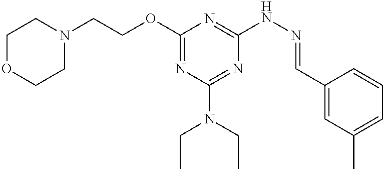 | Diethyl-[4-[N′-(3-methyl- benzylidene)-hydrazino]-6-(2- morpholin-4-yl-ethoxy)- [1,3,5]triazin-2-yl]-amine |
| 678 | 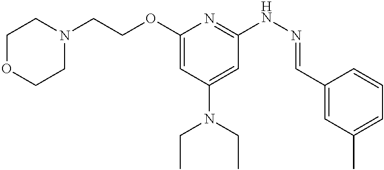 | Diethyl-[2-[N′-(3-methyl- benzylidene)-hydrazino]-6-(2- morpholin-4-yl-ethoxy)- pyridin-4-yl]-amine |
| 679 | 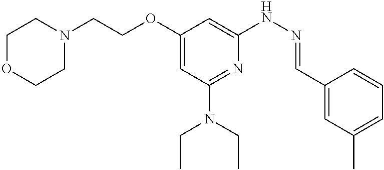 | Diethyl-[6-[N′-(3-methyl- benzylidene)-hydrazino]-4-(2- morpholin-4-yl-ethoxy)- pyridin-2-yl]-amine |
| 680 | 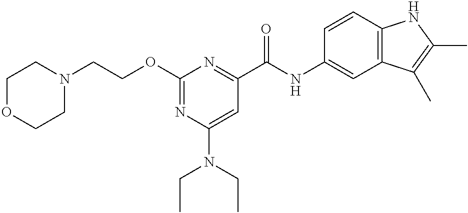 | 6-Diethylamino-2-(2- morpholin-4-yl-ethoxy)- pyrimidine-4-carboxylic acid (2,3-dimethyl-1H-indol-5-yl)- amide |
| 681 | 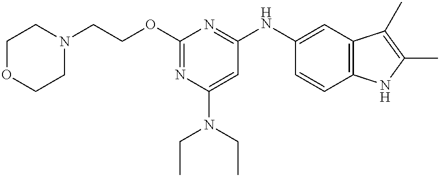 | 6-Diethylamino-2-(2- morpholin-4-yl-ethoxy)-4- [(2,3-dimethy-1H-indol-5-yl)- amino]-pyrimidine |
| 682 | 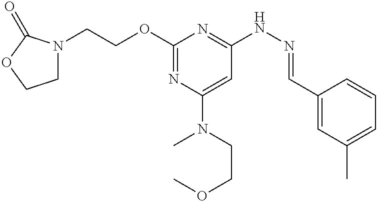 | 3-(2-{4-[(2-Methoxy-ethyl)- methyl-amino]-6-[N′-(3- methyl-benzylidene)- hydrazino]-pyrimidin-2- yloxy}-ethyl)-oxazolidin-2-one |
| 683 | 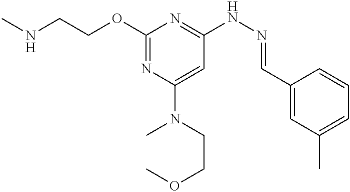 | (2-Methoxy-ethyl)-methyl-{2- (2-methylamino-ethoxy)-6-[N′- (3-methyl-benzylidene)- hydrazino]-pyrimidin-4-yl}- amine |
| 684 | 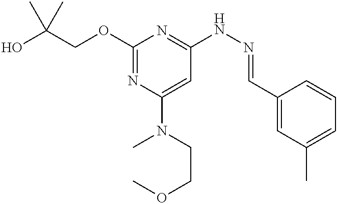 | 1-{4-[(2-Methoxy-ethyl)- methyl-amino]-6-[N′-(3- methyl-benzylidene)- hydrazino]-pyrimidin-2- yloxy}-2-methyl-propan-2-ol |
| 685 | 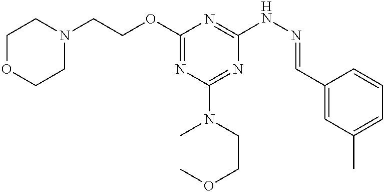 | (2-Methoxy-ethyl)-methyl-[4- [N′-(3-methyl-benzylidene)- hydrazino]-6-(2-morpholin-4- yl-ethoxy)-[1,3,5]triazin-2-yl]- amine |
| 686 | 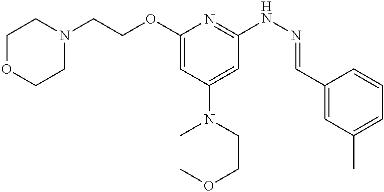 | (2-Methoxy-ethyl)-methyl-[2- [N′-(3-methyl-benzylidene)- hydrazino]-6-(2-morpholin-4- yl-ethoxy)-pyridin-4-yl]-amine |
| 687 | 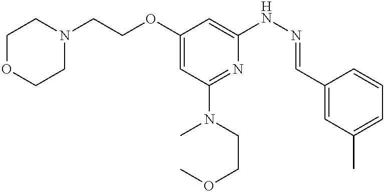 | (2-Methoxy-ethyl)-methyl-[6- [N′-(3-methyl-benzylidene)- hydrazino]-4-(2-morpholin-4- yl-ethoxy)-pyridin-2-yl]-amine |
| 688 | 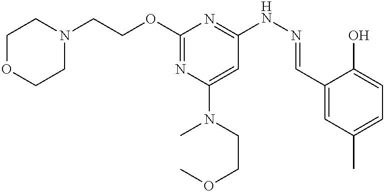 | 2-{[6-[(2-Methoxy-ethyl)- methyl-amino]-2-(2-morpholin- 4-yl-ethoxy)-pyrimidin-4-yl]- hydrazonomethyl}-4-methyl- phenol |
| 689 | 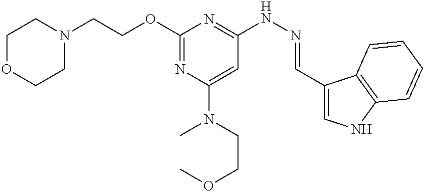 | [6-[N′-(1H-Indol-3- ylmethylene)-hydrazino]-2-(2- morpholin-4-yl-ethoxy)- pyrimidin-4-yl]-(2-methoxy- ethyl)-methyl-amine |
| 690 | 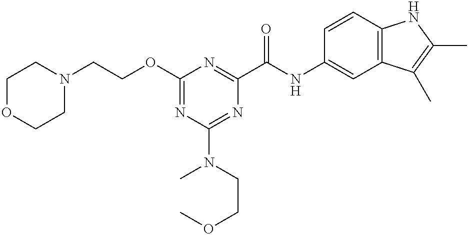 | 4-[(2-Methoxy-ethyl)-methyl- amino]-6-(2-morpholin-4-yl- ethoxy)-[1,3,5]triazine-2- carboxylic acid (2,3-dimethyl- 1H-indol-5-yl)-amide |
| 691 | 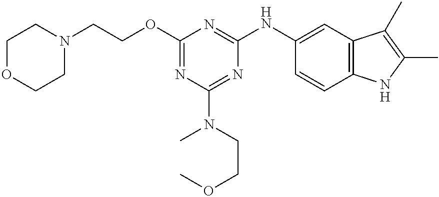 | N-(2,3-Dimethyl-1H-indol-5- yl)-N′-(2-methoxy-ethyl)-N′- methyl-6-(2-morpholin-4-yl- ethoxy)-[1,3,5]triazine-2,4- diamine |
| 692 | 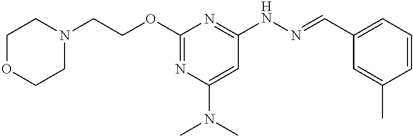 | Dimethyl-[6-[N′-(3-methyl- benzylidene)-hydrazino]-2-(2- morpholin-4-yl-ethoxy)- pyrimidin-4-yl]-amine |
| 693 | 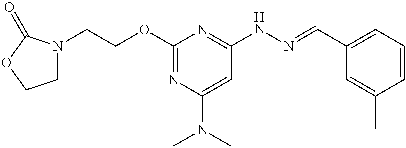 | 3-(2-{4-Dimethylamino-6-[N′- (3-methyl-benzylidene)- hydrazino]-pyrimidin-2- yloxy}-ethyl)-oxazolidin-2-one |
| 694 | 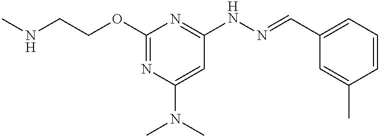 | Dimethyl-{2-(2-methylamino- ethoxy)-6-[N′-(3-methyl- benzylidene)-hydrazino]- pyrimidin-4-yl}-amine |
| 695 | 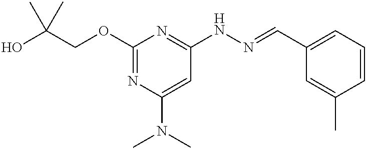 | 1-{4-Dimethylamino-6-[N′-(3- methyl-benzylidene)- hydrazino]-pyrimidin-2- yloxy}-2-methyl-propan-2-ol |
| 696 | 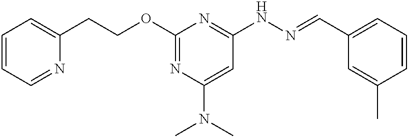 | Dimethyl-[6-[N′-(3-methyl- benzylidene)-hydrazino]-2-(2- pyridin-2-yl-ethoxy)- pyrimidin-4-yl]-amine |
| 697 | 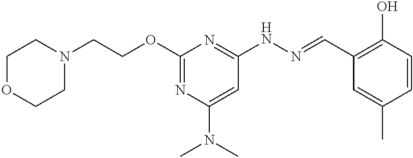 | 2-{[6-Dimethylamino-2-(2- morpholin-4-yl-ethoxy)- pyrimidin-4-yl]- hydrazonomethyl}-4-methyl- phenol |
| 698 | 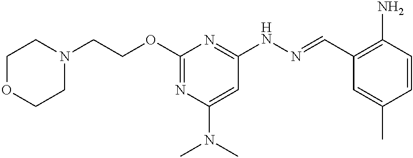 | [6-[N′-(2-Amino-5-methyl- benzylidene)-hydrazino]-2-(2- morpholin-4-yl-ethoxy)- pyrimidin-4-yl]-dimethyl- amine |
| 699 | 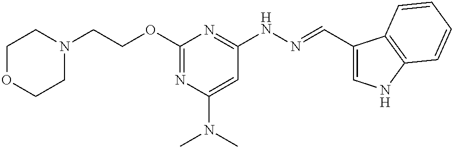 | [6-[N′-(1H-Indol-3- ylmethylene)-hydrazino]-2-(2- morpholin-4-yl-ethoxy)- pyrimidin-4-yl]-dimethyl- amine |
| 700 | 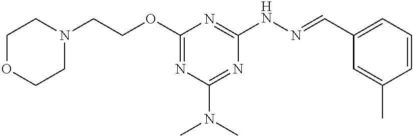 | Dimethyl-[4-[N′-(3-methyl- benzylidene)-hydrazino]-6-(2- morpholin-4-yl-ethoxy)- [1,3,5]triazin-2-yl]-amine |
| 701 | 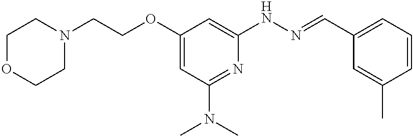 | Dimethyl-[6-[N′-(3-methyl- benzylidene)-hydrazino]-4-(2- morpholin-4-yl-ethoxy)- pyridin-2-yl]-amine |
| 702 | 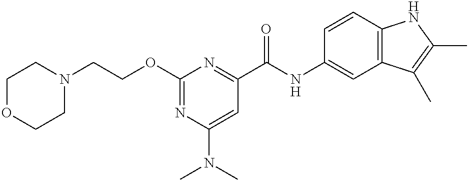 | 6-Dimethylamino-2-(2- morpholin-4-yl-ethoxy)- pyrimidine-4-carboxylic acid (2,3-dimethyl-1H-indol-5-yl)- amide |
| 703 | 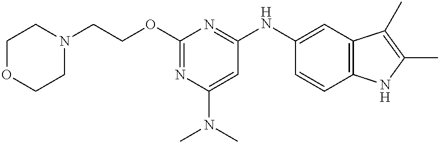 | 6-Dimethylamino-2-(2- morpholin-4-yl-ethoxy)-4- [(2,3-dimethyl-1H-indol-5-yl)- amino] pyrimidine |
| 704 | 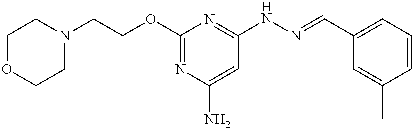 | 6-[N′-(3-Methyl-benzylidene)- hydrazino]-2-(2-morpholin-4- yl-ethoxy)-pyrimidin-4- ylamine |
| 705 | 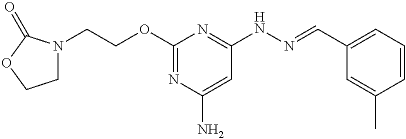 | 3-(2-{4-Amino-6-[N′-(3- methyl-benzylidene)- hydrazino]-pyrimidin-2- yloxy}-ethyl)-oxazolidin-2-one |
| 706 | 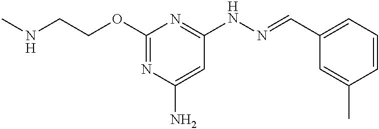 | 2-(2-Methylamino-ethoxy)-6- [N′-(3-methyl-benzylidene)- hydrazino]-pyrimidin-4- ylamine |
| 707 | 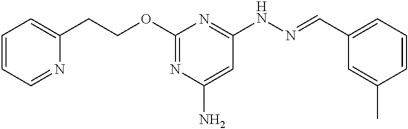 | 6-[N′-(3-Methyl-benzylidene)- hydrazino]-2-(2-pyridin-2-yl- ethoxy)-pyrimidin-4-ylamine |
| 708 | 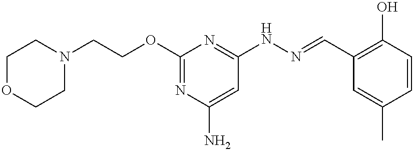 | 2-{[6-Amino-2-(2-morpholin- 4-yl-ethoxy)-pyrimidin-4-yl]- hydrazonomethyl}-4-methyl- phenol |
| 709 | 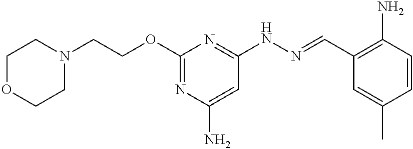 | 6-[N′-(2-Amino-5-methyl- benzylidene)-hydrazino]-2-(2- morpholin-4-yl-ethoxy)- pyrimidin-4-ylamine |
| 710 | 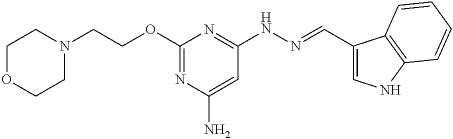 | 6-[N′-(1H-Indol-3- ylmethylene)-hydrazino]-2-(2- morpholin-4-yl-ethoxy)- pyrimidin-4-ylamine |
| 711 | 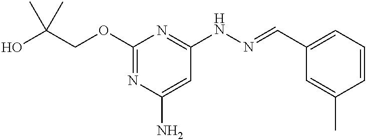 | 1-{4-Amino-6-[N′-(3-methyl- benzylidene)-hydrazino]- pyrimidin-2-yloxy}-2-methyl- propan-2- |
| 712 | 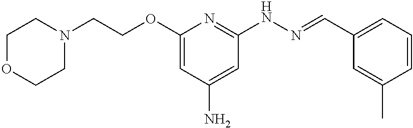 | 2-[N′-(3-Methyl-benzylidene)- hydrazino]-6-(2-morpholin-4- yl-ethoxy)-pyridin-4-ylamine |
| 713 | 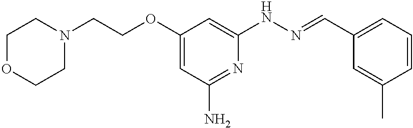 | 6-[N′-(3-Methyl-benzylidene)- hydrazino]-4-(2-morpholin-4- yl-ethoxy)-pyridin-2-ylamine |
| 714 | 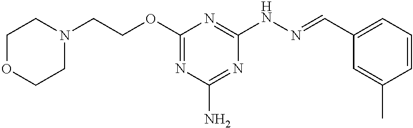 | 4-[N′-(3-Methyl-benzylidene)- hydrazino]-6-(2-morpholin-4- yl-ethoxy)-[1,3,5]triazin-2- ylamine |
| 715 | 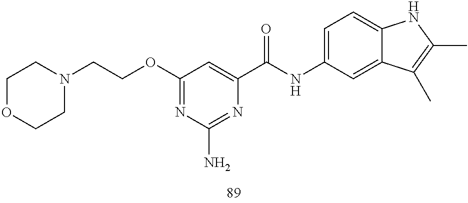 | 2-Amino-6-(2-morpholin-4-yl- ethoxy)-pyrimidine-4- carboxylic acid (2,3-dimethyl- 1H-indol-5-yl)-amide |
| 716 | 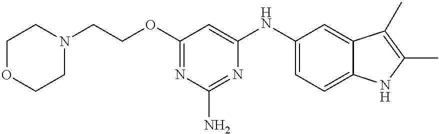 | N4-(2,3-Dimethyl-1H-indol-5- yl)-6-(2-morpholin-4-yl- ethoxy)-pyrimidine-2,4- diamine |
| 717 | 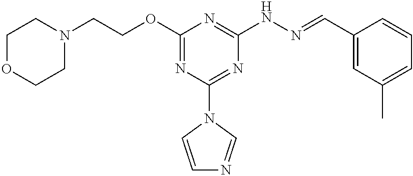 | N-[4-Imidazol-1-yl-6-(2- morpholin-4-yl-ethoxy)- [1,3,5]triazin-2-yl]-N′-(3- methyl-benzylidene)-hydrazine |
| 718 | 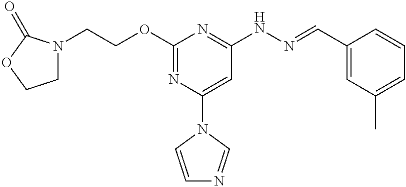 | 3-(2-{4-Imidazol-1-yl-6-[N′-(3- methyl-benzylidene)- hydrazino]-pyrimidin-2- yloxy}-ethyl)-oxazolidin-2-one |
| 719 | 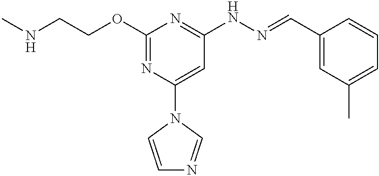 | (2-{4-Imidazol-1-yl-6-[N′-(3- methyl-benzylidene)- hydrazino]-pyrimidin-2- yloxy}-ethyl)-methyl-amine |
| 720 | 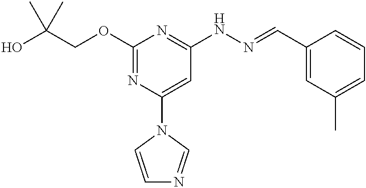 | 1-{4-Imidazol-1-yl-6-[N′-(3- methyl-benzylidene)- hydrazino]-pyrimidin-2- yloxy}-2-methyl-propan-2-ol |
| 721 | 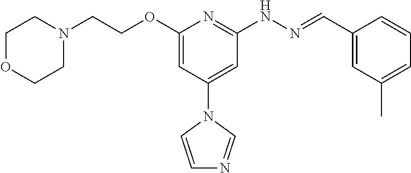 | N-[4-Imidazol-1-yl-6-(2- morpholin-4-yl-ethoxy)- pyridin-2-yl]-N′-(3-methyl- benzylidene)-hydrazine |
| 722 | 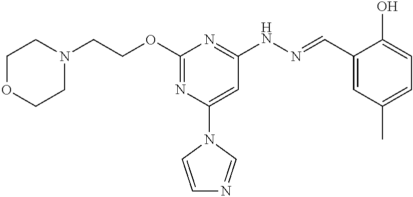 | 2-{[6-Imidazol-1-yl-2-(2- morpholin-4-yl-ethoxy)- pyrimidin-4-yl]- hydrazonomethyl}-4-methyl- phenol |
| 723 | 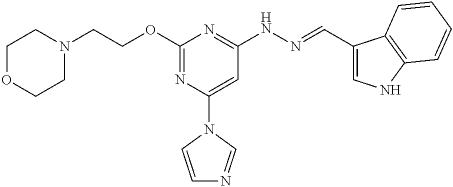 | N-[6-Imidazol-1-yl-2-(2- morpholin-4-yl-ethoxy)- pyrimidin-4-yl]-N′-(1H-indol- 3-ylmethylene)-hydrazine |
| 724 | 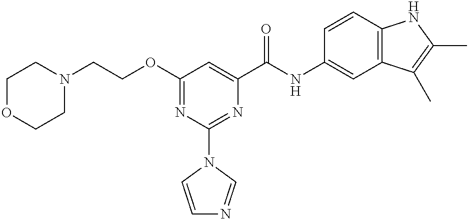 | 2-Imidazol-1-yl-6-(2- morpholin-4-yl-ethoxy)- pyrimidine-4-carboxylic acid (2,3-dimethyl-1H-indol-5-yl)- amide |
| 725 | 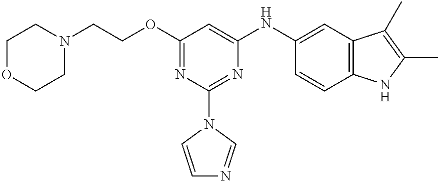 | (2,3-Dimethyl-1H-indol-5-yl)- [2-imidazol-1-yl-6-(2- morpholin-4-yl-ethoxy)- pyrimidin-4-yl]-amine |
| 726 | 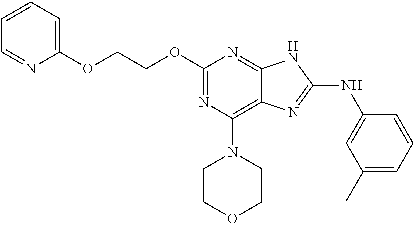 | {6-morpholin-4-yl-2-[2- (pyridin-2-yloxy)-ethoxy]-9H- purin-8-yl}-m-tolyl-amine |
| 727 | 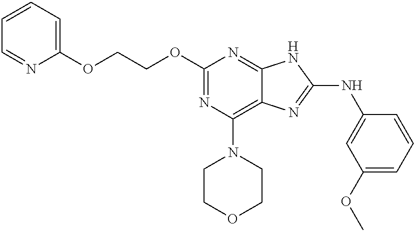 | (3-Methoxyphenyl)-{6- morpholin-4-yl-2-[2-(pyridin- 2-yloxy)-ethoxy]-9H-purin-8- yl}-amine |
| 728 | 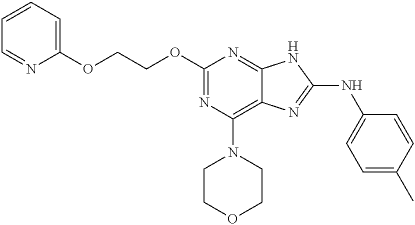 | {6-Morpholin-4-yl-2-[2- (pyridin-2-yloxy)-ethoxy]-9H- purin-8-yl}-p-tolyl-amine |
| 729 | 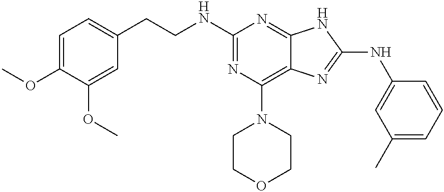 | N²-{2-(3,4-Dimethoxy- phenyl)-ethyl]-6-morpholin-4- yl-N⁸-m-tolyl-9H-purine-2,8- diamine |
| 730 | 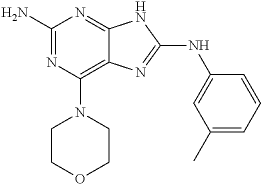 | 6-morpholin-4-yl-N⁸-m-tolyl- 9H-purine-2,8-diamine |
| 731 | 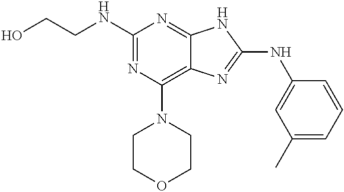 | 2-(6-morpholin-4-yl-8-m- tolylamino-9H-purin-2- ylamino)-ethanol |
| 732 | 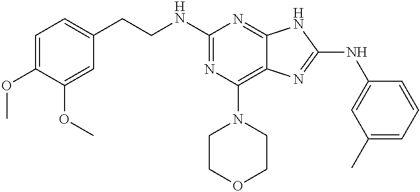 | N²-[2-(3,4-Dimethoxy-phenyl)- ethyl]-6-morpholin-4-yl-N⁸-m- tolyl-9H-purine-2,8-diamine |
| 733 | 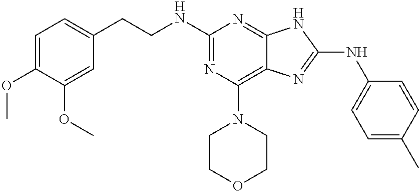 | N²-[2-(3,4-Dimethoxy-phenyl)- ethyl]-6-morpholin-4-yl-N⁸-m- tolyl-9H-purine-2,8-diamine |
| 734 | 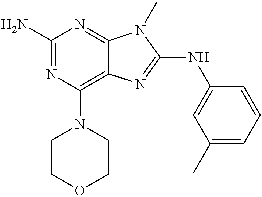 | 9-Methyl-6-morpholin-4-yl-N⁸- m-tolyl-9H-purine-2,8-diamine |
| 735 | 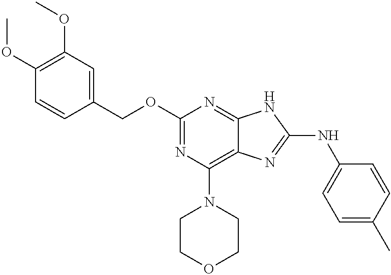 | [2-(3,4-dimethoxy-benzyloxy)- 6-morpholin-4-yl-9H-purine-8- yl-p-tolyl-amine |
| 736 | 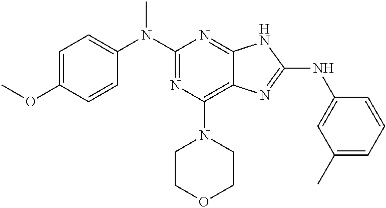 | N²-(4-methoxy-phenyl)-N²- methyl-6-morpholin-4-yl-N⁸- m-tolyl-9H-purine-2,8-diamine |
| 737 | 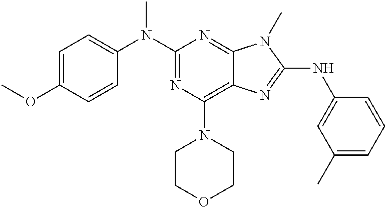 | N²-(4-methoxy-phenyl)-N²- methyl-9-methyl-6-morpholin- 4-yl-N⁸-m-tolyl-9H-purine-2,8- diamine |
| 738 | 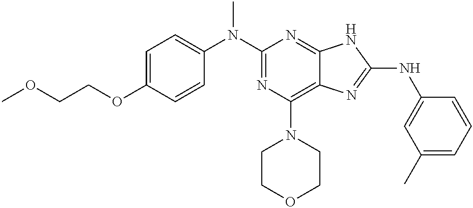 | N²-[4-(2-Methoxy-ethoxy- phenyl]-N²-methyl-6- morpholin-4-yl-N⁸-m-tolyl-9H- purine-2,8-diamine |
| 739 | 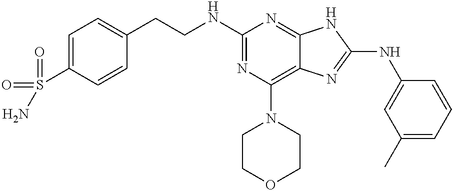 | 4-[2-(6-morpholin-4-yl-8-m- tolylamino-9H-purine-2- ylamino)-ethyl]- benzenesulfonamide |
| 740 | 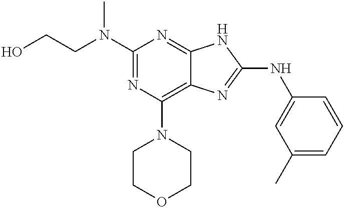 | 2-[Methyl-(6-morpholin-4-yl- 8-m-tolylamino-9H-purin-2- ylamino)-amino]-ethanol |
| 741 | 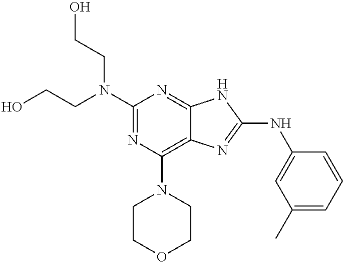 | 2-[(2-Hydroxy-ethyl)-(6- morpholin-4-yl-8-m- tolylamino-9H-purin-2- ylamino)-amino]-ethanol |
| 742 | 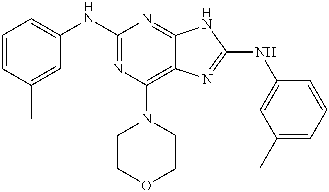 | 6-Morpholin-4-yl-N²,N⁸-di-m- tolyl-9H-purine-2,8-diamine |
| 743 | 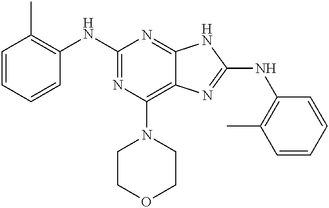 | 6-Morpholin-4-yl-N²,N⁸-di-o- tolyl-9H-purine-2,8-diamine |
| 744 | 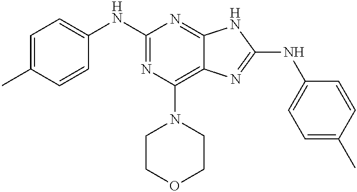 | 6-Morpholin-4-yl-N²,N⁸-di-p- tolyl-9H-purine-2,8-diamine |
| 745 | 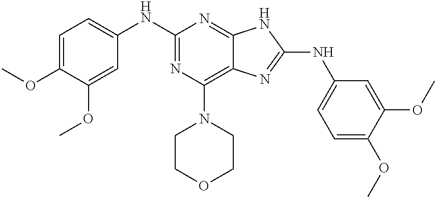 | N²,N⁸-bis-(3,4-dimethoxy- phenyl)-6-morpholin-4-yl-9H- purine-2,8-diamine |
| 746 | 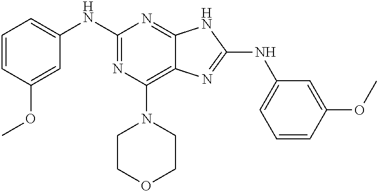 | N²,N⁸-bis-(3-methoxy-phenyl)- 6-morpholin-4-yl-9H-purine- 2,8-diamine |
| 747 | 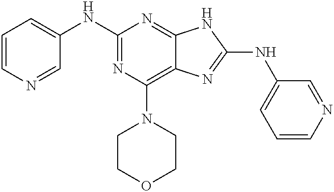 | 6-morpholin-4-yl-N²,N⁸-di- pyridine-9H-purine-2,8- diamine |
| 748 | 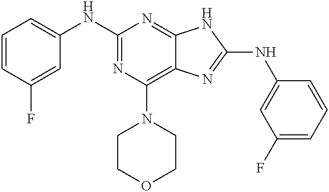 | N²,N⁸-bis-(3-fluoro-phenyl)-6- morpholin-4-yl-9H-purine-2,8- diamine |
| 749 | 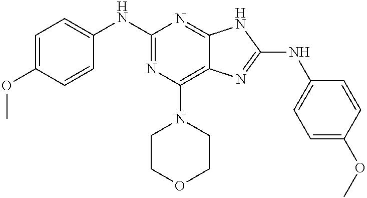 | N²,N⁸-bis-(4-methoxy-phenyl)- 6-morpholin-4-yl-9H-purine- 2,8-diamine |
| 750 | 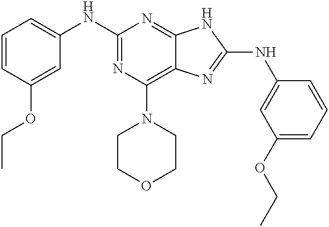 | N²,N⁸-bis-(3-ethoxy-phenyl)- 6-morpholin-4-yl-9H-purine- 2,8-diamine |
| 751 | 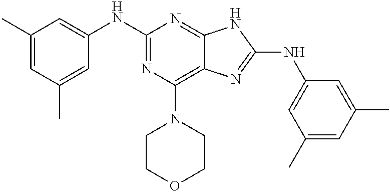 | N²,N⁸-bis-(3,5-dimethyl- phenyl)-6-morpholin-4-yl-9H- purine-2,8-diamine |
| 752 | 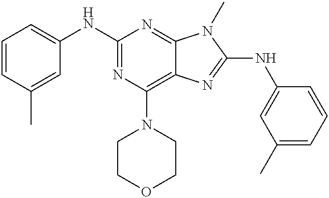 | 9-Methyl-6-morpholin-4-yl- N²,N⁸-di-m-tolyl-9H-purine- 2,8-diamine |
| 753 | 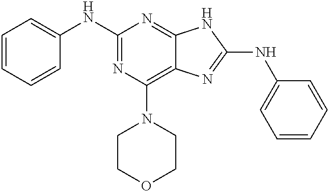 | 6-morpholin-4-yl-N²,N⁸- diphenyl-9H-purine-2,8- diamine |
| 754 | 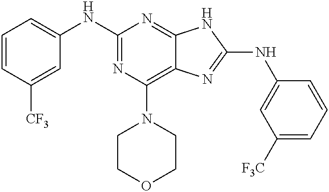 | 6-morpholin-4-yl-N²,N⁸-bis-(3- trifluoromethyl-phenyl)-9H- purine-2,8-diamine |
| 755 | 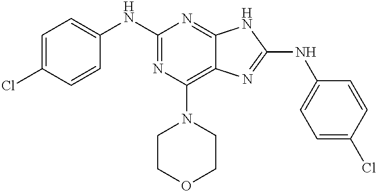 | 6-morpholin-4-yl-N²,N⁸-bis-(4- chloro-phenyl)-9H-purine-2,8- diamine |
| 756 | 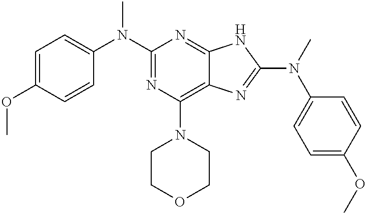 | N²,N⁸-bis-(4-methoxy-phenyl)- N²,N⁸-dimethyl-6- morpholin-4-yl-9H- purine-2,8-diamine |
| 757 | 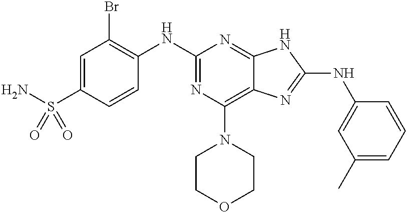 | 3-Bromo-4-(6-morpholin-4-yl- 8-m-tolylamino-9H-purin-2- ylamino)-benzenesulfonamide |
| 758 | 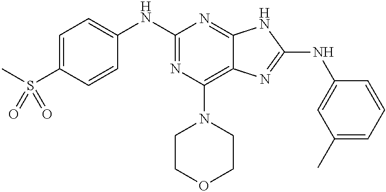 | N²-(4-Methansulfonyl-phenyl)- 6-morpholin-4-yl-N⁸-m-tolyl- 9H-purine-2,8-diamine |
| 759 | 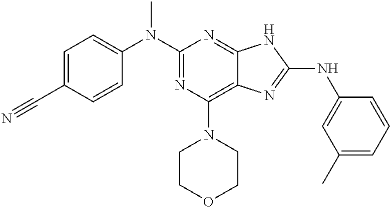 | 4-[Methyl-(6-morpholin-4-yl- 8-m-tolylamino-9H-purin-2- yl)-amino]-benzonitrile |
| 760 | 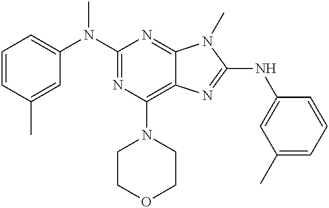 | 9,N²-Dimethyl-6-morpholin-4- yl-N²,N⁸-di-m-tolyl-9H-purine- 2,8-diamine |
| 761 | 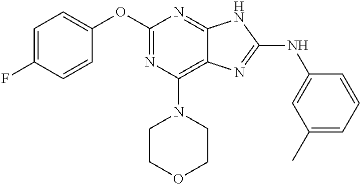 | [2-(4-Fluoro-phenoxy)-6- morpholin-4-yl-9H-purin-8-yl]- m-tolyl-amine |
| 762 | 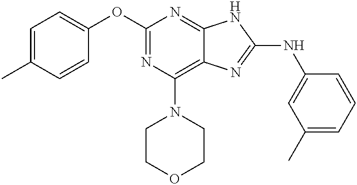 | 6-morpholin-4-yl-2-p-tolyloxy- 9H-purin-8-yl)-m-tolyl-amine |
| 763 | 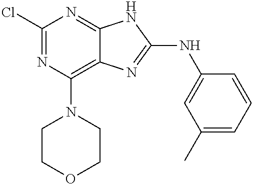 | 2-Chloro-6-morpholin-4-yl-9H- purin-8-yl)-m-tolyl-amine |
| 764 | 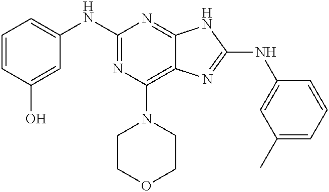 | 3-(6-morpholin-4-yl-8-m- tolylamino-9H-purin-2- ylamino)-phenol |
| 765 | 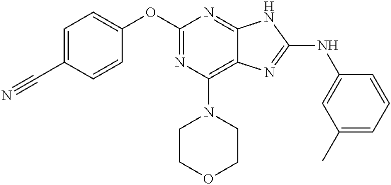 | 4-(6-morpholin-4-yl-8-m- tolylamino-9H-purin-2-yloxy)- benzonitrile |
| 766 | 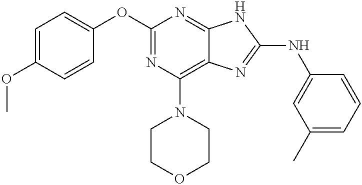 | [2-(4-Methoxy-phenoxy)-6- morpholin-4-yl-9H-purin-2-yl]- m-tolyl-amine |
| 767 | 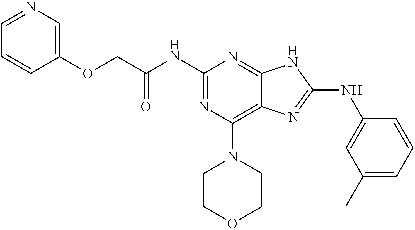 | N-(6-morpholin-4-yl-8-m- tolylamino-9H-purin-2-yl)-2- (pyridin-3-yloxy)-acetamide |
| 768 | 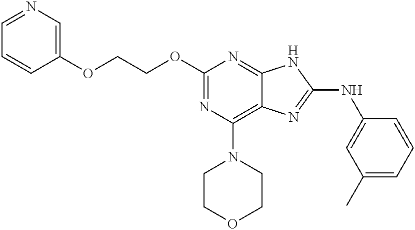 | {6-morpholin-4-yl-2-[2- pyridin-3-yloxy)-ethoxy]-9H- purin-2-yl}-m-toly-amine |
| 769 | 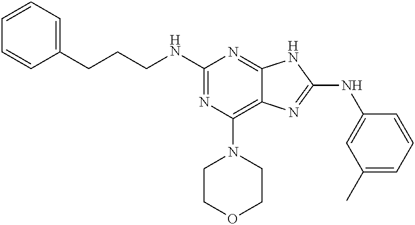 | 6-morpholin-4-yl-N²-(3- phenyl-propyl)-N⁸-m-tolyl-9H- purine-2,8-diamine |
| 780 | 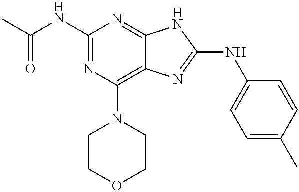 | N-(6-morpholin-4-yl-8-p- tolylamino-9H-purin-2-yl)- acetamide |
| 781 | 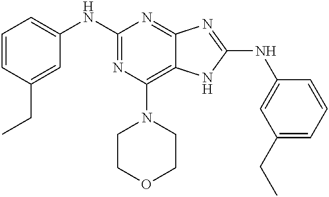 | N-2′,N-8′-Bis-(3-ethyl- phenyl)-6-morpholin-4-yl-7H- purine-2,8-diamine |
| 782 | 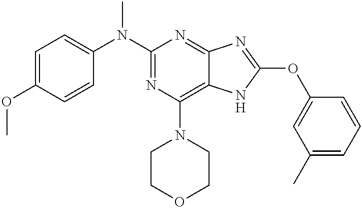 | (4-Methoxy-phenyl)-methyl-(6- morpholin-4-yl-8-m-tolyloxy- 7H-purin-2-yl)-amine |
| 783 | 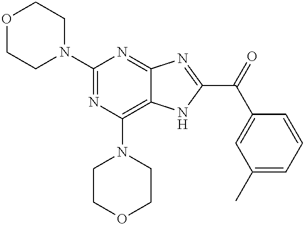 | (2,6-di-morpholin-4-yl-7H- purin-8-yl)-m-tolyl-methanone |
| 784 | 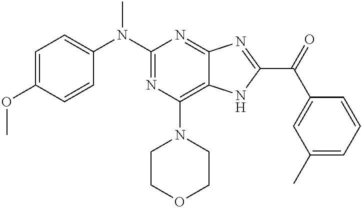 | {2-[(4-Methoxy-phenyl)- methyl-amino]-6-morpholin-4- yl-7H-purin-8-yl)-m-tolyl- methanone |
| 785 | 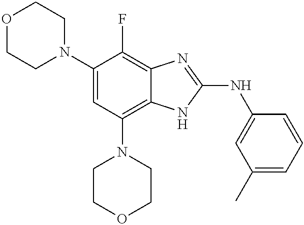 | (4-Fluoro-5,7-di-morpholin-4- yl-1H-benzoimidazol-2-yl)-m- tolyl-amine |
| 786 | 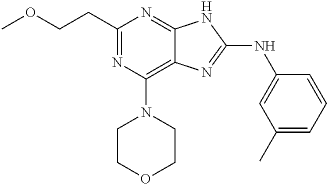 | [2-(2-methoxy-ethyl)-6- morpholin-4-yl-9H-purin-8- yl]-m-tolyl-amine |
| 787 | 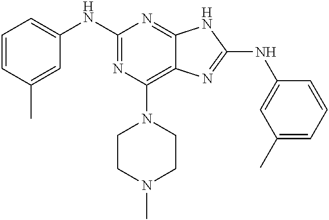 | N²,N⁸-bis-(3-methylphenyl)-6- (4-methylpiperidinyl)-9H- purine-2,8-diamine |
| 788 | 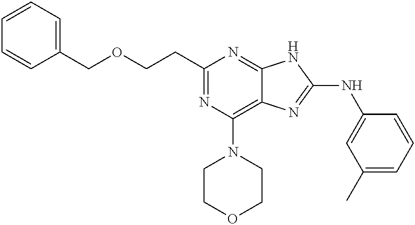 | [2-(2-Benzyloxy-ethyl)-6- morpholin-4-yl-9H-purin-8-yl]- m-tolyl-amine |
| 789 | 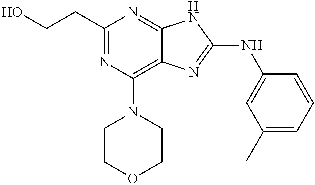 | 2-(6-morpholin-4-yl-8-m- tolylamino-9H-purin-2- yl)-ethanol |
| 790 | 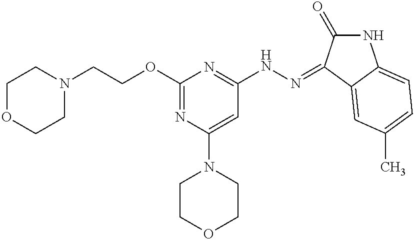 | 5-Methyl-3-{[6-morpholin-4- yl-2-(2-morpholin-4-yl- ethoxy)-pyrimidin-4-yl]- hydrazono}-1,3-dihydro-indol- 2-one |
| 791 | 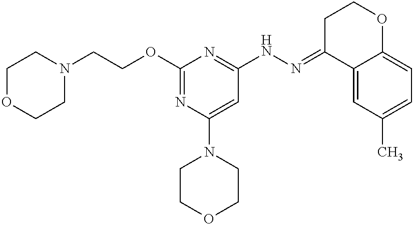 | N-(6-Methyl-chroman-4- ylidene)-N′-[6-morpholin-4-yl- 2-(2-morpholin-4-yl-ethoxy)- pyrimidin-4-yl]-hydrazine |
| 792 | 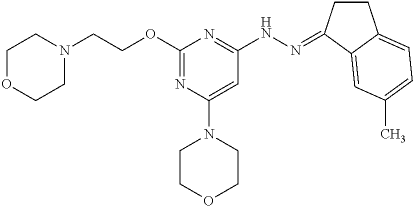 | N-(6-Methyl-indan-1-ylidene)- N′-[6-morpholin-4-yl-2-(2- morpholin-4-yl-ethoxy)- pyrimidin-4-yl]-hydrazine |
| 793 | 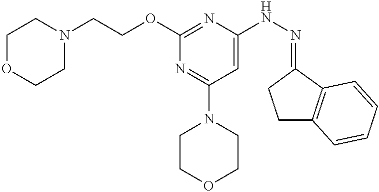 | N-(Indan-1-ylidene)-N′-[6- morpholin-4-yl-2-(2- morpholin-4-yl-ethoxy)- pyrimidin-4-yl]-hydrazine |
| 794 | 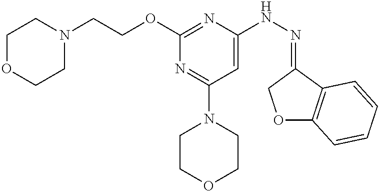 | N-(Benzofuran-3-ylidene)-N′- [6-morpholin-4-yl-2-(2- morpholin-4-yl-ethoxy)- pyrimidin-4-yl]-hydrazine |
| 795 | 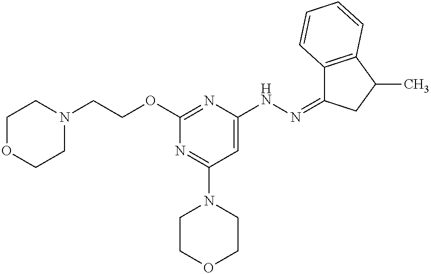 | N-(3-Methyl-indan-1-ylidene)- N′-[6-morpholin-4-yl-2-(2- morpholin-4-yl-ethoxy)- pyrimidin-4-yl]-hydrazine |
| 796 | 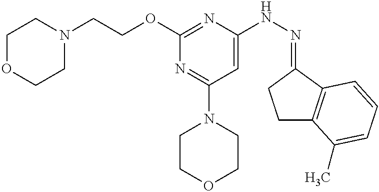 | N-(4-Methyl-indan-1-ylidene)- N′-[6-morpholin-4-yl-2-(2- morpholin-4-yl-ethoxy)- pyrimidin-4-yl]-hydrazine |
| 797 | 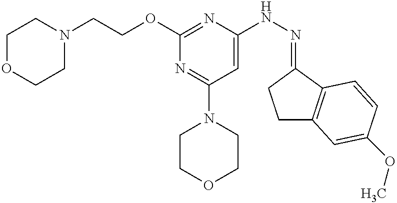 | N-(5-Methoxy-indan-1- ylidene)-N′-[6-morpholin-4-yl- 2-(2-morpholin-4-yl-ethoxy)- pyrimidin-4-yl]-hydrazine |
| 798 | 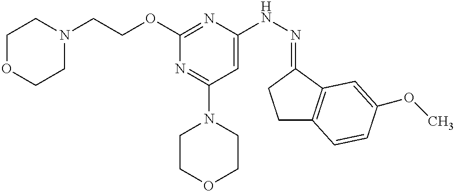 | N-(6-Methoxy-indan-1- ylidene)-N′-[6-morpholin-4-yl- 2-(2-morpholin-4-yl-ethoxy)- pyrimidin-4-yl]-hydrazine |
| 799 | 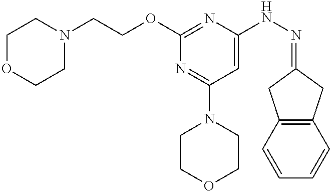 | N-(Indan-2-ylidene)-N′-[6- morpholin-4-yl-2-(2- morpholin-4-yl-ethoxy)- pyrimidin-4-yl]-hydrazine |
| 800 | 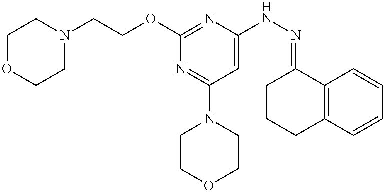 | N-(3,4-Dihydro-2H- naphthalen-1-ylidene)-N′-[6- morpholin-4-yl-2-(2- morpholin-4-yl-ethoxy)- pyrimidin-4-yl]-hydrazine |
| 801 | 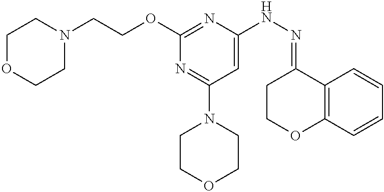 | N-(Chroman-4-ylidene)-N′-[6- morpholin-4-yl-2-(2- morpholin-4-yl-ethoxy)- pyrimidin-4-yl]-hydrazine |
| 802 | 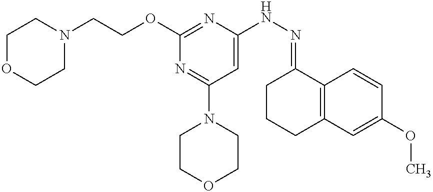 | N-(6-Methoxy-3,4-dihydro-2H- naphthalen-1-ylidene)-N′-[6- morpholin-4-yl-2-(2- morpholin-4-yl-ethoxy)- pyrimidin-4-yl]-hydrazine |
| 803 | 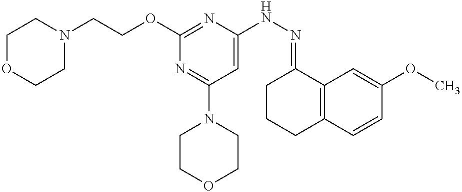 | N-(7-Methoxy-3,4-dihydro-2H- naphthalen-1-ylidene)-N′-[6- morpholin-4-yl-2-(2- morpholin-4-yl-ethoxy)- pyrimidin-4-yl]-hydrazine |
| 804 | 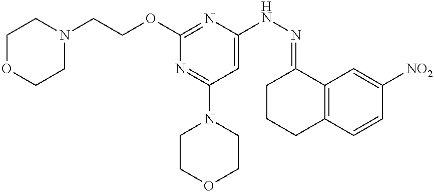 | N-(7-Nitro-3,4-dihydro-2H- naphthalen-1-ylidene)-N′-[6- morpholin-4-yl-2-(2- morpholin-4-yl-ethoxy)- pyrimidin-4-yl]-hydrazine |
| 805 | 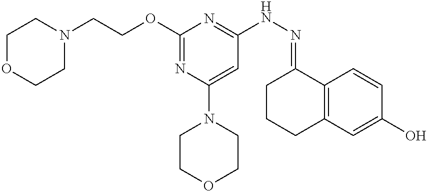 | N-(6-Hydroxy-3,4-dihydro-2H- naphthalen-1-ylidene)-N′-[6- morpholin-4-yl-2-(2- morpholin-4-yl-ethoxy)- pyrimidin-4-yl]-hydrazine |
| 806 | 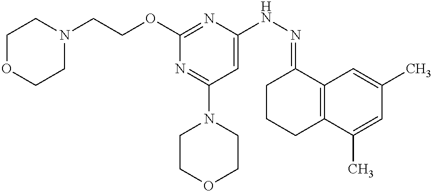 | N-(5,7-Dimethyl-3,4-dihydro- 2H-naphthalen-1-ylidene)-N′- [6-morpholin-4-yl-2-(2- morpholin-4-yl-ethoxy)- pyrimidin-4-yl]-hydrazine |
| 807 | 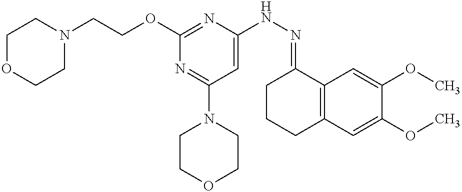 | N-(6,7-Dimethoxy-3,4- dihydro-2H-naphthalen-1- ylidene)-N′-[6-morpholin-4-yl- 2-(2-morpholin-4-yl-ethoxy)- pyrimidin-4-yl]-hydrazine |
| 808 | 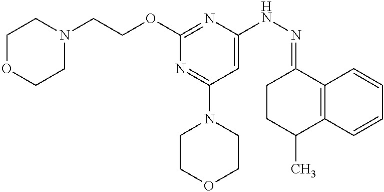 | N-(4-Methyl-3,4-dihydro-2H- naphthalen-1-ylidene)-N′-[6- morpholin-4-yl-2-(2- morpholin-4-yl-ethoxy)- pyrimidin-4-yl]-hydrazine |
| 809 | 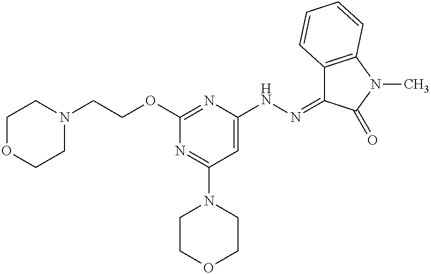 | 1-Methyl-3-{[6-morpholin-4- yl-2-(2-morpholin-4-yl- ethoxy)-pyrimidin-4-yl]- hydrazono}-1,3-dihydro-indol- 2-one |
| 810 | 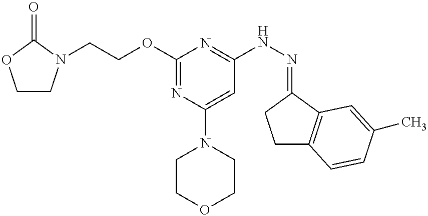 | 3-(2-{4-[N′-(6-Methyl-indan- 1-ylidene)-hydrazino]-6- morpholin-4-yl-pyrimidin-2- yloxy}-ethyl)-oxazolidin-2-one |
| 811 | 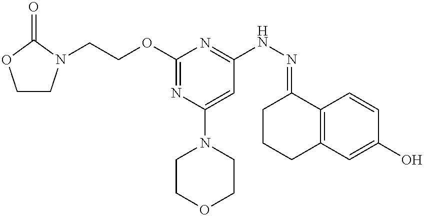 | 3-(2-{4-[N′-(6-Hydroxy-3,4- dihydro-2H-naphthalen-1- ylidene)-hydrazino]-6- morpholin-4-yl-pyrimidin-2- yloxy}-ethyl)-oxazolidin-2-one |
| 812 | 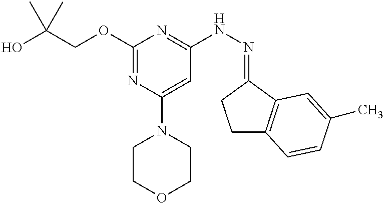 | 2-Methyl-1-{4-[N′-(6-methyl- indan-1-ylidene)-hydrazino]-6- morpholin-4-yl-pyrimidin-2- yloxy}-propan-2-ol |
| 813 | 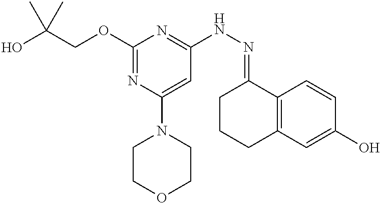 | 5-{[2-(2-Hydroxy-2-methyl- propoxy)-6-morpholin-4-yl- pyrimidin-4-yl]-hydrazono}- 5,6,7,8-tetrahydro-naphthalen- 2-ol |
| 814 | 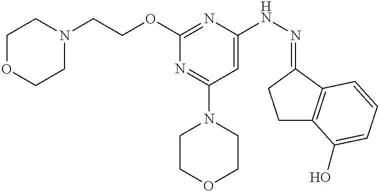 | N-(4-Hydroxy-indan-1- ylidene)-N′-[6-morpholin-4-yl- 2-(2-morpholin-4-yl-ethoxy)- pyrimidin-4-yl]-hydrazine |
| 815 | 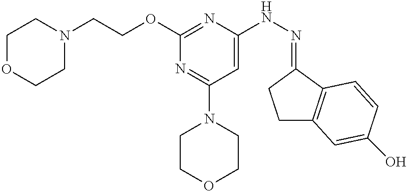 | N-(5-Hydroxy-indan-1- ylidene)-N′-[6-morpholin-4-yl- 2-(2-morpholin-4-yl-ethoxy)- pyrimidin-4-yl]-hydrazine |
| 816 | 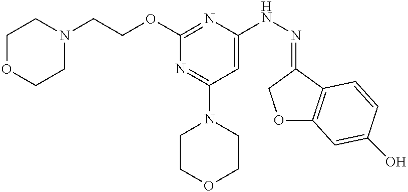 | 3-{[6-Morpholin-4-yl-2-(2- morpholin-4-yl-ethoxy)- pyrimidin-4-yl]-hydrazono}- 2,3-dihydro-benzofuran-6-ol |
| 817 | 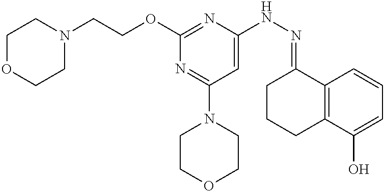 | N-(5-Hydroxy-3,4-dihydro- naphthalen-1-ylidene)-N′-[6- morpholin-4-yl-2-(2- morpholin-4-yl-ethoxy)- pyrimidin-4-yl]-hydrazine |
| 818 | 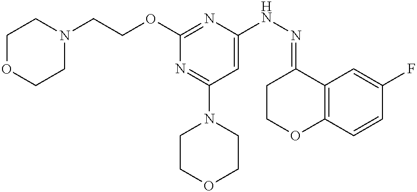 | N-(6-Fluoro-chroman-4- ylidene)-N′-[6-morpholin-4-yl- 2-(2-morpholin-4-yl-ethoxy)- pyrimidin-4-yl]-hydrazine |
| 819 | 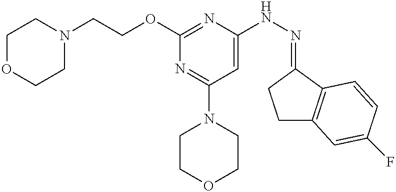 | N-(5-Fluoro-indan-1-ylidene)- N′-[6-morpholin-4-yl-2-(2- morpholin-4-yl-ethoxy)- pyrimidin-4-yl]-hydrazine |
| 820 | 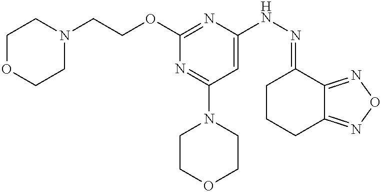 | N-(6,7-Dihydro-5H- benzo[1,2,5]oxadiazol-4- ylidene)-N′-[6-morpholin-4-yl- 2-(2-morpholin-4-yl-ethoxy)- pyrimidin-4-yl]-hydrazine |
| 821 | 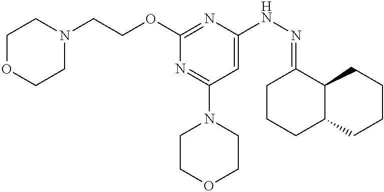 | N-[6-Morpholin-4-yl-2-(2- morpholin-4-yl-ethoxy)- pyrimidin-4-yl]-N′-(octahydro- naphthalen-1-ylidene)- hydrazine |
| 822 | 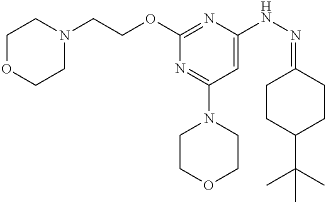 | N-(4-tert-Butyl- cyclohexylidene)-N′-[6- morpholin-4-yl-2-(2- morpholin-4-yl-ethoxy)- pyrimidin-4-yl]-hydrazine |
| 823 | 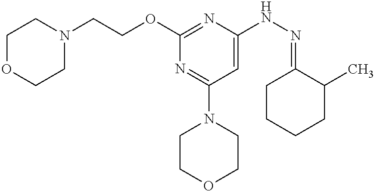 | N-(2-Methyl-cyclohexylidene)- N′-[6-morpholin-4-yl-2-(2- morpholin-4-yl-ethoxy)- pyrimidin-4-yl]-hydrazine |
| 824 | 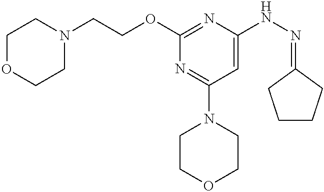 | N-Cyclopentylidene-N′-[6- morpholin-4-yl-2-(2- morpholin-4-yl-ethoxy)- pyrimidin-4-yl]-hydrazine |
| 825 | 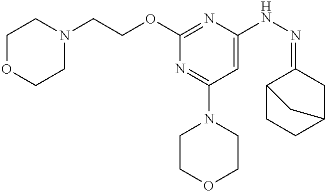 | N-Bicyclo[2.2.1]hept-2- ylidene-N′-[6-morpholin-4-yl- 2-(2-morpholin-4-yl-ethoxy)- pyrimidin-4-yl]-hydrazine |
| 826 | 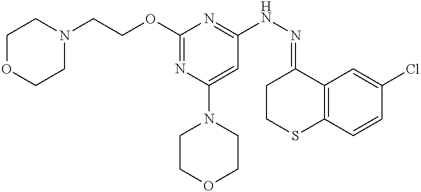 | N-(6-Chloro-thiochroman-4- ylidene)-N′-[6-morpholin-4-yl- 2-(2-morpholin-4-yl-ethoxy)- pyrimidin-4-yl]-hydrazine |
| 827 | 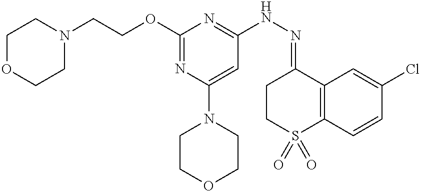 | N-(6-Chloro-1,1-dioxo-1λ⁶- thiochroman-4-ylidene)-N′-[6- morpholin-4-yl-2-(2- morpholin-4-yl-ethoxy)- pyrimidin-4-yl]-hydrazine |
| 828 | 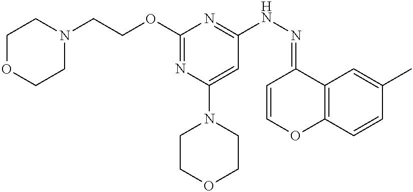 | N-(6-Methyl-chromen-4- ylidene)-N′-[6-morpholin-4-yl- 2-(2-morpholin-4-yl-ethoxy)- pyrimidin-4-yl]-hydrazine |
| 829 | 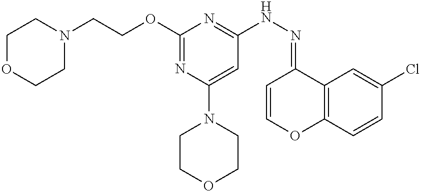 | N-(6-Chloro-chromen-4- ylidene)-N′-[6-morpholin-4-yl- 2-(2-morpholin-4-yl-ethoxy)- pyrimidin-4-yl]-hydrazine |

All of the features, specific embodiments and particular substituents disclosed herein may be combined in any combination. Each feature, embodiment or substituent disclosed in this specification may be replaced by an alternative feature, embodiment or substituent serving the same, equivalent, or similar purpose. In the case of chemical compounds, specific values can be combined in any combination resulting in a stable structure. Furthermore, specific values (whether preferred or not) for substituents in one type of chemical structure may be combined with values for other substituents (whether preferred or not) in the same or different type of chemical structure. Thus, unless expressly stated otherwise, each feature, embodiment or substituent disclosed is only an example of a generic series of equivalent or similar features feature, embodiments or substituents.

TABLE 2

| | | | Publication |
|---|---|---|---|
| Ser. No. | Filing Date | Publication No. | Date |
| U.S. Pat. No. 6,384,032 | Jun. 15, 2000 | | |
| U.S. Pat. No. 6,680,315 | Nov. 30, 2001 | | |
| U.S. Pat. No. 6,693,097 | Nov. 30, 2001 | | |
| U.S. Pat. No. 6,660,733 | Jul. 10, 2002 | | |
| U.S. Pat. No. 6,858,606 | Nov. 26, 2002 | | |
| U.S. Pat. No. 7,045,517 | Sep. 5, 2003 | | |
| U.S. Pat. No. 7,067,514 | Sep. 5, 2003 | | |
| U.S. Pat. No. 6,958,332 | Sep. 5, 2003 | | |
| U.S. Pat. No. 7,122,665 | Oct. 14, 2003 | | |
| U.S. Application No. | May 28, 2004 | 2008-0058297 | Mar. 6, 2008 |
| 10/561,025 | | | |
| U.S. Application No. | Jul. 1, 2005 | 2006-0063739 | Mar. 23, |
| 11/174,173 | | | 2006 |
| U.S. Pat. No. 7,338,951 | Nov. 10, 2004 | | |
| U.S. Application No. | Nov. 10, 2004 | 2005-0282809 | Dec. 22, |
| 10/985,716 | | | 2005 |
| U.S. Application No. | Nov. 10, 2004 | 2005-0250770 | Nov. 10, |
| 10/985,627 | | | 2005 |
| U.S. Application No. | Nov. 18, 2005 | 2006-0135518 | Jun. 22, |
| 11/282,926 | | | 2006 |
| U.S. Application No. | Nov. 10, 2005 | 2006-0122209 | Jun. 8, 2006 |
| 11/271,568 | | | |
| U.S. Application No. | Nov. 10, 2005 | 2006-0122156 | Jun. 8, 2006 |
| 11/271,704 | | | |
| U.S. Application No. | Nov. 10, 2005 | 2006-0233996 | Oct. 5, 2006 |
| 11/272,509 | | | |
| U.S. Pat. No. 7,470,685 | Jan. 21, 2005 | | |
| U.S. Application No. | Apr. 13, 2005 | 2005-0282802 | Dec. 22, |
| 11/105,818 | | | 2005 |
| U.S. Application No. | May 13, 2006 | 2006-0281711 | Dec. 14, |
| 11/433,603 | | | 2006 |
| U.S. Application No. | Oct. 27, 2006 | | |
| 12/110,317 | | | |

Methods for making the compounds of the invention have been disclosed in the U.S. patents and patent applications listed in Table 2. The entire teachings of these patents and patent applications are incorporated herein by reference.

As used herein, the term “alkyl” refers to a straight-chained or branched hydrocarbon group containing 1 to 12 carbon atoms. The term “lower alkyl” refers to a C1-C6 alkyl chain. Examples of alkyl groups include methyl, ethyl, n-propyl, isopropyl, tert-butyl, and n-pentyl. Alkyl groups may be optionally substituted with one or more substituents.

The term “alkenyl” refers to an unsaturated hydrocarbon chain that may be a straight chain or branched chain, containing 2 to 12 carbon atoms and at least one carbon-carbon double bond. Alkenyl groups may be optionally substituted with one or more substituents.

The term “alkynyl” refers to an unsaturated hydrocarbon chain that may be a straight chain or branched chain, containing the 2 to 12 carbon atoms and at least one carbon-carbon triple bond. Alkynyl groups may be optionally substituted with one or more substituents.

The sp² or sp carbons of an alkenyl group and an alkynyl group, respectively, may optionally be the point of attachment of the alkenyl or alkynyl groups.

The term “alkoxy,” as used herein, refers to an alkyl or a cycloalkyl group which is linked to another moiety though an oxygen atom. Alkoxy groups can be optionally substituted with one or more substituents.

The term “mercapto” refers to a —SH group.

The term “alkyl sulfanyl,” as used herein, refers to an alkyl or a cycloalkyl group which is linked to another moiety though a divalent sulfur atom. Alkyl sulfanyl groups can be optionally substituted with one or more substituents.

As used herein, the term “halogen” or “halo” means —F, —Cl, —Br or —I.

As used herein, the term “haloalkyl” means and alkyl group in which one or more (including all) the hydrogen radicals are replaced by a halo group, wherein each halo group is independently selected from —F, —Cl, —Br, and —I. The term “halomethyl” means a methyl in which one to three hydrogen radical(s) have been replaced by a halo group. Representative haloalkyl groups include trifluoromethyl, bromomethyl, 1,2-dichloroethyl, 4-iodobutyl, 2-fluoropentyl, and the like.

The term “cycloalkyl” refers to a hydrocarbon 3-8 membered monocyclic or 7-14 membered bicyclic ring system which is completely saturated ring. Cycloalkyl groups may be optionally substituted with one or more substituents. In one embodiment, 0, 1, 2, 3, or 4 atoms of each ring of a cycloalkyl group may be substituted by a substituent. Representative examples of cycloalkyl group include cyclopropyl, cyclopentyl, cyclohexyl, cyclobutyl, cycloheptyl, cyclooctyl, cyclononyl, cyclodecyl, and bicyclo[2.1.1]hexyl.

The term “cyclyl” refers to a hydrocarbon 3-8 membered monocyclic or 7-14 membered bicyclic ring system having at least one non-aromatic ring, wherein the non-aromatic ring has some degree of unsaturation. Cyclyl groups may be optionally substituted with one or more substituents. In one embodiment, 0, 1, 2, 3, or 4 atoms of each ring of a cyclyl group may be substituted by a substituent. Examples of cyclyl groups include cyclohexenyl, bicyclo[2.2.1]hept-2-enyl, dihydronaphthalenyl, benzocyclopentyl, cyclopentenyl, cyclopentadienyl, cyclohexenyl, cyclohexadienyl, cycloheptenyl, cycloheptadienyl, cycloheptatrienyl, cyclooctenyl, cyclooctadienyl, cyclooctatrienyl, cyclooctatetraenyl, cyclononenyl, cyclononadienyl, cyclodecenyl, cyclodecadienyl and the like.

The term “aryl” refers to a hydrocarbon monocyclic, bicyclic or tricyclic aromatic ring system. Aryl groups may be optionally substituted with one or more substituents. In one embodiment, 0, 1, 2, 3, 4, 5 or 6 atoms of each ring of an aryl group may be substituted by a substituent. Examples of aryl groups include phenyl, naphthyl, anthracenyl, fluorenyl, indenyl, azulenyl, and the like.

As used herein, the term “aralkyl” means an aryl group that is attached to another group by a (C₁-C₆)alkylene group. Aralkyl groups may be optionally substituted, either on the aryl portion of the aralkyl group or on the alkylene portion of the aralkyl group, with one or more substituent. Representative aralkyl groups include benzyl, 2-phenyl-ethyl, naphth-3-yl-methyl and the like.

As used herein, the term “alkylene” refers to an alkyl group that has two points of attachment. The term “(C₁-C₆)alkylene” refers to an alkylene group that has from one to six carbon atoms. Non-limiting examples of alkylene groups include methylene (—CH₂—), ethylene (—CH₂CH₂—), n-propylene (—CH₂CH₂CH₂—), isopropylene (—CH₂CH(CH₃)—), and the like. Alkylene groups may be optionally substituted.

As used herein, the term “cycloalkylene” refers to a cycloalkyl group that has two points of attachment. Cycloalkylene groups may be optionally substituted.

As used herein, the term “cyclylene” refers to a cyclyl group that has two points of attachment. Cyclylene groups may be optionally substituted.

As used herein, the term “arylene” refers to an aryl group that has two points of attachment. Arylene groups may be optionally substituted.

As used herein, the term “aralkylene” refers to an aralkyl group that has two points of attachment. Aralkylene groups may be optionally substituted.

The term “arylalkoxy” refers to an alkoxy substituted with an aryl.

The term “heteroaryl” refers to an aromatic 5-8 membered monocyclic, 8-12 membered bicyclic, or 11-14 membered tricyclic ring system having 1-4 ring heteroatoms if monocyclic, 1-6 heteroatoms if bicyclic, or 1-9 heteroatoms if tricyclic, said heteroatoms selected from O, N, or S, and the remainder ring atoms being carbon. Heteroaryl groups may be optionally substituted with one or more substituents. In one embodiment, 0, 1, 2, 3, or 4 atoms of each ring of a heteroaryl group may be substituted by a substituent. Examples of heteroaryl groups include pyridyl, 1-oxo-pyridyl, furanyl, benzo[1,3]dioxolyl, benzo[1,4]dioxinyl, thienyl, pyrrolyl, oxazolyl, oxadiazolyl, imidazolyl thiazolyl, isoxazolyl, quinolinyl, pyrazolyl, isothiazolyl, pyridazinyl, pyrimidinyl, pyrazinyl, triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, indolyl, tetrahydroindolyl, azaindolyl, imidazopyridyl, quinazolinyl, purinyl, pyrrolo[2,3]pyrimidinyl, pyrazolo[3,4]pyrimidinyl, and benzo(b)thienyl, 3H-thiazolo[2,3-c][1,2,4]thiadiazolyl, imidazo[1,2-d]-1,2,4-thiadiazolyl, imidazo[2,1-b]-1,3,4-thiadiazolyl, 1H,2H-furo[3,4-d]-1,2,3-thiadiazolyl, 1H-pyrazolo[5,1-c]-1,2,4-triazolyl, pyrrolo[3,4-d]-1,2,3-triazolyl, cyclopentatriazolyl, 3H-pyrrolo[3,4-c]isoxazolyl, 1H,3H-pyrrolo[1,2-c]oxazolyl, pyrrolo[2,1b]oxazolyl, and the like.

As used herein, the term “heteroaralkyl” or “heteroarylalkyl” means a heteroaryl group that is attached to another group by a (C₁-C₆)alkylene. Heteroaralkyl groups may be optionally substituted, either on the heteroaryl portion of the heteroaralkyl group or on the alkylene portion of the heteroaralkyl group, with one or more substituent. Representative heteroaralkyl groups include 2-(pyridin-4-yl)-propyl, 2-(thien-3-yl)-ethyl, imidazol-4-yl-methyl and the like.

As used herein, the term “heteroarylene” refers to a heteroaryl group that has two points of attachment. Heteroarylene groups may be optionally substituted.

As used herein, the term “heteroaralkylene” refers to a heteroaralkyl group that has two points of attachment. Heteroaralkylene groups may be optionally substituted.

The term “heterocycloalkyl” refers to a nonaromatic, completely saturated 3-8 membered monocyclic, 7-12 membered bicyclic, or 10-14 membered tricyclic ring system comprising 1-3 heteroatoms if monocyclic, 1-6 heteroatoms if bicyclic, or 1-9 heteroatoms if tricyclic, said heteroatoms selected from O, N, S, B, P or Si. Heterocycloalkyl groups may be optionally substituted with one or more substituents. In one embodiment, 0, 1, 2, 3, or 4 atoms of each ring of a heterocycloalkyl group may be substituted by a substituent. Representative heterocycloalkyl groups include piperidinyl, piperazinyl, 2-oxopiperazinyl, 2-oxopiperidinyl, 2-oxopyrrolidinyl, 4-piperidonyl, tetrahydropyranyl, tetrahydrothiopyranyl, tetrahydrothiopyranyl sulfone, morpholinyl, thiomorpholinyl, thiomorpholinyl sulfoxide, thiomorpholinyl sulfone, 1,3-dioxolane, tetrahydrofuranyl, tetrahydrothienyl, an thiirene.

The term “heterocyclyl” refers to a nonaromatic 5-8 membered monocyclic, 7-12 membered bicyclic, or 10-14 membered tricyclic ring system comprising 1-3 heteroatoms if monocyclic, 1-6 heteroatoms if bicyclic, or 1-9 heteroatoms if tricyclic, said heteroatoms selected from O, N, S, B, P or Si, wherein the nonaromatic ring system has some degree of unsaturation. Heterocyclyl groups may be optionally substituted with one or more substituents. In one embodiment, 0, 1, 2, 3, or 4 atoms of each ring of a heterocyclyl group may be substituted by a substituent. Examples of these groups include thiirenyl, thiadiazirinyl, dioxazolyl, 1,3-oxathiolyl, 1,3-dioxolyl, 1,3-dithiolyl, oxathiazinyl, dioxazinyl, dithiazinyl, oxadiazinyl, thiadiazinyl, oxazinyl, thiazinyl, 1,4-oxathiin,1,4-dioxin, 1,4-dithiin, 1H-pyranyl, oxathiepinyl, 5H-1,4-dioxepinyl, 5H-1,4-dithiepinyl, 6H-isoxazolo[2,3-d]1,2,4-oxadiazolyl, 7H-oxazolo[3,2-d]1,2,4-oxadiazolyl, and the like.

As used herein, the term “heterocycloalkylene” refers to a heterocycloalkyl group that has two points of attachment. Heterocycloalkylene groups may be optionally substituted.

As used herein, the term “heterocyclylene” refers to a heterocyclyl group that has two points of attachment. Heterocyclylene groups may be optionally substituted.

When a cycloalkyl, cyclyl, heterocycloalkyl, or heterocyclyl is fused to another ring (e.g., a cycloalkyl, cyclyl, heterocycloalkyl, heterocyclyl, aryl, heteroaryl), it shares two or more ring atoms, preferably two to four ring atoms, with the other ring.

The term “amino” refers to —NH₂. The term “alkylamino” refers to an amino in which one hydrogen is replaced by an alkyl group. The term “dialkylamino” refers to an amino in which each of the hydrogens is replaced by an independently selected alkyl group. The term “aminoalkyl” refers to an alkyl substituent which is further substituted with one or more amino groups.

The term “mercaptoalkyl” refers to an alkyl substituent which is further substituted with one or more mercapto groups.

The term “hydroxyalkyl” or “hydroxylalkyl” refers to an alkyl substituent which is further substituted with one or more hydroxy groups.

The term “sulfonylalkyl” refers to an alkyl substituent which is further substituted with one or more sulfonyl groups.

The term “sulfonylaryl” refers to an aryl substituent which is further substituted with one or more sulfonyl groups.

The term alkylcarbonyl refers to an —C(O)-alkyl.

The term “mercaptoalkoxy” refers to an alkoxy substituent which is further substituted with one or more mercapto groups.

The term “alkylcarbonylalkyl” refers to an alkyl substituent which is further substituted with —C(O)-alkyl. The alkyl or aryl portion of alkylamino, aminoalkyl, mercaptoalkyl, hydroxyalkyl, mercaptoalkoxy, sulfonylalkyl, sulfonylaryl, alkylcarbonyl, and alkylcarbonylalkyl may be optionally substituted with one or more substituents.

Suitable substituents for an alkyl, alkoxy, alkyl sulfanyl, alkylamino, dialkylamino, alkylene, alkenyl, alkynyl, cycloalkyl, cyclyl, heterocycloalkyl, heterocyclyl, aryl, aralkyl, heteroaryl, heteroaralkyl, cycloalkylene, cyclylene, heterocycloalkylene, heterocyclylene, arylene, aralkylene, heteroalkylene and heteroaryalkylene groups include any substituent which will form a stable compound of the invention. Examples of substituents for an alkyl, alkoxy, alkylsulfanyl, alkylamino, dialkylamino, alkylene, alkenyl, alkynyl, cycloalkyl, cyclyl, heterocycloalkyl, heterocyclyl, aryl, aralkyl, heteroaryl, heteroaralkyl, cycloalkylene, cyclylene, heterocycloalkylene, heterocyclylene, arylene, aralkylene, heteroalkylene and heteroaryalkylene include an optionally substituted alkyl, an optionally substituted alkoxy, an optionally substituted alkyl sulfanyl, an optionally substituted alkenyl, an optionally substituted alkynyl, an optionally substituted cycloalkyl, an optionally substituted cyclyl, an optionally substituted heterocyclyl, an optionally substituted heterocycloalkyl, an optionally substituted aryl, an optionally substituted heteroaryl, an optionally substituted aralkyl, an optionally substituted heteroaralkyl, a haloalkyl, halo, cyano, nitro, haloalkoxy, ═O, ═S, ═NR, —OR^{k}, —NR^{h}R^{j}, —SR^{k}, —C(O)R^{k}, —C(O)NR^{h}R^{j}, —NR^{k}C(O)R^{k}, —C(O)OR^{k}, —OC(O)R^{k}, —NR^{k}C(O)NR^{h}R^{j}, —OC(O)NR^{h}R^{j}, —NR^{k}C(O)OR^{k}, —C(NR)R^{k}, —C(NR)NR^{h}R^{j}, —NR^{k}C(NR)R^{k}, —C(NR)OR^{k}, —OC(NR)R^{k}, —NR^{k}C(NR)NR^{h}R^{j}, —OC(NR)NR^{h}R^{j}, —NR^{k}C(NR)OR^{k}, —C(S)R^{k}, —C(S)NR^{h}R^{j}, —NR^{k}C(S)R^{k}, —C(S)OR^{k}, —OC(S)R^{k}, —NR^{k}C(S)NR^{h}R^{j}, —OC(S)NR^{h}R^{j}, —NR^{k}C(S)OR^{k}, —C(O)SR^{k}, —SC(O)R^{k}, —S(O)ₚR^{k}, —S(O)ₚNR^{h}R^{j}, —OS(O)ₚR^{k}, —S(O)ₚOR^{k}, —OS(O)ₚOR^{k}, —P(O)(OR^{k})₂, —OP(O)(OR^{k})₂, —P(S)(OR^{k})₂, —SP(O)(OR^{k})₂, —P(O)(SR^{k})(OR^{k}), —OP(O)(SR^{k})(OR^{k}), —P(O)(SR^{k})₂, or —OP(O)(SR^{k})₂, wherein p is 1 or 2.

In addition, alkyl, cycloalkyl, alkylene, a heterocycloalkyl, a and any saturated portion of a alkenyl, a cyclyl, alkynyl, heterocyclyl, aralkyl, and heteroaralkyl groups, may also be substituted with ═O, ═S, or ═NR.

When a heterocyclyl, heteroaryl, or heteroaralkyl group contains a nitrogen atom, it may be substituted or unsubstituted. When a nitrogen atom in the aromatic ring of a heteroaryl group has a substituent the nitrogen may be a quaternary nitrogen.

Choices and combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject). Typically, such compounds are stable at a temperature of 40° C. or less, in the absence of excessive moisture, for at least one week. Such choices and combinations will be apparent to those of ordinary skill in the art and may be determined without undue experimentation.

As used herein, the term “lower” refers to a group having up to six atoms. For example, a “lower alkyl” refers to an alkyl radical having from 1 to 6 carbon atoms, and a “lower alkenyl” or “lower alkynyl” refers to an alkenyl or alkynyl radical having from 2 to 6 carbon atoms, respectively. A “lower alkoxy” or “lower alkyl sulfanyl” group refers to an alkoxy or alkyl sulfanyl group that has from 1 to 6 carbon atoms.

The compounds of the invention are defined herein by their chemical structures and/or chemical names. Where a compound is referred to by both a chemical structure and a chemical name, and the chemical structure and chemical name conflict, the chemical structure is determinative of the compound's identity.

The compounds of this invention include the compounds themselves, as well as their salts, solvate, clathrate, hydrate, polymorph, or prodrugs. As used herein, the term “pharmaceutically acceptable salt,” is a salt formed from, for example, an acid and a basic group of a compound of any one of the formulae disclosed herein. Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, besylate, gentisinate, fumarate, gluconate, glucaronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. The term “pharmaceutically acceptable salt” also refers to a salt prepared from a compound of any one of the formulae disclosed herein having an acidic functional group, such as a carboxylic acid functional group, and a pharmaceutically acceptable inorganic or organic base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl,N-ethylamine; diethylamine; triethylamine; mono-, bis-, or tris-(2-hydroxy-lower alkyl amines), such as mono-, bis-, or tris-(2-hydroxyethyl)amine, 2-hydroxy-tert-butylamine, or tris-(hydroxymethyl)methylamine, N,N,-di-lower alkyl-N-(hydroxy lower alkyl)-amines, such as N,N-dimethyl-N-(2-hydroxyethyl)amine, or tri-(2-hydroxyethyl)amine; N-methyl-D-glucamine; and amino acids such as arginine, lysine, and the like. The term “pharmaceutically acceptable salt” also refers to a salt prepared from a compound of any one of the formulae disclosed herein having a basic functional group, such as an amino functional group, and a pharmaceutically acceptable inorganic or organic acid. Suitable acids include hydrogen sulfate, citric acid, acetic acid, oxalic acid, hydrochloric acid (HCl), hydrogen bromide (HBr), hydrogen iodide (HI), nitric acid, hydrogen bisulfide, phosphoric acid, lactic acid, salicylic acid, tartaric acid, bitartratic acid, ascorbic acid, succinic acid, maleic acid, besylic acid, fumaric acid, gluconic acid, glucaronic acid, formic acid, benzoic acid, glutamic acid, methanesulfonic acid, ethanesulfonic acid, benzenesulfonic acid, and p-toluenesulfonic acid.

As used herein, the term “polymorph” means solid crystalline forms of a compound of the present invention or complex thereof. Different polymorphs of the same compound can exhibit different physical, chemical and/or spectroscopic properties. Different physical properties include, but are not limited to stability (e.g., to heat or light), compressibility and density (important in formulation and product manufacturing), and dissolution rates (which can affect bioavailability). Differences in stability can result from changes in chemical reactivity (e.g., differential oxidation, such that a dosage form discolors more rapidly when comprised of one polymorph than when comprised of another polymorph) or mechanical characteristics (e.g., tablets crumble on storage as a kinetically favored polymorph converts to thermodynamically more stable polymorph) or both (e.g., tablets of one polymorph are more susceptible to breakdown at high humidity). Different physical properties of polymorphs can affect their processing. For example, one polymorph might be more likely to form solvates or might be more difficult to filter or wash free of impurities than another due to, for example, the shape or size distribution of particles of it.

As used herein, the term “hydrate” means a compound of the present invention or a salt thereof, which further includes a stoichiometric or non-stoichiometric amount of water bound by non-covalent intermolecular forces.

As used herein, the term “clathrate” means a compound of the present invention or a salt thereof in the form of a crystal lattice that contains spaces (e.g., channels) that have a guest molecule (e.g., a solvent or water) trapped within.

As used herein and unless otherwise indicated, the term “prodrug” means a derivative of a compound that can hydrolyze, oxidize, or otherwise react under biological conditions (in vitro or in vivo) to provide a compound of this invention. Prodrugs may only become active upon such reaction under biological conditions, or they may have activity in their unreacted forms. Examples of prodrugs contemplated in this invention include, but are not limited to, analogs or derivatives of compounds of any one of the formulae disclosed herein that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of compounds of any one of the formulae disclosed herein that comprise —NO, —NO₂, —ONO, or —ONO₂ moieties. Prodrugs can typically be prepared using well-known methods, such as those described by 1 BURGER'S MEDICINAL CHEMISTRY AND DRUG DISCOVERY (1995) 172-178, 949-982 (Manfred E. Wolff ed., 5^{th} ed).

As used herein and unless otherwise indicated, the terms “biohydrolyzable amide”, “biohydrolyzable ester”, “biohydrolyzable carbamate”, “biohydrolyzable carbonate”, “biohydrolyzable ureide” and “biohydrolyzable phosphate analogue” mean an amide, ester, carbamate, carbonate, ureide, or phosphate analogue, respectively, that either: 1) does not destroy the biological activity of the compound and confers upon that compound advantageous properties in vivo, such as uptake, duration of action, or onset of action; or 2) is itself biologically inactive but is converted in vivo to a biologically active compound. Examples of biohydrolyzable amides include, but are not limited to, lower alkyl amides, α-amino acid amides, alkoxyacyl amides, and alkylaminoalkylcarbonyl amides. Examples of biohydrolyzable esters include, but are not limited to, lower alkyl esters, alkoxyacyloxy esters, alkyl acylamino alkyl esters, and choline esters. Examples of biohydrolyzable carbamates include, but are not limited to, lower alkylamines, substituted ethylenediamines, aminoacids, hydroxyalkylamines, heterocyclic and heteroaromatic amines, and polyether amines.

In addition, some of the compounds of this invention have one or more double bonds, or one or more asymmetric centers. Such compounds can occur as racemates, racemic mixtures, single enantiomers, individual diastereomers, diastereomeric mixtures, and cis- or trans- or E- or Z-double isomeric forms. All such isomeric forms of these compounds are expressly included in the present invention. The compounds of this invention may also be represented in multiple tautomeric forms, in such instances, the invention expressly includes all tautomeric forms of the compounds described herein (e.g., alkylation of a ring system may result in alkylation at multiple sites, the invention expressly includes all such reaction products). All such isomeric forms of such compounds are expressly included in the present invention. All crystal forms of the compounds described herein are expressly included in the present invention.

Further, the aforementioned compounds also include their N-oxides. The term “N-oxides” refers to one or more nitrogen atoms, when present in a heterocyclic or heteroaryl compound, are in N-oxide form, i.e., N→O. For example, in compounds of any one of the formula d or Table 1 when one of Q, U, or V is N, also included are compounds in which Q, U, or V, respectively, is N→O.

As used herein, the term “pharmaceutically acceptable solvate,” is a solvate formed from the association of one or more solvent molecules to one of the compounds of any of the formulae disclosed herein. The term solvate includes hydrates (e.g., hemi-hydrate, mono-hydrate, dihydrate, trihydrate, tetrahydrate, and the like).

The method can also include the step of identifying that the subject is in need of treatment for psoriasis. The identification can be in the judgment of a subject or a health professional and can be subjective (e.g., opinion) or objective (e.g., measurable by a test or a diagnostic method).

As noted above, one embodiment of the present invention is directed to treating subjects with psoriasis. “Treating a subject with a psoriasis” includes achieving, partially or substantially, one or more of the following: reduction in the size of psoriatic skin lesions (e.g., psoriatic plaques), reduction in the skin surface area covered by psoriatic skin lesions, reduction in the number of psoriatic skin lesions, and ameliorating or improving a clinical symptom or indicator associated with psoriasis (such as reducing the thickness of plaques, lengthening the epidermal cell cycle, reducing the number of monocytes, T cells, B cells and/or dendritic cells found in psoriatic skin lesions, or reducing the amount of IL-12 and/or IL-23 found in skin lesions). “Treating a subject with a psoriasis” can also include achieving, partially or substantially, improvement in the appearance of a patients psoriatic skin lesions (e.g., reduction in the exfoliation of skin lesions or skin lesions appear less red).

An “effective amount” is the quantity of compound in which a beneficial clinical outcome is achieved when the compound is administered to a subject with psoriasis. A “beneficial clinical outcome” includes a reduction in size of psoriatic skin lesions, reduction in the number of psoriatic skin lesions, reduction in the thickness of psoriatic plaques, reduction in the total skin area covered by psoriatic skin lesions, reduction in lymphocyte (e.g., monocytes, T cells, B cells and/or dendritic cells) infiltration of psoriatic skin lesion.

Pharmaceutical compositions for topical administration include one or more compounds disclosed herein and a pharmaceutically acceptable topical carrier. In general, compositions of the present invention contain about 0.005% to about 5% by weight of a compound of the invention; more typically, the compostions of the invention contain about 0.01% to about 2% of a compound of the invention; even more typically, the compostions of the invention contain about 0.05% to about 1% of a compound of the invention.

Compositions of the invention for topical administration can be in the form of a solution, spray, lotion, cream, gel or ointment. The preferred form of the composition depends upon the condition being treated and the desired therapeutic effect. For example, treatment of a moist, acutely inflamed rash (such as found in pustular psoriasis) is preferably treated with a lotion, whereas treatment of a chronic dry patch (such as found in psoriasis vulgaris) is often treated more effectively with a cream or ointment.

A pharmaceutically acceptable topical carrier may include any topical carrier known in the art that is compatable the compounds of the invention (e.g., the compositions are readily applied topically and are stable for a reasonable period of time, such as 1 week or more). Typically, pharmaceutically acceptable topical carriers include an organic component, such as an alcohol, ester, or amide, and water. In some embodiments, the compositions of the invention may optionally contain one or more penetration enhancer, opacifier, viscosity enhancer or humectant. Other ingredients that may be included in the compositions of the invention include, for example, one or more anti-itch agents; anti-foaming agents, buffers, neutralizing agents, and agents to adjust pH, coloring agents and decoloring agents, emollients, emulsifying agents, emulsion stabilizers, odorants (e.g., perfume or menthol), preservatives, antioxidants, chemical stabilizers, solvents, thickening, stiffening, and suspending agents.

An ointment may comprise a simple base of animal or vegetable oils or semi-solid hydrocarbons (oleaginous). Ointments may also comprise absorption ointment bases which absorb water to form emulsions. Ointment carriers may also be water soluble.

A gel is a semisolid emulsion that liquefies when applied to the skin. A gel is a composition that is relatively non-flowing at ambient temperature (about 25° C.). The term “gel” is intended to include semi-solid permutations gelled with high molecular weight polymers, e.g., carboxypolymethylene (Carbomer BP) or methylcellulose, and can be regarded as semi-plastic aqueous lotions. They are typically non-greasy, water miscible, easy to apply and wash off, and are especially suitable for treating hairy parts of the body.

A cream is a semisolid oil-in-water emulsion or water-in-oil emulsion. Oil in water creams are water miscible and are well absorbed into the skin. Water in oil (oily) creams are immiscible with water and, therefore, more difficult to remove from the skin.

The term “lotion” is art recognized and is intended to include those solutions typically used in dermatological applications. The lotions of the present invention may include clear solutions, as well as liquid suspensions and dispersions. Solid-in-liquid suspensions are preparations of finely divided, undissolved drugs or other particulate matter dispersed in liquid vehicles. These suspensions require shaking before application to ensure uniform distribution of solid in the vehicle. Liquid-in-liquid dispersions generally contain a higher water content than cream emulsions and are pourable. Lotions provide a protective, drying, and cooling effect and may act as a vehicle for other agents. The addition of alcohol increases the cooling effect. If an astringent, such as aluminum is present, it will precipitate protein and dry and seal exudating surfaces. Typically, a lotion contains at least about 15% by weight water, more preferably at least about 20%, still more preferably at least about 30%, and still more preferably about 40% to about 60% by weight water but no emulsifier.

Typically, the amount of water employed in the compositions of the invention is that which is effective to form an emulsion. It is generally preferred to use water which has been purified by processes such as deionization or reverse osmosis, to improve the batch-to-batch formulation inconsistencies which can be caused by dissolved solids in the water supply. The amount of water in emulsions or other compositions of the invention can range from about 5 to 95 weight percent, preferably from about 15 to 85 percent, more preferably in the range of about 45 to about 75 percent.

The organic component of a pharmaceutically acceptable topical carrier is typically a pharmaceutically acceptable alcohol, ester or amide. Typical alcohols that can be used in the compositions of the invention include isopropy alcohol, propylene glycol, ethanol, ethylene glycol, polyethylene glycol, glycerol, octanol, benzyl alcohol, sorbitol, and mannitol. Typical esters that can be used in the compostions of the invention include isopropyl myristate and esters of polyethylene glycol, such as polyethylene glycol monolaurate. Typical amides include N,N-dimethylamide, N-methyl-2-pyrrolidone and polyvinyl-pyrrolidone, urea, dimethylacetamide (DMA), 2-pyrrolidone, 1-methyl-2-pyrrolidone. The organic component may also be dimethylsulfoxide, ethanolamine, diethanolamine and triethanolamine.

In some embodiments, viscosity enhancers and/or emulsion stabilizers may be included in the compositions of the invention to provide a desirable viscosity and/or consistency for topical administration. Exemplary emulsion stabilizers and viscosity enhancers include carbomer 934, carbomer 934P, carbomer 940, cetearyl alcohol, cetostearyl alcohol, cetyl alcohol, cetyl stearyl alcohol, dextrin, diglycerides, disodium edetate, edetate disodium, glycerides, glyceryl monostearate, glyceryl stearate, hydroxypropyl cellulose, monoglycerides, plasticized hydrocarbon gel, polyethylene glycol 300, polyethylene glycol 400, polyethylene glycol 1450, polyethylene glycol 8000, polyethylene glycols, propylene glycol stearate and stearyl alcohol.

In some embodiments, one or more penetration enhancers may be included in the compositions of the invention. The addition of unsaturated fatty acids such as oleic acid or the use of chemicals such as AZONEs has been reported to enhance percutaneous penetration.

Many of the ingredients described herein are also known in the art “excipients.” Examples of excipients useful in the formulation of the present invention include, without being limited by, those in Table 2.

TABLE II
Excipients

| Ingredient | Characteristics | Vendor/Trade Name |
|---|---|---|
| Benzyl Alcohol | Alpha-hydroxytoluene | Various |
| Glyceryl Monooleate | Mixture of the glycerides of oleic acid and | Various |
| | other fatty acids | |
| Hydroxypropyl | C₅₆H₁₀₃O₃₀ (emulsifier) | Aqualon |
| Methylcellulose | | |
| Polyethylene Glycol 300, | Super refined polyethylene glycol polymer of | DOW |
| 400, 4000 | the general formula | |
| | HOCH₂(CH₂OCH₂)ₘCH₂OH, where m = | |
| | Average molecular weight. | |
| Polysorbate 80 | Super refined polyoxyethylene 20 sorbitan | Croda |
| | monooleate. Partial fatty acid ester of Sorbitol | |
| | and its anhydride copolymerized with 20 | |
| | moles of ethylene oxide for each mole of | |
| | Sorbitol and its anhydride. | |
| Poloxamer 127, 407 | Series of closely related block copolymers of | BASF |
| | ethylene oxide and propylene oxide | |
| | conforming to the general formula | |
| | HO(C₂H₄O)ₐ(C₃H₆O)_{b}(C₂H₄O)ₐH. | |
| Cetostearyl alcohol | Mixture of solid aliphatic alcohols consisting | Various |
| | mainly of stearyl (C₁₈H₃₈O) and cetyl | |
| | (C₁₆H₃₄O) alcohols. | |
| Mineral Oil | Mixture of refined liquid saturated aliphatic | Various |
| | (C₁₄-C₁₈) and cyclic hydrocarbons obtained | |
| | from petroleum. | |
| Propylene Glycol | C₃H₈O₂ (solvent, extractant, and preservative). | Various |
| Cremophor A6: | Non-ionic emulsifier produced by reacting | BASF |
| Ceteareth-6 (&) Stearyl | higher saturated fatty alcohols with ethylene | |
| Alcohol | oxide. Also contains free fatty alcohol. | |
| *Cremophor A25: | Non-ionic emulsifier produced by reacting | BASF |
| Ceteareth-25 | higher saturated fatty alcohols with ethylene | |
| | oxide. | |
| Cremophor RH 40 | Material obtained by reaction of ethylene | BASF |
| | oxide with castor oil. Hydrophobic | |
| | constituents 83%, Hydrophilic part 17%. | |
| Transcutol | Diethylene glycol monoethyl ether | Gattefosse |
| | (solubilizer/solvent). | |
| Povidone | Synthetic polymer, consisting essentially of | ISP |
| | linear 1-vinyl-2-pyrrolidinone groups, the | |
| | differing degree of polymerization of which | |
| | results in polymers of various molecular | |
| | weights. | |
| Isopropyl alcohol | C₃H₈O (disinfectant, solvent). | Spectrum |
| Ethanol, Undenatured | C₂H₆O (antimicrobial preservative, | Spectrum |
| | disinfectant, skin penetrant, solvent). | |
| Carbomer 980 | Synthetic high-molecular weight polymers of | Lubrizol |
| | acrylic acid that are crosslinked with either | |
| | allyl sucrose or allyl ethers of pentaerythritol. | |
| Sorbitan Monooleate | C₂₄H₄₄O₆ (emulsifying agent, nonionic | Various |
| | surfactant, solubilizing agent, wetting and | |
| | disbursing/suspending agent). | |
| Eumulgin B1 | Polyethylene glycol ether of cetearyl alcohol, | Cognis |
| | is a non-ionic emulsifier | |
| Eumulgade 1000 | cetearyl alcohol and ceteareth-20 | Cognis |
| HEC | Partially substituted poly (hydroxyethyl) ether | Aqualon |
| | of cellulose. | |
| Isopropyl Myristate | C₁₇H₃₄O₂ (emollient, oleaginous vehicle, skin | Protameen |
| | penetrant, solvent). | |
| Dibutyl Adipate | C₈H₁₆O₂ (emollient, oleaginous vehicle, skin | Protameen |
| | penetrant, solvent). | |
| Miglyol 812 | Mixture of triglycerides of saturated fatty | Sasol |
| | acids. | |
| PEG-6 Caprilic/Capric | Polyglycolized medium chain triglyceride | Gattefosse (Labrasol) |
| Glyceride (Labrasol) | derived from coconut oil. HLB ~14. | |
| Titanium Dioxide | TiO₂ (coating agent, opacifier, pigment). | Spectrum |
| Methyparaben | C₈H₈O₃ (antimicrobial preservative). | Spectrum |
| Propylparaben | C₁₀H₁₂O₃ (antimicrobial preservative). | Spectrum |
| BHA | C₁₁H₁₆O₂ (antioxidant). | Spectrum |
| BHT | C₁₅H₂₄O (antioxidant). | Spectrum |
| Ascorbyl Palmitate | C₂₂H₃₈O₇ (antioxidant). | BASF |
| Benzophenone-4 | C₁₄H₁₂O₃ | BASF |
| Uvinul A Plus | Diethylamino Hydroxybenzoyl Hexyl | BASF |
| | Benzoate | |
| Uvinul M40 | Benzophenone-4, C₁₄H₁₂O₃ | BASF |
| T-Lite | TiO₂ | BASF |
| Z-Cote HP-1 | Zinc Oxide, ZnO | BASF |
| Z-Cote Max | Zinc Oxide, ZnO | BASF |

Propylene glycol has been shown to enhance percutaneous absorption and may be added at a variable proportion up to 70% by weight. Proportions of 0-50% by weight are suitable for preparations that are required to have low absorption, while proportions of 50-70% by weight are suitable for preparations that are required to have high absorption.

Examples of other suitable enhancers include, but are not limited to, ethers such as diethylene glycol monoethyl ether (available commercially as Transcutol™) and diethylene glycol monomethyl ether; surfactants such as sodium laurate, sodium lauryl sulfate, cetyltrimethylammonium bromide, benzalkonium chloride, Poloxamer (231, 182, 184), Tween (20, 40, 60, 80), and lecithin (U.S. Pat. No. 4,783,450, the entire teachings of which are incorporated herein by reference); alcohols such as ethanol, propanol, octanol, benzyl alcohol, and the like; polyethylene glycol and esters thereof such as polyethylene glycol monolaurate (PEGML; see, e.g., U.S. Pat. No. 4,568,343, the entire teachings of which are incorporated herein by reference); amides and other nitrogenous compounds such as urea, dimethylacetamide (DMA), dimethylformamide (DMF), 2-pyrrolidone, 1-methyl-2-pyrrolidone, ethanolamine, diethanolamine and triethanolamine; terpenes; alkanones; and organic acids, particularly citric acid and succinic acid. Sulfoxides such as dimethyl sulfoxides (DMSO) may also be used.

Lipophilic penetration enhancers typically referred to as “plasticizing” enhancers may also be used in the compositions of the invention. Typically, lipophilic penetration enhancers have a molecular weight in the range of about 150 to 1000, an aqueous solubility of less than about 1 wt. %, preferably less than about 0.5 wt. %, and most preferably less than about 0.2 wt. %. The Hildebrand solubility parameter a of plasticizing enhancers is in the range of about 2.5 to about 10, preferably in the range of about 5 to about 10. Such enhancers are described in International Patent Application No. PCT/US00/34483, published Jun. 21, 2001 as WO 01/43775 A2. In some embodiments, lipophilic enhancers such as fatty esters, fatty alcohols, and fatty ethers are preferred. Examples of fatty acid esters include methyl laurate, ethyl oleate, propylene glycol monolaurate, propylene glycerol dilaurate, glycerol monolaurate, glycerol monooleate, isopropyl n-decanoate, and octyldodecyl myristate. Examples of fatty alcohols include stearyl alcohol and oleyl alcohol, while fatty ethers include compounds wherein a diol or triol, preferably a C₂-C₄ alkane diol or triol, are substituted with one or two fatty ether substituents.

Additional permeation enhancers will be known to those of ordinary skill in the art of topical drug delivery, and/or are described in the pertinent texts and literature. See, e.g., Percutaneous Penetration Enhancers, Smith et al., eds. (CRC Press, 1995), the entire teachings of which are incorporated herein by reference.

In some embodiments, the compounds of the invention can degrade in the presence of ultraviolet (UV) light. Therefore, in some embodiments, it is desirable to include one or more opacifier which can partially or totally shield the compounds of the invention from UV light. Opacifiers which may be included in the topical compositions of the present invention include any pharmaceutically acceptable opacifier. Such opacifiers include, for example, titanium dioxide, talc, zinc oxide, magnesium stearate, calcium carbonate, behenic acid, and cetyl alcohol. Preferably, the opacifier is titanium dioxide. The amount of opacifier present in the compositions of the invention may be from about 0.05 weight percent to about 5 weight percent, based upon the weight of the composition. Preferably, the opacifier is present in an amount from about 0.1 weight percent to about 3 weight percent.

Pharmaceutical compositions of the invention may also include one or more humectants. A humectant is a moistening agent that promotes retention of water due to its hygroscopic properties. Exemplary humectants include glycerine, polymeric glycols such as polyethylene glycol and polypropylene glycol, mannitol, sorbitol and urea. One or more humectants can optionally be included in compositions of the invention in amounts from about 1 to 10 weight percent.

Pharmaceutical compositions of the invention may also include one or more emollients. An emollient is an oleaginous or oily substance which helps to smooth and soften the skin, and may also reduce its roughness, flaking, cracking or irritation. Typical suitable emollients include mineral oil having a viscosity in the range of 50 to 500 centipoise (cps), lanolin oil, coconut oil, cocoa butter, olive oil, almond oil, macadamia nut oil, aloe extracts such as aloe vera lipoquinone, synthetic jojoba oils, natural sonora jojoba oils, safflower oil, corn oil, liquid lanolin, cottonseed oil and peanut oil. In some embodiments, the emollient is a cocoglyceride, which is a mixture of mono, di and triglycerides of cocoa oil, sold under the trade name of Myritol 331 from Henkel KGaA, or Dicaprylyl Ether available under the trade name Cetiol OE from Henkel KGaA or a C₁₂-C₁₅ Alkyl Benzoate sold under the trade name Finsolv^{TN} from Finetex. Another suitable emollient is DC 200 Fluid 350, a silicone fluid, available Dow Coming Corp.

Other suitable emollients include squalane, castor oil, polybutene, sweet almond oil, avocado oil, calophyllum oil, ricin oil, vitamin E acetate, olive oil, silicone oils such as dimethylopolysiloxane and cyclomethicone, linolenic alcohol, oleyl alcohol, the oil of cereal germs such as the oil of wheat germ, isopropyl palmitate, octyl palmitate, isopropyl myristate, hexadecyl stearate, butyl stearate, decyl oleate, acetyl glycerides, the octanoates and benzoates of (C₁₂-C₁₅) alcohols, the octanoates and decanoates of alcohols and polyalcohols such as those of glycol and glyceryl, ricinoleates esters such as isopropyl adipate, hexyl laurate and octyl dodecanoate, dicaprylyl maleate, hydrogenated vegetable oil, phenyltrimethicone, jojoba oil and aloe vera extract.

Other suitable emollients which are solids or semi-solids at ambient temperatures may be used. Such solid or semi-solid cosmetic emollients include glyceryl dilaurate, hydrogenated lanolin, hydroxylated lanolin, acetylated lanolin, petrolatum, isopropyl lanolate, butyl myristate, cetyl myristate, myristyl myristate, myristyl lactate, cetyl alcohol, isostearyl alcohol and isocetyl lanolate. One or more emollients can optionally be included in the present invention ranging in amounts from about 1 percent to about 10 percent by weight, preferably about 5 percent by weight.

Topical pharmaceutical compositions of the invention may optionally contain drying agents. Drying agents generally promote rapid drying of moist areas and coats the skin for protection and healing. In particular, it acts to prevent irritation of the involved area and water loss from the skin layer by forming a physical barrier on the skin. Preferred drying agents include calamine; zinc containing drying agents such as zinc oxide, zinc acetate, zinc stearate, zinc sulfate, copper sulfate, kaolin, potassium permanganate, Burow's aluminum solution, talc, starches such as wheat and corn starch, silver nitrate, and acetic acid.

Compositions of the present invention optionally comprise an anti-itch agent such as phenol, camphor, menthol, benzocaine, diphenylhydramine or pramoxine. In general, the concentration of these anti-itch agents in the composition will be about 0.3 wt % to about 1 wt % for menthol, camphor and phenol; about 0.5 wt. % to about 20 wt % benzocaine; about 0.1 wt. % to about 20 wt %, more preferably about 0.5 wt % to about 5 wt. %, and still more preferably about 1 wt % to about 2 wt % for diphenylhydramine; and about 0.1 wt. % to about 20 wt %, more preferably about 0.5 wt % to about 5 wt. %, and still more preferably about 1 wt % for pramoxine. When an anti-itch agent is included, particularly if the anti-itch agent is diphenylhydramine or pramoxine, the composition preferably additionally comprises zinc acetate (about 0.01 wt % to about 5 wt. %, more preferably about 0.05 wt. % to about 3 wt. %, and still more preferably about 0.1 wt. % to about 1 wt. % zinc acetate).

Compositions of the present invention may also include a wide range of other optional ingredients including, antifoaming agents; buffers, neutralizing agents and agents to adjust pH; coloring agents and decoloring agents; emulsifying agents; odorants; preservatives, antioxidants, chemical stabilizers; solvents; and thickening, stiffening and suspending agents. Exemplary antifoaming agents include cyclomethicone, dimethicone (e.g., dimethicone 350) and simethicone. Exemplary buffers, neutralizing agents and agents to adjust pH include ammonium hydroxide, citric acid, diisopropanolamine, hydrochloric acid, lactic acid, monobasic sodium phosphate, sodium citrate, sodium hydroxide, sodium phosphate, triethanolamine, and trolamine. Exemplary emulsifying agents include aluminum starch octenylsuccinate, ammonium hydroxide, amphoteric-9, beeswax, synthetic beeswax, carbomer 934, carbomer 934P, carbomer 940, ceteareth-20, ceteareth-30, cetearyl alcohol, ceteth 20, cetyl alcohol, cholesterol, cyclomethicone, diglycerides, dimethicone (e.g., dimethicone 350), disodium monooleamidosulfosuccinate, NF emulsifying wax, fatty acid pentaerythritol ester, glycerides, glyceryl monooleate, glyceryl monostearate, lanolin, lanolin alcohol, hydrogenated lanolin, magnesium stearate, mineral oil, monoglycerides, polyethylene glycol, PEG 100 stearate, polyethylene glycol 6000 distearate, polyethylene glycol 1000 monoacetyl ether, polyethylene glycol monostearate, polyethylene glycol 400 monostearate, polyoxyethylene glycol fatty alcohol ethers, polyoxyl 20 cetostearyl ether, polyoxyl 40 stearate, polysorbate 20, polysorbate 40, polysorbate 60, polysorbate 80, polysorbates, PPG-26 oleate, propylene glycol stearate, quaternium-15, simethicone, sodium laureth sulfate, sodium lauryl sulfate, sorbitan esters, sorbitan monolaurate, sorbitan monooleate, sorbitan monopalmitate, sorbitan monostearate, sorbitan palmitate, sorbitan sesquioleate, steareth-2, steareth-100, stearic acid, stearyl alcohol, triethanolamine and trolamine.

Exemplary preservatives, antioxidants, and chemical stabilizers include alcohol, benzyl alcohol, butylated hydroxyanisole, butylated hydroxytoluene, butylparaben, calcium acetate, caster oil, chlorocresol, 4-chloro-m-cresol, citric acid, disodium edetate, Dowicil 200 (Dow), edetate disodium, ethoxylated alcohol, ethyl alcohol, glycerin, Glydant Plus (Lonza), 1,2,6-hexanetriol, Kathon CG (Rohm & Haas), Liquid Germall Plus (ISP Sutton Labs), Liquipar (ISP Sutton Labs), methylparaben, parabens, potassium sorbate, propyl gallate, propylene glycol, propylparaben, sodium bisulfite, sodium citrate, sodium metabisulfite, sorbic acid, tannic acid, triglycerides of saturated fatty acids, Ucarcide (Union Carbide), and zinc stearate. Exemplary solvents include alcohol, castor oil, diisopropyl adipate, ethoxylated alcohol, ethyl alcohol, fatty alcohol citrate, glycerin, 1,2,6-hexanetriol, hexylene glycol, isopropyl alcohol, isopropyl myristate, isopropyl palmitate, mineral oil, phosphoric acid, polyethylene glycol 300, polyethylene glycol 400, polyethylene glycol 1450, polyethylene glycol 8000, polyethylene glycol 1000 monoacetyl ether, polyethylene glycol monostearate, polyethylene glycol 400 monostearate, polyethylene glycols, polyoxyl 20 cetostearyl ether, polyoxypropylene 15-stearyl ether, polysorbate 20, polysorbate 40, polysorbate 60, polysorbate 80, polysorbates, propylene carbonate, propylene glycol, purified water, and SD alcohol 40, triglycerides of saturated fatty acids. Exemplary thickening, stiffening and suspending agents include aluminum stearate, beeswax, synthetic beeswax, carbomer 934, carbomer 934P, carbomer 940, cetostearyl alcohol, cetyl alcohol, cetyl esters wax, dextrin, glyceryl monostearate, hydroxypropyl cellulose, kaolin, paraffin, petrolatum, polyethylene, propylene glycol stearate, starch, stearyl alcohol, wax, white wax, xanthan gum, and bentonite.

Other agents which may be added to the composition of the present invention include, for example, aloe, arachis oil, benzoic acid, cocoa butter (up to about 70% by weight); coenzyme Q10 (Aubiquinone@), Q10, dimethicone, eucalyptus oil; resorcinol (up to about 5% by weight); retinol; retinyl palmitate; retinyl acetate; fennel extract; whey protein; ceramide; silicone (about 1% to about 50% by weight); alpha-hydroxy acids, beta-hydroxy acids, sorbitol, vitamin A (about 500 International Units per gram to about 300,000 International Units per gram provided, for example, in the form of fish liver oil, cod liver oil or shark liver oil), vitamin B (including panthenol and beta-carotene), vitamin C, vitamin D (about 50 International Units per gram to about 500 International Units per gram), vitamin E (about 20 International Units per gram to about 500 International Units per gram), and vitamin K. Unless otherwise indicated, the composition will generally contain less than about 5% by weight and typically less than about 1% by weight of the ingredients listed in this paragraph.

The carrier in the pharmaceutical composition must be “acceptable” in the sense of being compatible with the active ingredient of the formulation (and preferably, capable of stabilizing it) and not deleterious to the subject to be treated.

As used herein, the terms “animal”, “subject,” “mammal” and “patient”, include, but are not limited to, a cow, monkey, horse, sheep, pig, chicken, turkey, quail, cat, dog, mouse, rat, rabbit, guinea pig and human (preferably, a human).

Without further elaboration, it is believed that the above description has adequately enabled the present invention. The following specific embodiments are, therefore, to be construed as merely illustrative, and not limitative of the remainder of the disclosure in any way whatsoever. All of the references and publications cited herein are hereby incorporated by reference in their entirety.

### Example 1

### Psoriasis Study

Background

Psoriasis is a T cell-driven disease, with type I (IFN-γ-producing) T cells predominating in lesional skin. Psoriasis affects about 2.6% of the US population and therefore might be the most common T cell-mediated inflammatory disease in humans. A central role for IFN-γ as an inflammatory regulator is suggested from array-based gene expression studies that have identified increased expression of STAT1 and more than 20 genes controlled by this transcription factor. IFN-γ production is augmented by IL-12 (a 70-kD heterodimer formed from p40 and p35 subunits) and IL-23 (a newly described cytokine composed of a unique p19 subunit and a p40 subunit shared with IL-12).

A recent study to examine psoriasis skin lesions for gene expression by quantitative reverse transcription polymerase chain reaction revealed a reliable increase in p19 and p40 mRNA in lesional skin (22.3- and 11.6-fold increase, respectively, compared with nonlesional skin). Visualization by immunohistochemistry with anti-p40 monoclonal antibody showed that p40 protein was detected in dendritic profile within psoriatic lesion, but not in uninvolved skin. Cells isolated from psoriatic lesions showed p19 mRNA expression both in cells of monocytes (CD14⁺, CD11c⁺, CD83⁻) and mature dendritic cell (CD14⁻, CD11c⁺, CD83⁺) lineage. Various studies have shown increased numbers of activated Langerhans cells (CD83⁺) and interstitial dendritic cells (CD14⁻, CD11c⁺). Clinical response to treatment with anti-CD11a monoclonal antibody (Efalizumab) correlated better with decreases in CD11c⁺ dendritic cells than with decreases in CD3⁺ T cells. Also, decreases in CD83 and CD11c immunostaining were observed in responders of Alefacept therapy. In summary, the levels of IL-12/23 and monocytes/dendritic cells that have ability to produce these cytokines are critical in modulating psoriasis, suggesting that the reduction leads to attenuation of the disease.

Clinical Study

A multicenter, randomized, open-label, multiple oral dose outpatient study was done in patients with moderate to severe chronic plaque psoriasis. Patients received Compound 5021 mg or 35 mg orally twice daily (BID), 35 mg or 70 mg orally once per day (QD). Patients remained on study medication for a period of 12 weeks.

Lesional and nonlesional skin biopsies were collected from adults with chronic plaque-type psoriasis before and after treatment with Compound 50. The biopsies were processed using the QIAGEN RNeasy kit. Using a rotor-stator tissue homogenizer (saw-tooth rotor; PowerGen 700), frozen specimens were homogenized in buffer (RLT; QIAGEN) for 30-60 s and spun in a microcentrifuge for 3 min at room temperature to pellet debris. The supernatant was loaded onto a QIAGEN minicolumn and spun for 15s at high speed. A series of washes and DNase digestion was followed by final elution of RNA from the column. RNA was quantitated by UV spectrophotometry. The primers and probes for the TaqMan RT-PCR assays for IL-12/23 p40, IL-23 p19, and IL-10 were generated using the Primer Express algorithm version 1.0 from published sequences (National Center for Biotechnology Information). All primers and probes were synthesized by Applied Biosystems-PerkinElmer. RT-PCR reactions were performed according to the manufacturer's directions (EZ PCR Core Reagents; TaqMan and Applied Biosystems). The human acidic ribosomal protein gene (hARP), a housekeeping gene, was used to normalize each sample and each gene.

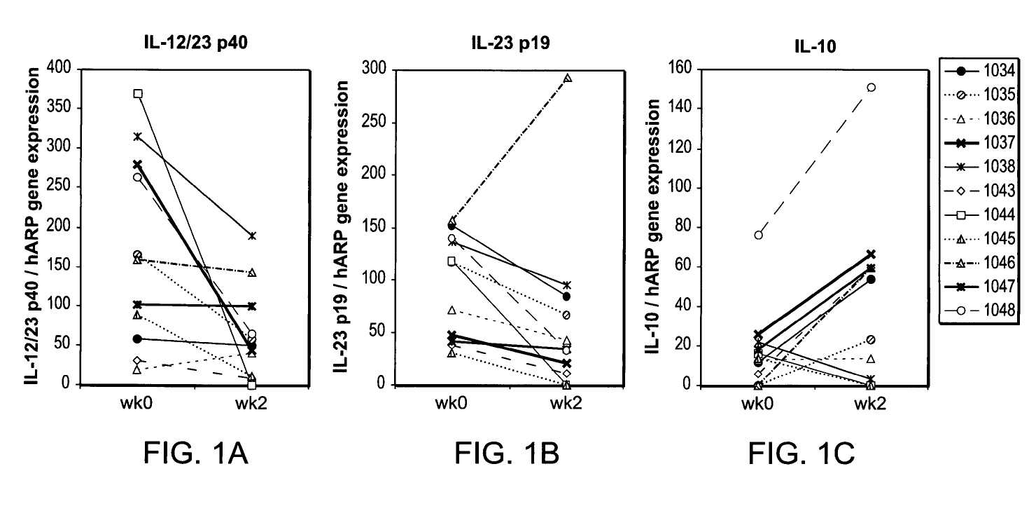

Gene expression levels at week 2 (wk2) were compared to pre-treatment levels in lesional psoriatic skin (wk0). As shown in FIGS. 1A and 1B, the expression of the genes encoding the p19 subunit of IL-23 and the p40 subunit shared by IL-12 and IL-23 were both reduced at week 2, respectively, demonstrate that Compound 50 inhibits expression of IL-12 and IL-23. In the same biopsy samples, an increase in mRNA IL-10, an anti-inflammatory cytokine, was observed (FIG. 1C). These results indicate that the reduction in gene expression is selective to IL-23 p19 and IL-12/23 p40

Tissue sections of skin biopsies were stained with hematoxylin (Fisher) and eosin (Shandon, Pittsburgh) and purified mouse anti-human mAbs to K16 (Sigma), CD3 (Becton Dickinson), CD11c (BD Pharmingen). Biotin-labeled horse anti-mouse antibody (Vector Laboratories) was amplified with avidin-biotin complex (Vector Laboratories) and developed with chromogen 3-amino-9-ethylcarbazole (Sigma Aldrich). Epidermal thickness measures were computed by using National Institutes of Health software (NIH IMAGE 6.1), and positive cells were counted manually by using computer-assisted image analysis.

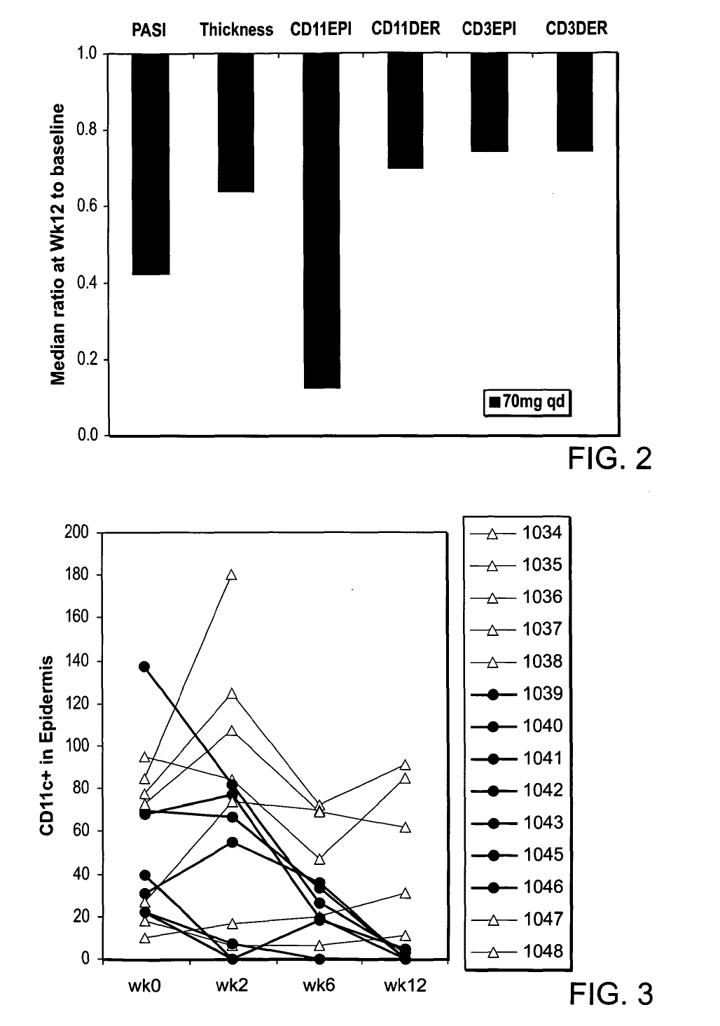

A marked decrease in CD11c⁺ cells in epidermis (CD11EPI in FIG. 2) was observed at week 12 in patients receiving 70 mg of Compound 50 once a day, suggesting that the suppression of IL-12 and IL-23 expression leads to a decrease in the cell populations to produce the cytokines from psoriatic skin (FIG. 2). There were significant but less pronounced decreases in CD11c⁺ cells in dermis (CD11DER) and CD3⁺ cells in epidermis and dermis (CD3EPI and CD3DER). The decrease in CD11c⁺ cells in epidermis nearly reached 100% in all of responder patients who were defined by histological improvement in the biopsy samples at week 12, while all of nonresponders showed no significant reduction in the count (FIG. 3). This data suggests that the disease was highly correlated with altered numbers of CD11c⁺ cells, further supporting the attenuation of the disease by suppressing IL-12 and/or IL-23 expression and decreasing monocytes and/or DCs in psoriatic lesion after treatment with Compound 50.

### Example 2

### Pre-Formulation Work

For the pre-formulation work Compound 50 was mixed with varying ratios of excipients commonly encountered in topical formulations. Table III below lists the excipients selected and the ratios of API to excipient under study.

### A. Study:

The Compound 50 Pre-Formulation Studies began with the addition of each individual excipient to 500 mg of the Active Pharmaceutical Ingredient (API). Table I below explains the detail ratio between each excipient and API used for this study.

The samples, once prepared in scintillation vials, were kept at 40° C. and 60° C. for over one month. The samples were analyzed by the method (not validated) developed by the analytical support group.

The results in Tables III-V were analyzed with respect to Compound 50 standard to calculate % recovery of API in the presence of each excipient. Samples 17-40° C., 18-40° C., had a small peak at ˜19 min. which could be an impurity. Similar results in table V for samples 5-60° C., 6-60° C., 7-60° C., 8-60° C., 10-60° C., 17-60° C., and 18-60° C. were obtained which needs to be further analyzed.

TABLE III
Experimental Design

| | Compound 50 Pre-Formulation | | Drug | Excipient | Drug | Excipient |
|---|---|---|---|---|---|---|
| | Excipient | Manufacturer | Ratio | | Actual Wt (g) | |
| 1 | Benzyl Alcohol | Spectrum | 5 | 0.5 | 0.5 | 0.050 |
| 2 | Glyceryl Monooleate | Croda | 5 | 5 | 0.5 | 0.500 |
| 3 | HPMC | DOW | 5 | 1 | 0.5 | 0.100 |
| 4 | PEG-300 | Croda | 5 | 20 | 0.5 | 2.000 |
| 5 | PEG-400 | Croda | 5 | 20 | 0.5 | 2.000 |
| 6 | Polysorbate 80 | Croda | 5 | 5 | 0.5 | 0.500 |
| 7 | Propylene Glycol | Spectrum | 5 | 20 | 0.5 | 2.000 |
| 8 | PVP | ISP | 5 | 10 | 0.5 | 1.000 |
| 9 | Sorbitan Monooleate | Uniqema | 5 | 5 | 0.5 | 0.500 |
| 10 | Miglyol 812 | Sasol | 5 | 20 | 0.5 | 2.000 |
| 11 | Lutrol F127 | BASF | 5 | 20 | 0.5 | 2.000 |
| 12 | Cetostearyl Alcohol | BASF | 5 | 5 | 0.5 | 0.500 |
| 13 | Cremophor A6 | BASF | 5 | 5 | 0.5 | 0.500 |
| 14 | Cremophor A25 | BASF | 5 | 5 | 0.5 | 0.500 |
| 15 | Mineral Oil | Spectrum/Sunoco | 5 | 10 | 0.5 | 1.000 |
| 16 | Propylparaben | Spectrum | 5 | 0.18 | 0.5 | 0.018 |
| 17 | Methylparaben | Spectrum | 5 | 0.02 | 0.5 | 0.002 |
| 18 | Transcutol | Gattfosse | 5 | 20 | 0.5 | 2.000 |
| 19 | TiO2 | Sensient | 5 | 5 | 0.5 | 0.500 |

TABLE IV
1-month 40° C. API-Excipient Compatibility Results

| | | API Peak | Time | 1st Impurity peak | | % Deg. | % Recovery |
|---|---|---|---|---|---|---|---|
| Excipient | Sample | Area [uV * s [symbol] | [min] | Area [uV * sec] | RRT | w/Main peak | (%) |
| Benzyl Alcohol | 1-40C | 2504059 | 19.73 | | | | 74.21 |
| Glyceryl Monooleate | 2-40C | 1550761 | 19.75 | | | | 46.35 |
| HPMC | 3-40C | 2550434 | 19.66 | | | | 76.63 |
| PEG-300 | 5-40C | 4107687 | 19.72 | | | | 123.72 |
| PEG-400 | 6-40C | 4234431 | 19.76 | | | | 125.98 |
| Polysorbate 80 | 7-40C | 3287401 | 19.79 | | | | 99.11 |
| Propylene Glycol | 8-40C | 3634550 | 19.80 | | | | 107.98 |
| PVP | 9-40C | 2786996 | 19.71 | | | | 84.16 |
| Sorbitan Monooleate | 10-40C | 697750 | 19.65 | | | | 20.78 |
| Miglyol 812 | 11-40C | 2373795 | 19.61 | | | | 70.64 |
| Lutrol F127 | 12-40C | 2223926 | 19.70 | | | | 67.06 |
| Cetostearyl Alcohol | 13-40C | 5627487 | 19.72 | | | | 167.72 |
| Cremophor A6 | 14-40C | 2962689 | 19.67 | | | | 87.85 |
| Cremophor A25 | 15-40C | 2686941 | 19.66 | | | | 80.13 |
| Mineral Oil | 16-40C | 1269417 | 19.70 | | | | 38.19 |
| Propylparaben | 17-40C | 2177811 | 19.68 | 195133 | 0.78 | 8.96% | 64.66 |
| Methylparaben | 18-40C | 2616638 | 19.73 | 8817 | 0.52 | 0.34% | 77.77 |
| Transcutol | 19-40C | 1290524 | 19.75 | | | | 38.5 |

[symbol]

indicates data missing or illegible when filed

TABLE V
1-month 60° C. API-Excipient Compatibility Results

| | Area [uV * se [symbol] | | [min] | Area [uV * sec] | [min] | w/Main Peak | |
|---|---|---|---|---|---|---|---|
| Benzyl Alcohol | 1-60C | 3260975 | 19.39 | | | | 98.42 |
| Glyceryl Monooleate | 2-60C | 1949655 | 19.36 | | | | 59.07 |
| HPMC | 3-60C | 2505673 | 19.4 | | | | 75.88 |
| PEG-300 | 5-60C | 3693742 | 19.37 | 18856 | 0.60 | 0.51% | 112.35 |
| PEG-400 | 6-60C | 3446501 | 19.33 | 64564 | 0.60 | 1.87% | 104.35 |
| Polysorbate 80 | 7-60C | 3500381 | 19.27 | 45566 | 0.60 | 1.30% | 105.71 |
| Propylene Glycol | 8-60C | 3007580 | 19.3 | 3396 | 0.60 | 0.11% | 91.26 |
| PVP | 9-60C | 2079175 | 19.29 | | | | 63.45 |
| Sorbitan Monooleate | 10-60C | 631173 | 19.34 | 52083 | 0.59 | 8.25% | 19.24 |
| Miglyol 812 | 11-60C | 1941779 | 19.33 | | | | 59 |
| Lutrol F127 | 12-60C | 5439 | 19.31 | | | | 0.17 |
| Cetostearyl Alcohol | 13-60C | 4835 | 19.23 | | | | 0.15 |
| Cremophor A6 | 14-60C | 0 | 0 | | | | 0 |
| Cremophor A25 | 15-60C | 726685 | 19.36 | | | | 22 |
| Mineral Oil | 16-60C | 1792342 | 19.33 | | | | 54.52 |
| Propylparaben | 17-60C | 4535874 | 19.44 | 288913 | 0.78 | 6.37% | 137.72 |
| Methylparaben | 18-60C | 1782001 | 19.26 | 244967 | 0.51 | 13.75% | 53.99 |

[symbol]

indicates data missing or illegible when filed

### Example 3

### Gel Cream Formulations

Gel creams were formulated by dissolving the API in Propylene Glycol at 70-75° C. Separately, a solution of Poloxamer 407 in water is also heated to 70° C. Both phases are combined at high temperature. Myglyol 812 was added and the entire mixture was cooled to RT with mixing. In these systems Propylene Glycol at 20% also serves as preservative. The literature reported that Propylene Glycol is a preservative at 15% or higher.

The gel-creams at 0.15% and 1% did not show visible crystals when observed at 40× after 2 months at RT, however the 2.0% gel-cream did show visible crystals after 2 months.

TABLE VI
Gel Creams

| | | | |
|---|---|---|---|
| | | % | |
| Formulation | Ingredient | w/w | Function |

1% Gel Cream

| | | | |
|---|---|---|---|
| FD04-02A | Compound 50 | 1.00 | API |
| | Medium Chain Triglycerie | 10.00 | Opacifier, emollient |
| | (Miglyol 812) | | |
| | Propylene Glycol | 20.00 | Solvent, Preservative |
| | Poloxamer 407 | 15.00 | Stabilizer, Gellant |
| | Purified Water USP | 54.00 | Vehicle |

0.15% Gel Cream

| | | | |
|---|---|---|---|
| FD04-02B | Compound 50 | 0.15 | API |
| | Medium Chain Triglycerie | 10.00 | Opacifier, emollient |
| | (Miglyol 812) | | |
| | Propylene Glycol | 20.00 | Solvent, Preservative |
| | Poloxamer 407 | 15.00 | Stabilizer, Gelaant |
| | Purified Water USP | 54.85 | Vehicle |

2% Gel Cream

| | | | |
|---|---|---|---|
| FD04-03 | Compound 50 | 2.00 | API |
| | Medium Chain Triglycerie | 10.00 | Opacifier, emollient |
| | (Miglyol 812) | | |
| | Propylene Glycol | 20.00 | Solvent, Preservative |
| | Poloxamer 407 | 15.00 | Stabilizer, Gelaant |
| | Purified Water USP | 53.00 | Vehicle |

Alternative Formulations Include:

| | Formulation | | | | |
|---|---|---|---|---|---|
| | FD10-20 | FD10-21 | FD10-22 | FD10-23 | FD10-24 |
| Compound 50 | 0.15 | 5.00 | 5.00 | 0.15 | 3.00 |
| Propylene Glycol | 29.00 | 29.00 | 29.00 | 29.00 | 29.00 |
| PEG-300 | 35.05 | 30.20 | 30.18 | 35.03 | 32.18 |
| Diethylene Glycol Monoethyl | 20.00 | 20.00 | 20.00 | 20.00 | 20.00 |
| Ether | | | | | |
| Carbomer 940 | 2.50 | 2.50 | 2.50 | 2.50 | 2.50 |
| Isopropyl Myristate | 6.00 | 6.00 | 6.00 | 6.00 | 6.00 |
| BHA | | | 0.02 | 0.02 | 0.02 |
| BHT | 0.10 | 0.10 | 0.10 | 0.10 | 0.10 |
| Titanium Dioxide | 0.20 | 0.20 | 0.20 | 0.20 | 0.20 |
| Isopropyl Alcohol | 7.00 | 7.00 | 7.00 | 7.00 | 7.00 |
| Total | 100.00 | 100.00 | 100.00 | 100.00 | 100.00 |
| | Formulation | | | | |
| | FD04-70 | FD04-71 | FD04-72 | FD04-73 | FD04-79 |
| Compound 50 | 0.15 | 0.15 | 1.00 | 5.00 | 3.00 |
| Propylene Glycol | 26.00 | 26.00 | 26.00 | 26.00 | 26.00 |
| PEG-300 | 38.50 | 38.20 | 37.35 | 33.35 | 35.35 |
| Diethylene Glycol Monoethyl | 20.00 | 20.00 | 20.00 | 20.00 | 20.00 |
| Ether | | | | | |
| Carbomer 980 | 2.20 | 2.50 | 2.50 | 2.50 | 2.50 |
| Isopropyl Mysristate | 6.00 | 6.00 | 6.00 | 6.00 | 6.00 |
| BHT | 0.05 | 0.05 | 0.05 | 0.05 | 0.05 |
| Titanium Dioxide | 0.10 | 0.10 | 0.10 | 0.10 | 0.10 |
| Isopropyl Alcohol | 7.00 | 7.00 | 7.00 | 7.00 | 7.00 |
| Total | 100.00 | 100.00 | 100.00 | 100.00 | 100.00 |

### Example 4

### Creams

Creams were formulated using a standard pharmaceutical cream base. Propylene Glycol was present at 10% as solubilizer. The two emulsifiers Cremophor A6 and Cremophor A25 were also used as solubilizers.

This preparation consisted of mixing two phases at 70-75° C.: An oil phase (Cetostaeryl Alcohol, Cremophor A6, Cremophor A25, Mineral Oil and Propylparaben) and a water phase (Water, and Methylparaben). After emulsification, the mixture was mixed while cooling to room temperature (RT). Formulation FD04-07 was physically stable after 6 weeks at RT. Formulation FD04-24 was physically stable after 2 weeks at RT.

TABLE VII
Creams

| Formulation | | % w/w | Function |
|---|---|---|---|
| | 1% Cream | | |
| FD04-07 | Compound 50 | 1.00 | API |
| | Cetostearyl Alcohol | 7.00 | Consistency |
| | Cremophor A6 (Steareth-6 and | 1.50 | Emulsifier |
| | Stearyl Alcohol) | | |
| | Cremophor A25 (Ceteareth-25) | 1.50 | Emulsifier |
| | Mineral Oil | 12.00 | Occlusivness |
| | Propylene Glycol | 10.00 | Solvent |
| | Methyl Paraben | 0.12 | Preservative |
| | Propyl Paraben | 0.02 | Preservative |
| | Water | 66.86 | Vehicle |
| | Total | 100.00 | |
| | 0.15% Cream | | |
| FD04-24 | Compound 50 | 0.15 | API |
| | Cetostearyl Alcohol | 7.00 | Consistency |
| | Cremophor A6 (Steareth-6 and | 1.50 | Emulsifier |
| | Stearyl Alcohol) | | |
| | Cremophor A25 (Ceteareth-25) | 1.50 | Emulsifier |
| | Mineral Oil | 12.00 | Occlusivness |
| | Propylene Glycol | 10.00 | Solvent |
| | Methyl Paraben | 0.12 | Preservative |
| | Propyl Paraben | 0.02 | Preservative |
| | Water | 67.71 | Vehicle |
| | Total | 100.00 | |

### Example 5

### Gel Formulations

The gel below was formulated by combining as solvents Propylene Glycol, PEG-300 and Diethylene Glycol Monoethyl Ether. The solvents were gelled with Hydroxypropyl-cellulose. The gel was heated to 70-75° C. The drug was added and dissolved. Then the mixture is cooled to below 38° C. and the Water and the Isopropyl Myristate were added.

TABLE VIII
FD04-29B Gel

| Formulation | 1% Gel | % w/w | Function |
|---|---|---|---|
| FD04-29B | Propylene Glycol | 29.40 | Solvent, Preservative |
| | PEG-300 | 22.35 | Solvent |
| | Diethylene Glycol | 19.50 | Solvent |
| | Monoethyl Ether | | |
| | Compound 50 | 1.00 | API |
| | Hydroxypropylcellulose | 1.25 | Gellant |
| | HF Pharma | | |
| | Water | 24.50 | Vehicle |
| | Isopropyl Myristate | 2.00 | Emollient |
| | Total | 100.00 | |

The following formulation is based on a Poloxamer 407 gel system. Diethylene Glycol Monoethyl Ether was used as a co-solvent. In addition, Cremophor A6 was used as a coupling agent. Initially this formula appeared to have Compound 50 in solution; however, crystals were visible after 2 weeks at RT.

TABLE IX
FD04-16 Gel

| Formulation | 2% Gel | % w/w | Function |
|---|---|---|---|
| FD04-16 | Compound 50 | 2.00 | API |
| | Cremophor A6 | 30.0 | Coupling |
| | Proplene Glycol | 30.0 | Solvent, Preservative |
| | Water | 22.5 | Vehicle |
| | Diethylene Glycol | 13.0 | Co-solvent |
| | Monoethyl Ether | | |
| | Poloxamer 407 | 2.5 | Stabilizer |
| | Total | 100.00 | |

The gel below is similar to Gel FD04-29B. The main difference being that this formulation has no water. Isopropyl Myristate provided some emolliency but the gel still seemed to be somewhat stringy.

TABLE X
FD04-33 Gel

| Formulation | 2% Gel | % w/w | Function |
|---|---|---|---|
| FD04-33 | Propylene Glycol | 52.40 | Solvent, Preservative |
| | PEG-300 | 22.35 | Solvent, Preservative |
| | Diethylene Glycol | 19.50 | Solvent, Preservative |
| | Monoethyl Ether | | |
| | Compound 50 | 2.00 | IPA |
| | Hydroxypropylcellulose | 1.25 | Gellant |
| | HF Pharma | | |
| | Isopropyl Myristate | 2.50 | Emollient |
| | Total | 100.0 | |

| | Formulation | | | | | |
|---|---|---|---|---|---|---|
| | FD04-34 | FD04-61 | FD04-62 | FD04-63 | FD04-65 | FD04-76 |
| Compound 50 | 52.90 | 2.00 | 5.00 | 0.15 | 1.00 | 1.00 |
| Propylene Glycol | 22.35 | 25.00 | 25.00 | 25.25 | 25.25 | 25.25 |
| PEG-300 | 19.50 | 37.45 | 34.45 | 39.35 | 38.50 | 38.50 |
| Diethylene Glycol | 2.00 | 20.00 | 20.00 | 20.00 | 20.00 | 20.00 |
| Monoethyl Ether | | | | | | |
| Hydroxypropylcellulose | 1.25 | | | | | |
| HF | | | | | | |
| Carbomer 980 | | 2.50 | 2.50 | 2.20 | 2.20 | 2.20 |
| Isopropyl Mysristate | 2.00 | 3.00 | 3.00 | 3.00 | 3.00 | 3.00 |
| BHT | | 0.05 | 0.05 | 0.05 | 0.05 | 0.05 |
| Isopropyl Alcohol | | 10.00 | 10 | 10 | 10 | 10 |
| Total | 100.00 | 100.00 | 100.00 | 100.00 | 100.00 | 100.00 |
| | Formulation | | | | | |
| | FD10-09 | FD10-10 | FD10-11 | FD10-12 | FD10-13 | FD10-14 |
| Compound 50 | 5.00 | 0.15 | 0.15 | 0.15 | 5.00 | 5.00 |
| Propylene Glycol | 26.25 | 26.25 | 26.25 | 26.25 | 26.25 | 26.25 |
| PEG-300 | 33.45 | 38.28 | 38.35 | 38.25 | 33.45 | 33.43 |
| Diethylene Glycol | 20.00 | 20.00 | 20.00 | 20.00 | 20.00 | 20.00 |
| Monoethyl Ether | | | | | | |
| Carbomer 980 | 2.20 | 2.20 | 2.20 | 2.20 | 2.20 | 2.20 |
| Isopropyl Mysristate | 3.00 | 3.00 | 3.00 | 3.00 | 3.00 | 3.00 |
| BHA | | 0.02 | | | | 0.02 |
| BHT | 0.1 | 0.1 | | 0.10 | 0.10 | 0.10 |
| Ascorbyl Palmitate | | | 0.05 | 0.05 | | |
| Isopropyl Alcohol | 10 | 10 | 10 | 10 | 10 | 10 |
| Total | 100.00 | 100.00 | 100.00 | 100.00 | 100.00 | 100.00 |

### Example 5

### Solution Formulations

In general the solution formulations used the basic solubility information previously generated and describe above. Whenever possible an amount of water and Poloxamer 407 as stabilizer were added. It was noted that the formulations at 0.15% and 1% contained about 25% Water. Although it was not initially possible to add significant amount of water to formulations containing more than 1% API, formulation FD04-35 was able to use a combination of solvents that produced a solution of 5% API with 21% Water.

TABLE XI
Solutions

| Formula- | | | |
|---|---|---|---|
| tion | | % W/W | Function |
| | 1% Solution | | |
| FD04-09 | Compound 50 | 1.00 | API |
| | Propylene Glycol | 30.00 | Solvent |
| | Cremophor A-25 | 3.00 | Solubilizer |
| | PEG-300 | 20.00 | Solvent |
| | Polysorbate 80 | 5.00 | Solubilizer |
| | Diethylene Glycol Monoethyl Ether | 10.00 | Solvent |
| | Benzyl Alcohol | 1.00 | Preservative |
| | Poloxamaer 407 | 4.50 | Stabilizer |
| | Water | 25.50 | Vehicle |
| | Total | 100.00 | |
| | 2% Solution | | |
| FD04-22 | Compound 50 | 2.00 | API |
| | Propylene Glycol | 40.00 | Solvent |
| | Cremophor A-25 | 2.00 | Solubilizer |
| | PEG-300 | 35.00 | Solvent |
| | Polysorbate 80 | 5.00 | Solubilizer |
| | Diethylene Glycol Monoethyl Ether | 15.00 | Solvent |
| | Benzyl Alcohol | 1.00 | Preservative |
| | Total | 100.00 | |
| | 0.15% Solution | | |
| FD04-23 | Compound 50 | 0.15 | API |
| | Propylene Glycol | 29.57 | Solvent |
| | Cremophor A-25 | 3.00 | Solubilizer |
| | PEG-300 | 20.00 | Solvent |
| | Polysorbate 80 | 5.00 | Solubilizer |
| | Diethylene Glycol Monoethyl Ether | 10.00 | Solvent |
| | Benzyl Alcohol | 1.00 | Preservative |
| | Poloxamaer 407 | 4.69 | Stabilizer |
| | Water | 26.59 | Vehicle |
| | Total | 100.00 | |
| | 3% Solution | | |
| FD04-26 | Compound 50 | 3.00 | API |
| | PEG-300 | 80.00 | Solvent |
| | Diethylene Glycol Monoethyl Ether | 17.00 | Solvent |
| | Total | 100.00 | |
| | 5% Solution | | |
| FD04-35 | Compound 50 | 5.26 | API |
| | PEG-300 | 21.05 | Solvent |
| | Diethylene Glycol Monoethyl Ether | 52.63 | Solvent |
| | Water | 21.05 | Vehicle |
| | Total | 100.0 | |

| | Formulation | | | | | | |
|---|---|---|---|---|---|---|---|
| | FD04-89 | FD04-90 | FD04-91 | FD04-92A | FD04-92B | FD04-93 | FD04-99 |
| Compound | 5.00 | 5.00 | 5.00 | 3.00 | 1.00 | 0.15 | 5.00 |
| 50 | | | | | | | |
| PEG-300 | 54.99 | 54.99 | 54.99 | 56.99 | 58.99 | 59.84 | 54.69 |
| Polysorbate | | 5.00 | 5.00 | 5.00 | 5.00 | 5.00 | 5.00 |
| 80 | | | | | | | |
| Diethylene | 25.00 | 25.00 | 25.00 | 25.00 | 25.00 | 25.00 | 25.00 |
| Glycol | | | | | | | |
| Monoethyl | | | | | | | |
| Ether | | | | | | | |
| Carbomer | | | | | | | 0.30 |
| 980 | | | | | | | |
| BHT | 0.01 | 0.01 | 0.01 | 0.01 | 0.010 | 0.01 | 0.01 |
| Ethyl | 10.00 | 10.00 | 10.00 | 10.00 | 10.00 | 10.00 | 10.00 |
| Alcohol | | | | | | | |
| Poloxamer | 5.00 | | | | | | |
| F127 | | | | | | | |
| Total | 100 | 100 | 100 | 100 | 100 | 100 | 100 |

### Example 6

### Effect of Transcutol in Formulations

Below are a series of formulations with more or less levels of Transcutol. To guarantee solubility, Propylene Glycol and PEG-6 Caprilic/Capric Glyceride (Labrasol) were used as solvents in the formulations where Trancutol was completely removed or was present at low levels. This series of formulations are described below:

TABLE XII
All formulations at 5% of Compound 50

| | Formulation | | | | |
|---|---|---|---|---|---|
| | SA412- | SA412- | SA412- | SA412- | SA412- |
| | 03-01 | 03-02 | 03-03 | 03-04 | 03-05 |
| Compound 50 | 5.00 | 5.00 | 5.00 | 5.00 | 5.00 |
| PEG-4000 | 20.00 | 20.00 | 20.00 | 20.00 | 20.00 |
| PEG-300 | 59.88 | 57.88 | 56.88 | 54.88 | 59.88 |
| Transcutol | 0.00 | 2.00 | 0.00 | 2.00 | 10.00 |
| Propylene Glycol | 10.00 | 10.00 | 0.00 | 0.00 | 0.00 |
| Labrasol* | 0.00 | 0.00 | 13.00 | 13.00 | 0.00 |
| BHT | 0.10 | 0.10 | 0.10 | 0.10 | 0.100 |
| BHA | 0.02 | 0.02 | 0.02 | 0.02 | 0.020 |
| Polysorbate 80 | 5.0 | 5.0 | 5.0 | 5.0 | 5.0 |
| Total | 100 | 100 | 100 | 100 | 100 |

These formulations were prepared in April 2008 and placed on stability at room temperature and at 40° C./75% RH. The results of this study are presented below:

TABLE XIII
Stability Results- Assay

| | Formulation | | | | |
|---|---|---|---|---|---|
| | SA412- | SA412- | SA412- | SA412- | SA412- |
| 5% Assay | 03-01 | 03-02 | 03-03 | 03-04 | 03-05 |
| PEG Gel RT, Initial | 102.7 | 97.4 | 93.2 | 92.2 | 95.0 |
| 1-month RT | 103.5 | 97.5 | 92.2 | 91.3 | 96.3 |
| 1-month, | 100.2 | 96.4 | 91.5 | 90.6 | 97.6 |
| 40 C./75% RH | | | | | |
| 2-month RT | * | * | * | * | * |
| 2-month, | 95.3 | 90.5 | 87.6 | 82.2 | 90.4 |
| 40 C./75% RH | | | | | |
| 3-month RT | NR | NR | NR | NR | 96.3 |
| 3-month, | 94.6 | 89.8 | 85.4 | 84.6 | 94.3 |
| 40 C./75% RH | | | | | |

### Example 7

### Manufacturing Process Development

The most important step in the preparation of formulations of Compound 50 is the solubilization of the API in the solvent system. Both dosage forms; solutions and gels have a solvent system composed of one or more solvents or solubilizers. In addition the API must be added to the solvent system which must be mixed vigorously and heated to 65° C.-77° C. For solubility to be achieved; the mixing and heating conditions must be maintained for 40-60 minutes. Once the API has completely dissolved, then the mixture can be slowly cooled down to RT. In both cases the antioxidant(s) BHA and BHT need to also be added when the solvents are at high temperature. Like the drug; both BHA and BHT are not water soluble. In the case of the solution where there is 10% Ethanol, the alcohol is added when the materials have cooled down below 30° C.

A. Preparation of Solutions:

1. Mix Polysorbate 80 and Polyethylene Glycol 300.

2. Add Transcutol and BHT and continue mixing until the BHT dissolves.

3. Add Compound 50, mix and heat the materials to 73-77° C. (Dissolution may take 40 to 60 minutes).

4. Continue mixing and maintain temperature until the API dissolves.

5. Mix and cool down to 30° C. Add ethanol and mix to uniformity.

6. QS to final weight with Polyethylene Glycol 300.

B. Preparation of the PEG Gels:

1. Mix Transcutol and 90% of the Polyethylene Glycol 300.

2. Mix and heat the solvents to 60-65° C.

3. Maintain temperature and mixing conditions and add BHA and BHT. Mix until the powders dissolve.

4. Mix Polysorbate 80 and 5% of the amount of Polyethylene Glycol 300. Disperse in this mixture Compound 50.

5. Add Step 4 to Step 3 with constant mixing at 60-65° C. Use the remaining Polyethylene Glycol 300 to rinse the container used to disperse the Compound 50. Add the rinse to the beaker containing Step 3. (Dissolution may take 40-60 minutes).

6. Melt separately PEG-4000 to 60-65° C. and add the melt to Step 5.

7. Mix and cool to 30° C. and QS with Polyethylene Glycol 300.

### Other Embodiments

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

1. A composition for topical administration for the treatment of psoriasis, the composition comprising:
(a) a compound represented by formula (I): 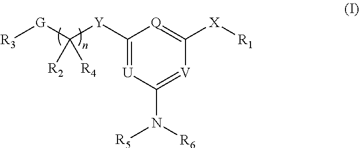
or a pharmaceutically acceptable salt thereof, wherein:
R₁ is optionally substituted aryl, optionally substituted heteroaryl, or a group represented by the following formula: 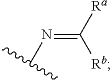
R₂ and R₄, for each occurrence, are independently, H, an optionally substituted alkyl, an optionally substituted alkylcarbonyl, —OR^{k}, —SR^{k}, —NR^{h}R^{j}, hydroxylalkyl, —C(O)R^{c}, —OC(O)R^{c}, —SC(O)R^{c}, —NR^{k}C(O)R^{c}, —C(S)R^{c}, —OC(S)R^{c}, —SC(S)R^{c}, —NR^{k}C(S)R^{c}, —C(NR)R^{c}, —OC(NR)R^{c}, —SC(NR)R^{c}, —NR^{k}C(NR)R^{c}, —SO₂R^{c}, —S(O)R^{c}, —NR^{k}SO₂R^{c}, —OS(O)₂R^{c}, —OP(O)R^{c}R^{c}, —P(O)R^{c}R^{c}, halo, haloalkyl, aminoalkyl, mercaptoalkyl, cyano, nitro, nitroso, azide, an optionally substituted alkylcarbonylalkyl, an optionally substituted cyclyl, an optionally substituted cycloalkyl, an optionally substituted heterocyclyl, an optionally substituted heterocycloalkyl, an optionally substituted aryl, an optionally substituted aralkyl, an optionally substituted heteroaryl, an optionally substituted heteroaralkyl, or isothionitro; or R₂ and R₄ taken together are ═O, ═S, or ═NR;
R₃ is R^{g};
R₅ and R₆ are each, independently, H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl; or R₅ and R₆ taken together with the N to which they are attached is an optionally substituted heterocyclyl, an optionally substituted heterocycloalkyl, or an optionally substituted heteroaryl;
X is O, S, S(O), S(O)₂, or NR^{k};
Y is (CH(R^{g}))ₘ, C(O), C(NR), O, S, S(O), S(O)₂, N(R^{k}), or absent;
G is a bond, —C(O)NR^{k}NR^{k}—, —NR^{k}NR^{k}C(O)—, —NR^{k}N═CR^{k}—, —CR^{k}═NNR^{k}—, —NR^{k}NR^{k}—, —N(OH)—, —NR^{k}O—, —ONR^{k}—, —O(O)—, —C(NR)—, —NR^{k}C(O)—, —C(O)NR^{k}—, —OC(O)—, —C(O)O—, —OC(O)O—, —NR^{k}C(O)O—, —OC(O)NR^{k}—, —NR^{k}C(S)O—, —OC(S)NR^{k}—, —NR^{k}—C(NR)—NR^{k}—, —NR^{k}—C(O)—NR^{k}—, —NR^{k}—C(S)—NR^{k}—, —NR^{k}—S(O)₂—NR^{k}—, —P(O)(R^{c})—, —P(O)(R^{c})O—, —OP(O)(R^{c})—, —OP(O)(R^{c})O—, an optionally substituted cycloalkylene, an optionally substituted cyclylene, an optionally substituted heterocycloalkylene, an optionally substituted heterocyclylene, an optionally substituted arylene, an optionally substituted aralkylene, an optionally substituted heteroarylene, an optionally substituted heteroaralkylene, an optionally substituted heteroarylene-NR^{k}—, an optionally substituted heteroarylene-S—, an optionally substituted heteroaralkylene-O—, —Si(OR^{k})₂—, —B(OR^{k})—, —C(NR)—NR^{k}—, —NR^{k}—CR^{g}R^{g}—C(O)—, —C(O)—ONR^{k}—, —C(O)—NR^{k}O—, —C(S)—ONR^{k}—, —C(S)—NR^{k}O—, —C(NR)—ONR^{k}—, —C(NR)—NR^{k}O—, —OS(O)₂—NR^{k}NR^{k}—, —OC(O)—NR^{k}NR^{k}—, —OC(S)—NR^{k}NR^{k}—, —OC(NR)—NR^{k}NR^{k}—, —NR^{k}NR^{k}S(O)₂O—, —NR^{k}NR^{k}C(S)O—, —NR^{k}NR^{k}C(NR)O—, —OP(O)(R^{c})O—, —NR^{k}P(O)(R^{c})O—, —OP(O)(R^{c})NR^{k}—, —NR^{k}P(O)(R^{c})NR^{k}—, —P(O)(R^{c})NR^{k}—, —NR^{k}P(O)(R^{c})—, —O-alkylene-heterocycloalkylene-NR^{k}—, —NR^{k}—CHR^{g}—C(O)—NR^{k}—CHR^{g}—C(O)—, —NR^{k}—CHR^{g}—C(O)—, —NR^{k}—C(O)—CHR^{g}—, or —C(O)—NR^{k}—CHR^{g}—C(O)—; and
each of Q, U, and V are independently N or CR^{g}, wherein at least one of Q, U, or V is N; and each CR^{g} may be the same or different;
R, for each occurrence, is independently H, an optionally substituted alkyl, an optionally substituted cycloalkyl, an optionally substituted cyclyl, an optionally substituted heterocycloalkyl, an optionally substituted heterocyclyl, an optionally substituted heteroaryl, an optionally substituted aralkyl, an optionally substituted heteroaralkyl, —C(O)R^{c}, —OR^{k}, —SR^{k}, —NR^{h}R^{j}, hydroxylalkyl, nitro, cyano, haloalkyl, aminoalkyl, or —S(O)₂R^{c};
each of R^{a} and R^{b}, independently, is H, optionally substituted alkyl, an optionally substituted cycloalkyl, an optionally substituted cyclyl, an optionally substituted heterocycloalkyl, an optionally substituted heterocyclyl, optionally substituted aryl, or optionally substituted heteroaryl;
R^{c}, for each occurrence, is independently, H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl, haloalkyl, —OR^{k}, —SR^{k}, —NR^{h}R^{j}, hydroxylalkyl, alkylcarbonylalkyl, mercaptoalkyl, aminoalkyl, sulfonylalkyl, sulfonylaryl, or thioalkoxy;
R^{g}, for each occurrence, is independently, H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl, haloalkyl, —OR^{k}, —SR^{k}, hydroxylalkyl, alkylcarbonylalkyl, mercaptoalkyl, aminoalkyl, sulfonylalkyl, sulfonylaryl, thioalkoxy, —C(O)R^{c}, —OC(O)R^{c}, —SC(O)R^{c}, —NR^{k}C(O)R^{c}, —C(S)R^{c}, —OC(S)R^{c}, —SC(S)R^{c}, —NR^{k}C(S)R^{c}, —C(NR)R^{c}, —OC(NR)R^{c}, —SC(NR)R^{c}, —NR^{k}C(NR)R^{c}, —SO₂R^{c}, —S(O)R^{c}, —NR^{k}SO₂R^{c}, —OS(O)₂R^{c}, —OP(O)R^{c}R^{c}, —P(O)R^{c}R^{c}, halo, aminoalkyl, mercaptoalkyl, cyano, nitro, nitroso, or azide;
R^{h} and R^{j}, for each occurrence, are independently H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl; or R^{h} and R^{j} taken together with the N to which they are attached is an optionally substituted heterocyclyl, an optionally substituted heterocycloalkyl, or an optionally substituted heteroaryl;
R^{k}, for each occurrence, is independently H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, or an optionally substituted heteroaryl;
n is 0, 1, 2, 3, 4, 5, 6 or 7; and
m is 0, 1, 2, 3, or 4; and
(b) a pharmaceutically acceptable topical carrier.

2.-6. (canceled)

7. The composition of claim 1, wherein the pharmaceutically acceptable topical carrier comprises water and one or more organic component selected from the group consisting of isopropyl alcohol, propylene glycol, ethanol, ethylene glycol, polyethylene glycol, glycerol, octanol, benzyl alcohol, sorbitol, mannitol, isopropyl myristate, polyethylene glycol monolaurate, N,N-dimethylamide, N-methyl-2-pyrrolidone and polyvinyl-pyrrolidone, urea, dimethylacetamide (DMA), 2-pyrrolidone, 1-methyl-2-pyrrolidone, dimethylsulfoxide, ethanolamine, diethanolamine and triethanolamine.

8.-9. (canceled)

10. The composition of claim 1, further comprising one or more penetration enhancer selected from the group consisting of diethylene glycol monoethyl ether, diethylene glycol monomethyl ether, sodium laurate, sodium lauryl sulfate, cetyltrimethylammonium bromide, benzalkonium chloride, Poloxamer 231, Poloxamer 182, Poloxamer 184, Tween 20, Tween 40, Tween 60, Tween 80, lecithin, ethanol, propanol, octanol, benzyl alcohol, polyethylene glycol, polyethylene glycol monolaurate, urea, dimethylacetamide (DMA), dimethylformamide (DMF), 2-pyrrolidone, 1-methyl-2-pyrrolidone, ethanolamine, diethanolamine, triethanolamine a terpene; an alkanones, citric acid, succinic acid, dimethyl sulfoxides (DMSO), methyl laurate, ethyl oleate, propylene glycol monolaurate, propylene glycerol dilaurate, glycerol monolaurate, glycerol monooleate, isopropyl n-decanoate, octyldodecyl myristate, stearyl alcohol, oleyl alcohol C₂-C₄ alkane diol substituted with one or two fatty ether substituents and a C₂-C₄ triol substituted with one or two fatty ether substituents.

11. (canceled)

12. The composition of claim 1, further comprising one or more viscosity enhancer selected from the group consisting of carbomer 934, carbomer 934P, carbomer 940, cetearyl alcohol, cetostearyl alcohol, cetyl alcohol, cetyl stearyl alcohol, dextrin, diglycerides, disodium edetate, edetate disodium, glycerides, glyceryl monostearate, glyceryl stearate, hydroxypropyl cellulose, monoglycerides, plasticized hydrocarbon gel, polyethylene glycol 300, polyethylene glycol 400, polyethylene glycol 1450, polyethylene glycol 8000, polyethylene glycols, propylene glycol stearate and stearyl alcohol.

13. (canceled)

14. The composition of claim 1, further comprising one or more opacifier selected from the group consisting of titanium dioxide, talc, zinc oxide, magnesium stearate, calcium carbonate, behenic acid, and cetyl alcohol.

15. The composition of claim 14, wherein the one or more opacifiers comprise about 0.05% to about 5% by weight of the composition.

16. (canceled)

17. The composition of claim 1, further comprising one or more humectant selected from the group consisting of glycerine, polymeric glycols, polyethylene glycol and polypropylene glycol, mannitol, sorbitol and urea.

18. The composition of claim 17, wherein the one or more humectants comprise about 1% to about 10% by weight of the composition.

19. (canceled)

20. The composition of claim 1, further comprising one or more anti-itch agent selected from the group consisting of menthol, camphor, phenol, benzocaine, diphenylhydramine and pramoxine.

21. (canceled)

22. The composition of claim 1, further comprising one or more emollient selected from the group consisting of mineral oil, lanolin oil, coconut oil, cocoa butter, olive oil, almond oil, macadamia nut oil, aloe extracts, synthetic jojoba oils, natural sonora jojoba oils, safflower oil, corn oil, liquid lanolin, cottonseed oil, peanut oil, Myritol 331, dicaprylyl ether, a C₁₂-C₁₅ alkyl benzoate, DC 200 Fluid 350, a silicone fluid, squalane, castor oil, polybutene, avocado oil, calophyllum oil, ricin oil, vitamin E acetate, olive oil, silicone oils, dimethylopolysiloxane, cyclomethicone, linolenic alcohol, oleyl alcohol, the oil of cereal germs, isopropyl palmitate, octyl palmitate, isopropyl myristate, hexadecyl stearate, butyl stearate, decyl oleate, acetyl glycerides, the octanoates and benzoates of (C₁₂-C₁₅) alcohols, the octanoates and decanoates of alcohols and polyalcohols, ricinoleates, isopropyl adipate, hexyl laurate and octyl dodecanoate, dicaprylyl maleate, hydrogenated vegetable oil, phenyltrimethicone, jojoba oil, aloe vera extract, glyceryl dilaurate, hydrogenated lanolin, hydroxylated lanolin, acetylated lanolin, petrolatum, isopropyl lanolate, butyl myristate, cetyl myristate, myristyl myristate, myristyl lactate, cetyl alcohol, isostearyl alcohol, and isocetyl lanolate.

23. The composition of claim 22, wherein the one or more emollient comprise about 1% to about 10% by weight of the composition.

24. (canceled)

25. The composition of claim 1, further comprising one or more drying agent selected from the group consisting of calamine, zinc oxide, zinc acetate, zinc stearate, zinc sulfate, copper sulfate, kaolin, potassium permanganate, Burow's aluminum solution, talc, wheat starch, corn starch, silver nitrate, and acetic acid.

26. (canceled)

27. The composition of claim 1, further comprising one or more emulsifying agents selected from the group consisting of aluminum starch octenylsuccinate, ammonium hydroxide, amphoteric-9, beeswax, synthetic beeswax, carbomer 934, carbomer 934P, carbomer 940, ceteareth-20, ceteareth-30, cetearyl alcohol, ceteth 20, cetyl alcohol, cholesterol, cyclomethicone, diglycerides, dimethicone, disodium monooleamidosulfosuccinate, NF emulsifying wax, fatty acid pentaerythritol ester, glycerides, glyceryl monooleate, glyceryl monostearate, lanolin, lanolin alcohol, hydrogenated lanolin, magnesium stearate, mineral oil, monoglycerides, polyethylene glycol, PEG 100 stearate, polyethylene glycol 6000 distearate, polyethylene glycol 1000 monoacetyl ether, polyethylene glycol monostearate, polyethylene glycol 400 monostearate, polyoxyethylene glycol fatty alcohol ethers, polyoxyl 20 cetostearyl ether, polyoxyl 40 stearate, polysorbate 20, polysorbate 40, polysorbate 60, polysorbate 80, polysorbates, PPG-26 oleate, propylene glycol stearate, quaternium-15, simethicone, sodium laureth sulfate, sodium lauryl sulfate, sorbitan esters, sorbitan monolaurate, sorbitan monooleate, sorbitan monopalmitate, sorbitan monostearate, sorbitan palmitate, sorbitan sesquioleate, steareth-2, steareth-100, stearic acid, stearyl alcohol, triethanolamine and trolamine.

28.-29. (canceled)

30. The composition of claim 1, wherein the compound comprises about 0.0005% to about 0.5% by weight of the composition.

31. The composition of claim 1, wherein the composition is in the form of a gel, a cream or an ointment.

32.-33. (canceled)

34. A method of treating psoriasis in a patient in need thereof, comprising contacting one or more psoriatic skin lesion with a composition of claim 1.
